(19) United States
(12) Patent Application Publication
(10) Pub. No.: US 2025/0122322 A1
(43) Pub. Date: Apr. 17, 2025

(54) ZWITTERIONIC COMPOUND, AND BLOCKING AGENT DISSOCIATION CATALYST FOR BLOCKED ISOCYANATE, BLOCKED ISOCYANATE COMPOSITION CONTAINING BLOCKING AGENT DISSOCIATION CATALYST, THERMOSETTING RESIN COMPOSITION, CURED PRODUCT AND METHOD FOR MANUFACTURING SAME, AND CARBONATE COMPOUND

(71) Applicant: KOEI CHEMICAL COMPANY, LIMITED, Chiba (JP)

(72) Inventors: Kouya SUGAWARA, Chiba (JP); Motoyoshi MIYAGI, Chiba (JP); Mitsuki ONODA, Chiba (JP)

(73) Assignee: KOEI CHEMICAL COMPANY, LIMITED, Chiba (JP)

(21) Appl. No.: 18/291,355

(22) PCT Filed: Jul. 29, 2022
(86) PCT No.: PCT/JP2022/029390

§ 371 Date: Jan. 23, 2024

(30) Foreign Application Priority Data
Jul. 30, 2021 (JP) 2021-126319

(51) Int. Cl. C08G 18/18; C07C 273/02; C07C 275/14; C07C 275/26; C08G 18/42; C08G 18/80

(52) U.S. Cl. CPC C08G 18/1875; C07C 273/02; C07C 275/14; C07C 275/26; C08G 18/42; C08G 18/8087

(57) ABSTRACT

This invention provides a blocking agent dissociation catalyst for blocked isocyanates comprising a zwitterionic compound represented by the following formula (1):

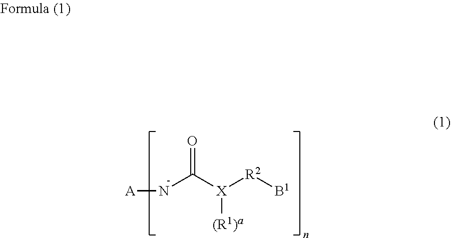

wherein A, R¹, R², X, B¹, and a are as defined in the specification.

### TECHNICAL FIELD

The present invention relates to a zwitterionic compound, a blocking agent dissociation catalyst for blocked isocyanates, a blocked isocyanate composition comprising the blocking agent dissociation catalyst, a thermosetting resin composition, a cured product and a method for producing the same, and a carbonate compound.

### BACKGROUND ART

Blocked isocyanates are compounds obtained by reaction of an isocyanate with a blocking agent containing active hydrogen groups that are capable of reacting with isocyanate groups. Blocked isocyanates are inactive at ordinary temperatures with highly reactive isocyanate groups being blocked by a blocking agent, and heating causes dissociation of the blocking agent to regenerate the isocyanate groups. Blocked isocyanates having such properties are more excellent in storage stability and easier to handle than isocyanates. Taking advantage of this feature, for example, they are widely used as raw materials for one-component polyurethane resins, which are obtained by curing polyol and isocyanate components, in applications such as paints and adhesives.

As described above, blocked isocyanates require heating to dissociate blocking agents; however, in recent years, there has been a demand for a lower dissociation temperature of blocking agents to reduce energy consumption and costs. Therefore, attempts have been made to use catalysts to lower the dissociation temperature of blocking agents of blocked isocyanates. Metal organic acid salts are known as such blocking agent dissociation catalysts. As a metal organic acid salt, an organotin catalyst, such as dibutyltin dilaurate, is often used (NPL 1). However, when the inventors used dibutyltin dilaurate as a blocking agent dissociation catalyst to measure the curing time of a thermosetting resin composition containing a polyol and a block isocyanate at 120° C., the composition was not cured even after 90 minutes, and its catalytic activity was not satisfactory (see Comparative Example 1). Furthermore, organotin catalysts are highly toxic and pose a problem in terms of toxicity in environment and human body. There has already been a movement mainly in Europe to regulate the use of organotin catalysts in the production of polyurethane resin, and there has been a demand for a catalyst that can replace the organotin catalysts.

### CITATION LIST

### Non-Patent Literature

NPL 1: “Latest Application Technology of Liquid Polyurethane” published by Chunichisha Co., Ltd., 1989, pp. 262-265

### SUMMARY OF INVENTION

### Technical Problem

The present invention was made in light of the above background art. An object of the present invention is to provide a catalyst that achieves excellent low-temperature dissociation of a blocking agent of a blocked isocyanate. Another object is to provide a blocked isocyanate composition comprising the blocking agent dissociation catalyst, a thermosetting resin composition comprising the blocked isocyanate composition and having excellent low-temperature curing properties, a cured product, and a method for producing the cured product.

### Solution to Problem

The present inventors conducted extensive research to solve the above problem, and found that excellent low-temperature dissociation was achieved when using a zwitterionic compound represented by formula (1) as a blocking agent dissociation catalyst for blocked isocyanates. The present invention has thus been completed.

The present invention provides the following zwitterionic compound, blocking agent dissociation catalyst for blocked isocyanates, blocked isocyanate composition comprising the blocking agent dissociation catalyst, thermosetting resin composition, and cured product and method for producing the same.

[1]

A blocking agent dissociation catalyst for blocked isocyanates comprising a zwitterionic compound represented by the following formula (1):

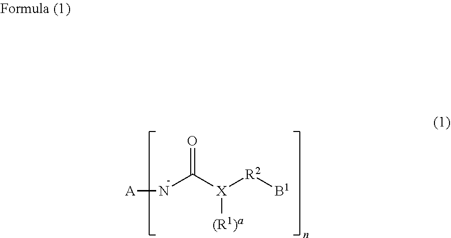

wherein
R¹ represents a hydrogen atom or a substituted or unsubstituted hydrocarbon group,
X represents a nitrogen atom or an oxygen atom,
a represents 0 or 1, wherein a represents 1 when X represents a nitrogen atom, and a represents 0 when X represents an oxygen atom,
R² represents a substituted or unsubstituted divalent hydrocarbon group,
B¹ represents a cationic group represented by formula (2) or formula (3),
n represents an integer of 1 or more, and
A represents an n-valent substituted or unsubstituted hydrocarbon group;

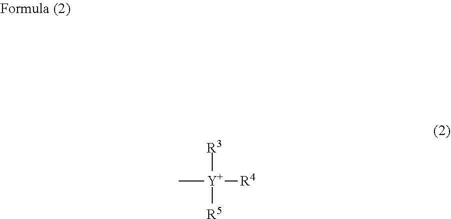

wherein
Y⁺ represents a nitrogen cation or a phosphorus cation, and
R³, R⁴, and R⁵ are the same or different, and each represents a substituted or unsubstituted hydrocarbon group, wherein some or all of R³, R⁴, and R⁵ may be bonded together to form one or more ring structures;

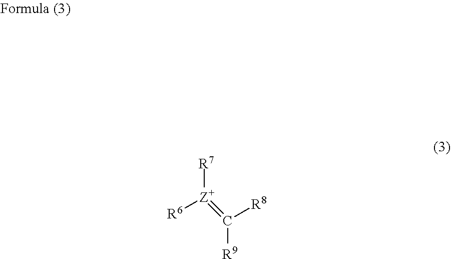

wherein
R⁸ represents a bonding hand with R², a hydrogen atom, a substituted or unsubstituted hydrocarbon group, or a substituted or unsubstituted hydrocarbon group with a bonding hand with R², R⁶, R⁷, and R⁹ are the same or different, and each represents a bonding hand with R², a substituted or unsubstituted hydrocarbon group, or a substituted or unsubstituted hydrocarbon group with a bonding hand with R², wherein any one of R⁶, R⁷, R⁸, and R⁹ represents a bonding hand with R² or a substituted or unsubstituted hydrocarbon group with a bonding hand with R², wherein some or all of R⁶, R⁷, R⁸, and R⁹ may be bonded together to form one or more ring structures, and wherein when some or all of R⁶, R⁷, R⁸, and R⁹ are bonded together to form one or more ring structures, and when a group or groups that do not form the ring structures from among R⁶, R⁷, R⁸, and R⁹ do not represent a bonding hand with R² or a substituted or unsubstituted hydrocarbon group with a bonding hand with R², then one of the ring structures has a bonding hand for binding to R², and
Z⁺ represents a nitrogen cation or a phosphorus cation.
   [2]

The blocking agent dissociation catalyst for blocked isocyanates according to [1],
wherein
Y⁺ represents a nitrogen cation, or
the cationic group represented by formula (3) is a group represented by formula (3-1), formula (3-2), formula (3-1a), or formula (3-2a);

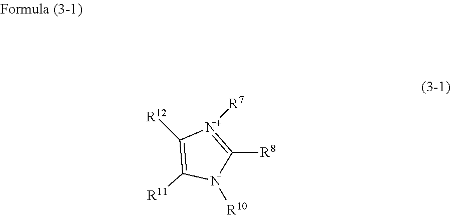

wherein
R⁷ represents a bonding hand with R², a substituted or unsubstituted hydrocarbon group, or a substituted or unsubstituted hydrocarbon group with a bonding hand with R³, and R⁸, R¹⁰, R¹¹, and R¹² are the same or different, and each represents a bonding hand with R², a hydrogen atom, a substituted or unsubstituted hydrocarbon group, or a substituted or unsubstituted hydrocarbon group with a bonding hand with R², wherein any one of R⁷, R⁸, R¹⁰, R¹¹, and R¹² represents a bonding hand for binding to R² or a substituted or unsubstituted hydrocarbon group with a bonding hand with R², and wherein when R⁷, R⁸, R¹⁰, R¹¹, and R¹² each represent a substituted or unsubstituted hydrocarbon group, some or all of R⁷, R⁸, R¹⁰, R¹¹, and R¹² may be bonded together to form one or more ring structures;

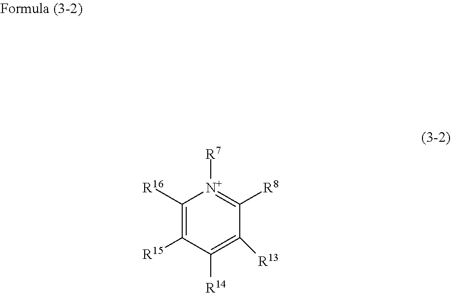

wherein
R⁷ represents a bonding hand with R², a substituted or unsubstituted hydrocarbon group, or a substituted or unsubstituted hydrocarbon group with a bonding hand with R², and R⁸, R¹³, R¹⁴, R¹⁵, and R¹⁶ are the same or different, and each represents a bonding hand with R², a hydrogen atom, a substituted or unsubstituted hydrocarbon group, or a substituted or unsubstituted hydrocarbon group with a bonding hand with R², wherein any one of R⁷, R⁸, R¹³, R¹⁴, R¹⁵, and R¹⁶ represents a bonding hand for binding to R² or a substituted or unsubstituted hydrocarbon group with a bonding hand with R², and wherein when R⁷, R⁸, R¹³, R¹⁴, R¹⁵, and R¹⁶ each represent a substituted or unsubstituted hydrocarbon group, some or all of R⁷, R⁸, R¹³, R¹⁴, R¹⁵, and R¹⁶ may be bonded together to form one or more ring structures;

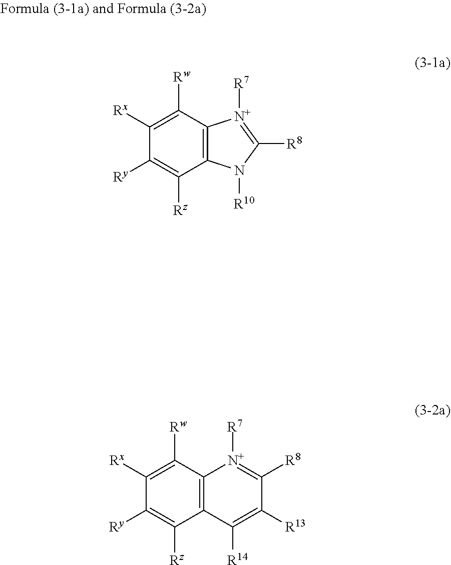

wherein
R⁷, R⁸, R¹⁰, R¹³, and R¹⁴ are as defined above, and
R^{w}, R^{x}, R^{y}, and R^{z} each represent a bonding hand with R², a hydrogen atom, or a C₁-C₂₀ hydrocarbon group.
   [3]

The blocking agent dissociation catalyst for blocked isocyanates according to [1] or [2], wherein A represents an n-valent substituted or unsubstituted aliphatic hydrocarbon group, an n-valent substituted or unsubstituted alicyclic hydrocarbon group, an n-valent substituted or unsubstituted aromatic hydrocarbon group, or an n-valent substituted or unsubstituted aromatic aliphatic hydrocarbon group.

[4]

The blocking agent dissociation catalyst for blocked isocyanates according to any one of [1] to [3], wherein n represents 1 to 20.

[5]

The blocking agent dissociation catalyst for blocked isocyanates according to any one of [1] to [4], wherein X represents a nitrogen atom.

[6]

A blocked isocyanate composition comprising the blocking agent dissociation catalyst of any one of [1] to [5] and a blocked isocyanate compound.

[7]

The blocked isocyanate composition according to [6], wherein the blocked isocyanate compound is a blocked isocyanate compound blocked with at least one blocking agent selected from the group consisting of alcohol compounds, phenol compounds, amine compounds, lactam compounds, oxime compounds, keto-enol compounds, active methylene compounds, pyrazole compounds, triazole compounds, imide compounds, mercaptan compounds, imine compounds, urea compounds, and diaryl compounds.

[8]

The blocked isocyanate composition according to [6] or [7], wherein the blocked isocyanate compound is a blocked isocyanate compound blocked with a fluorinated alcohol compound.

[9]

A thermosetting resin composition comprising the blocked isocyanate composition of any one of [6] to [8] and a compound having an isocyanate-reactive group.

[10]

The thermosetting resin composition according to [9], wherein the compound having an isocyanate-reactive group is a polyol compound.

[11]

A cured product obtained by curing the thermosetting resin composition of [9] or [10].

[12]

A method for producing a cured product, comprising curing the thermosetting resin composition of [9] or [10] by heating.

[13]

A zwitterionic compound represented by the following formula (1):

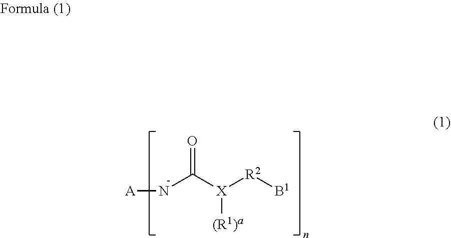

wherein
R¹ represents a hydrogen atom or a substituted or unsubstituted hydrocarbon group,
X represents a nitrogen atom or an oxygen atom,
a represents 0 or 1, wherein a represents 1 when X represents a nitrogen atom, and a represents 0 when X represents an oxygen atom,
R² represents a substituted or unsubstituted divalent hydrocarbon group,
B¹ represents a cationic group represented by formula (2) or formula (3),
n represents an integer of 1 or more, and

A represents an n-valent substituted or unsubstituted hydrocarbon group;

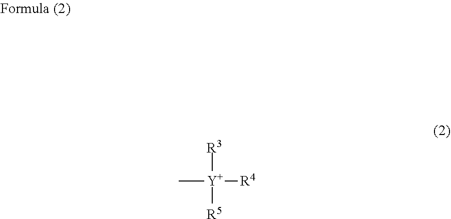

wherein
Y⁺ represents a nitrogen cation or a phosphorus cation, and
R³, R⁴, and R⁵ are the same or different, and each represents a substituted or unsubstituted hydrocarbon group, wherein some or all of R³, R⁴, and R⁵ may be bonded together to form one or more ring structures;

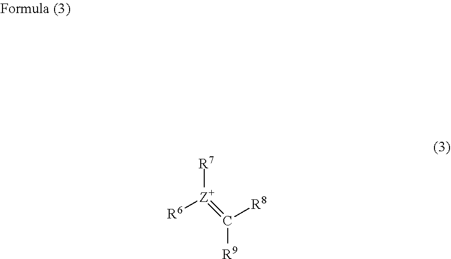

wherein
R⁸ represents a bonding hand with R², a hydrogen atom, a substituted or unsubstituted hydrocarbon group, or a substituted or unsubstituted hydrocarbon group with a bonding hand with R²,
R⁶, R⁷, and R⁹ are the same or different, and each represents a bonding hand with R², a substituted or unsubstituted hydrocarbon group, or a substituted or unsubstituted hydrocarbon group with a bonding hand with R², wherein any one of R⁶, R⁷, R⁸, and R⁹ represents a bonding hand with R² or a substituted or unsubstituted hydrocarbon group with a bonding hand with R², wherein some or all of R⁶, R⁷, R⁸, and R⁹ may be bonded together to form one or more ring structures, and wherein when some or all of R⁶, R⁷, R⁸, and R⁹ are bonded together to form one or more ring structures, and when a group or groups that do not form the ring structures from among R⁶, R⁷, R⁸, and R⁹ do not represent a bonding hand with R² or a substituted or unsubstituted hydrocarbon group with a bonding hand with R², then one of the ring structures has a bonding hand for binding to R², and
Z⁺ represents a nitrogen cation or a phosphorus cation.
   [14]

The zwitterionic compound according to [13],
wherein
Y⁺ represents a nitrogen cation, or
the cationic group represented by formula (3) is a group represented by formula (3-1), formula (3-2), formula (3-1a), or formula (3-2a):

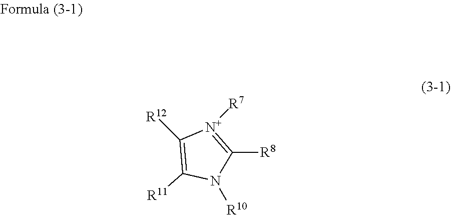

wherein
R⁷ represents a bonding hand with R², a substituted or unsubstituted hydrocarbon group, or a substituted or unsubstituted hydrocarbon group with a bonding hand with R², and R⁸, R¹⁰, R¹¹, and R¹² are the same or different, and each represents a bonding hand with R², a hydrogen atom, a substituted or unsubstituted hydrocarbon group, or a substituted or unsubstituted hydrocarbon group with a bonding hand with R², wherein any one of R⁷, R⁸, R¹⁰, R¹¹, and R¹² represents a bonding hand for binding to R² or a substituted or unsubstituted hydrocarbon group with a bonding hand with R², and wherein when R⁷, R⁸, R¹⁰, R¹¹, and R¹² each represent a substituted or unsubstituted hydrocarbon group, some or all of R⁷, R⁸, R¹⁰, R¹¹, and R¹² may be bonded together to form one or more ring structures;

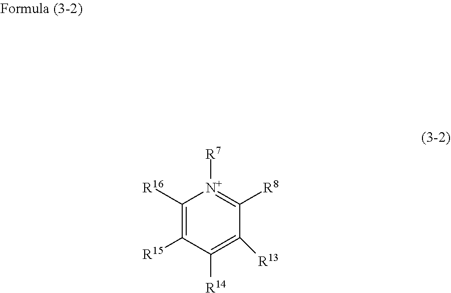

wherein
R⁷ represents a bonding hand with R², a substituted or unsubstituted hydrocarbon group, or a substituted or unsubstituted hydrocarbon group with a bonding hand with R², and R⁸, R¹³, R¹⁴, R¹⁵, and R¹⁶ are the same or different, and each represents a bonding hand with R², a hydrogen atom, a substituted or unsubstituted hydrocarbon group, or a substituted or unsubstituted hydrocarbon group with a bonding hand with R², wherein any one of R⁷, R⁸, R¹³, R¹⁴, R¹⁵, and R¹⁶ represents a bonding hand for binding to R² or a substituted or unsubstituted hydrocarbon group with a bonding hand with R², and wherein when R⁷, R⁸, R¹³, R¹⁴, R¹⁵, and R¹⁶ each represent a substituted or unsubstituted hydrocarbon group, some or all of R⁷, R⁸, R¹³, R¹⁴, R¹⁵, and R¹⁶ may be bonded together to form one or more ring structures;

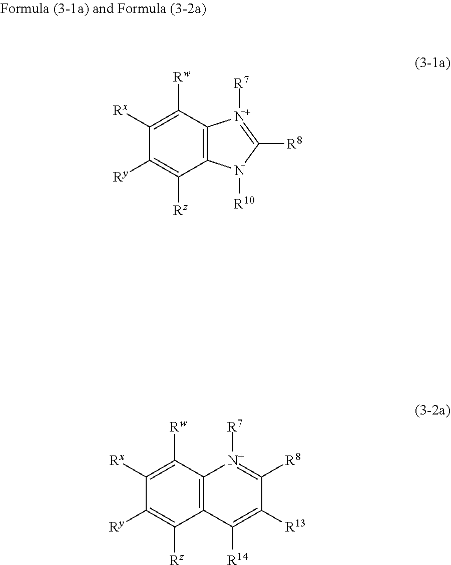

wherein
R⁷, R⁸, R¹⁰, R¹³, and R¹⁴ are as defined above, and
R^{w}, R^{x}, R^{y}, and R^{z} each represent a bonding hand with R², a hydrogen atom, or a C₁-C₂₀ hydrocarbon group.
   [15]

The zwitterionic compound according to [13] or [14], wherein A represents an n-valent substituted or unsubstituted aliphatic hydrocarbon group, an n-valent substituted or unsubstituted alicyclic hydrocarbon group, an n-valent substituted or unsubstituted aromatic hydrocarbon group, or an n-valent substituted or unsubstituted aromatic aliphatic hydrocarbon group.

[16]

The zwitterionic compound according to any one of [13] to [15], where n represents 1 to 20.

[17]

The zwitterionic compound according to any one of [13] to [16], wherein X represents a nitrogen atom.

[18]

The zwitterionic compound according to any one of [13] to [17], wherein R² represents a substituted or unsubstituted divalent alkylene group.

[19]

A method for producing a zwitterionic compound represented by formula (1), comprising stirring a compound represented by formula (8) in a solvent:

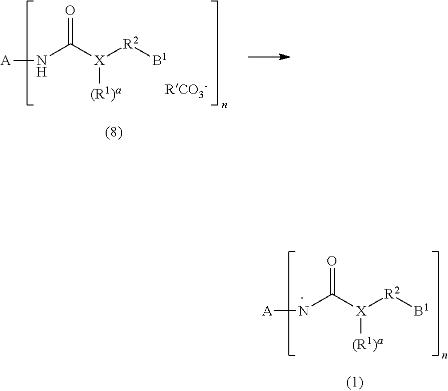

wherein
R′ represents a hydrogen atom or a substituted or unsubstituted hydrocarbon group,
R¹ represents a hydrogen atom or a substituted or unsubstituted hydrocarbon group,
X represents a nitrogen atom or an oxygen atom,
a represents 0 or 1, wherein a represents 1 when X represents a nitrogen atom, and a represents 0 when X represents an oxygen atom,
R² represents a substituted or unsubstituted divalent hydrocarbon group,
B¹ represents a cationic group represented by formula (2) or formula (3),
n represents an integer of 1 or more, and
A represents an n-valent substituted or unsubstituted hydrocarbon group;

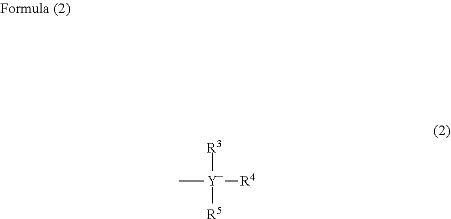

wherein
Y⁺ represents a nitrogen cation or a phosphorus cation, and
R³, R⁴, and R⁵ are the same or different, and each represents a substituted or unsubstituted hydrocarbon group, wherein some or all of R³, R⁴, and R⁵ may be bonded together to form one or more ring structures;

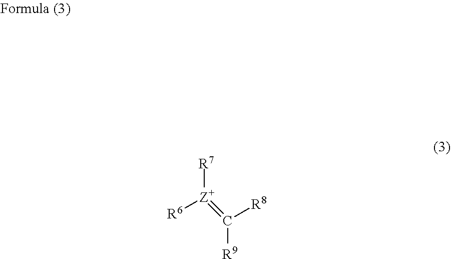

wherein
R⁸ represents a bonding hand with R², a hydrogen atom, a substituted or unsubstituted hydrocarbon group, or a substituted or unsubstituted hydrocarbon group with a bonding hand with R², R⁶, R⁷, and R⁹ are the same or different, and each represents a bonding hand with R², a substituted or unsubstituted hydrocarbon group, or a substituted or unsubstituted hydrocarbon group with a bonding hand with R², wherein any one of R⁶, R⁷, R⁸, and R⁹ represents a bonding hand with R² or a substituted or unsubstituted hydrocarbon group with a bonding hand with R², wherein some or all of R⁶, R⁷, R⁸, and R⁹ may be bonded together to form one or more ring structures, and wherein when some or all of R⁶, R⁷, R⁸, and R⁹ are bonded together to form one or more ring structures, and when a group or groups that do not form the ring structures from among R⁶, R⁷, R⁸, and R⁹ do not represent a bonding hand with R² or a substituted or unsubstituted hydrocarbon group with a bonding hand with R², then one of the ring structures has a bonding hand for binding to R², and
Z⁺ represents a nitrogen cation or a phosphorus cation.
   [20]

The method according to [19], further comprising reacting a compound represented by formula (6) with an ester carbonate compound represented by formula (7a) or formula (7b) to produce the compound represented by formula (8):

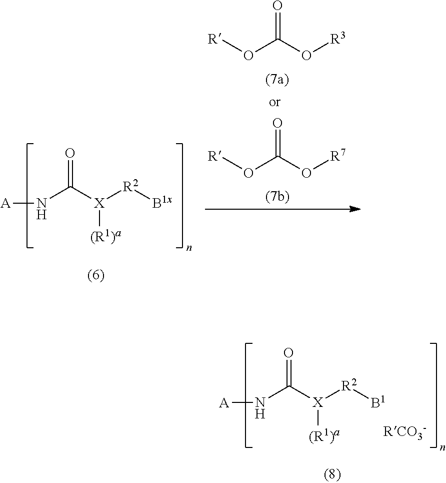

wherein
B^{1x} represents a group represented by the following formula (10) or formula (11), wherein when B^{1x} represents a group represented by formula (10), an ester carbonate compound represented by formula (7a) is used, and when Bix represents a group represented by the following formula (11), an ester carbonate compound represented by formula (7b) is used,
R′ represents a hydrogen atom, a substituted or unsubstituted hydrocarbon group, R³, or R⁷,
R³ represents a substituted or unsubstituted hydrocarbon group,
R⁷ represents a substituted or unsubstituted hydrocarbon group, and
R¹, R², R′, B¹, A, a, X, and n are as defined above;

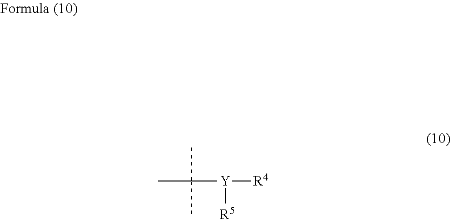

wherein
Y represents a nitrogen atom or a phosphorus atom, and
R⁴ and R⁵ are as defined above;

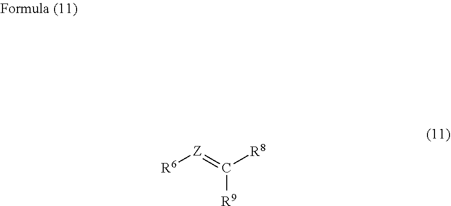

wherein
Z represents a nitrogen atom or a phosphorus atom, and
R⁶, R⁸, and R⁹ are as defined above.
   [21]

The method according to [19], further comprising reacting a compound represented by the following formula (9) with an isocyanate compound represented by formula (5) to obtain the compound represented by formula (8):

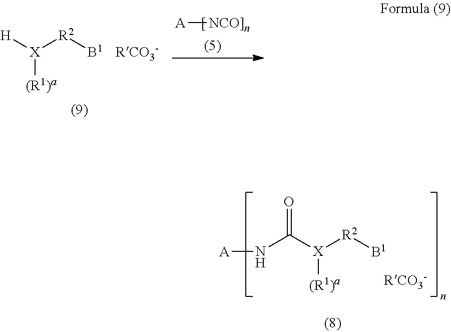

wherein A, R′, a, n, X, B¹, R¹, and R² are as defined above.
   [22]

A compound represented by the following formula (8):

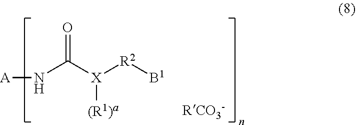

wherein
R′ represents a hydrogen atom or a substituted or unsubstituted hydrocarbon group,
R¹ represents a hydrogen atom or a substituted or unsubstituted hydrocarbon group,
X represents a nitrogen atom or an oxygen atom,
a represents 0 or 1, wherein a represents 1 when X represents a nitrogen atom, and a represents 0 when X represents an oxygen atom,
R² represents a substituted or unsubstituted divalent hydrocarbon group,
B¹ represents a cationic group represented by formula (2) or formula (3),
n represents an integer of 1 or more, and

A represents an n-valent substituted or unsubstituted hydrocarbon group;

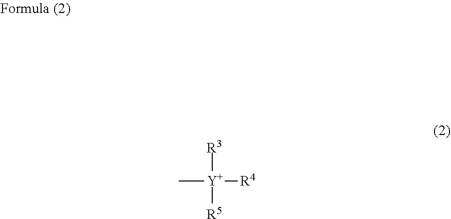

wherein
Y⁺ represents a nitrogen cation or a phosphorus cation, and
R³, R⁴, and R⁵ are the same or different, and each represents a substituted or unsubstituted hydrocarbon group, wherein some or all of R³, R⁴, and R⁵ may be bonded together to form one or more ring structures;

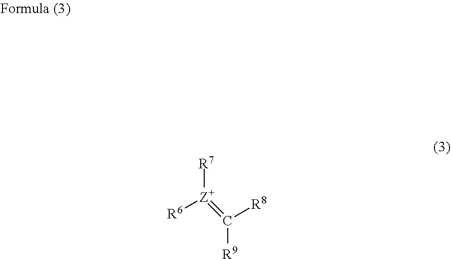

wherein
R⁸ represents a bonding hand with R², a hydrogen atom, a substituted or unsubstituted hydrocarbon group, or a substituted or unsubstituted hydrocarbon group with a bonding hand with R², R⁶, R⁷, and R⁹ are the same or different, and each represents a bonding hand with R², a substituted or unsubstituted hydrocarbon group, or a substituted or unsubstituted hydrocarbon group with a bonding hand with R², wherein any one of R⁶, R⁷, R⁶, and R⁹ represents a bonding hand with R² or a substituted or unsubstituted hydrocarbon group with a bonding hand with R², wherein some or all of R⁶, R⁷, R⁸, and R⁹ may be bonded together to form one or more ring structures, and wherein when some or all of R⁶, R⁷, R⁸, and R⁹ are bonded together to form one or more ring structures, and when a group or groups that do not form the ring structures from among R⁶, R⁷, R⁸, and R⁹ do not represent a bonding hand with R² or a substituted or unsubstituted hydrocarbon group with a bonding hand with R², then one of the ring structures has a bonding hand for binding to R², and
Z⁺ represents a nitrogen cation or a phosphorus cation.

### Advantageous Effects of Invention

Provided is a catalyst that achieves excellent low-temperature dissociation of a blocking agent of a blocked isocyanate. Further provided are a blocked isocyanate composition comprising the blocking agent dissociation catalyst, a thermosetting resin composition comprising the blocked isocyanate composition and having excellent low-temperature curing properties, and a cured product and a method for producing the same.

### BRIEF DESCRIPTION OF DRAWINGS

FIG. 1 shows the results of ¹H-NMR analysis in Production Example A-3 of the present application.

FIG. 2 shows the results of IR analysis in Production Example A-3 of the present application.

FIG. 3 shows the results of ¹H-NMR analysis in Production Example A-4 of the present application.

FIG. 4 shows the results of IR analysis in Production Example A-4 of the present application.

### DESCRIPTION OF EMBODIMENTS

### Blocking Agent Dissociation Catalyst for Blocked Isocyanates

As a blocking agent dissociation catalyst for blocked isocyanates of the present invention, a zwitterionic compound represented by formula (1) (hereinafter referred to as “the zwitterionic compound (1)”) can be used.

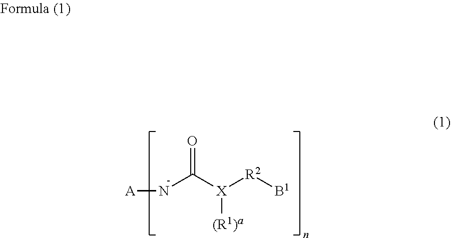

(In the formula,
R¹ represents a hydrogen atom or a substituted or unsubstituted hydrocarbon group,
X represents a nitrogen atom or an oxygen atom,
a represents 0 or 1, wherein a represents 1 when X represents a nitrogen atom, and a represents 0 when X represents an oxygen atom,
R² represents a substituted or unsubstituted divalent hydrocarbon group,
B¹ represents a cationic group represented by formula (2) or formula (3),
n represents an integer of 1 or more, and

A represents an n-valent substituted or unsubstituted hydrocarbon group);

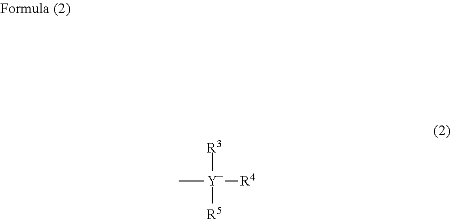

(In the formula,
Y⁺ represents a nitrogen cation or a phosphorus cation, and
R³, R⁴, and R⁵ are the same or different, and each represents a substituted or unsubstituted hydrocarbon group, wherein some or all of R³, R⁴, and R⁵ may be bonded together to form one or more ring structures);

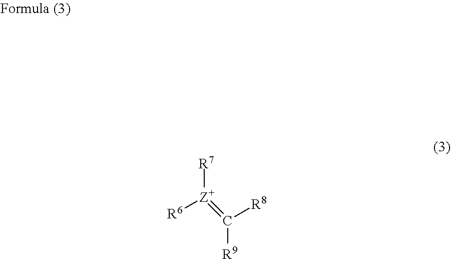

(In the formula,
R⁸ represents a bonding hand with R², a hydrogen atom, a substituted or unsubstituted hydrocarbon group, or a substituted or unsubstituted hydrocarbon group with a bonding hand with R², R⁶, R⁷, and R⁹ are the same or different, and each represents a bonding hand with R², a substituted or unsubstituted hydrocarbon group, or a substituted or unsubstituted hydrocarbon group with a bonding hand with R², wherein any one of R⁶, R⁷, R⁸, and R⁹ represents a bonding hand with R² or a substituted or unsubstituted hydrocarbon group with a bonding hand with R², wherein some or all of R⁶, R⁷, R⁸, and R⁹ may be bonded together to form one or more ring structures, and wherein when some or all of R⁶, R⁷, R⁸, and R⁹ are bonded together to form one or more ring structures, and when a group or groups that do not form the ring structures from among R⁶, R⁷, R⁸, and R⁹ do not represent a bonding hand with R² or a substituted or unsubstituted hydrocarbon group with a bonding hand with R², then one of the ring structures has a bonding hand for binding to R², and
Z⁺ represents a nitrogen cation or a phosphorus cation.)

In one embodiment, in formula (1), formula (2), and formula (3), when R¹, R³, R⁴, R⁵, R⁶, R⁷, R⁸, and R⁹ each represent a substituted or unsubstituted hydrocarbon group, it is preferably a substituted or unsubstituted C₁-C₁₀₀ hydrocarbon group, more preferably a substituted or unsubstituted C₁-C₅₀ hydrocarbon group, and particularly preferably a substituted or unsubstituted C₁-C₃₀ hydrocarbon group.

In another embodiment, in formula (1), formula (2), formula (3), formula (3-1), and formula (3-2), R¹, R³, R⁴, R⁵, R⁶, R⁷, R⁸, and R⁹ each represent a substituted or unsubstituted aliphatic hydrocarbon group, alicyclic hydrocarbon group, aromatic hydrocarbon group, or aromatic aliphatic hydrocarbon group,
preferably a substituted or unsubstituted C₁-C₁₀₀ aliphatic hydrocarbon group, alicyclic hydrocarbon group, aromatic hydrocarbon group, or aromatic aliphatic hydrocarbon group, more preferably a substituted or unsubstituted C₁-C₅₀ aliphatic hydrocarbon group, alicyclic hydrocarbon group, aromatic hydrocarbon group, or aromatic aliphatic hydrocarbon group, and particularly preferably a substituted or unsubstituted C₁-C₃₀ aliphatic hydrocarbon group, alicyclic hydrocarbon group, aromatic hydrocarbon group, or aromatic aliphatic hydrocarbon group.

The unsubstituted hydrocarbon group is, for example, a methyl, ethyl, propyl, isopropyl, butyl, sec-butyl, tert-butyl, pentyl, hexyl, heptyl, decyl, dodecyl, octadecyl, cyclopropyl, cyclopentyl, cyclohexyl, phenyl, naphthyl, benzyl, phenethyl, tolyl, or allyl group.

The unsubstituted aliphatic hydrocarbon group is, for example, a methyl, ethyl, propyl, isopropyl, butyl, sec-butyl, tert-butyl, pentyl, hexyl, heptyl, decyl, dodecyl, octadecyl, or allyl group.

The unsubstituted alicyclic hydrocarbon group is, for example, a cyclopropyl, cyclopentyl, or cyclohexyl group.

The unsubstituted aromatic hydrocarbon group is, for example, a phenyl, naphthyl, or tolyl group.

The unsubstituted aromatic aliphatic hydrocarbon group is, for example, a benzyl or phenethyl group.

In the present specification, examples of substituents include halogen atoms, such as fluorine, chlorine, bromine, and iodine; alkylamino groups, such as methylamino; dialkylamino groups, such as dimethylamino; alkoxy groups, such as methoxy and ethoxy; aryloxy groups, such as phenoxy and naphthyloxy; aralkyloxy groups, such as benzyloxy and naphthylmethoxy; halogenated alkyl groups, such as trifluoromethyl; a nitro group, a cyano group, a sulfonyl group, alkylcarbonylamino groups, alkyloxycarbonylamino groups, (alkylamino) carbonylamino groups, (dialkylamino) carbonylamino groups, and the like.

Each monovalent or divalent hydrocarbon group of R¹ to R¹⁶ may be substituted with at least one heteroatom, such as oxygen, nitrogen, or sulfur. When each hydrocarbon group of R¹ to R¹⁶ is substituted with at least one heteroatom, such as oxygen, nitrogen, or sulfur, the hydrocarbon group has at least one group, such as —O—, —N<, —NH—, —S—, or —SO₂—, and the hydrocarbon chain is interrupted by such a group.

Examples of the alkyl moiety of the above alkylamino groups, dialkylamino groups, alkoxy groups, halogenated alkyl groups, alkylcarbonylamino groups, alkyloxycarbonylamino groups, (alkylamino) carbonylamino groups, and (dialkylamino) carbonylamino groups include linear or branched C₁-C₁₂ alkyl groups, such as methyl, ethyl, propyl, isopropyl, n-butyl, isobutyl, sec-butyl, tert-butyl, pentyl, 1-ethylpentyl, heptyl, octyl, and 2-ethylhexyl. The number of carbon atoms in the alkyl group is preferably 1 to 8, and more preferably 1 or 2.

Examples of the aryl moiety of the above aryloxy groups include C₆-C₁₀ aryl groups. Specific examples of the aryl moiety include a phenyl group, a naphthyl group, and the like.

Examples of the aralkyl moiety of the above aralkyloxy groups include C₇-C₁₂ aralkyl groups. Specific examples of the aralkyl moiety include a benzyl group, a naphthylmethyl group, and the like.

In formula (1), R² represents a substituted or unsubstituted divalent hydrocarbon group, preferably a substituted or unsubstituted C₁-C₁₀₀ divalent hydrocarbon group, more preferably a substituted or unsubstituted C₁-C₅₀ divalent hydrocarbon group, and particularly preferably a substituted or unsubstituted C₁-C₃₀ divalent hydrocarbon group.

In another embodiment, R² represents a substituted or unsubstituted divalent aliphatic hydrocarbon group, alicyclic hydrocarbon group, aromatic hydrocarbon group, or aromatic aliphatic hydrocarbon group,
preferably a substituted or unsubstituted C₁-C₁₀₀ divalent aliphatic hydrocarbon group, alicyclic hydrocarbon group, aromatic hydrocarbon group, or aromatic aliphatic hydrocarbon group,
more preferably a substituted or unsubstituted C₁-C₅₀ divalent aliphatic hydrocarbon group, alicyclic hydrocarbon group, aromatic hydrocarbon group, or aromatic aliphatic hydrocarbon group, and
particularly preferably a substituted or unsubstituted C₁-C₃₀ divalent aliphatic hydrocarbon group, alicyclic hydrocarbon group, aromatic hydrocarbon group, or aromatic aliphatic hydrocarbon group.

In yet another embodiment, in formula (1), R² represents a hydrogen atom or a substituted or unsubstituted divalent alkylene group, preferably a substituted or unsubstituted C₁-C₁₀₀ divalent alkylene group, more preferably a substituted or unsubstituted C₁-C₅₀ divalent alkylene group, and particularly preferably a substituted or unsubstituted C₁-C₃₀ divalent alkylene group.

In formula (1), B¹ represents a cationic group represented by formula (2) or formula (3).

In formula (2), Y⁺ represents a nitrogen cation or a phosphorus cation, and preferably a nitrogen cation.

In formula (2), R³, R⁴, and R⁵ each represent a substituted or unsubstituted hydrocarbon group. The substituted or unsubstituted hydrocarbon group is preferably a substituted or unsubstituted C₁-C₁₀₀ hydrocarbon group, more preferably a substituted or unsubstituted C₁-C₅₀ hydrocarbon group, and particularly preferably a substituted or unsubstituted C₁-C₃₀ hydrocarbon group.

In another embodiment, in formula (2), each substituted or unsubstituted hydrocarbon group of R³, R⁴ and R⁵ is a substituted or unsubstituted aliphatic hydrocarbon group, alicyclic hydrocarbon group, aromatic hydrocarbon group, or aromatic aliphatic hydrocarbon group,
preferably a substituted or unsubstituted C₁-C₁₀₀ aliphatic hydrocarbon group, alicyclic hydrocarbon group, aromatic hydrocarbon group, or aromatic aliphatic hydrocarbon group,
more preferably a substituted or unsubstituted C₁-C₅₀ aliphatic hydrocarbon group, alicyclic hydrocarbon group, aromatic hydrocarbon group, or aromatic aliphatic hydrocarbon group, and
particularly preferably a substituted or unsubstituted C₁-C₃₀ aliphatic hydrocarbon group, alicyclic hydrocarbon group, aromatic hydrocarbon group, or aromatic aliphatic hydrocarbon group.

Examples of the cationic group represented by formula (2) include trimethylammonium, triethylammonium, tripropylammonium, tributylammonium, tripentylammonium, trihexylammonium, triheptylammonium, trioctylammonium, trinonylammonium, tri(decyl)ammonium, N-ethyl-N, N-dimethylammonium, N, N-dimethyl-N-propylammonium, N-butyl-N, N-dimethylammonium, N, N-diethyl-N decylammonium, N, N-diethyl-N-eicosylammonium, N, N-dibutyl-N-pentylammonium, N, N-dibutyl-N-hexylammonium, N, N-dibutyl-N-heptylammonium, N, N-dibutyl-N-octylammonium, N, N-dibutyl-N-nonylammonium, N, N-dibutyl-N-decylammonium, N, N-dibutyl-N-eicosylammonium, N-ethyl-N-methyl-N-propylammonium, N-butyl-N-ethyl-N-methylammonium, trimethylphosphonium, triethylphosphonium, tripropylphosphonium, tributylphosphonium, tripentylphosphonium, trihexylphosphonium, triheptylphosphonium, trioctylphosphonium, trinonylphosphonium, tri(decyl)phosphonium, tributyl octylphosphonium, di-n-butyl-n-hexylphosphonium, dibutyl-dodecylphosphonium, dihexyl tetradecylphosphonium, and the like. Preferred are trimethylammonium, triethylammonium, tripropylammonium, tributylammonium, tripentylammonium, and trihexylammonium, and particularly preferred is trimethylammonium.

In formula (3), Z⁺ represents a nitrogen cation or a phosphorus cation, and preferably a nitrogen cation.

In formula (3), R⁸ represents a bonding hand with R², a hydrogen atom, a substituted or unsubstituted hydrocarbon group, or a substituted or unsubstituted hydrocarbon group with a bonding hand with R², R⁶, R⁷, and R⁹ are the same or different, and each represents a bonding hand with R², a substituted or unsubstituted hydrocarbon group, or a substituted or unsubstituted hydrocarbon group with a bonding hand with R². The substituted or unsubstituted hydrocarbon group is preferably a substituted or unsubstituted C₁-C₁₀₀ hydrocarbon group, more preferably a substituted or unsubstituted C₁-C₅₀ hydrocarbon group, and particularly preferably a substituted or unsubstituted C₁-C₃₀ hydrocarbon group.

In another embodiment, in formula (3), when R⁶, R⁷, R⁸, and R⁹ each represent a substituted or unsubstituted hydrocarbon group, the substituted or unsubstituted hydrocarbon group is a substituted or unsubstituted aliphatic hydrocarbon group, alicyclic hydrocarbon group, aromatic hydrocarbon group, or aromatic aliphatic hydrocarbon group,
preferably a substituted or unsubstituted C₁-C₁₀₀ aliphatic hydrocarbon group, alicyclic hydrocarbon group, aromatic hydrocarbon group, or aromatic aliphatic hydrocarbon group,
more preferably a substituted or unsubstituted C₁-C₅₀ aliphatic hydrocarbon group, alicyclic hydrocarbon group, aromatic hydrocarbon group, or aromatic aliphatic hydrocarbon group, and
particularly preferably a substituted or unsubstituted C₁-C₃₀ aliphatic hydrocarbon group, alicyclic hydrocarbon group, aromatic hydrocarbon group, or aromatic aliphatic hydrocarbon group.

In yet another embodiment, in formula (3), when R⁸, R⁷, R⁸, and R⁹ each represent a substituted or unsubstituted hydrocarbon group with a bonding hand with R², it is preferably a substituted or unsubstituted C₁-C₁₀₀ hydrocarbon group with a bonding hand with R², more preferably a substituted or unsubstituted C₁-C₅₀ hydrocarbon group having a bonding hand with R², and particularly preferably a substituted or unsubstituted C₁-C₃₀ hydrocarbon group with a bonding hand with R².

In yet another embodiment, in formula (3), R⁶, R⁷, R⁸, and R⁹ each represent a substituted or unsubstituted aliphatic hydrocarbon group with a bonding hand with R², alicyclic hydrocarbon group with a bonding hand with R², aromatic hydrocarbon group with a bonding hand with R², or aromatic aliphatic hydrocarbon group with a bonding hand with R², preferably a substituted or unsubstituted C₁-C₁₀₀ aliphatic hydrocarbon group with a bonding hand with R², alicyclic hydrocarbon group with a bonding hand with R², aromatic hydrocarbon group with a bonding hand with R², or aromatic aliphatic hydrocarbon group with a bonding hand with R², more preferably a substituted or unsubstituted C₁-C₅₀, aliphatic hydrocarbon group with a bonding hand with R², alicyclic hydrocarbon group with a bonding hand with R², aromatic hydrocarbon group with a bonding hand with R², or aromatic aliphatic hydrocarbon group with a bonding hand with R², and particularly preferably a substituted or unsubstituted C₁-C₃₀ aliphatic hydrocarbon group with a bonding hand with R², alicyclic hydrocarbon group with a bonding hand with R², aromatic hydrocarbon group with a bonding hand with R², or aromatic aliphatic hydrocarbon group with a bonding hand with R².

In formula (3), some or all of R⁶, R⁷, R⁸, and R⁹ may be bonded together to form one or more ring structures. For example, when R⁶ and R⁹ are bonded together to form a ring structure, the structure can be represented by, for example, the following formula (3a), formula (3b), or formula (3c).

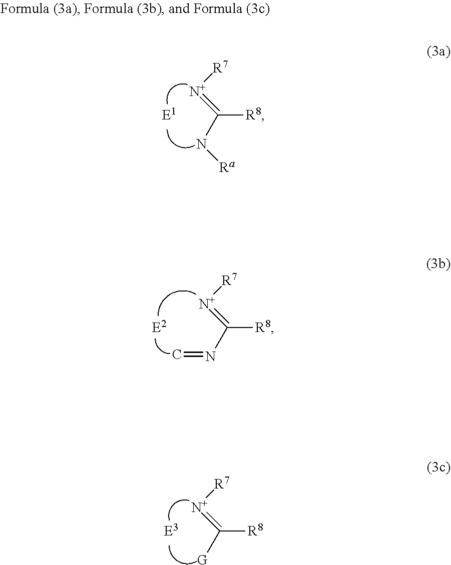

(In the formulas, R⁷ and R⁸ are as defined above, R^{a} represents a bonding hand with R², a hydrogen atom, a substituted or unsubstituted hydrocarbon group, or a substituted or unsubstituted hydrocarbon group with a bonding hand with R², and E¹, E², E³ each represent a substituted or unsubstituted divalent hydrocarbon group or a substituted or unsubstituted divalent hydrocarbon group with a bonding hand with R², and G represents an oxygen atom or a sulfur atom.)

When E¹, E², and E³ each represent a substituted divalent hydrocarbon group or a substituted divalent hydrocarbon group with a bonding hand with R², the substituent is preferably bonded to the carbon atom constituting the ring. The number of substituents bonded to E¹, E², or E³ is one, two, or three.

In E¹, E², and E³, when the divalent hydrocarbon group in the divalent hydrocarbon group or divalent hydrocarbon group with a bonding hand with R² is a substituted divalent hydrocarbon group, the substituted divalent hydrocarbon group may be substituted with at least one heteroatom, such as oxygen, nitrogen, or sulfur. When each hydrocarbon group of E¹, E², and E³ is substituted with at least one heteroatom, such as oxygen, nitrogen, or sulfur, the hydrocarbon group has at least one group, such as —O—, —N<, —NH—, or —S—, and the divalent hydrocarbon chain is interrupted by such a group.

In R^{a}, the substituted or unsubstituted hydrocarbon group is preferably a substituted or unsubstituted C₁-C₁₀₀ hydrocarbon group, more preferably a substituted or unsubstituted C₁-C₅₀ hydrocarbon group, and particularly preferably a substituted or unsubstituted C₁-C₃₀ hydrocarbon group.

In another embodiment, when R^{a} represents a substituted or unsubstituted hydrocarbon group, the substituted or unsubstituted hydrocarbon group is a substituted or unsubstituted aliphatic hydrocarbon group, alicyclic hydrocarbon group, aromatic hydrocarbon group, or aromatic aliphatic hydrocarbon group,
preferably a substituted or unsubstituted C₁-C₁₀₀ aliphatic hydrocarbon group, alicyclic hydrocarbon group, aromatic hydrocarbon group, or aromatic aliphatic hydrocarbon group,
more preferably a substituted or unsubstituted C₁-C₅₀ aliphatic hydrocarbon group, alicyclic hydrocarbon group, aromatic hydrocarbon group, or aromatic aliphatic hydrocarbon group, and
particularly preferably a substituted or unsubstituted C₁-C₃₀ aliphatic hydrocarbon group, alicyclic hydrocarbon group, aromatic hydrocarbon group, or aromatic aliphatic hydrocarbon group.

In yet another embodiment, when R^{a} represents a substituted or unsubstituted hydrocarbon group with a bonding hand with R², it is preferably a substituted or unsubstituted C₁-C₁₀₀ hydrocarbon group with a bonding hand with R², more preferably a substituted or unsubstituted C₁-C₅₀ hydrocarbon group with a bonding hand with R², and particularly preferably a substituted or unsubstituted C₁-C₃₀ hydrocarbon group with a bonding hand with R².

In yet another embodiment, R^{a} represents a substituted or unsubstituted aliphatic hydrocarbon group with a bonding hand with R², alicyclic hydrocarbon group with a bonding hand with R², aromatic hydrocarbon group with a bonding hand with R², or aromatic aliphatic hydrocarbon group with a bonding hand with R², preferably a substituted or unsubstituted C₁-C₁₀₀ aliphatic hydrocarbon group with a bonding hand with R², alicyclic hydrocarbon group with a bonding hand with R², aromatic hydrocarbon group with a bonding hand with R³, or aromatic aliphatic hydrocarbon group with a bonding hand with R², more preferably a substituted or unsubstituted C₁-C₅₀ aliphatic hydrocarbon group with a bonding hand with R², alicyclic hydrocarbon group with a bonding hand with R², aromatic hydrocarbon group with a bonding hand with R², or aromatic aliphatic hydrocarbon group with a bonding hand with R², and particularly preferably a substituted or unsubstituted C₁-C₃₀ aliphatic hydrocarbon group with a bonding hand with R², alicyclic hydrocarbon group with a bonding hand with R², aromatic hydrocarbon group with a bonding hand with R², or aromatic aliphatic hydrocarbon group with a bonding hand with R².

In E¹, E², and E³, the substituted or unsubstituted divalent hydrocarbon group is preferably a substituted or unsubstituted C₁-C₂₀ divalent hydrocarbon group, more preferably a substituted or unsubstituted C₁-C₁₂ divalent hydrocarbon group, and particularly preferably a substituted or unsubstituted C₁-C₆ divalent hydrocarbon group.

In another embodiment, when E¹, E³, and E³ each represent a substituted or unsubstituted divalent hydrocarbon group, the substituted or unsubstituted divalent hydrocarbon group is a substituted or unsubstituted divalent aliphatic hydrocarbon group, alicyclic hydrocarbon group, aromatic hydrocarbon group, or aromatic aliphatic hydrocarbon group,
preferably a substituted or unsubstituted C₁-C₂₀ divalent aliphatic hydrocarbon group, alicyclic hydrocarbon group, aromatic hydrocarbon group, or aromatic aliphatic hydrocarbon group,
more preferably a substituted or unsubstituted C₁-C₁₂ divalent aliphatic hydrocarbon group, alicyclic hydrocarbon group, aromatic hydrocarbon group, or aromatic aliphatic hydrocarbon group, and
particularly preferably a substituted or unsubstituted C₁-C₆ divalent aliphatic hydrocarbon group, alicyclic hydrocarbon group, aromatic hydrocarbon group, or aromatic aliphatic hydrocarbon group.

In yet another embodiment, when E¹, E², and E³ each represent a substituted or unsubstituted hydrocarbon group with a bonding hand with R², it is preferably a substituted or unsubstituted C₁-C₂₀ divalent hydrocarbon group with a bonding hand with R²,
more preferably a substituted or unsubstituted C₁-C₁₂ divalent hydrocarbon group with a bonding hand with R², and
particularly preferably a substituted or unsubstituted C₁-C₆ divalent hydrocarbon group with a bonding hand with R².

In yet another embodiment, E¹, E², and E³ each represent a substituted or unsubstituted divalent aliphatic hydrocarbon group with a bonding hand with R², alicyclic hydrocarbon group with a bonding hand with R², aromatic hydrocarbon group with a bonding hand with R², or aromatic aliphatic hydrocarbon group with a bonding hand with R²,
preferably a substituted or unsubstituted C₁-C₂₀ divalent aliphatic hydrocarbon group with a bonding hand with R², alicyclic hydrocarbon group with a bonding hand with R², aromatic hydrocarbon group with a bonding hand with R², or aromatic aliphatic hydrocarbon group with a bonding hand with R²,
more preferably a substituted or unsubstituted C₁-C₁₂ divalent aliphatic hydrocarbon group with a bonding hand with R², alicyclic hydrocarbon group with a bonding hand with R², aromatic hydrocarbon group with a bonding hand with R², or aromatic aliphatic hydrocarbon group with a bonding hand with R², and
particularly preferably a substituted or unsubstituted C₁-C₆ divalent aliphatic hydrocarbon group with a bonding hand with R², alicyclic hydrocarbon group with a bonding hand with R², aromatic hydrocarbon group with a bonding hand with R², or aromatic aliphatic hydrocarbon group with a bonding hand with R².

Although the above is an example of a case in which R⁶ and R⁹ are bonded together to form a ring structure, a ring structure can also be formed in a similar manner in cases in which R⁶ and R⁷, R⁷ and R⁸, and R⁸ and R⁹ are bonded together to form a ring structure.

The cationic group represented by formula (3) is preferably a cationic group represented by the following formula (3-1) or formula (3-2), and particularly preferably a cationic group represented by the following formula (3-1).

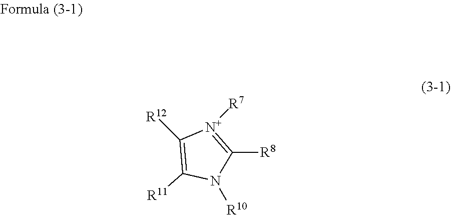

(R⁷ represents a bonding hand with R² or a substituted or unsubstituted hydrocarbon group, and
R⁸, R¹⁰, R¹¹, and R¹² are the same or different, and each represents a bonding hand with R², a hydrogen atom, a substituted or unsubstituted hydrocarbon group, or a substituted or unsubstituted hydrocarbon group with a bonding hand with R², wherein any one of R⁷, R⁸, R¹⁰, R¹¹, and R¹² represents a bonding hand for binding to R² or a substituted or unsubstituted hydrocarbon group with a bonding hand with R², and wherein when R⁷, R⁸, R¹⁰, R¹¹, and R¹² each represent a substituted or unsubstituted hydrocarbon group, some or all of R⁷, R⁸, R¹⁰, R¹¹, and R¹² may be bonded together to form one or more ring structures);

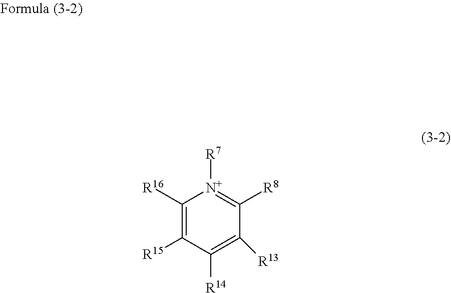

(R⁷ represents a bonding hand with R² or a substituted or unsubstituted hydrocarbon group, and
R⁸, R¹³, R¹⁴, R¹⁵, and R¹⁶ are the same or different, and each represents a bonding hand with R², a hydrogen atom, a substituted or unsubstituted hydrocarbon group, or a substituted or unsubstituted hydrocarbon group with a bonding hand with R², wherein any one of R⁷, R⁸, R¹³, R¹⁴, R¹⁵, and R¹⁶ represents a bonding hand for binding to R² or a substituted or unsubstituted hydrocarbon group with a bonding hand with R³, and wherein when R⁷, R⁸, R¹³, R¹⁴, R¹⁵, and R¹⁶ each represent a substituted or unsubstituted hydrocarbon group, some or all of R⁷, R⁸, R¹³, R¹⁴, R¹⁵, and R¹⁶ may be bonded together to form one or more ring structures.)

In formula (3-1), R⁷ represents a bonding hand with R² or a substituted or unsubstituted hydrocarbon group. R⁸, R¹⁰, R¹¹, and R¹² are the same or different, and each represents a bonding hand with R², a hydrogen atom, a substituted or unsubstituted hydrocarbon group, or a substituted or unsubstituted hydrocarbon group with a bonding hand with R². The substituted or unsubstituted hydrocarbon group is preferably a substituted or unsubstituted C₁-C₁₀₀ hydrocarbon group, more preferably a substituted or unsubstituted C₁-C₅₀ hydrocarbon group, and particularly preferably a substituted or unsubstituted C₁-C₃₀ hydrocarbon group.

In another embodiment, in formula (3-1), when R⁷, R⁸, R¹⁰, R¹¹, and R¹² each represent a substituted or unsubstituted hydrocarbon group, the substituted or unsubstituted hydrocarbon group is a substituted or unsubstituted aliphatic hydrocarbon group, alicyclic hydrocarbon group, aromatic hydrocarbon group, or aromatic aliphatic hydrocarbon group,
preferably a substituted or unsubstituted C₁-C₁₀₀ aliphatic hydrocarbon group, alicyclic hydrocarbon group, aromatic hydrocarbon group, or aromatic aliphatic hydrocarbon group, more preferably a substituted or unsubstituted C₁-C₅₀ aliphatic hydrocarbon group, alicyclic hydrocarbon group, aromatic hydrocarbon group, or aromatic aliphatic hydrocarbon group, and
particularly preferably a substituted or unsubstituted C₁-C₃₀ aliphatic hydrocarbon group, alicyclic hydrocarbon group, aromatic hydrocarbon group, or aromatic aliphatic hydrocarbon group.

In yet another embodiment, in formula (3-1), when R⁷, R⁸, R¹⁰, R¹¹, and R¹² each represent a substituted or unsubstituted hydrocarbon group with a bonding hand with R², it is preferably a substituted or unsubstituted C₁-C₁₀₀ hydrocarbon group with a bonding hand with R², more preferably a substituted or unsubstituted C₁-C₅₀ hydrocarbon group with a bonding hand with R², and particularly preferably a substituted or unsubstituted C₁-C₃₀ hydrocarbon group with a bonding hand with R².

In yet another embodiment, in formula (3-1), R⁷, R⁸, R¹⁰, R¹¹, and R¹² each represent a substituted or unsubstituted aliphatic hydrocarbon group with a bonding hand with R², alicyclic hydrocarbon group with a bonding hand with R², aromatic hydrocarbon group with a bonding hand with R², or aromatic aliphatic hydrocarbon group with a bonding hand with R², preferably a substituted or unsubstituted C₁-C₁₀₀ aliphatic hydrocarbon group with a bonding hand with R², alicyclic hydrocarbon group with a bonding hand with R², aromatic hydrocarbon group with a bonding hand with R², or aromatic aliphatic hydrocarbon group with a bonding hand with R², more preferably a substituted or unsubstituted C₁-C₅₀ aliphatic hydrocarbon group with a bonding hand with R², alicyclic hydrocarbon group with a bonding hand with R², aromatic hydrocarbon group with a bonding hand with R², or aromatic aliphatic hydrocarbon group with a bonding hand with R², and particularly preferably a substituted or unsubstituted C₁-C₃₀ aliphatic hydrocarbon group with a bonding hand with R², alicyclic hydrocarbon group with a bonding hand with R², aromatic hydrocarbon group with a bonding hand with R², or aromatic aliphatic hydrocarbon group with a bonding hand with R².

Specific examples of the cationic group represented by formula (3-1) include 1,3-dimethylimidazolium, 1-ethyl-3-methylimidazolium, 1-methyl-3 propylimidazolium, 1-butyl-3-methylimidazolium, 1-methyl-3-pentylimidazolium, 1-hexyl-3-methylimidazolium, 1-heptyl-3-methylimidazolium, 1-methyl-3-octylimidazolium, 1-methyl-3-nonylimidazolium, 1-decyl-3-methylimidazolium, 1-allyl-3-methylimidazolium, 1-allyl-3-ethylimidazolium, 1-(2-methoxyethyl)-3-methylimidazolium, 1-(2-ethoxyethyl)-3-methylimidazolium, 1-ethyl-3-(2-methoxyethyl)imidazolium, 1-ethyl-3-(2-ethoxyethyl)imidazolium, 1,3-di(tert-butyl)imidazolium, 1,3-bis(1,1-dimethylethyl)imidazolium, 1,3-bis(1,1-dimethylpropyl)imidazolium, 1,3-bis(1,1,3,3-tetramethylbutyl)imidazolium, 1,3-bis(1-methyl-1-phenylethyl)imidazolium, 1,3-bis(1,1-dimethyl-2-phenylethyl)imidazolium, 1,3-bis(1-adamantyl)imidazolium, and the like.

In formula (3-2), R⁷ represents a bonding hand with R⁴, a substituted or unsubstituted hydrocarbon group, or a substituted or unsubstituted hydrocarbon group with a bonding hand with R². R⁸, R¹³, R¹⁴, R¹⁵, and R¹⁶ are the same or different, and each represents a bonding hand with R², a hydrogen atom, a substituted or unsubstituted hydrocarbon group, or a substituted or unsubstituted hydrocarbon group with a bonding hand with R². The substituted or unsubstituted hydrocarbon group is preferably a substituted or unsubstituted C₁-C₁₀₀ hydrocarbon group, more preferably a substituted or unsubstituted C₁-C₅₀ hydrocarbon group, and particularly preferably a substituted or unsubstituted C₁-C₃₀ hydrocarbon group.

In another embodiment, in formula (3-2), when R⁷, R⁸, R¹³, R¹⁴, R¹⁵, and R¹⁶ each represent a substituted or unsubstituted hydrocarbon group, the substituted or unsubstituted hydrocarbon group is a substituted or unsubstituted aliphatic hydrocarbon group, alicyclic hydrocarbon group, aromatic hydrocarbon group, or aromatic aliphatic hydrocarbon group,
preferably a substituted or unsubstituted C₁-C₁₀₀ aliphatic hydrocarbon group, alicyclic hydrocarbon group, aromatic hydrocarbon group, or aromatic aliphatic hydrocarbon group,
more preferably a substituted or unsubstituted C₁-C₅₀ aliphatic hydrocarbon group, alicyclic hydrocarbon group, aromatic hydrocarbon group, or aromatic aliphatic hydrocarbon group, and
particularly preferably a substituted or unsubstituted C₁-C₃₀ aliphatic hydrocarbon group, alicyclic hydrocarbon group, aromatic hydrocarbon group, or aromatic aliphatic hydrocarbon group.

In yet another embodiment, in formula (3-2), when R⁷, R⁸, R¹³, R¹⁴, R¹⁵, and R¹⁶ each represent a substituted or unsubstituted hydrocarbon group with a bonding hand with R², it is preferably a substituted or unsubstituted C₁-C₁₀₀ hydrocarbon group with a bonding hand with R², more preferably a substituted or unsubstituted C₁-C₅₀ hydrocarbon group with a bonding hand with R², and particularly preferably a substituted or unsubstituted C₁-C₃₀ hydrocarbon group with a bonding hand with R².

In yet another embodiment, in formula (3-2), R⁷, R⁸, R¹³, R¹⁴, R¹⁵, and R¹⁶ each represent a substituted or unsubstituted aliphatic hydrocarbon group with a bonding hand with R², alicyclic hydrocarbon group with a bonding hand with R², aromatic hydrocarbon group with a bonding hand with R², or aromatic aliphatic hydrocarbon group with a bonding hand with R²,
preferably a substituted or unsubstituted C₁-C₁₀₀ aliphatic hydrocarbon group with a bonding hand with R², alicyclic hydrocarbon group with a bonding hand with R², aromatic hydrocarbon group with a m bonding hand with R⁸, or aromatic aliphatic hydrocarbon group with a bonding hand with R²,
more preferably a substituted or unsubstituted C₁-C₅₀ aliphatic hydrocarbon group with a bonding hand with R², alicyclic hydrocarbon group with a bonding hand with R², aromatic hydrocarbon group with a bonding hand with R², or aromatic aliphatic hydrocarbon group with a bonding hand with R², and
particularly preferably a substituted or unsubstituted C₁-C₃₀ aliphatic hydrocarbon group with a bonding hand with R², alicyclic hydrocarbon group with a bonding hand with R², aromatic hydrocarbon group with a bonding hand with R², or aromatic aliphatic hydrocarbon group with a bonding hand with R².

Specific examples of the cationic group represented by formula (3-2) include 1-methylpyridinium, 1-ethylpyridinium, 1-propylpyridinium, 1-butylpyridinium, 1-pentylpyridinium, 1-hexylpyridinium, 1-heptylpyridinium, 1-octylpyridinium, 1-nonylpyridinium, 1-decylpyridinium, 1-hexadecylpyridinium, 1-allylpyridinium, 1-(2-methoxyethyl)pyridinium, 1-(2-ethoxyethyl)pyridinium, and the like.

R¹, R³, R⁴, R⁵, R⁶, R⁷, R⁸, R⁹, R¹⁰, R¹¹, R¹², R¹³, R¹⁴, R¹⁵, and R¹⁶ may form a ring structure together with the carbon atom, nitrogen atom, or phosphorus atom to which they are bonded.

For example, when R¹¹ and R¹² or R¹⁵ and R¹⁶ form a ring structure together with the carbon atoms to which they are bonded, for example, a benzimidazolium ring structure or a quinolinium ring structure shown in the following formula (3-1a) or formula (3-2a) can be formed.

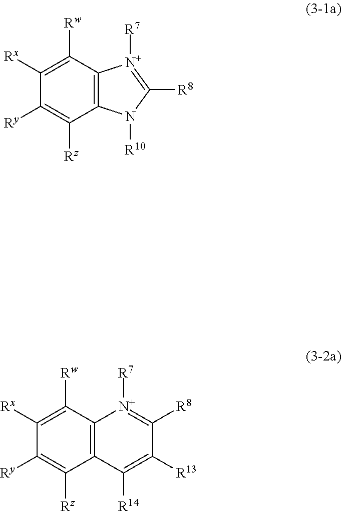

(In the formulas, R⁷, R⁸, R¹⁰, R¹³, and R¹⁴ are as defined above, and R^{w}, R^{x}, R^{y}, and R^{z} each represent a bonding hand with R², a hydrogen atom, or a C₁-C₂₀ hydrocarbon group.)

Although the above is an example of a case in which R¹¹ and R¹² or R¹⁵ and R¹⁶ form a ring structure, a ring structure can also be formed in a similar manner in cases of R¹ and R³, R¹ and R⁷, R¹ and R⁸, R³ and R⁴, R⁷ and R⁸, R⁸ and R⁹, R⁸ and R¹⁰, R¹⁰ and R¹¹, R¹² and R⁷, and R¹³ and R¹⁴.

In formula (1), A represents a substituted or unsubstituted n-valent hydrocarbon group, and preferably a substituted or unsubstituted n-valent C₁-C₃₀ aliphatic hydrocarbon group, a substituted or unsubstituted n-valent C₃-C₃₀ alicyclic hydrocarbon group, a substituted or unsubstituted n-valent C₆-C₂₀₀ aromatic hydrocarbon group, or a substituted or unsubstituted n-valent C₇-C₂₀₀ aromatic aliphatic hydrocarbon group.

The “n-valent hydrocarbon group” refers to a group obtained by removing n hydrogens from a hydrocarbon group. The “n-valent aliphatic hydrocarbon group” refers to a group obtained by removing n hydrogens from an aliphatic hydrocarbon group. The “n-valent alicyclic hydrocarbon group” refers to a group obtained by removing n hydrogens from an alicyclic hydrocarbon group. The “n-valent aromatic hydrocarbon group” refers to a group obtained by removing n hydrogens from an aromatic hydrocarbon group. The “n-valent aromatic aliphatic hydrocarbon group” refers to a group obtained by removing n hydrogens from an aromatic aliphatic hydrocarbon group.

In the present specification, the “substituted or unsubstituted hydrocarbon groups” include (i) a hydrocarbon group that may have a substituent, (ii) a hydrocarbon group that may be substituted with a heteroatom, and (iii) a hydrocarbon group having a substituent and substituted with a heteroatom.

Further, the “substituted or unsubstituted aliphatic hydrocarbon groups” include (iv) an aliphatic hydrocarbon group that may have a substituent, (v) an aliphatic hydrocarbon group that may be substituted with a heteroatom, and (vi) an aliphatic hydrocarbon group having a substituent and substituted with a heteroatom.

Further, the “substituted or unsubstituted alicyclic hydrocarbon groups” include (vii) an alicyclic hydrocarbon group that may have a substituent, (viii) an alicyclic hydrocarbon group that may be substituted with a heteroatom, and (ix) an alicyclic hydrocarbon group having a substituent and substituted with a heteroatom.

Further, the “substituted or unsubstituted aromatic hydrocarbon groups” include (x) an aromatic hydrocarbon group that may have a substituent, (xi) an aromatic hydrocarbon group that may be substituted with a heteroatom, and (xii) an aromatic hydrocarbon group having a substituent and substituted with a heteroatom.

Further, the “substituted or unsubstituted aromatic aliphatic hydrocarbon groups” include (xiii) an aromatic aliphatic hydrocarbon group that may have a substituent, (xiv) an aromatic aliphatic hydrocarbon group that may be substituted with a heteroatom, and (xv) an aromatic aliphatic hydrocarbon group having a substituent and substituted with a heteroatom.

In the present specification, the “substituted or unsubstituted n-valent hydrocarbon groups” include (i) an n-valent hydrocarbon group that may have a substituent, (ii) an n-valent hydrocarbon group that may be substituted with a heteroatom, and (iii) an n-valent hydrocarbon group having a substituent and substituted with a heteroatom.

Further, the “substituted or unsubstituted n-valent aliphatic hydrocarbon groups” include (iv) an n-valent aliphatic hydrocarbon group that may have a substituent, (v) an n-valent aliphatic hydrocarbon group that may be substituted with a heteroatom, and (vi) an n-valent aliphatic hydrocarbon group having a substituent and substituted with a heteroatom.

Further, the “substituted or unsubstituted n-valent alicyclic hydrocarbon groups” include (vii) an n-valent alicyclic hydrocarbon group that may have a substituent, (viii) an n-valent alicyclic hydrocarbon group that may be substituted with a heteroatom, and (ix) an n-valent alicyclic hydrocarbon group having a substituent and substituted with a heteroatom.

Further, the “substituted or unsubstituted n-valent aromatic hydrocarbon groups” include (x) an n-valent aromatic hydrocarbon group that may have a substituent, (xi) an n-valent aromatic hydrocarbon group that may be substituted with a heteroatom, and (xii) an n-valent aromatic hydrocarbon group having a substituent and substituted with a heteroatom.

Further, the “substituted or unsubstituted n-valent aromatic aliphatic hydrocarbon groups” include (xiii) an n-valent aromatic aliphatic hydrocarbon group that may have a substituent, (xiv) an n-valent aromatic aliphatic hydrocarbon group that may be substituted with a heteroatom, and (xv) an n-valent aromatic aliphatic hydrocarbon group having a substituent and substituted with a heteroatom.

In A, examples of the unsubstituted n-valent hydrocarbon group include groups obtained by removing n hydrogen atoms from methane, ethane, propane, isopropane, butane, sec-butane, tert-butane, pentane, hexane, heptane, decane, dodecane, octadecane, cyclopropane, cyclopentane, cyclohexane, benzene, naphthalene, toluene, phenylethane, and propylene. In terms of toluene and phenylethane, either one or more hydrogen atoms of the aromatic ring or one or more hydrogen atoms of the methyl group or ethyl group, or both, may have been removed. However, when n represents 2 or more, hydrogen atoms for removal are those bonding to different carbon atoms.

In another embodiment, in formula (1), formula (5), formula (6), and formula (8), A represents a hydrocarbon group, excluding the following isocyanate groups of isocyanate compounds of (i) to (v). In the present specification, isocyanates include monofunctional and polyfunctional isocyanates.
(i) Aliphatic isocyanates
(ii) Alicyclic isocyanates
(iii) Aromatic isocyanates
(iv) Aromatic aliphatic isocyanates
(v) Modified isocyanates formed from at least one member selected from the group consisting of aliphatic isocyanates, alicyclic isocyanates, aromatic isocyanates, and aromatic aliphatic isocyanates

In the present specification, specific examples of preferred groups represented by A are shown below.

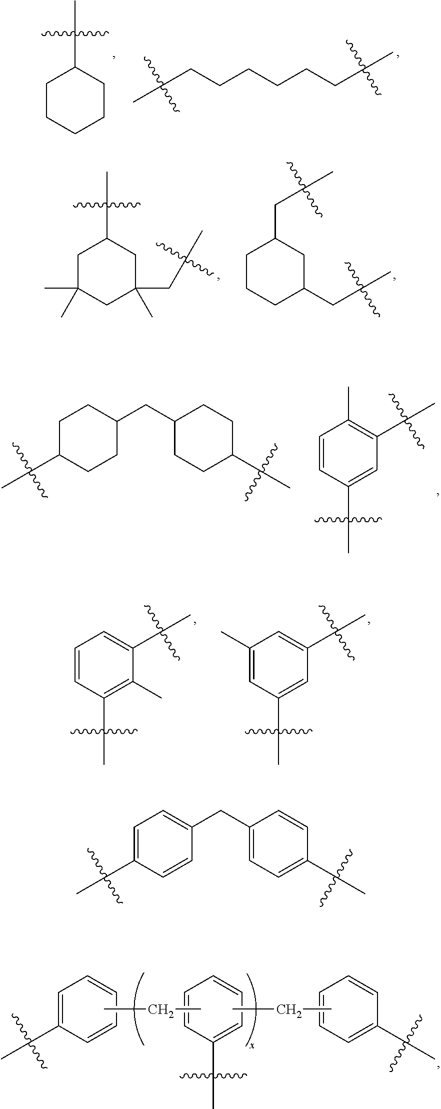

x represents an integer of 0 or more and 20 or less, and preferably an integer of 1 or more and 20 or less.

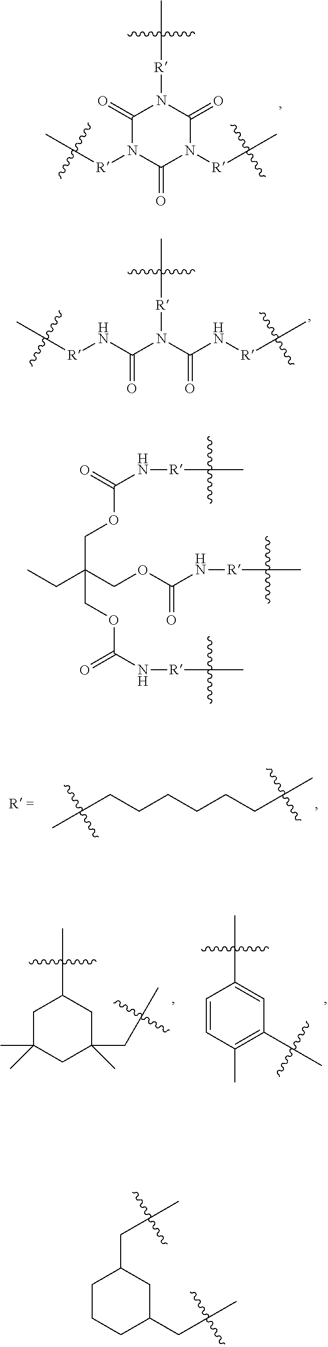

In formula (1), formula (2), and formula (3), in R¹, R², R³, R⁴, R⁵, R⁶, R⁷, R⁸, R⁹, and A, the number of substituents in the hydrocarbon group, aliphatic hydrocarbon group, alicyclic hydrocarbon group, aromatic hydrocarbon group, and aromatic aliphatic hydrocarbon group can be 1 to 5, preferably 1 to 3, and more preferably 1 or 2.

In formula (1), X represents a nitrogen atom or an oxygen atom, and preferably a nitrogen atom.

In formula (1), n represents an integer of 1 or more, preferably an integer of 1 to 20, more preferably 1 to 6, even more preferably 1 to 4, and particularly preferably 1 or 2.

Specific examples of the zwitterionic compound (1) are shown below. However, the present invention is not limited thereto. In the following specific examples, Et represents an ethyl group, Pr represents a propyl group, ⁱPr represents an isopropyl group, Bu represents a butyl group, and Bnz represents a benzyl group.

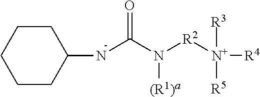

| X(R [symbol] ) [symbol] | R [symbol] | R [symbol] | R [symbol] | R [symbol] | |
|---|---|---|---|---|---|
| NH | CH [symbol] | CH [symbol] | CH [symbol] | CH [symbol] | (1-2-1a-1) |
| NH | CH [symbol] | CH [symbol] | CH [symbol] | Et | (1-2-2a-1) |
| NH | CH [symbol] | CH [symbol] | CH [symbol] | ⁱPr | (1-2-3a-1) |
| NH | CH [symbol] | CH [symbol] | CH [symbol] | Bu | (1-2-4a-1) |
| NH | CH [symbol] | CH [symbol] | CH [symbol] | Bnz | (1-2-5a-1) |
| NH | CH [symbol] | CH [symbol] | Et | Et | (1-2-6a-1) |
| NH | CH [symbol] | CH [symbol] | CH [symbol] CH [symbol] OCH [symbol] | CH [symbol] CH [symbol] OCH [symbol] | (1-2-7a-1) |
| NH | CH [symbol] | CH [symbol] | Pr | Bu | (1-2-8a-1) |
| NH | CH [symbol] | Et | Et | Et | (1-2-9a-1) |
| NH | CH [symbol] | ⁱPr | ⁱPr | ⁱPr | (1-2-10a-1) |
| NH | CH [symbol] | Bu | Bu | Bu | (1-2-11a-1) |
| NH | CH [symbol] CH [symbol] CH [symbol] | CH [symbol] | CH [symbol] | CH [symbol] | (1-2-12a-1) |
| NH | CH [symbol] CH [symbol] CH [symbol] | CH [symbol] | CH [symbol] | Et | (1-2-13a-1) |
| NH | CH [symbol] CH [symbol] CH [symbol] | CH [symbol] | CH [symbol] | ⁱPr | (1-2-14a-1) |
| NH | CH [symbol] CH [symbol] CH [symbol] | CH [symbol] | CH [symbol] | Bu | (1-2-15a-1) |
| NH | CH [symbol] CH [symbol] CH [symbol] | CH [symbol] | CH [symbol] | Bnz | (1-2-16a-1) |
| NH | CH [symbol] CH [symbol] CH [symbol] | CH [symbol] | Et | Et | (1-2-17a-1) |
| NH | CH [symbol] CH [symbol] CH [symbol] | CH [symbol] | CH [symbol] CH [symbol] OCH [symbol] | CH [symbol] CH [symbol] OCH [symbol] | (1-2-18a-1) |
| NH | CH [symbol] CH [symbol] CH [symbol] | CH [symbol] | Pr | Bu | (1-2-19a-1) |
| NH | CH [symbol] CH [symbol] CH [symbol] | Et | Et | Et | (1-2-20a-1) |
| NH | CH [symbol] CH [symbol] CH [symbol] | ⁱPr | ⁱPr | ⁱPr | (1-2-21a-1) |
| NH | CH [symbol] CH [symbol] CH [symbol] | Bu | Bu | Bu | (1-2-22a-1) |
| NH | CH [symbol] (CH [symbol] ) [symbol] CH [symbol] | CH [symbol] | CH [symbol] | CH [symbol] | (1-2-23a-1) |
| NH | CH [symbol] (CH [symbol] ) [symbol] CH [symbol] | CH [symbol] | CH [symbol] | Et | (1-2-24a-1) |
| NH | CH [symbol] (CH [symbol] ) [symbol] CH [symbol] | CH [symbol] | CH [symbol] | ⁱPr | (1-2-25a-1) |
| NH | CH [symbol] (CH [symbol] ) [symbol] CH [symbol] | CH [symbol] | CH [symbol] | Bu | (1-2-26a-1) |
| NH | CH [symbol] (CH [symbol] ) [symbol] CH [symbol] | CH [symbol] | CH [symbol] | Bnz | (1-2-27a-1) |
| NH | CH [symbol] (CH [symbol] ) [symbol] CH [symbol] | CH [symbol] | Et | Et | (1-2-28a-1) |
| NH | CH [symbol] (CH [symbol] ) [symbol] CH [symbol] | CH [symbol] | CH [symbol] CH [symbol] | CH [symbol] CH [symbol] OCH [symbol] | (1-2-29a-1) |
| NH | CH [symbol] (CH [symbol] ) [symbol] CH [symbol] | CH [symbol] | Pr | Bu | (1-2-30a-1) |
| NH | CH [symbol] (CH [symbol] ) [symbol] CH [symbol] | Et | Et | Et | (1-2-31a-1) |
| NH | CH [symbol] (CH [symbol] ) [symbol] CH [symbol] | ⁱPr | ⁱPr | ⁱPr | (1-2-32a-1) |
| NH | CH [symbol] (CH [symbol] ) [symbol] CH [symbol] | Bu | Bu | Bu | (1-2-33a-1) |

[symbol]

indicates data missing or illegible when filed

| X(R [symbol] ) [symbol] | R [symbol] | R [symbol] | R [symbol] | R [symbol] | |
|---|---|---|---|---|---|
| N(CH [symbol] ) | CH [symbol] | CH [symbol] | CH [symbol] | CH [symbol] | (1-2-1b-1) |
| N(CH [symbol] ) | CH [symbol] | CH [symbol] | CH [symbol] | Et | (1-2-2b-1) |
| N(CH [symbol] ) | CH [symbol] | CH [symbol] | CH [symbol] | ⁱPr | (1-2-3b-1) |
| N(CH [symbol] ) | CH [symbol] | CH [symbol] | CH [symbol] | Bu | (1-2-4b-1) |
| N(CH [symbol] ) | CH [symbol] | CH [symbol] | CH [symbol] | Bnz | (1-2-5b-1) |
| N(CH [symbol] ) | CH [symbol] | CH [symbol] | Et | Et | (1-2-6b-1) |
| N(CH [symbol] ) | CH [symbol] | CH [symbol] | CH [symbol] CH [symbol] OCH [symbol] | CH [symbol] CH [symbol] OCH [symbol] | (1-2-7b-1) |
| N(CH [symbol] ) | CH [symbol] | CH [symbol] | Pr | Bu | (1-2-8b-1) |
| N(CH [symbol] ) | CH [symbol] | Et | Et | Et | (1-2-9b-1) |
| N(CH [symbol] ) | CH [symbol] | ⁱPr | ⁱPr | ⁱPr | (1-2-10b-1) |
| N(CH [symbol] ) | CH [symbol] | Bu | Bu | Bu | (1-2-11b-1) |
| N(CH [symbol] ) | CH [symbol] CH [symbol] CH [symbol] | CH [symbol] | CH [symbol] | CH [symbol] | (1-2-12b-1) |
| N(CH [symbol] ) | CH [symbol] CH [symbol] CH [symbol] | CH [symbol] | CH [symbol] | Et | (1-2-13b-1) |
| N(CH [symbol] ) | CH [symbol] CH [symbol] CH [symbol] | CH [symbol] | CH [symbol] | ⁱPr | (1-2-14b-1) |
| N(CH [symbol] ) | CH [symbol] CH [symbol] CH [symbol] | CH [symbol] | CH [symbol] | Bu | (1-2-15b-1) |
| N(CH [symbol] ) | CH [symbol] CH [symbol] CH [symbol] | CH [symbol] | CH [symbol] | Bnz | (1-2-16b-1) |
| N(CH [symbol] ) | CH [symbol] CH [symbol] CH [symbol] | CH [symbol] | Et | Et | (1-2-17b-1) |
| N(CH [symbol] ) | CH [symbol] CH [symbol] CH [symbol] | CH [symbol] | CH [symbol] CH [symbol] OCH [symbol] | CH [symbol] CH [symbol] OCH [symbol] | (1-2-18b-1) |
| N(CH [symbol] ) | CH [symbol] CH [symbol] CH [symbol] | CH [symbol] | Pr | Bu | (1-2-19b-1) |
| N(CH [symbol] ) | CH [symbol] CH [symbol] CH [symbol] | Et | Et | Et | (1-2-20b-1) |
| N(CH [symbol] ) | CH [symbol] CH [symbol] CH [symbol] | ⁱPr | ⁱPr | ⁱPr | (1-2-21b-1) |
| N(CH [symbol] ) | CH [symbol] CH [symbol] CH [symbol] | Bu | Bu | Bu | (1-2-22b-1) |
| N(CH [symbol] ) | CH [symbol] (CH [symbol] ) [symbol] CH [symbol] | CH [symbol] | CH [symbol] | CH [symbol] | (1-2-23b-1) |
| N(CH [symbol] ) | CH [symbol] (CH [symbol] ) [symbol] CH [symbol] | CH [symbol] | CH [symbol] | Et | (1-2-24b-1) |
| N(CH [symbol] ) | CH [symbol] (CH [symbol] ) [symbol] CH [symbol] | CH [symbol] | CH [symbol] | ⁱPr | (1-2-25b-1) |
| N(CH [symbol] ) | CH [symbol] (CH [symbol] ) [symbol] CH [symbol] | CH [symbol] | CH [symbol] | Bu | (1-2-26b-1) |
| N(CH [symbol] ) | CH [symbol] (CH [symbol] ) [symbol] CH [symbol] | CH [symbol] | CH [symbol] | Bnz | (1-2-27b-1) |
| N(CH [symbol] ) | CH [symbol] (CH [symbol] ) [symbol] CH [symbol] | CH [symbol] | Et | Et | (1-2-28b-1) |
| N(CH [symbol] ) | CH [symbol] (CH [symbol] ) [symbol] CH [symbol] | CH [symbol] | CH [symbol] CH [symbol] | CH [symbol] CH [symbol] OCH [symbol] | (1-2-29b-1) |
| N(CH [symbol] ) | CH [symbol] (CH [symbol] ) [symbol] CH [symbol] | CH [symbol] | Pr | Bu | (1-2-30b-1) |
| N(CH [symbol] ) | CH [symbol] (CH [symbol] ) [symbol] CH [symbol] | Et | Et | Et | (1-2-31b-1) |
| N(CH [symbol] ) | CH [symbol] (CH [symbol] ) [symbol] CH [symbol] | ⁱPr | ⁱPr | ⁱPr | (1-2-32b-1) |
| N(CH [symbol] ) | CH [symbol] (CH [symbol] ) [symbol] CH [symbol] | Bu | Bu | Bu | (1-2-33b-1) |

[symbol]

indicates data missing or illegible when filed

| X(R [symbol] ) [symbol] | R [symbol] | R [symbol] | R [symbol] | R [symbol] | |
|---|---|---|---|---|---|
| O | CH [symbol] | CH [symbol] | CH [symbol] | CH [symbol] | (1-2-1c-1) |
| O | CH [symbol] | CH [symbol] | CH [symbol] | Et | (1-2-2c-1) |
| O | CH [symbol] | CH [symbol] | CH [symbol] | ⁱPr | (1-2-3c-1) |
| O | CH [symbol] | CH [symbol] | CH [symbol] | Bu | (1-2-4c-1) |
| O | CH [symbol] | CH [symbol] | CH [symbol] | Bnz | (1-2-5c-1) |
| O | CH [symbol] | CH [symbol] | Et | Et | (1-2-6c-1) |
| O | CH [symbol] | CH [symbol] | CH [symbol] CH [symbol] OCH [symbol] | CH [symbol] CH [symbol] OCH [symbol] | (1-2-7c-1) |
| O | CH [symbol] | CH [symbol] | Pr | Bu | (1-2-8c-1) |
| O | CH [symbol] | Et | Et | Et | (1-2-9c-1) |
| O | CH [symbol] | ⁱPr | ⁱPr | ⁱPr | (1-2-10c-1) |
| O | CH [symbol] | Bu | Bu | Bu | (1-2-11c-1) |
| O | CH [symbol] CH [symbol] CH [symbol] | CH [symbol] | CH [symbol] | CH [symbol] | (1-2-12c-1) |
| O | CH [symbol] CH [symbol] CH [symbol] | CH [symbol] | CH [symbol] | Et | (1-2-13c-1) |
| O | CH [symbol] CH [symbol] CH [symbol] | CH [symbol] | CH [symbol] | ⁱPr | (1-2-14c-1) |
| O | CH [symbol] CH [symbol] CH [symbol] | CH [symbol] | CH [symbol] | Bu | (1-2-15c-1) |
| O | CH [symbol] CH [symbol] CH [symbol] | CH [symbol] | CH [symbol] | Bnz | (1-2-16c-1) |
| O | CH [symbol] CH [symbol] CH [symbol] | CH [symbol] | Et | Et | (1-2-17c-1) |
| O | CH [symbol] CH [symbol] CH [symbol] | CH [symbol] | CH [symbol] CH [symbol] OCH [symbol] | CH [symbol] CH [symbol] OCH [symbol] | (1-2-18c-1) |
| O | CH [symbol] CH [symbol] CH [symbol] | CH [symbol] | Pr | Bu | (1-2-19c-1) |
| O | CH [symbol] CH [symbol] CH [symbol] | Et | Et | Et | (1-2-20c-1) |
| O | CH [symbol] CH [symbol] CH [symbol] | ⁱPr | ⁱPr | ⁱPr | (1-2-21c-1) |
| O | CH [symbol] CH [symbol] CH [symbol] | Bu | Bu | Bu | (1-2-22c-1) |
| O | CH [symbol] (CH [symbol] ) [symbol] CH [symbol] | CH [symbol] | CH [symbol] | CH [symbol] | (1-2-23c-1) |
| O | CH [symbol] (CH [symbol] ) [symbol] CH [symbol] | CH [symbol] | CH [symbol] | Et | (1-2-24c-1) |
| O | CH [symbol] (CH [symbol] ) [symbol] CH [symbol] | CH [symbol] | CH [symbol] | ⁱPr | (1-2-25c-1) |
| O | CH [symbol] (CH [symbol] ) [symbol] CH [symbol] | CH [symbol] | CH [symbol] | Bu | (1-2-26c-1) |
| O | CH [symbol] (CH [symbol] ) [symbol] CH [symbol] | CH [symbol] | CH [symbol] | Bnz | (1-2-27c-1) |
| O | CH [symbol] (CH [symbol] ) [symbol] CH [symbol] | CH [symbol] | Et | Et | (1-2-28c-1) |
| O | CH [symbol] (CH [symbol] ) [symbol] CH [symbol] | CH [symbol] | CH [symbol] CH [symbol] | CH [symbol] CH [symbol] OCH [symbol] | (1-2-29c-1) |
| O | CH [symbol] (CH [symbol] ) [symbol] CH [symbol] | CH [symbol] | Pr | Bu | (1-2-30c-1) |
| O | CH [symbol] (CH [symbol] ) [symbol] CH [symbol] | Et | Et | Et | (1-2-31c-1) |
| O | CH [symbol] (CH [symbol] ) [symbol] CH [symbol] | ⁱPr | ⁱPr | ⁱPr | (1-2-32c-1) |
| O | CH [symbol] (CH [symbol] ) [symbol] CH [symbol] | Bu | Bu | Bu | (1-2-33c-1) |

[symbol]

indicates data missing or illegible when filed

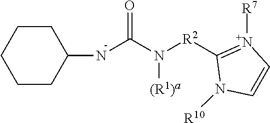

| X(R [symbol] ) [symbol] | R² | R⁷ | R¹⁰ | |
|---|---|---|---|---|
| NH | CH [symbol] | CH₃ | CH₃ | (1-3-1a-1) |
| NH | CH₂ | CH₃ | Et | (1-3-2a-1) |
| NH | CH [symbol] | CH₃ | ⁱPr | (1-3-3a-1) |
| NH | CH₂ | CH₃ | Bu | (1-3-4a-1) |
| NH | CH₂ | CH₃ | Bnz | (1-3-5a-1) |
| NH | CH [symbol] | CH₃ | CH₂CH₂OCH₃ | (1-3-6a-1) |
| NH | CH₂ | Et | Et | (1-3-7a-1) |
| NH | CH₂CH [symbol] | CH₃ | CH₃ | (1-3-8a-1) |
| NH | CH₂CH₂ | CH₃ | Et | (1-3-9a-1) |
| NH | CH₂CH₂ | CH₃ | ⁱPr | (1-3-10a-1) |
| NH | CH₂CH₂ | CH₃ | Bu | (1-3-11a-1) |
| NH | CH₂CH [symbol] | CH₃ | Bnz | (1-3-12a-1) |
| NH | CH₂CH₂ | CH₃ | CH₂CH₂OCH₃ | (1-3-13a-1) |
| NH | CH₂CH₂ | Et | Et | (1-3-14a-1) |
| NH | CH₂(CH₂)₄CH₂ | CH₃ | CH₃ | (1-3-15a-1) |
| NH | CH₂(CH₂)₄CH [symbol] | CH₃ | Et | (1-3-16a-1) |
| NH | CH₂(CH₂)₄CH₂ | CH₃ | ⁱPr | (1-3-17a-1) |
| NH | CH₂(CH₂)₄CH₂ | CH₃ | Bu | (1-3-18a-1) |
| NH | CH [symbol] (CH₂)₄CH [symbol] | CH₃ | Bnz | (1-3-19a-1) |
| NH | CH₂(CH₂)₄CH₂ | CH₃ | CH₂CH [symbol] OCH₃ | (1-3-20a-1) |
| NH | CH₂(CH₂)₄CH₂ | Et | Et | (1-3-21a-1) |

[symbol]

indicates data missing or illegible when filed

| X(R [symbol] ) [symbol] | R² | R⁷ | R¹⁰ | |
|---|---|---|---|---|
| N(CH₃) | CH₂ | CH₃ | CH₃ | (1-3-1b-1) |
| N(CH₃) | CH₂ | CH₃ | Et | (1-3-2b-1) |
| N(CH₃) | CH₂ | CH₃ | ⁱPr | (1-3-3b-1) |
| N(CH₃) | CH₂ | CH₃ | Bu | (1-3-4b-1) |
| N(CH₃) | CH₂ | CH₃ | Bnz | (1-3-5b-1) |
| N(CH₃) | CH₂ | CH₃ | CH₂CH₂OCH₃ | (1-3-6b-1) |
| N(CH₃) | CH₂ | Et | Et | (1-3-7b-1) |
| N(CH₃) | CH₂CH₂ | CH₃ | CH₃ | (1-3-8b-1) |
| N(CH₃) | CH₂CH₂ | CH₃ | Et | (1-3-9b-1) |
| N(CH₃) | CH₂CH₂ | CH₃ | ⁱPr | (1-3-10b-1) |
| N(CH₃) | CH₂CH₂ | CH₃ | Bu | (1-3-11b-1) |
| N(CH₃) | CH₂CH₂ | CH₃ | Bnz | (1-3-12b-1) |
| N(CH₃) | CH₂CH₂ | CH₃ | CH₂CH₂OCH₃ | (1-3-13b-1) |
| N(CH₃) | CH₂CH₂ | Et | Et | (1-3-146-1) |
| N(CH₃) | CH₂(CH₂)₄CH₂ | CH₃ | CH₃ | (1-3-15b-1) |
| N(CH₃) | CH [symbol] (CH₂)₄CH₂ | CH₃ | Et | (1-3-16b-1) |
| N(CH₃) | CH [symbol] (CH₂)₄CH₂ | CH₃ | ⁱPr | (1-3-17b-1) |
| N(CH₃) | CH₂(CH₂)₄CH₂ | CH₃ | Bu | (1-3-18b-1) |
| N(CH₃) | CH₂(CH₂)₄CH₂ | CH₃ | Bnz | (1-3-19b-1) |
| N(CH₃) | CH₂(CH₂)₄CH₂ | CH₃ | CH₂CH₂OCH₃ | (1-3-20b-1) |
| N(CH₃) | CH₂(CH₂)₄CH₂ | Et | Et | (1-3-21b-1) |

[symbol]

indicates data missing or illegible when filed

| X(R [symbol] ) [symbol] | R² | R⁷ | R¹⁰ | |
|---|---|---|---|---|
| O | CH₂ | CH₃ | CH₃ | (1-3-1c-1) |
| O | CH₂ | CH₃ | Et | (1-3-2c-1) |
| O | CH₂ | CH₃ | ⁱPr | (1-3-3c-1) |
| O | CH₂ | CH₃ | Bu | (1-3-4c-1) |
| O | CH₂ | CH₃ | Bnz | (1-3-5c-1) |
| O | CH₂ | CH₃ | CH₂CH₂OCH₃ | (1-3-6c-1) |
| O | CH₂ | Et | Et | (1-3-7c-1) |
| O | CH₂CH₂ | CH₃ | CH₃ | (1-3-8c-1) |
| O | CH₂CH₂ | CH₃ | Et | (1-3-9c-1) |
| O | CH₂CH₂ | CH₃ | ⁱPr | (1-3-10c-1) |
| O | CH₂CH₂ | CH₃ | Bu | (1-3-11c-1) |
| O | CH₂CH₂ | CH₃ | Bnz | (1-3-12c-1) |
| O | CH₂CH₂ | CH₃ | CH₂CH₂OCH₃ | (1-3-13c-1) |
| O | CH₂CH₂ | Et | Et | (1-3-14c-1) |
| O | CH₂(CH₂)₄CH₂ | CH₃ | CH₃ | (1-3-15c-1) |
| O | CH₂(CH₂)₄CH₂ | CH₃ | Et | (1-3-16c-1) |
| O | CH₂(CH₂)₄CH₂ | CH₃ | ⁱPr | (1-3-17c-1) |
| O | CH₂(CH₂)₄CH₂ | CH₃ | Bu | (1-3-18c-1) |
| O | CH₂(CH₂)₄CH₂ | CH₃ | Bnz | (1-3-19c-1) |
| O | CH₂(CH₂)₄CH₂ | CH₃ | CH₂CH₂OCH₃ | (1-3-20c-1) |
| O | CH₂(CH₂)₄CH₂ | Et | Et | (1-3-21c-1) |

[symbol]

indicates data missing or illegible when filed

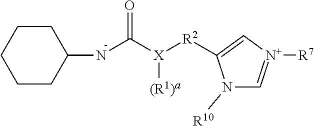

| X(R [symbol] ) [symbol] | R² | R⁷ | R¹⁰ | |
|---|---|---|---|---|
| NH | CH₂ | CH₃ | CH₃ | (1-3-22a-1) |
| NH | CH₂ | CH₃ | Et | (1-3-23a-1) |
| NH | CH₂ | CH₃ | ⁱPr | (1-3-24a-1) |
| NH | CH₂ | CH₃ | Bu | (1-3-25a-1) |
| NH | CH [symbol] | CH₃ | Bnz | (1-3-26a-1) |
| NH | CH₂ | CH₃ | CH₂CH₂OCH₃ | (1-3-27a-1) |
| NH | CH₂ | Et | Et | (1-3-28a-1) |
| NH | CH₂CH [symbol] | CH₃ | CH₃ | (1-3-29a-1) |
| NH | CH₂CH [symbol] | CH₃ | Et | (1-3-30a-1) |
| NH | CH₂CH₂ | CH₃ | ⁱPr | (1-3-31a-1) |
| NH | CH₂CH₂ | CH₃ | Bu | (1-3-32a-1) |
| NH | CH₂CH [symbol] | CH₃ | Bnz | (1-3-33a-1) |
| NH | CH₂CH₂ | CH₃ | CH₂CH₂OCH₃ | (1-3-34a-1) |
| NH | CH₂CH₂ | Et | Et | (1-3-35a-1) |
| NH | CH₂(CH₂)₄CH₂ | CH₃ | CH₃ | (1-3-36a-1) |
| NH | CH₂(CH₂)₄CH [symbol] | CH₃ | Et | (1-3-37a-1) |
| NH | CH [symbol] (CH₂)₄CH₂ | CH₃ | ⁱPr | (1-3-38a-1) |
| NH | CH [symbol] (CH₂)₄CH₂ | CH₃ | Bu | (1-3-39a-1) |
| NH | CH₂(CH₂)₄CH₂ | CH₃ | Bnz | (1-3-40a-1) |
| NH | CH₂(CH [symbol] ) [symbol] CH₂ | CH₃ | CH₂CH₂OCH₃ | (1-3-41a-1) |
| NH | CH [symbol] (CH₂)₄CH₂ | Et | Et | (1-3-42a-1) |

[symbol]

indicates data missing or illegible when filed

| X(R¹) [symbol] | R² | R⁷ | R¹⁰ | |
|---|---|---|---|---|
| N(CH₃) | CH₂ | CH₃ | CH₃ | (1-3-22b-1) |
| N(CH₃) | CH₂ | CH₃ | Et | (1-3-23b-1) |
| N(CH₃) | CH₂ | CH₃ | ⁱPr | (1-3-24b-1) |
| N(CH₃) | CH₂ | CH₃ | Bu | (1-3-25b-1) |
| N(CH₃) | CH₂ | CH₃ | Bnz | (1-3-26b-1) |
| N(CH₃) | CH₂ | CH₃ | CH₂CH₂OCH₃ | (1-3-27b-1) |
| N(CH₃) | CH₂ | Et | Et | (1-3-28b-1) |
| N(CH₃) | CH₂CH₂ | CH₃ | CH₃ | (1-3-29b-1) |
| N(CH₃) | CH₂CH₂ | CH₃ | Et | (1-3-30b-1) |
| N(CH₃) | CH₂CH₂ | CH₃ | ⁱPr | (1-3-31b-1) |
| N(CH₃) | CH₂CH₂ | CH₃ | Bu | (1-3-32b-1) |
| N(CH₃) | CH₂CH₂ | CH₃ | Bnz | (1-3-33b-1) |
| N(CH₃) | CH₂CH₂ | CH₃ | CH₂CH₂OCH₃ | (1-3-34b-1) |
| N(CH₃) | CH₂CH₂ | Et | Et | (1-3-35b-1) |
| N(CH₃) | CH₂(CH₂)₄CH₂ | CH₃ | CH₃ | (1-3-36b-1) |
| N(CH₃) | CH₂(CH₂)₄CH₂ | CH₃ | Et | (1-3-37b-1) |
| N(CH₃) | CH₂(CH₂)₄CH₂ | CH₃ | ⁱPr | (1-3-38b-1) |
| N(CH₃) | CH₂(CH₂)₄CH₂ | CH₃ | Bu | (1-3-39b-1) |
| N(CH₃) | CH₂(CH₂)₄CH₂ | CH₃ | Bnz | (1-3-40b-1) |
| N(CH₃) | CH₂(CH₂)₄CH₂ | CH₃ | CH₂CH₂OCH₃ | (1-3-41b-1) |
| N(CH₃) | CH₂(CH₂)₄CH₂ | Et | Et | (1-3-42b-1) |

[symbol]

indicates data missing or illegible when filed

| X(R¹) [symbol] | R² | R⁷ | R¹⁰ | |
|---|---|---|---|---|
| O | CH₂ | CH₃ | CH₃ | (1-3-22c-1) |
| O | CH₂ | CH₃ | Et | (1-3-23c-1) |
| O | CH₂ | CH₃ | ⁱPr | (1-3-24c-1) |
| O | CH₂ | CH₃ | Bu | (1-3-25c-1) |
| O | CH₂ | CH₃ | Bnz | (1-3-26c-1) |
| O | CH₂ | CH₃ | CH₂CH₂OCH₃ | (1-3-27c-1) |
| O | CH₂ | Et | Et | (1-3-28c-1) |
| O | CH₂CH₂ | CH₃ | CH₃ | (1-3-29c-1) |
| O | CH₂CH₂ | CH₃ | Et | (1-3-30c-1) |
| O | CH₂CH₂ | CH₃ | ⁱPr | (1-3-31c-1) |
| O | CH₂CH₂ | CH₃ | Bu | (1-3-32c-1) |
| O | CH₂CH₂ | CH₃ | Bnz | (1-3-33c-1) |
| O | CH₂CH₂ | CH₃ | CH₂CH₂OCH₃ | (1-3-34c-1) |
| O | CH₂CH₂ | Et | Et | (1-3-35c-1) |
| O | CH₂(CH₂)₄CH₂ | CH₃ | CH₃ | (1-3-36c-1) |
| O | CH₂(CH₂)₄CH₂ | CH₃ | Et | (1-3-37c-1) |
| O | CH₂(CH₂)₄CH₂ | CH₃ | ⁱPr | (1-3-38c-1) |
| O | CH₂(CH₂)₄CH₂ | CH₃ | Bu | (1-3-39c-1) |
| O | CH₂(CH₂)₄CH₂ | CH₃ | Bnz | (1-3-40c-1) |
| O | CH₂(CH₂)₄CH₂ | CH₃ | CH₂CH₂OCH₃ | (1-3-41c-1) |
| O | CH₂(CH₂)₄CH₂ | Et | Et | (1-3-42c-1) |

[symbol]

indicates data missing or illegible when filed

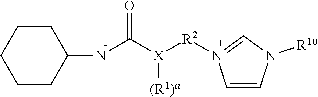

| X(R¹) [symbol] | R² | R¹⁰ | |
|---|---|---|---|
| NH | CH₂ | CH₃ | (1-3-43a-1) |
| NH | CH₂ | Et | (1-3-44a-1) |
| NH | CH₂ | Pr | (1-3-45a-1) |
| NH | CH₂ | Bu | (1-3-46a-1) |
| NH | CH₂CH₂ | CH₃ | (1-3-47a-1) |
| NH | CH₂CH₂ | Et | (1-3-48a-1) |
| NH | CH₂CH₂ | Pr | (1-3-49a-1) |
| NH | CH₂CH₂ | Bu | (1-3-50a-1) |
| NH | CH₂(CH₂)₄CH₂ | CH₃ | (1-3-51a-1) |
| NH | CH₂(CH₂)₄CH₂ | Et | (1-3-52a-1) |
| NH | CH₂(CH₂)₄CH₂ | Pr | (1-3-53a-1) |
| NH | CH₂(CH₂)₄CH₂ | Bu | (1-3-54a-1) |

[symbol]

indicates data missing or illegible when filed

| X(R¹) [symbol] | R² | R¹⁰ | |
|---|---|---|---|
| N(CH₃) | CH₂ | CH₃ | (1-3-43b-1) |
| N(CH₃) | CH₂ | Et | (1-3-44b-1) |
| N(CH₃) | CH₂ | Pr | (1-3-45b-1) |
| N(CH₃) | CH₂ | Bu | (1-3-46b-1) |
| N(CH₃) | CH₂CH₂ | CH₃ | (1-3-47b-1) |
| N(CH₃) | CH₂CH₂ | Et | (1-3-48b-1) |
| N(CH₃) | CH₂CH₂ | Pr | (1-3-49b-1) |
| N(CH₃) | CH₂CH₂ | Bu | (1-3-50b-1) |
| N(CH₃) | CH₂(CH₂)₄CH₂ | CH₃ | (1-3-51b-1) |
| N(CH₃) | CH₂(CH₂)₄CH₂ | Et | (1-3-52b-1) |
| N(CH₃) | CH₂(CH₂)₄CH₂ | Pr | (1-3-53b-1) |
| N(CH₃) | CH₂(CH₂)₄CH₂ | Bu | (1-3-54b-1) |

[symbol]

indicates data missing or illegible when filed

| X(R¹) [symbol] | R² | R¹⁰ | |
|---|---|---|---|
| O | CH₂ | CH₃ | (1-3-43c-1) |
| O | CH₂ | Et | (1-3-44c-1) |
| O | CH₂ | Pr | (1-3-45c-1) |
| O | CH₂ | Bu | (1-3-46c-1) |
| O | CH₂CH₂ | CH₃ | (1-3-47c-1) |
| O | CH₂CH₂ | Et | (1-3-48c-1) |
| O | CH₂CH₂ | Pr | (1-3-49c-1) |
| O | CH₂CH₂ | Bu | (1-3-50c-1) |
| O | CH₂(CH₂)₄CH₂ | CH₃ | (1-3-51c-1) |
| O | CH₂(CH₂)₄CH₂ | Et | (1-3-52c-1) |
| O | CH₂(CH₂)₄CH₂ | Pr | (1-3-53c-1) |
| O | CH₂(CH₂)₄CH₂ | Bu | (1-3-54c-1) |

[symbol]

indicates data missing or illegible when filed

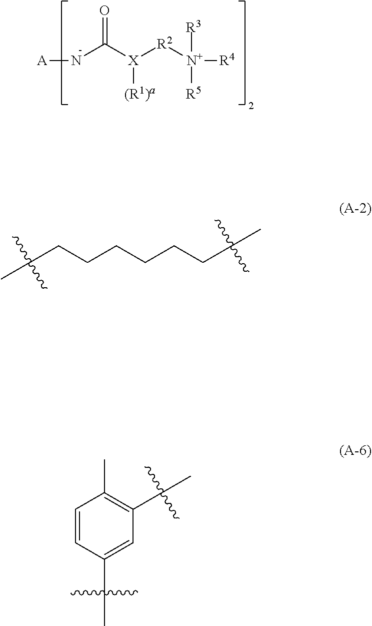

| A | X (R [symbol] ) [symbol] | R [symbol] | R [symbol] | R [symbol] | R [symbol] | |
|---|---|---|---|---|---|---|
| A-2 | NH | CH [symbol] | CH [symbol] | CH [symbol] | CH [symbol] | (1-2-1a-2) |
| A-2 | NH | CH [symbol] | CH [symbol] | CH [symbol] | Br | (1-2-2a-2) |
| A-2 | NH | CH [symbol] | CH [symbol] | CH [symbol] | [symbol] Pr | (1-2-3a-2) |
| A-2 | NH | CH [symbol] | CH [symbol] | CH [symbol] | [symbol] | (1-2-4a-2) |
| A-2 | NH | CH [symbol] | CH [symbol] | CH [symbol] | [symbol] | (1-2-5a-2) |
| A-2 | NH | CH [symbol] | CH [symbol] | Et | [symbol] | (1-2-6a-2) |
| A-2 | NH | CH [symbol] | CH [symbol] | CH [symbol] CH [symbol] OCH [symbol] | CH [symbol] CH [symbol] OCH [symbol] | (1-2-7a-2) |
| A-2 | NH | CH [symbol] | CH [symbol] | Pr | [symbol] | (1-2-8a-2) |
| A-2 | NH | CH [symbol] | Et | Et | [symbol] | (1-2-9a-2) |
| A-2 | NH | CH [symbol] | [symbol] Pr | [symbol] Pr | [symbol] | (1-2-10a-2) |
| A-2 | NH | CH [symbol] | Bu | Bu | [symbol] | (1-2-11a-2) |
| A-2 | NH | CH [symbol] CH [symbol] CH [symbol] | CH [symbol] | CH [symbol] | [symbol] | (1-2-12a-2) |
| A-2 | NH | CH [symbol] CH [symbol] CH [symbol] | CH [symbol] | CH [symbol] | [symbol] | (1-2-13a-2) |
| A-2 | NH | CH [symbol] CH [symbol] CH [symbol] | CH [symbol] | CH [symbol] | [symbol] | (1-2-14a-2) |
| A-2 | NH | CH [symbol] CH [symbol] CH [symbol] | CH [symbol] | CH [symbol] | [symbol] | (1-2-15a-2) |
| A-2 | NH | CH [symbol] CH [symbol] CH [symbol] | CH [symbol] | CH [symbol] | [symbol] | (1-2-16a-2) |
| A-2 | NH | CH [symbol] CH [symbol] CH [symbol] | CH [symbol] | Et | [symbol] | (1-2-17a-2) |
| A-2 | NH | CH [symbol] CH [symbol] CH [symbol] | CH [symbol] | CH [symbol] CH [symbol] OCH [symbol] | CH [symbol] CH [symbol] OCH [symbol] | (1-2-18a-2) |
| A-2 | NH | CH [symbol] CH [symbol] CH [symbol] | CH [symbol] | Pr | [symbol] | (1-2-19a-2) |
| A-2 | NH | CH [symbol] CH [symbol] CH [symbol] | Et | Et | [symbol] | (1-2-20a-2) |
| A-2 | NH | CH [symbol] CH [symbol] CH [symbol] | [symbol] Pr | [symbol] Pr | [symbol] | (1-2-21a-2) |
| A-2 | NH | CH [symbol] CH [symbol] CH [symbol] | Bu | Bu | [symbol] | (1-2-22a-2) |
| A-2 | NH | CH [symbol] (CH [symbol] ) [symbol] CH [symbol] | CH [symbol] | CH [symbol] | [symbol] | (1-2-23a-2) |
| A-2 | NH | CH [symbol] (CH [symbol] ) [symbol] CH [symbol] | CH [symbol] | CH [symbol] | [symbol] | (1-2-24a-2) |
| A-2 | NH | CH [symbol] (CH [symbol] ) [symbol] CH [symbol] | CH [symbol] | CH [symbol] | [symbol] | (1-2-25a-2) |
| A-2 | NH | CH [symbol] (CH [symbol] ) [symbol] CH [symbol] | CH [symbol] | CH [symbol] | [symbol] | (1-2-26a-2) |
| A-2 | NH | CH [symbol] (CH [symbol] ) [symbol] CH [symbol] | CH [symbol] | CH [symbol] | [symbol] | (1-2-27a-2) |
| A-2 | NH | CH [symbol] (CH [symbol] ) [symbol] CH [symbol] | CH [symbol] | Et | [symbol] | (1-2-28a-2) |
| A-2 | NH | CH [symbol] (CH [symbol] ) [symbol] CH [symbol] | CH [symbol] | CH [symbol] CH [symbol] OCH [symbol] | CH [symbol] CH [symbol] OCH [symbol] | (1-2-29a-2) |
| A-2 | NH | CH [symbol] (CH [symbol] ) [symbol] CH [symbol] | CH [symbol] | Pr | Bu | (1-2-30a-2) |

[symbol]

indicates data missing or illegible when filed

| | | | | | | |
|---|---|---|---|---|---|---|
| A-2 | NH | CH₂(CH₂) ₄CH₂ | Et | Et | Et | (1-2-31a-2) |
| A-2 | NH | CH₂(CH₂) ₄CH₂ | [symbol] Pr | [symbol] Pr | [symbol] Pr | (1-2-32a-2) |
| A-2 | NH | CH₂(CH₂) ₄CH₂ | Bu | Bu | Bu | (1-2-33a-2) |

[symbol]

indicates data missing or illegible when filed

| A | X (R [symbol] )\hat{ } | R [symbol] | R [symbol] | R [symbol] | R [symbol] | |
|---|---|---|---|---|---|---|
| A-2 | N(CH [symbol] ) | CH [symbol] | CH [symbol] | CH [symbol] | CH [symbol] | (1-2-1b-2) |
| A-2 | N(CH [symbol] ) | CH [symbol] | CH [symbol] | CH [symbol] | Et | (1-2-2b-2) |
| A-2 | N(CH [symbol] ) | CH [symbol] | CH [symbol] | CH [symbol] | [symbol] Pr | (1-2-3b-2) |
| A-2 | N(CH [symbol] ) | CH [symbol] | CH [symbol] | CH [symbol] | Bu | (1-2-4b-2) |
| A-2 | N(CH [symbol] ) | CH [symbol] | CH [symbol] | CH [symbol] | [symbol] | (1-2-5b-2) |
| A-2 | N(CH [symbol] ) | CH [symbol] | CH [symbol] | Et | Et | (1-2-6b-2) |
| A-2 | N(CH [symbol] ) | CH [symbol] | CH [symbol] | CH [symbol] CH [symbol] OCH [symbol] | CH [symbol] CH [symbol] OCH [symbol] | (1-2-7b-2) |
| A-2 | N(CH [symbol] ) | CH [symbol] | CH [symbol] | Pr | Bu | (1-2-8b-2) |
| A-2 | N(CH [symbol] ) | CH [symbol] | Et | Et | Et | (1-2-9b-2) |
| A-2 | N(CH [symbol] ) | CH [symbol] | [symbol] Pr | [symbol] Pr | [symbol] Pr | (1-2-10b-2) |
| A-2 | N(CH [symbol] ) | CH [symbol] | Bu | Bu | Bu | (1-2-11b-2) |
| A-2 | N(CH [symbol] ) | CH [symbol] CH [symbol] CH | CH [symbol] | CH [symbol] | CH [symbol] | (1-2-12b-2) |
| A-2 | N(CH [symbol] ) | CH [symbol] CH [symbol] CH | CH [symbol] | CH [symbol] | Et | (1-2-13b-2) |
| A-2 | N(CH [symbol] ) | CH [symbol] CH [symbol] CH | CH [symbol] | CH [symbol] | [symbol] Pr | (1-2-14b-2) |
| A-2 | N(CH [symbol] ) | CH [symbol] CH [symbol] CH | CH [symbol] | CH [symbol] | Bu | (1-2-15b-2) |
| A-2 | N(CH [symbol] ) | CH [symbol] CH [symbol] CH | CH [symbol] | CH [symbol] | [symbol] | (1-2-16b-2) |
| A-2 | N(CH [symbol] ) | CH [symbol] CH [symbol] CH | CH [symbol] | Et | Et | (1-2-17b-2) |
| A-2 | N(CH [symbol] ) | CH [symbol] CH [symbol] CH | CH [symbol] | CH [symbol] CH [symbol] OCH [symbol] | CH [symbol] CH [symbol] OCH [symbol] | (1-2-18b-2) |
| A-2 | N(CH [symbol] ) | CH [symbol] CH [symbol] CH | CH [symbol] | Pr | Bu | (1-2-19b-2) |
| A-2 | N(CH [symbol] ) | CH [symbol] CH [symbol] CH | Et | Et | Et | (1-2-20b-2) |
| A-2 | N(CH [symbol] ) | CH [symbol] CH [symbol] CH | [symbol] Pr | [symbol] Pr | [symbol] Pr | (1-2-21b-2) |
| A-2 | N(CH [symbol] ) | CH [symbol] CH [symbol] CH | Bu | Bu | Bu | (1-2-22b-2) |
| A-2 | N(CH [symbol] ) | CH [symbol] (CH [symbol] ) [symbol] CH [symbol] | CH [symbol] | CH [symbol] | CH [symbol] | (1-2-23b-2) |
| A-2 | N(CH [symbol] ) | CH [symbol] (CH [symbol] ) [symbol] CH [symbol] | CH [symbol] | CH [symbol] | Et | (1-2-24b-2) |
| A-2 | N(CH [symbol] ) | CH [symbol] (CH [symbol] ) [symbol] CH [symbol] | CH [symbol] | CH [symbol] | [symbol] Pr | (1-2-25b-2) |
| A-2 | N(CH [symbol] ) | CH [symbol] (CH [symbol] ) [symbol] CH [symbol] | CH [symbol] | CH [symbol] | Bu | (1-2-26b-2) |
| A-2 | N(CH [symbol] ) | CH [symbol] (CH [symbol] ) [symbol] CH [symbol] | CH [symbol] | CH [symbol] | [symbol] | (1-2-27b-2) |
| A-2 | N(CH [symbol] ) | CH [symbol] (CH [symbol] ) [symbol] CH [symbol] | CH [symbol] | Et | Et | (1-2-28b-2) |
| A-2 | N(CH [symbol] ) | CH [symbol] (CH [symbol] ) [symbol] CH [symbol] | CH [symbol] | CH [symbol] CH [symbol] OCH [symbol] | CH [symbol] CH [symbol] OCH [symbol] | (1-2-29b-2) |
| A-2 | N(CH [symbol] ) | CH [symbol] (CH [symbol] ) [symbol] CH [symbol] | CH [symbol] | Pr | [symbol] | (1-2-30b-2) |
| A-2 | N(CH [symbol] ) | CH [symbol] (CH [symbol] ) [symbol] CH [symbol] | Et | Et | Et | (1-2-31b-2) |
| A-2 | N(CH [symbol] ) | CH [symbol] (CH [symbol] ) [symbol] CH [symbol] | [symbol] Pr | [symbol] Pr | [symbol] Pr | (1-2-32b-2) |
| A-2 | N(CH [symbol] ) | CH [symbol] (CH [symbol] ) [symbol] CH [symbol] | Bu | Bu | Bu | (1-2-33b-2) |

[symbol]

indicates data missing or illegible when filed

| [symbol] | X (R [symbol] ) [symbol] | R [symbol] | R [symbol] | R [symbol] | R [symbol] | |
|---|---|---|---|---|---|---|
| A-2 | 0 | CH [symbol] | CH [symbol] | CH [symbol] | CH [symbol] | (1-2-1c-2) |
| A-2 | 0 | CH [symbol] | CH [symbol] | CH [symbol] | Et | (1-2-2c-2) |
| A-2 | 0 | CH [symbol] | CH [symbol] | CH [symbol] | [symbol] Pr | (1-2-3c-2) |
| A-2 | 0 | CH [symbol] | CH [symbol] | CH [symbol] | Bu | (1-2-4c-2) |
| A-2 | 0 | CH [symbol] | CH [symbol] | CH [symbol] | [symbol] | (1-2-5c-2) |
| A-2 | 0 | CH [symbol] | CH [symbol] | Et | Et | (1-2-6c-2) |
| A-2 | 0 | CH [symbol] | CH [symbol] | CH [symbol] CH [symbol] OCH [symbol] | CH [symbol] CH [symbol] OCH [symbol] | (1-2-7c-2) |
| A-2 | 0 | CH [symbol] | CH [symbol] | Pr | Bu | (1-2-8c-2) |
| A-2 | 0 | CH [symbol] | Et | Et | Et | (1-2-9c-2) |
| A-2 | 0 | CH [symbol] | [symbol] Pr | [symbol] Pr | [symbol] Pr | (1-2-10c-2) |
| A-2 | 0 | CH [symbol] | Bu | Bu | Bu | (1-2-11c-2) |
| A-2 | 0 | CH [symbol] CH [symbol] CH [symbol] | CH [symbol] | CH [symbol] | CH [symbol] | (1-2-12c-2) |
| A-2 | 0 | CH [symbol] CH [symbol] CH [symbol] | CH [symbol] | CH [symbol] | Et | (1-2-13c-2) |
| A-2 | 0 | CH [symbol] CH [symbol] CH [symbol] | CH [symbol] | CH [symbol] | [symbol] Pr | (1-2-14c-2) |
| A-2 | 0 | CH [symbol] CH [symbol] CH [symbol] | CH [symbol] | CH [symbol] | Bu | (1-2-15c-2) |
| A-2 | 0 | CH [symbol] CH [symbol] CH [symbol] | CH [symbol] | CH [symbol] | [symbol] | (1-2-16c-2) |
| A-2 | 0 | CH [symbol] CH [symbol] CH [symbol] | CH [symbol] | Et | Et | (1-2-17c-2) |
| A-2 | 0 | CH [symbol] CH [symbol] CH [symbol] | CH [symbol] | CH [symbol] CH [symbol] OCH [symbol] | CH [symbol] CH [symbol] OCH [symbol] | (1-2-18c-2) |
| A-2 | 0 | CH [symbol] CH [symbol] CH [symbol] | CH [symbol] | Pr | Bu | (1-2-19c-2) |
| A-2 | 0 | CH [symbol] CH [symbol] CH [symbol] | Et | Et | Et | (1-2-20c-2) |
| A-2 | 0 | CH [symbol] CH [symbol] CH [symbol] | [symbol] Pr | [symbol] Pr | [symbol] Pr | (1-2-21c-2) |
| A-2 | 0 | CH [symbol] CH [symbol] CH [symbol] | Bu | Bu | Bu | (1-2-22c-2) |
| A-2 | 0 | CH [symbol] (CH [symbol] ) [symbol] CH [symbol] | CH [symbol] | CH [symbol] | CH [symbol] | (1-2-23c-2) |
| A-2 | 0 | CH [symbol] (CH [symbol] ) [symbol] CH [symbol] | CH [symbol] | CH [symbol] | Et | (1-2-24c-2) |
| A-2 | 0 | CH [symbol] (CH [symbol] ) [symbol] CH [symbol] | CH [symbol] | CH [symbol] | [symbol] Pr | (1-2-25c-2) |
| A-2 | 0 | CH [symbol] (CH [symbol] ) [symbol] CH [symbol] | CH [symbol] | CH [symbol] | Bu | (1-2-26c-2) |
| A-2 | 0 | CH [symbol] (CH [symbol] ) [symbol] CH [symbol] | CH [symbol] | CH [symbol] | [symbol] | (1-2-27c-2) |
| A-2 | 0 | CH [symbol] (CH [symbol] ) [symbol] CH [symbol] | CH [symbol] | Et | Et | (1-2-28c-2) |
| A-2 | 0 | CH [symbol] (CH [symbol] ) [symbol] CH [symbol] | CH [symbol] | CH [symbol] CH [symbol] OCH [symbol] | CH [symbol] CH [symbol] OCH [symbol] | (1-2-29c-2) |
| A-2 | 0 | CH [symbol] (CH [symbol] ) [symbol] CH [symbol] | CH [symbol] | Pr | Bu | (1-2-30c-2) |
| A-2 | 0 | CH [symbol] (CH [symbol] ) [symbol] CH [symbol] | Et | Et | Et | (1-2-31c-2) |
| A-2 | 0 | CH [symbol] (CH [symbol] ) [symbol] CH [symbol] | [symbol] Pr | [symbol] Pr | [symbol] Pr | (1-2-32c-2) |
| A-2 | 0 | CH [symbol] (CH [symbol] ) [symbol] CH [symbol] | Bu | Bu | Bu | (1-2-33c-2) |

[symbol]

indicates data missing or illegible when filed

| A | X (R [symbol] )\hat{ } | R [symbol] | R [symbol] | R [symbol] | R [symbol] | |
|---|---|---|---|---|---|---|
| A-6 | NH | CH [symbol] | CH [symbol] | CH [symbol] | CH [symbol] | (1-2-1a-6) |
| A-6 | NH | CH [symbol] | CH [symbol] | CH [symbol] | Et | (1-2-2a-6) |
| A-6 | NH | CH [symbol] | CH [symbol] | CH [symbol] | [symbol] Pr | (1-2-3a-6) |
| A-6 | NH | CH [symbol] | CH [symbol] | CH [symbol] | Bu | (1-2-4a-6) |
| A-6 | NH | CH [symbol] | CH [symbol] | CH [symbol] | [symbol] | (1-2-5a-6) |
| A-6 | NH | CH [symbol] | CH [symbol] | Et | Et | (1-2-6a-6) |
| A-6 | NH | CH [symbol] | CH [symbol] | CH [symbol] CH [symbol] OCH [symbol] | CH [symbol] CH [symbol] OCH [symbol] | (1-2-7a-6) |
| A-6 | NH | CH [symbol] | CH [symbol] | Pr | Bu | (1-2-8a-6) |
| A-6 | NH | CH [symbol] | Et | Et | Et | (1-2-9a-6) |
| A-6 | NH | CH [symbol] | [symbol] Pr | [symbol] Pr | [symbol] Pr | (1-2-10a-6) |
| A-6 | NH | CH [symbol] | CH [symbol] | Bu | Bu | (1-2-11a-6) |
| A-6 | NH | CH [symbol] CH [symbol] CH [symbol] | CH [symbol] | CH [symbol] | CH [symbol] | (1-2-12a-6) |
| A-6 | NH | CH [symbol] CH [symbol] CH [symbol] | CH [symbol] | CH [symbol] | Et | (1-2-13a-6) |
| A-6 | NH | CH [symbol] CH [symbol] CH [symbol] | CH [symbol] | CH [symbol] | [symbol] Pr | (1-2-14a-6) |
| A-6 | NH | CH [symbol] CH [symbol] CH [symbol] | CH [symbol] | CH [symbol] | Bu | (1-2-15a-6) |
| A-6 | NH | CH [symbol] CH [symbol] CH [symbol] | CH [symbol] | CH [symbol] | [symbol] | (1-2-16a-6) |
| A-6 | NH | CH [symbol] CH [symbol] CH [symbol] | CH [symbol] | Et | Et | (1-2-17a-6) |
| A-6 | NH | CH [symbol] CH [symbol] CH [symbol] | CH [symbol] | CH [symbol] CH [symbol] OCH [symbol] | CH [symbol] CH [symbol] OCH [symbol] | (1-2-18a-6) |
| A-6 | NH | CH [symbol] CH [symbol] CH [symbol] | CH [symbol] | Pr | Bu | (1-2-19a-6) |
| A-6 | NH | CH [symbol] CH [symbol] CH [symbol] | Et | Et | Et | (1-2-20a-6) |
| A-6 | NH | CH [symbol] CH [symbol] CH [symbol] | [symbol] Pr | [symbol] Pr | [symbol] Pr | (1-2-21a-6) |
| A-6 | NH | CH [symbol] CH [symbol] CH [symbol] | Bu | Bu | Bu | (1-2-22a-6) |
| A-6 | NH | CH [symbol] (CH [symbol] ) [symbol] CH [symbol] | CH [symbol] | CH [symbol] | CH [symbol] | (1-2-23a-6) |
| A-6 | NH | CH [symbol] (CH [symbol] ) [symbol] CH [symbol] | CH [symbol] | CH [symbol] | Et | (1-2-24a-6) |
| A-6 | NH | CH [symbol] (CH [symbol] ) [symbol] CH [symbol] | CH [symbol] | CH [symbol] | [symbol] Pr | (1-2-25a-6) |
| A-6 | NH | CH [symbol] (CH [symbol] ) [symbol] CH [symbol] | CH [symbol] | CH [symbol] | Bu | (1-2-26a-6) |
| A-6 | NH | CH [symbol] (CH [symbol] ) [symbol] CH [symbol] | CH [symbol] | CH [symbol] | [symbol] | (1-2-27a-6) |
| A-6 | NH | CH [symbol] (CH [symbol] ) [symbol] CH [symbol] | CH [symbol] | Et | Et | (1-2-28a-6) |
| A-6 | NH | CH [symbol] (CH [symbol] ) [symbol] CH [symbol] | CH [symbol] | CH [symbol] CH [symbol] OCH [symbol] | CH [symbol] CH [symbol] OCH [symbol] | (1-2-29a-6) |
| A-6 | NH | CH [symbol] (CH [symbol] ) [symbol] CH [symbol] | CH [symbol] | Pr | Bu | (1-2-30a-6) |
| A-6 | NH | CH [symbol] (CH [symbol] ) [symbol] CH [symbol] | Et | Et | Et | (1-2-31a-6) |
| A-6 | NH | CH [symbol] (CH [symbol] ) [symbol] CH [symbol] | [symbol] Pr | [symbol] Pr | [symbol] Pr | (1-2-32a-6) |
| A-6 | NH | CH [symbol] (CH [symbol] ) [symbol] CH [symbol] | Bu | Bu | Bu | (1-2-33a-6) |

[symbol]

indicates data missing or illegible when filed

| A | X (R [symbol] )\hat{ } | R [symbol] | R [symbol] | R [symbol] | R [symbol] | |
|---|---|---|---|---|---|---|
| A-6 | N(CH [symbol] ) | CH [symbol] | CH [symbol] | CH [symbol] | CH [symbol] | (1-2-1b-6) |
| A-6 | N(CH [symbol] ) | CH [symbol] | CH [symbol] | CH [symbol] | Et | (1-2-2b-6) |
| A-6 | N(CH [symbol] ) | CH [symbol] | CH [symbol] | CH [symbol] | [symbol] Pr | (1-2-3b-6) |
| A-6 | N(CH [symbol] ) | CH [symbol] | CH [symbol] | CH [symbol] | Bu | (1-2-4b-6) |
| A-6 | N(CH [symbol] ) | CH [symbol] | CH [symbol] | CH [symbol] | [symbol] | (1-2-5b-6) |
| A-6 | N(CH [symbol] ) | CH [symbol] | CH [symbol] | Et | Et | (1-2-6b-6) |
| A-6 | N(CH [symbol] ) | CH [symbol] | CH [symbol] | CH [symbol] CH [symbol] OCH [symbol] | CH [symbol] CH [symbol] OCH [symbol] | (1-2-7b-6) |
| A-6 | N(CH [symbol] ) | CH [symbol] | CH [symbol] | Pr | Bu | (1-2-8b-6) |
| A-6 | N(CH [symbol] ) | CH [symbol] | Et | Et | Et | (1-2-9b-6) |
| A-6 | N(CH [symbol] ) | CH [symbol] | [symbol] Pr | [symbol] Pr | [symbol] Pr | (1-2-10b-6) |
| A-6 | N(CH [symbol] ) | CH [symbol] | Bu | Bu | Bu | (1-2-11b-6) |
| A-6 | N(CH [symbol] ) | CH [symbol] CH [symbol] CH [symbol] | CH [symbol] | CH [symbol] | CH [symbol] | (1-2-12b-6) |
| A-6 | N(CH [symbol] ) | CH [symbol] CH [symbol] CH [symbol] | CH [symbol] | CH [symbol] | Et | (1-2-13b-6) |
| A-6 | N(CH [symbol] ) | CH [symbol] CH [symbol] CH [symbol] | CH [symbol] | CH [symbol] | [symbol] Pr | (1-2-14b-6) |
| A-6 | N(CH [symbol] ) | CH [symbol] CH [symbol] CH [symbol] | CH [symbol] | CH [symbol] | Bu | (1-2-15b-6) |
| A-6 | N(CH [symbol] ) | CH [symbol] CH [symbol] CH [symbol] | CH [symbol] | CH [symbol] | [symbol] | (1-2-16b-6) |
| A-6 | N(CH [symbol] ) | CH [symbol] CH [symbol] CH [symbol] | CH [symbol] | Et | Et | (1-2-17b-6) |
| A-6 | N(CH [symbol] ) | CH [symbol] CH [symbol] CH [symbol] | CH [symbol] | CH [symbol] CH [symbol] OCH [symbol] | CH [symbol] CH [symbol] OCH [symbol] | (1-2-18b-6) |
| A-6 | N(CH [symbol] ) | CH [symbol] CH [symbol] CH [symbol] | CH [symbol] | Pr | Bu | (1-2-19b-6) |
| A-6 | N(CH [symbol] ) | CH [symbol] CH [symbol] CH [symbol] | Et | Et | Et | (1-2-20b-6) |
| A-6 | N(CH [symbol] ) | CH [symbol] CH [symbol] CH [symbol] | [symbol] Pr | [symbol] Pr | [symbol] Pr | (1-2-21b-6) |
| A-6 | N(CH [symbol] ) | CH [symbol] CH [symbol] CH [symbol] | Bu | Bu | Bu | (1-2-22b-6) |
| A-6 | N(CH [symbol] ) | CH [symbol] (CH [symbol] ) [symbol] CH [symbol] | CH [symbol] | CH [symbol] | CH [symbol] | (1-2-23b-6) |
| A-6 | N(CH [symbol] ) | CH [symbol] (CH [symbol] ) [symbol] CH [symbol] | CH [symbol] | CH [symbol] | Et | (1-2-24b-6) |
| A-6 | N(CH [symbol] ) | CH [symbol] (CH [symbol] ) [symbol] CH [symbol] | CH [symbol] | CH [symbol] | [symbol] Pr | (1-2-25b-6) |
| A-6 | N(CH [symbol] ) | CH [symbol] (CH [symbol] ) [symbol] CH [symbol] | CH [symbol] | CH [symbol] | Bu | (1-2-26b-6) |
| A-6 | N(CH [symbol] ) | CH [symbol] (CH [symbol] ) [symbol] CH [symbol] | CH [symbol] | CH [symbol] | [symbol] | (1-2-27b-6) |
| A-6 | N(CH [symbol] ) | CH [symbol] (CH [symbol] ) [symbol] CH [symbol] | CH [symbol] | Et | Et | (1-2-28b-6) |
| A-6 | N(CH [symbol] ) | CH [symbol] (CH [symbol] ) [symbol] CH [symbol] | CH [symbol] | CH [symbol] CH [symbol] OCH [symbol] | CH [symbol] CH [symbol] OCH [symbol] | (1-2-29b-6) |
| A-6 | N(CH [symbol] ) | CH [symbol] (CH [symbol] ) [symbol] CH [symbol] | CH [symbol] | Pr | Bu | (1-2-30b-6) |
| A-6 | N(CH [symbol] ) | CH [symbol] (CH [symbol] ) [symbol] CH [symbol] | Et | Et | Et | (1-2-31b-6) |
| A-6 | N(CH [symbol] ) | CH [symbol] (CH [symbol] ) [symbol] CH [symbol] | [symbol] Pr | [symbol] Pr | [symbol] Pr | (1-2-32b-6) |
| A-6 | N(CH [symbol] ) | CH [symbol] (CH [symbol] ) [symbol] CH [symbol] | Bu | Bu | Bu | (1-2-33b-6) |

[symbol]

indicates data missing or illegible when filed

| A | X (R [symbol] ) [symbol] | R [symbol] | R [symbol] | R [symbol] | R [symbol] | |
|---|---|---|---|---|---|---|
| A-6 | 0 | CH [symbol] | CH [symbol] | CH [symbol] | CH [symbol] | (1-2-1e-6) |
| A-6 | 0 | CH [symbol] | CH [symbol] | CH [symbol] | Et | (1-2-2e-6) |
| A-6 | 0 | CH [symbol] | CH [symbol] | CH [symbol] | [symbol] Pr | (1-2-3e-6) |
| A-6 | 0 | CH [symbol] | CH [symbol] | CH [symbol] | Bu | (1-2-4e-6) |
| A-6 | 0 | CH [symbol] | CH [symbol] | CH [symbol] | [symbol] | (1-2-5e-6) |
| A-6 | 0 | CH [symbol] | CH [symbol] | Et | Et | (1-2-6e-6) |
| A-6 | 0 | CH [symbol] | CH [symbol] | CH [symbol] CH [symbol] OCH [symbol] | CH [symbol] CH [symbol] OCH [symbol] | (1-2-7e-6) |
| A-6 | 0 | CH [symbol] | CH [symbol] | Pr | Bu | (1-2-8e-6) |
| A-6 | 0 | CH [symbol] | Et | Et | Et | (1-2-9e-6) |
| A-6 | 0 | CH [symbol] | [symbol] Pr | [symbol] Pr | [symbol] Pr | (1-2-10e-6) |
| A-6 | 0 | CH [symbol] | Bu | Bu | Bu | (1-2-11e-6) |
| A-6 | 0 | CH [symbol] CH [symbol] CH [symbol] | CH [symbol] | CH [symbol] | CH [symbol] | (1-2-12e-6) |
| A-6 | 0 | CH [symbol] CH [symbol] CH [symbol] | CH [symbol] | CH [symbol] | Et | (1-2-13e-6) |
| A-6 | 0 | CH [symbol] CH [symbol] CH [symbol] | CH [symbol] | CH [symbol] | [symbol] Pr | (1-2-14e-6) |
| A-6 | 0 | CH [symbol] CH [symbol] CH [symbol] | CH [symbol] | CH [symbol] | Bu | (1-2-15e-6) |
| A-6 | 0 | CH [symbol] CH [symbol] CH [symbol] | CH [symbol] | CH [symbol] | [symbol] | (1-2-16e-6) |
| A-6 | 0 | CH [symbol] CH [symbol] CH [symbol] | CH [symbol] | Et | Et | (1-2-17e-6) |
| A-6 | 0 | CH [symbol] CH [symbol] CH [symbol] | CH [symbol] | CH [symbol] CH [symbol] OCH [symbol] | CH [symbol] CH [symbol] OCH [symbol] | (1-2-18e-6) |
| A-6 | 0 | CH [symbol] CH [symbol] CH [symbol] | CH [symbol] | Pr | Bu | (1-2-19e-6) |
| A-6 | 0 | CH [symbol] CH [symbol] CH [symbol] | Et | Et | Et | (1-2-20e-6) |
| A-6 | 0 | CH [symbol] CH [symbol] CH [symbol] | [symbol] Pr | [symbol] Pr | [symbol] Pr | (1-2-21e-6) |
| A-6 | 0 | CH [symbol] CH [symbol] CH [symbol] | Bu | Bu | Bu | (1-2-22e-6) |
| A-6 | 0 | CH [symbol] (CH [symbol] ) [symbol] CH [symbol] | CH [symbol] | CH [symbol] | CH [symbol] | (1-2-23e-6) |
| A-6 | 0 | CH [symbol] (CH [symbol] ) [symbol] CH [symbol] | CH [symbol] | CH [symbol] | Et | (1-2-24e-6) |
| A-6 | 0 | CH [symbol] (CH [symbol] ) [symbol] CH [symbol] | CH [symbol] | CH [symbol] | [symbol] Pr | (1-2-25e-6) |
| A-6 | 0 | CH [symbol] (CH [symbol] ) [symbol] CH [symbol] | CH [symbol] | CH [symbol] | Bu | (1-2-26e-6) |
| A-6 | 0 | CH [symbol] (CH [symbol] ) [symbol] CH [symbol] | CH [symbol] | CH [symbol] | [symbol] | (1-2-27e-6) |
| A-6 | 0 | CH [symbol] (CH [symbol] ) [symbol] CH [symbol] | CH [symbol] | Et | Et | (1-2-28e-6) |
| A-6 | 0 | CH [symbol] (CH [symbol] ) [symbol] CH [symbol] | CH [symbol] | CH [symbol] CH [symbol] OCH [symbol] | CH [symbol] CH [symbol] OCH [symbol] | (1-2-29e-6) |
| A-6 | 0 | CH [symbol] (CH [symbol] ) [symbol] CH [symbol] | CH [symbol] | Pr | Bu | (1-2-30e-6) |
| A-6 | 0 | CH [symbol] (CH [symbol] ) [symbol] CH [symbol] | Et | Et | Et | (1-2-31e-6) |
| A-6 | 0 | CH [symbol] (CH [symbol] ) [symbol] CH [symbol] | [symbol] Pr | [symbol] Pr | [symbol] Pr | (1-2-32e-6) |
| A-6 | 0 | CH [symbol] (CH [symbol] ) [symbol] CH [symbol] | Bu | Bu | Bu | (1-2-33e-6) |

[symbol]

indicates data missing or illegible when filed

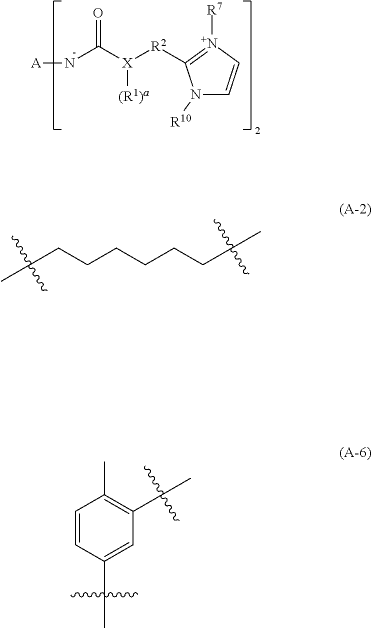

| A | X(R [symbol] ) [symbol] | R [symbol] | R [symbol] | R [symbol] | |
|---|---|---|---|---|---|
| A-2 | NH | CH [symbol] | CH [symbol] | CH [symbol] | (1-3-1a-2) |
| A-2 | NH | CH [symbol] | CH [symbol] | Et | (1-3-2a-2) |
| A-2 | NH | CH [symbol] | CH [symbol] | ⁱPr | (1-3-3a-2) |
| A-2 | NH | CH [symbol] | CH [symbol] | Bu | (1-3-4a-2) |
| A-2 | NH | CH [symbol] | CH [symbol] | Bnz | (1-3-5a-2) |
| A-2 | NH | CH [symbol] | CH [symbol] | CH [symbol] CH [symbol] OCH [symbol] | (1-3-6a-2) |
| A-2 | NH | CH [symbol] | Et | Et | (1-3-7a-2) |
| A-2 | NH | CH [symbol] CH [symbol] | CH [symbol] | CH [symbol] | (1-3-8a-2) |
| A-2 | NH | CH [symbol] CH [symbol] | CH [symbol] | Et | (1-3-9a-2) |
| A-2 | NH | CH [symbol] CH [symbol] | CH [symbol] | ⁱPr | (1-3-10a-2) |
| A-2 | NH | CH [symbol] CH [symbol] | CH [symbol] | Bu | (1-3-11a-2) |
| A-2 | NH | CH [symbol] CH [symbol] | CH [symbol] | Bnz | (1-3-12a-2) |
| A-2 | NH | CH [symbol] CH [symbol] | CH [symbol] | CH [symbol] CH [symbol] OCH [symbol] | (1-3-13a-2) |
| A-2 | NH | CH [symbol] CH [symbol] | Et | Et | (1-3-14a-2) |
| A-2 | NH | CH [symbol] (CH [symbol] ) [symbol] CH [symbol] | CH [symbol] | CH [symbol] | (1-3-15a-2) |
| A-2 | NH | CH [symbol] (CH [symbol] ) [symbol] CH [symbol] | CH [symbol] | Et | (1-3-16a-2) |
| A-2 | NH | CH [symbol] (CH [symbol] ) [symbol] CH [symbol] | CH [symbol] | ⁱPr | (1-3-17a-2) |
| A-2 | NH | CH [symbol] (CH [symbol] ) [symbol] CH [symbol] | CH [symbol] | Bu | (1-3-18a-2) |
| A-2 | NH | CH [symbol] (CH [symbol] ) [symbol] CH [symbol] | CH [symbol] | Bnz | (1-3-19a-2) |
| A-2 | NH | CH [symbol] (CH [symbol] ) [symbol] CH [symbol] | CH [symbol] | CH [symbol] CH [symbol] OCH [symbol] | (1-3-20a-2) |
| A-2 | NH | CH [symbol] (CH [symbol] ) [symbol] CH [symbol] | Et | Et | (1-3-21a-2) |
| A-6 | NH | CH [symbol] | CH [symbol] | CH [symbol] | (1-3-1a-6) |
| A-6 | NH | CH [symbol] | CH [symbol] | Et | (1-3-2a-6) |
| A-6 | NH | CH [symbol] | CH [symbol] | ⁱPr | (1-3-3a-6) |
| A-6 | NH | CH [symbol] | CH [symbol] | Bu | (1-3-4a-6) |
| A-6 | NH | CH [symbol] | CH [symbol] | Bnz | (1-3-5a-6) |
| A-6 | NH | CH [symbol] | CH [symbol] | CH [symbol] CH [symbol] OCH [symbol] | (1-3-6a-6) |
| A-6 | NH | CH [symbol] | Et | Et | (1-3-7a-6) |
| A-6 | NH | CH₂CH₂ | CH₃ | CH₃ | (1-3-8a-6) |
| A-6 | NH | CH₂CH₂ | CH₃ | Et | (1-3-9a-6) |
| A-6 | NH | CH₂CH₂ | CH₃ | ⁱPr | (1-3-10a-6) |
| A-6 | NH | CH₂CH [symbol] | CH₃ | Bu | (1-3-11a-6) |
| A-6 | NH | CH₂CH₂ | CH₃ | Bnz | (1-3-12a-6) |
| A-6 | NH | CH₂CH₂ | CH₃ | CH₂CH₂OCH₃ | (1-3-13a-6) |
| A-6 | NH | CH₂CH₂ | Et | Et | (1-3-14a-6) |
| A-6 | NH | CH₂(CH₂)₄CH₂ | CH₃ | CH₃ | (1-3-15a-6) |
| A-6 | NH | CH₂(CH₂)₄CH₂ | CH3 | Et | (1-3-16a-6) |
| A-6 | NH | CH₂(CH₂)₄CH₂ | CH3 | ⁱPr | (1-3-17a-6) |
| A-6 | NH | CH₂(CH₂)₄CH₂ | CH3 | Bu | (1-3-18a-6) |
| A-6 | NH | CH₂(CH₂)₄CH₂ | CH3 | Bnz | (1-3-19a-6) |
| A-6 | NH | CH₂(CH₂)₄CH₂ | CH3 | CH₂CH [symbol] OCH₃ | (1-3-20a-6) |
| A-6 | NH | CH₂(CH₂)₄CH₂ | Et | Et | (1-3-21a-6) |

[symbol]

indicates data missing or illegible when filed

| A | X(R [symbol] ) [symbol] | R [symbol] | R [symbol] | R [symbol] | |
|---|---|---|---|---|---|
| A-2 | N(CH₃) | CH₂ | CH₃ | CH₃ | (1-3-1b-2) |
| A-2 | N(CH₃) | CH₂ | CH₃ | Et | (1-3-2b-2) |
| A-2 | N(CH₃) | CH₂ | CH₃ | ⁱPr | (1-3-3b-2) |
| A-2 | N(CH₃) | CH₂ | CH₃ | Bu | (1-3-4b-2) |
| A-2 | N(CH₃) | CH₂ | CH₃ | Bnz | (1-3-5b-2) |
| A-2 | N(CH₃) | CH₂ | CH₃ | CH₂CH₂OCH₃ | (1-3-6b-2) |
| A-2 | N(CH₃) | CH₂ | Et | Et | (1-3-7b-2) |
| A-2 | N(CH₃) | CH₂CH₂ | CH₃ | CH₃ | (1-3-8b-2) |
| A-2 | N(CH₃) | CH [symbol] CH₂ | CH₃ | Et | (1-3-9b-2) |
| A-2 | N(CH₃) | CH₂CH₂ | CH₃ | ⁱPr | (1-3-10b-2) |
| A-2 | N(CH₃) | CH₂CH₂ | CH₃ | Bu | (1-3-11b-2) |
| A-2 | N(CH₃) | CH₂CH₂ | CH₃ | Bnz | (1-3-12b-2) |
| A-2 | N(CH₃) | CH [symbol] CH₂ | CH₃ | CH₂CH₂OCH₃ | (1-3-13b-2) |
| A-2 | N(CH₃) | CH₂CH₂ | Et | Et | (1-3-14b-2) |
| A-2 | N(CH₃) | CH [symbol] (CH₂)₄CH₂ | CH₃ | CH₃ | (1-3-15b-2) |
| A-2 | N(CH₃) | CH₂(CH₂)₄CH₂ | CH₃ | Et | (1-3-16b-2) |
| A-2 | N(CH₃) | CH₂(CH₂)₄CH₂ | CH₃ | ⁱPr | (1-3-17b-2) |
| A-2 | N(CH₃) | CH [symbol] (CH₂)₄CH₂ | CH₃ | Bu | (1-3-18b-2) |
| A-2 | N(CH₃) | CH₂(CH₂)₄CH₂ | CH₃ | Bnz | (1-3-19b-2) |
| A-2 | N(CH₃) | CH₂(CH₂)₄CH₂ | CH₃ | CH₂CH₂OCH₃ | (1-3-20b-2) |
| A-2 | N(CH₃) | CH [symbol] (CH₂)₄CH₂ | Et | Et | (1-3-21b-2) |

[symbol]

indicates data missing or illegible when filed

| A | X(R¹) [symbol] | R² | R⁷ | R¹⁰ | |
|---|---|---|---|---|---|
| A-6 | N(CH₃) | CH₂ | CH₃ | CH₃ | (1-3-1b-6) |
| A-6 | N(CH₃) | CH₂ | CH₃ | Et | (1-3-2b-6) |
| A-6 | N(CH₃) | CH₂ | CH₃ | ⁱPr | (1-3-3b-6) |
| A-6 | N(CH₃) | CH₂ | CH₃ | Bu | (1-3-4b-6) |
| A-6 | N(CH₃) | CH₂ | CH₃ | Bnz | (1-3-5b-6) |
| A-6 | N(CH₃) | CH₂ | CH₃ | CH₂CH₂OCH₃ | (1-3-6b-6) |
| A-6 | N(CH₃) | CH₂ | Et | Et | (1-3-7b-6) |
| A-6 | N(CH₃) | CH₂CH [symbol] | CH₃ | CH₃ | (1-3-8b-6) |
| A-6 | N(CH₃) | CH₂CH₂ | CH₃ | Et | (1-3-9b-6) |
| A-6 | N(CH₃) | CH₂CH₂ | CH₃ | ⁱPr | (1-3-10b-6) |
| A-6 | N(CH₃) | CH₂CH [symbol] | CH₃ | Bu | (1-3-11b-6) |
| A-6 | N(CH₃) | CH₂CH [symbol] | CH₃ | Bnz | (1-3-12b-6) |
| A-6 | N(CH₃) | CH₂CH₂ | CH₃ | CH₂CH₂OCH₃ | (1-3-13b-6) |
| A-6 | N(CH₃) | CH₂CH₂ | Et | Et | (1-3-14b-6) |
| A-6 | N(CH₃) | CH₂(CH₂)₄CH₂ | CH₃ | CH₃ | (1-3-15b-6) |
| A-6 | N(CH₃) | CH₂(CH₂)₄CH₂ | CH₃ | Et | (1-3-16b-6) |
| A-6 | N(CH₃) | CH₂(CH₂)₄CH₂ | CH₃ | ⁱPr | (1-3-17b-6) |
| A-6 | N(CH₃) | CH₂(CH₂)₄CH₂ | CH₃ | Bu | (1-3-18b-6) |
| A-6 | N(CH₃) | CH₂(CH₂)₄CH₂ | CH₃ | Bnz | (1-3-19b-6) |
| A-6 | N(CH₃) | CH₂(CH₂)₄CH₂ | CH₃ | CH₂CH₂OCH₃ | (1-3-20b-6) |
| A-6 | N(CH₃) | CH₂(CH₂)₄CH₂ | Et | Et | (1-3-21b-6) |

[symbol]

indicates data missing or illegible when filed

| A | X(R¹) [symbol] | R² | R⁷ | R¹⁰ | |
|---|---|---|---|---|---|
| A-2 | O | CH₂ | CH₃ | CH₃ | (1-3-1c-2) |
| A-2 | O | CH₂ | CH₃ | Et | (1-3-2c-2) |
| A-2 | O | CH₂ | CH₃ | ⁱPr | (1-3-3c-2) |
| A-2 | O | CH₂ | CH₃ | Bu | (1-3-4c-2) |
| A-2 | O | CH₂ | CH₃ | Bnz | (1-3-5c-2) |
| A-2 | O | CH₂ | CH₃ | CH₂CH₃OCH₃ | (1-3-6c-2) |
| A-2 | O | CH₂ | Et | Et | (1-3-7c-2) |
| A-2 | O | CH₂CH [symbol] | CH₃ | CH₃ | (1-3-8c-2) |
| A-2 | O | CH₂CH₂ | CH₃ | Et | (1-3-9c-2) |
| A-2 | O | CH₂CH₂ | CH₃ | ⁱPr | (1-3-10c-2) |
| A-2 | O | CH₂CH₂ | CH₃ | Bu | (1-3-11c-2) |
| A-2 | O | CH₂CH₂ | CH₃ | Bnz | (1-3-12c-2) |
| A-2 | O | CH₂CH₂ | CH₃ | CH₃CH₂OCH₃ | (1-3-13c-2) |
| A-2 | O | CH₂CH₂ | Et | Et | (1-3-14c-2) |
| A-2 | O | CH₂(CH₂)₄CH₂ | CH₃ | CH₃ | (1-3-15c-2) |
| A-2 | O | CH₂(CH₂)₄CH₂ | CH₃ | Et | (1-3-16c-2) |
| A-2 | O | CH₂(CH₂)₄CH₂ | CH₃ | ⁱPr | (1-3-17c-2) |
| A-2 | O | CH₂(CH₂)₄CH₂ | CH₃ | Bu | (1-3-18c-2) |
| A-2 | O | CH₂(CH₂)₄CH₂ | CH₃ | Bnz | (1-3-19c-2) |
| A-2 | O | CH₂(CH₂)₄CH₂ | CH₃ | CH₂CH₃OCH₃ | (1-3-20c-2) |
| A-2 | O | CH₂(CH₂)₄CH₂ | Et | Et | (1-3-21c-2) |

[symbol]

indicates data missing or illegible when filed

| A | X(R¹) [symbol] | R² | R⁷ | R¹⁰ | |
|---|---|---|---|---|---|
| A-6 | O | CH₂ | CH₃ | CH₃ | (1-3-1c-6) |
| A-6 | O | CH₂ | CH₃ | Et | (1-3-2c-6) |
| A-6 | O | CH₂ | CH₃ | ⁱPr | (1-3-3c-6) |
| A-6 | O | CH₂ | CH₃ | Bu | (1-3-4c-6) |
| A-6 | O | CH₂ | CH₃ | Bnz | (1-3-5c-6) |
| A-6 | O | CH₂ | CH₃ | CH₂CH₂OCH₃ | (1-3-6c-6) |
| A-6 | O | CH₂ | Et | Et | (1-3-7c-6) |
| A-6 | O | CH₂CH₂ | CH₃ | CH₃ | (1-3-8c-6) |
| A-6 | O | CH₂CH₂ | CH₃ | Et | (1-3-9c-6) |
| A-6 | O | CH₂CH₂ | CH₃ | ⁱPr | (1-3-10c-6) |
| A-6 | O | CH₂CH₂ | CH₃ | Bu | (1-3-11c-6) |
| A-6 | O | CH₂CH₂ | CH₃ | Bnz | (1-3-12c-6) |
| A-6 | O | CH₂CH₂ | CH₃ | CH₂CH₂OCH₃ | (1-3-13c-6) |
| A-6 | O | CH₂CH₂ | Et | Et | (1-3-14c-6) |
| A-6 | O | CH₂(CH₂)₄CH₂ | CH₃ | CH₃ | (1-3-15c-6) |
| A-6 | O | CH₂(CH₂)₄CH [symbol] | CH₃ | Et | (1-3-16c-6) |
| A-6 | O | CH₂(CH₂)₄CH₂ | CH₃ | ⁱPr | (1-3-17c-6) |
| A-6 | O | CH₂(CH₂)₄CH₂ | CH₃ | Bu | (1-3-18c-6) |
| A-6 | O | CH₂(CH₂) [symbol] CH₂ | CH₃ | Bnz | (1-3-19c-6) |
| A-6 | O | CH₂(CH₂)₄CH₂ | CH₃ | CH₂CH₂OCH₃ | (1-3-20c-6) |
| A-6 | O | CH₂(CH₂)₄CH [symbol] | Et | Et | (1-3-21c-6) |

[symbol]

indicates data missing or illegible when filed

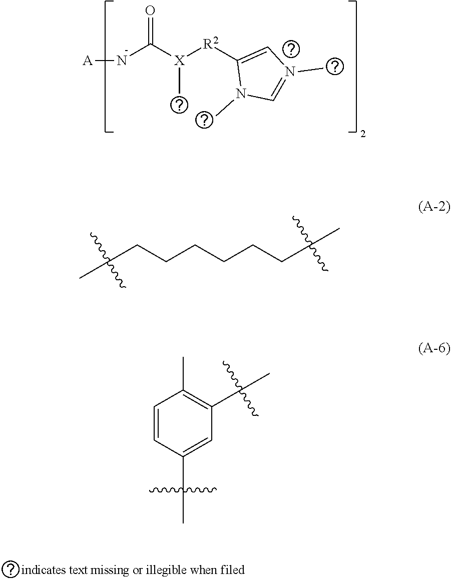

| A | X(R¹) [symbol] | R² | R⁷ | R¹⁰ | |
|---|---|---|---|---|---|
| A-2 | NH | CH [symbol] | CH [symbol] | CH [symbol] | (1-3-22a-2) |
| A-2 | NH | CH [symbol] | CH [symbol] | Et | (1-3-23a-2) |
| A-2 | NH | CH [symbol] | CH [symbol] | ⁱPr | (1-3-24a-2) |
| A-2 | NH | CH [symbol] | CH [symbol] | Bu | (1-3-25a-2) |
| A-2 | NH | CH [symbol] | CH [symbol] | Bnz | (1-3-26a-2) |
| A-2 | NH | CH [symbol] | CH [symbol] | CH [symbol] CH [symbol] OCH [symbol] | (1-3-27a-2) |
| A-2 | NH | CH [symbol] | Et | Et | (1-3-28a-2) |
| A-2 | NH | CH [symbol] CH [symbol] | CH [symbol] | CH [symbol] | (1-3-29a-2) |
| A-2 | NH | CH [symbol] CH [symbol] | CH [symbol] | Et | (1-3-30a-2) |
| A-2 | NH | CH [symbol] CH [symbol] | CH [symbol] | ⁱPr | (1-3-31a-2) |
| A-2 | NH | CH [symbol] CH [symbol] | CH [symbol] | Bu | (1-3-32a-2) |
| A-2 | NH | CH [symbol] CH [symbol] | CH [symbol] | Bnz | (1-3-33a-2) |
| A-2 | NH | CH [symbol] CH [symbol] | CH [symbol] | CH [symbol] CH [symbol] OCH [symbol] | (1-3-34a-2) |
| A-2 | NH | CH [symbol] CH [symbol] | Et | Et | (1-3-35a-2) |
| A-2 | NH | CH [symbol] (CH [symbol] ) [symbol] CH [symbol] | CH [symbol] | CH [symbol] | (1-3-36a-2) |
| A-2 | NH | CH [symbol] (CH [symbol] ) [symbol] CH [symbol] | CH [symbol] | Et | (1-3-37a-2) |
| A-2 | NH | CH [symbol] (CH [symbol] ) [symbol] CH [symbol] | CH [symbol] | ⁱPr | (1-3-38a-2) |
| A-2 | NH | CH [symbol] (CH [symbol] ) [symbol] CH [symbol] | CH [symbol] | Bu | (1-3-39a-2) |
| A-2 | NH | CH [symbol] (CH [symbol] ) [symbol] CH [symbol] | CH [symbol] | Bnz | (1-3-40a-2) |
| A-2 | NH | CH [symbol] (CH [symbol] ) [symbol] CH [symbol] | CH [symbol] | CH [symbol] CH [symbol] OCH [symbol] | (1-3-41a-2) |
| A-2 | NH | CH [symbol] (CH [symbol] ) [symbol] CH [symbol] | Et | Et | (1-3-42a-2) |
| A-6 | NH | CH [symbol] | CH [symbol] | CH [symbol] | (1-3-22a-6) |
| A-6 | NH | CH [symbol] | CH [symbol] | Et | (1-3-23a-6) |
| A-6 | NH | CH [symbol] | CH [symbol] | ⁱPr | (1-3-24a-6) |
| A-6 | NH | CH [symbol] | CH [symbol] | Bu | (1-3-25a-6) |
| A-6 | NH | CH [symbol] | CH [symbol] | Bnz | (1-3-26a-6) |
| A-6 | NH | CH [symbol] | CH [symbol] | CH [symbol] CH [symbol] OCH [symbol] | (1-3-27a-6) |
| A-6 | NH | CH [symbol] | Et | Et | (1-3-28a-6) |
| A-6 | NH | CH [symbol] CH [symbol] | CH [symbol] | CH [symbol] | (1-3-29a-6) |
| A-6 | NH | CH₂CH₂ | CH₃ | Et | (1-3-30a-6) |
| A-6 | NH | CH₂CH₂ | CH₃ | ⁱPr | (1-3-31a-6) |
| A-6 | NH | CH₂CH₂ | CH₃ | Bu | (1-3-32a-6) |
| A-6 | NH | CH₂CH₂ | CH₃ | Bnz | (1-3-33a-6) |
| A-6 | NH | CH₂CH₂ | CH₃ | CH₂CH₂OCH₃ | (1-3-34a-6) |
| A-6 | NH | CH₂CH₂ | Et | Et | (1-3-35a-6) |
| A-6 | NH | CH₂(CH₂)₄CH₂ | CH₃ | CH₃ | (1-3-36a-6) |
| A-6 | NH | CH₂(CH₂)₄CH₂ | CH₃ | Et | (1-3-37a-6) |
| A-6 | NH | CH₂(CH₂)₄CH₂ | CH₃ | ⁱPr | (1-3-38a-6) |
| A-6 | NH | CH₂(CH₂)₄CH₂ | CH₃ | Bu | (1-3-39a-6) |
| A-6 | NH | CH₂(CH₂)₄CH₂ | CH₃ | Bnz | (1-3-40a-6) |
| A-6 | NH | CH₂(CH₂)₄CH₂ | CH₃ | CH₂CH₂OCH₃ | (1-3-41a-6) |
| A-6 | NH | CH₂(CH₂)₄CH₂ | Et | Et | (1-3-42a-6) |

[symbol]

indicates data missing or illegible when filed

| A | X(R [symbol] ) [symbol] | R [symbol] | R [symbol] | R [symbol] | |
|---|---|---|---|---|---|
| A-2 | N(CH [symbol] ) | CH [symbol] | CH [symbol] | CH [symbol] | (1-3-22b-2) |
| A-2 | N(CH [symbol] ) | CH [symbol] | CH [symbol] | Et | (1-3-23b-2) |
| A-2 | N(CH [symbol] ) | CH [symbol] | CH [symbol] | ⁱPr | (1-3-24b-2) |
| A-2 | N(CH [symbol] ) | CH [symbol] | CH [symbol] | Bu | (1-3-25b-2) |
| A-2 | N(CH [symbol] ) | CH [symbol] | CH [symbol] | Bnz | (1-3-26b-2) |
| A-2 | N(CH [symbol] ) | CH [symbol] | CH [symbol] | CH [symbol] CH [symbol] OCH [symbol] | (1-3-27b-2) |
| A-2 | N(CH [symbol] ) | CH [symbol] | Et | Et | (1-3-28b-2) |
| A-2 | N(CH [symbol] ) | CH [symbol] CH [symbol] | CH [symbol] | CH [symbol] | (1-3-29b-2) |
| A-2 | N(CH [symbol] ) | CH [symbol] CH [symbol] | CH [symbol] | Et | (1-3-30b-2) |
| A-2 | N(CH [symbol] ) | CH [symbol] CH [symbol] | CH [symbol] | ⁱPr | (1-3-31b-2) |
| A-2 | N(CH [symbol] ) | CH [symbol] CH [symbol] | CH [symbol] | Bu | (1-3-32b-2) |
| A-2 | N(CH [symbol] ) | CH [symbol] CH [symbol] | CH [symbol] | Bnz | (1-3-33b-2) |
| A-2 | N(CH [symbol] ) | CH [symbol] CH [symbol] | CH [symbol] | CH [symbol] CH [symbol] OCH [symbol] | (1-3-34b-2) |
| A-2 | N(CH [symbol] ) | CH [symbol] CH [symbol] | Et | Et | (1-3-35b-2) |
| A-2 | N(CH [symbol] ) | CH [symbol] (CH [symbol] ) [symbol] CH [symbol] | CH [symbol] | CH [symbol] | (1-3-36b-2) |
| A-2 | N(CH [symbol] ) | CH [symbol] (CH [symbol] ) [symbol] CH [symbol] | CH [symbol] | Et | (1-3-37b-2) |
| A-2 | N(CH [symbol] ) | CH [symbol] (CH [symbol] ) [symbol] CH [symbol] | CH [symbol] | ⁱPr | (1-3-38b-2) |
| A-2 | N(CH [symbol] ) | CH [symbol] (CH [symbol] ) [symbol] CH [symbol] | CH [symbol] | Bu | (1-3-39b-2) |
| A-2 | N(CH [symbol] ) | CH [symbol] (CH [symbol] ) [symbol] CH [symbol] | CH [symbol] | Bnz | (1-3-40b-2) |
| A-2 | N(CH [symbol] ) | CH [symbol] (CH [symbol] ) [symbol] CH [symbol] | CH [symbol] | CH [symbol] CH [symbol] OCH [symbol] | (1-3-41b-2) |
| A-2 | N(CH [symbol] ) | CH [symbol] (CH [symbol] ) [symbol] CH [symbol] | Et | Et | (1-3-42b-2) |
| A-6 | N(CH [symbol] ) | CH [symbol] | CH [symbol] | CH [symbol] | (1-3-22b-6) |
| A-6 | N(CH [symbol] ) | CH [symbol] | CH [symbol] | Et | (1-3-23b-6) |
| A-6 | N(CH [symbol] ) | CH [symbol] | CH [symbol] | ⁱPr | (1-3-24b-6) |
| A-6 | N(CH [symbol] ) | CH [symbol] | CH [symbol] | Bu | (1-3-25b-6) |
| A-6 | N(CH [symbol] ) | CH [symbol] | CH [symbol] | Bnz | (1-3-26b-6) |
| A-6 | N(CH [symbol] ) | CH [symbol] | CH [symbol] | CH [symbol] CH [symbol] OCH [symbol] | (1-3-27b-6) |
| A-6 | N(CH [symbol] ) | CH [symbol] | Et | Et | (1-3-28b-6) |
| A-6 | N(CH [symbol] ) | CH [symbol] CH [symbol] | CH [symbol] | CH [symbol] | (1-3-29b-6) |
| A-6 | N(CH [symbol] ) | CH [symbol] CH [symbol] | CH [symbol] | Et | (1-3-30b-6) |
| A-6 | N(CH [symbol] ) | CH [symbol] CH [symbol] | CH [symbol] | ⁱPr | (1-3-32b-6) |
| A-6 | N(CH [symbol] ) | CH [symbol] CH [symbol] | CH [symbol] | Bu | (1-3-33b-6) |
| A-6 | N(CH [symbol] ) | CH [symbol] CH [symbol] | CH [symbol] | CH [symbol] CH [symbol] OCH [symbol] | (1-3-34b-6) |
| A-6 | N(CH [symbol] ) | CH [symbol] CH [symbol] | Et | Et | (1-3-35b-6) |
| A-6 | N(CH [symbol] ) | CH [symbol] (CH [symbol] ) [symbol] CH [symbol] | CH [symbol] | CH [symbol] | (1-3-36b-6) |
| A-6 | N(CH [symbol] ) | CH [symbol] (CH [symbol] ) [symbol] CH [symbol] | CH [symbol] | Et | (1-3-37b-6) |
| A-6 | N(CH [symbol] ) | CH₂(CH₂)₄CH₂ | CH₃ | ⁱPr | (1-3-38b-6) |
| A-6 | N(CH [symbol] ) | CH₂(CH₂)₄CH₂ | CH₃ | Bu | (1-3-39b-6) |
| A-6 | N(CH [symbol] ) | CH₂(CH₂)₄CH₂ | CH₃ | Bnz | (1-3-40b-6) |
| A-6 | N(CH [symbol] ) | CH₂(CH₂)₄CH₂ | CH₃ | CH₂CH₂OCH₃ | (1-3-41b-6) |
| A-6 | N(CH [symbol] ) | CH₂(CH₂)₄CH₂ | Et | Et | (1-3-42b-6) |

[symbol]

indicates data missing or illegible when filed

| A | X(R¹) [symbol] | R² | R⁷ | R¹⁰ | |
|---|---|---|---|---|---|
| A-2 | O | CH₂ | CH₃ | CH₃ | (1-3-22c-2) |
| A-2 | O | CH₂ | CH₃ | Et | (1-3-23c-2) |
| A-2 | O | CH₂ | CH₃ | ⁱPr | (1-3-24c-2) |
| A-2 | O | CH₂ | CH₃ | Bu | (1-3-25c-2) |
| A-2 | O | CH₂ | CH₃ | Bnz | (1-3-26c-2) |
| A-2 | O | CH₂ | CH₃ | CH₂CH₂OCH₃ | (1-3-27c-2) |
| A-2 | O | CH₂ | Et | Et | (1-3-28c-2) |
| A-2 | O | CH₂CH₂ | CH₃ | CH₃ | (1-3-29c-2) |
| A-2 | O | CH₂CH₂ | CH₃ | Et | (1-3-30c-2) |
| A-2 | O | CH₂CH₂ | CH₃ | ⁱPr | (1-3-31c-2) |
| A-2 | O | CH₂CH₂ | CH₃ | Bu | (1-3-32c-2) |
| A-2 | O | CH₂CH₂ | CH₃ | Bnz | (1-3-33c-2) |
| A-2 | O | CH₂CH₂ | CH₃ | CH₂CH₂OCH₃ | (1-3-34c-2) |
| A-2 | O | CH₂CH₂ | Et | Et | (1-3-35c-2) |
| A-2 | O | CH₂(CH₂)₄CH₂ | CH₃ | CH₃ | (1-3-36c-2) |
| A-2 | O | CH₂(CH₂)₄CH₂ | CH₃ | Et | (1-3-37c-2) |
| A-2 | O | CH₂(CH₂)₄CH₂ | CH₃ | ⁱPr | (1-3-38c-2) |
| A-2 | O | CH₂(CH₂)₄CH₂ | CH₃ | Bu | (1-3-39c-2) |
| A-2 | O | CH₂(CH₂)₄CH₂ | CH₃ | Bnz | (1-3-40c-2) |
| A-2 | O | CH₂(CH₂)₄CH₂ | CH₃ | CH₂CH₂OCH₃ | (1-3-41c-2) |
| A-2 | O | CH₂(CH₂)₄CH₂ | Et | Et | (1-3-42c-2) |

[symbol]

indicates data missing or illegible when filed

| A | X(R¹) [symbol] | R [symbol] | R⁷ | R¹⁰ | |
|---|---|---|---|---|---|
| A-6 | O | CH₂ | CH₃ | CH₃ | (1-3-22c-6) |
| A-6 | O | CH₂ | CH₃ | Et | (1-3-23c-6) |
| A-6 | O | CH₂ | CH₃ | ⁱPr | (1-3-24c-6) |
| A-6 | O | CH₂ | CH₃ | Bu | (1-3-25c-6) |
| A-6 | O | CH₂ | CH₃ | Bnz | (1-3-26c-6) |
| A-6 | O | CH₂ | CH₃ | CH₂CH₂OCH₃ | (1-3-27c-6) |
| A-6 | O | CH₂ | Et | Et | (1-3-28c-6) |
| A-6 | O | CH₂CH₂ | CH₃ | CH₃ | (1-3-29c-6) |
| A-6 | O | CH₂CH₂ | CH₃ | Et | (1-3-30c-6) |
| A-6 | O | CH₂CH₂ | CH₃ | ⁱPr | (1-3-31c-6) |
| A-6 | O | CH₂CH₂ | CH₃ | Bu | (1-3-32c-6) |
| A-6 | O | CH₂CH₂ | CH₃ | Bnz | (1-3-33c-6) |
| A-6 | O | CH₂CH₂ | CH₃ | CH₂CH₂OCH₃ | (1-3-34c-6) |
| A-6 | O | CH₂CH₂ | Et | Et | (1-3-35c-6) |
| A-6 | O | CH₂(CH₂)₄CH₂ | CH₃ | CH₃ | (1-3-36c-6) |
| A-6 | O | CH₂(CH₂)₄CH₂ | CH₃ | Et | (1-3-37c-6) |
| A-6 | O | CH₂(CH₂)₄CH₂ | CH₃ | ⁱPr | (1-3-38c-6) |
| A-6 | O | CH₂(CH₂)₄CH₂ | CH₃ | Bu | (1-3-39c-6) |
| A-6 | O | CH₂(CH₂)₄CH₂ | CH₃ | Bnz | (1-3-40c-6) |
| A-6 | O | CH₂(CH₂)₄CH₂ | CH₃ | CH₂CH₂OCH₃ | (1-3-41c-6) |
| A-6 | O | CH₂(CH₂)₄CH₂ | Et | Et | (1-3-42c-6) |

[symbol]

indicates data missing or illegible when filed

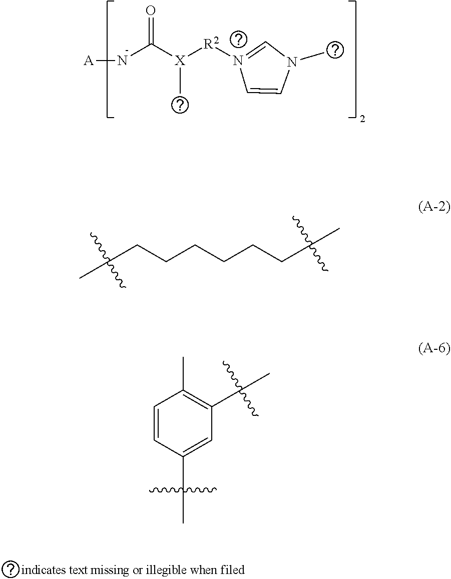

| A | X(R [symbol] ) [symbol] | R [symbol] | R [symbol] | |
|---|---|---|---|---|
| A-2 | NH | CH [symbol] | CH [symbol] | (1-3-43a-2) |
| A-2 | NH | CH [symbol] | Et | (1-3-44a-2) |
| A-2 | NH | CH [symbol] | Pr | (1-3-45a-2) |
| A-2 | NH | CH [symbol] | Bu | (1-3-46a-2) |
| A-2 | NH | CH [symbol] CH [symbol] | CH [symbol] | (1-3-47a-2) |
| A-2 | NH | CH [symbol] CH [symbol] | Et | (1-3-48a-2) |
| A-2 | NH | CH [symbol] CH [symbol] | Pr | (1-8-49a-2) |
| A-2 | NH | CH [symbol] CH [symbol] | Bu | (1-3-50a-2) |
| A-2 | NH | CH [symbol] (CH [symbol] ) [symbol] CH [symbol] | CH [symbol] | (1-3-51a-2) |
| A-2 | NH | CH [symbol] (CH [symbol] ) [symbol] CH [symbol] | Et | (1-3-52a-2) |
| A-2 | NH | CH [symbol] (CH [symbol] ) [symbol] CH [symbol] | Pr | (1-3-53a-2) |
| A-2 | NH | CH [symbol] (CH [symbol] ) [symbol] CH [symbol] | Bu | (1-3-54a-2) |
| A-2 | NH | CH [symbol] | CH [symbol] | (1-3-43a-6) |
| A-2 | NH | CH [symbol] | Et | (1-3-44a-6) |
| A-2 | NH | CH [symbol] | Pr | (1-3-45a-6) |
| A-2 | NH | CH [symbol] | Bu | (1-8-46a-6) |
| A-2 | NH | CH [symbol] CH [symbol] | CH [symbol] | (1-3-47a-6) |
| A-2 | NH | CH [symbol] CH [symbol] | Et | (1-3-48a-6) |
| A-2 | NH | CH [symbol] CH [symbol] | Pr | (1-3-49a-6) |
| A-2 | NH | CH [symbol] CH [symbol] | Bu | (1-8-50a-6) |
| A-2 | NH | CH [symbol] (CH [symbol] ) [symbol] CH [symbol] | CH [symbol] | (1-3-51a-6) |
| A-2 | NH | CH [symbol] (CH [symbol] ) [symbol] CH [symbol] | Et | (1-3-52a-6) |
| A-2 | NH | CH [symbol] (CH [symbol] ) [symbol] CH [symbol] | Pr | (1-3-53a-6) |
| A-2 | NH | CH [symbol] (CH [symbol] ) [symbol] CH [symbol] | Bu | (1-3-54a-6) |

[symbol]

indicates data missing or illegible when filed

| A | X(R¹) [symbol] | R² | R¹⁰ | |
|---|---|---|---|---|
| A-2 | N(CH₃) | CH₂ | CH₃ | (1-3-43b-2) |
| A-2 | N(CH₃) | CH₂ | Et | (1-3-44b-2) |
| A-2 | N(CH₃) | CH₂ | Pr | (1-3-45b-2) |
| A-2 | N(CH₃) | CH₂ | Bu | (1-3-46b-2) |
| A-2 | N(CH₃) | CH₂CH₂ | CH₃ | (1-3-47b-2) |
| A-2 | N(CH₃) | CH₂CH₂ | Et | (1-3-48b-2) |
| A-2 | N(CH₃) | CH₂CH [symbol] | Pr | (1-3-49b-2) |
| A-2 | N(CH₃) | CH₂CH₂ | Bu | (1-3-50b-2) |
| A-2 | N(CH₃) | CH₂(CH₂)₄CH₂ | CH₃ | (1-3-51b-2) |
| A-2 | N(CH₃) | CH₂(CH [symbol] )₄CH₂ | Et | (1-3-52b-2) |
| A-2 | N(CH₃) | CH₂(CH₂)₄CH [symbol] | Pr | (1-3-53b-2) |
| A-2 | N(CH₃) | CH₂(CH₂)₄CH₂ | Bu | (1-3-54b-2) |
| A-6 | N(CH₃) | CH₂ | CH₃ | (1-3-43b-6) |
| A-6 | N(CH₃) | CH₂ | Et | (1-3-44b-6) |
| A-6 | N(CH₃) | CH₂ | Pr | (1-3-45b-6) |
| A-6 | N(CH₃) | CH₂ | Bu | (1-3-46b-6) |
| A-6 | N(CH₃) | CH₂CH₂ | CH₃ | (1-3-47b-6) |
| A-6 | N(CH₃) | CH₂CH₂ | Et | (1-3-48b-6) |
| A-6 | N(CH₃) | CH₂CH₂ | Pr | (1-3-49b-6) |
| A-6 | N(CH₃) | CH₂CH₂ | Bu | (1-3-50b-6) |
| A-6 | N(CH₃) | CH₂(CH₂)₄CH₂ | CH₃ | (1-3-51b-6) |
| A-6 | N(CH₃) | CH₂(CH₂)₄CH₂ | Et | (1-3-52b-6) |
| A-6 | N(CH₃) | CH₂(CH₂)₄CH [symbol] | Pr | (1-3-53b-6) |
| A-6 | N(CH₃) | CH₂(CH₂)₄CH₂ | Bu | (1-3-54b-6) |

[symbol]

indicates data missing or illegible when filed

| A | X(R¹) [symbol] | R² | R¹⁰ | |
|---|---|---|---|---|
| A-2 | O | CH₂ | CH₃ | (1-3-43c-2) |
| A-2 | O | CH₂ | Et | (1-3-44c-2) |
| A-2 | O | CH₂ | Pr | (1-3-45c-2) |
| A-2 | O | CH₂ | Bu | (1-3-46c-2) |
| A-2 | O | CH₂CH₂ | CH₃ | (1-3-47c-2) |
| A-2 | O | CH₂CH₂ | Et | (1-3-48c-2) |
| A-2 | O | CH₂CH₂ | Pr | (1-3-49c-2) |
| A-2 | O | CH₂CH₂ | Bu | (1-3-50c-2) |
| A-2 | O | CH₂(CH₂)₄CH₂ | CH₃ | (1-3-51c-2) |
| A-2 | O | CH₂(CH₂)₄CH₂ | Et | (1-3-52c-2) |
| A-2 | O | CH [symbol] (CH₂)₄CH₂ | Pr | (1-3-53c-2) |
| A-2 | O | CH₂(CH₂)₄CH₂ | Bu | (1-3-54c-2) |
| A-6 | O | CH₂ | CH₃ | (1-3-43c-6) |
| A-6 | O | CH₂ | Et | (1-3-44c-6) |
| A-6 | O | CH₂ | Pr | (1-3-45c-6) |
| A-6 | O | CH [symbol] | Bu | (1-3-46c-6) |
| A-6 | O | CH₂CH₂ | CH₃ | (1-3-47c-6) |
| A-6 | O | CH₂CH₂ | Et | (1-3-48c-6) |
| A-6 | O | CH₂CH [symbol] | Pr | (1-3-49c-6) |
| A-6 | O | CH₂CH₂ | Bu | (1-3-50c-6) |
| A-6 | O | CH₂(CH₂)₄CH₂ | CH₃ | (1-3-51c-6) |
| A-6 | O | CH₂(CH₂)₄CH₂ | Et | (1-3-52c-6) |
| A-6 | O | CH₂(CH₂)₄CH₂ | Pr | (1-3-53c-6) |
| A-6 | O | CH₂(CH₂)₄CH₂ | Bu | (1-3-54c-6) |

[symbol]

indicates data missing or illegible when filed

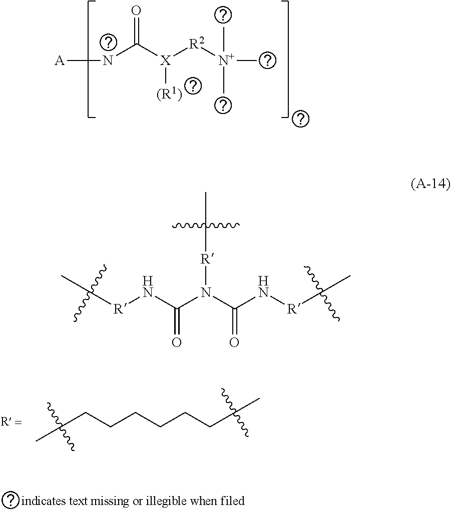

| A | X (R [symbol] ) [symbol] | R [symbol] | R [symbol] | R [symbol] | R [symbol] | |
|---|---|---|---|---|---|---|
| A-14 | NH | CH [symbol] | CH [symbol] | CH [symbol] | CH [symbol] | (1-2-1a-14) |
| A-14 | NH | CH [symbol] | CH [symbol] | CH [symbol] | Et | (1-2-2a-14) |
| A-14 | NH | CH [symbol] | CH [symbol] | CH [symbol] | [symbol] Pr | (1-2-3a-14) |
| A-14 | NH | CH [symbol] | CH [symbol] | CH [symbol] | Bu | (1-2-4a-14) |
| A-14 | NH | CH [symbol] | CH [symbol] | CH [symbol] | [symbol] | (1-2-5a-14) |
| A-14 | NH | CH [symbol] | CH [symbol] | Et | Et | (1-2-6a-14) |
| A-14 | NH | CH [symbol] | CH [symbol] | CH [symbol] CH [symbol] OCH [symbol] | CH [symbol] CH [symbol] OCH [symbol] | (1-2-7a-14) |
| A-14 | NH | CH [symbol] | CH [symbol] | Pr | Bu | (1-2-8a-14) |
| A-14 | NH | CH [symbol] | Et | Et | Et | (1-2-9a-14) |
| A-14 | NH | CH [symbol] | [symbol] Pr | [symbol] Pr | [symbol] Pr | (1-2-10a-14) |
| A-14 | NH | CH [symbol] | Bu | Bu | Bu | (1-2-11a-14) |
| A-14 | NH | CH [symbol] CH [symbol] CH [symbol] | CH [symbol] | CH [symbol] | CH [symbol] | (1-2-12a-14) |
| A-14 | NH | CH [symbol] CH [symbol] CH [symbol] | CH [symbol] | CH [symbol] | Et | (1-2-13a-14) |
| A-14 | NH | CH [symbol] CH [symbol] CH [symbol] | CH [symbol] | CH [symbol] | [symbol] Pr | (1-2-14a-14) |
| A-14 | NH | CH [symbol] CH [symbol] CH [symbol] | CH [symbol] | CH [symbol] | Bu | (1-2-15a-14) |
| A-14 | NH | CH [symbol] CH [symbol] CH [symbol] | CH [symbol] | CH [symbol] | [symbol] | (1-2-16a-14) |
| A-14 | NH | CH [symbol] CH [symbol] CH [symbol] | CH [symbol] | Et | Et | (1-2-17a-14) |
| A-14 | NH | CH [symbol] CH [symbol] CH [symbol] | CH [symbol] | CH [symbol] CH [symbol] OCH [symbol] | CH [symbol] CH [symbol] OCH [symbol] | (1-2-18a-14) |
| A-14 | NH | CH [symbol] CH [symbol] CH [symbol] | CH [symbol] | Pr | Bu | (1-2-19a-14) |
| A-14 | NH | CH [symbol] CH [symbol] CH [symbol] | Et | Et | Et | (1-2-20a-14) |
| A-14 | NH | CH [symbol] CH [symbol] CH [symbol] | [symbol] Pr | [symbol] Pr | [symbol] Pr | (1-2-21a-14) |
| A-14 | NH | CH [symbol] CH [symbol] CH [symbol] | Bu | Bu | Bu | (1-2-22a-14) |
| A-14 | NH | CH [symbol] (CH [symbol] ) [symbol] CH [symbol] | CH [symbol] | CH [symbol] | CH [symbol] | (1-2-23a-14) |
| A-14 | NH | CH [symbol] (CH [symbol] ) [symbol] CH [symbol] | CH [symbol] | CH [symbol] | Et | (1-2-24a-14) |
| A-14 | NH | CH [symbol] (CH [symbol] ) [symbol] CH [symbol] | CH [symbol] | CH [symbol] | [symbol] Pr | (1-2-25a-14) |
| A-14 | NH | CH [symbol] (CH [symbol] ) [symbol] CH [symbol] | CH [symbol] | CH [symbol] | Bu | (1-2-26a-14) |
| A-14 | NH | CH [symbol] (CH [symbol] ) [symbol] CH [symbol] | CH [symbol] | CH [symbol] | [symbol] | (1-2-27a-14) |
| A-14 | NH | CH [symbol] (CH [symbol] ) [symbol] CH [symbol] | CH [symbol] | Et | Et | (1-2-28a-14) |
| A-14 | NH | CH [symbol] (CH [symbol] ) [symbol] CH [symbol] | CH [symbol] | CH [symbol] CH [symbol] OCH [symbol] | CH [symbol] CH [symbol] OCH [symbol] | (1-2-29a-14) |

[symbol]

indicates data missing or illegible when filed

| | | | | | | |
|---|---|---|---|---|---|---|
| A-14 | NH | CH₂(CH₂) ₄CH₂ | CH₃ | Pr | Bu | (1-2-30a-14) |
| A-14 | NH | CH₂(CH₂) ₄CH₂ | Et | Et | Et | (1-2-31a-14) |
| A-14 | NH | CH₂(CH₂) ₄CH₂ | [symbol] Pr | [symbol] Pr | [symbol] Pr | (1-2-32a-14) |
| A-14 | NH | CH₂(CH₂) ₄CH₂ | Bu | Bu | Bu | (1-2-33a-14) |

[symbol]

indicates data missing or illegible when filed

| A | X (R [symbol] ) [symbol] | R [symbol] | R [symbol] | R [symbol] | R [symbol] | |
|---|---|---|---|---|---|---|
| A-14 | N(CH3) | CH [symbol] | CH [symbol] | CH [symbol] | CH [symbol] | (1-2-1b-14) |
| A-14 | N(CH3) | CH [symbol] | CH [symbol] | CH [symbol] | Et | (1-2-2b-14) |
| A-14 | N(CH3) | CH [symbol] | CH [symbol] | CH [symbol] | [symbol] Pr | (1-2-3b-14) |
| A-14 | N(CH3) | CH [symbol] | CH [symbol] | CH [symbol] | Bu | (1-2-4b-14) |
| A-14 | N(CH3) | CH [symbol] | CH [symbol] | CH [symbol] | [symbol] | (1-2-5b-14) |
| A-14 | N(CH3) | CH [symbol] | CH [symbol] | Et | Et | (1-2-6b-14) |
| A-14 | N(CH3) | CH [symbol] | CH [symbol] | CH [symbol] CH [symbol] OCH [symbol] | CH [symbol] CH [symbol] OCH [symbol] | (1-2-7b-14) |
| A-14 | N(CH3) | CH [symbol] | CH [symbol] | Pr | Bu | (1-2-8b-14) |
| A-14 | N(CH3) | CH [symbol] | Et | Et | Et | (1-2-9b-14) |
| A-14 | N(CH3) | CH [symbol] | [symbol] Pr | [symbol] Pr | [symbol] Pr | (1-2-10b-14) |
| A-14 | N(CH3) | CH [symbol] | Bu | Bu | Bu | (1-2-11b-14) |
| A-14 | N(CH3) | CH [symbol] CH [symbol] CH [symbol] | CH [symbol] | CH [symbol] | CH [symbol] | (1-2-12b-14) |
| A-14 | N(CH3) | CH [symbol] CH [symbol] CH [symbol] | CH [symbol] | CH [symbol] | Et | (1-2-13b-14) |
| A-14 | N(CH3) | CH [symbol] CH [symbol] CH [symbol] | CH [symbol] | CH [symbol] | [symbol] Pr | (1-2-14b-14) |
| A-14 | N(CH3) | CH [symbol] CH [symbol] CH [symbol] | CH [symbol] | CH [symbol] | Bu | (1-2-15b-14) |
| A-14 | N(CH3) | CH [symbol] CH [symbol] CH [symbol] | CH [symbol] | CH [symbol] | [symbol] | (1-2-16b-14) |
| A-14 | N(CH3) | CH [symbol] CH [symbol] CH [symbol] | CH [symbol] | Et | Et | (1-2-17b-14) |
| A-14 | N(CH3) | CH [symbol] CH [symbol] CH [symbol] | CH [symbol] | CH [symbol] CH [symbol] OCH [symbol] | CH [symbol] CH [symbol] OCH [symbol] | (1-2-18b-14) |
| A-14 | N(CH3) | CH [symbol] CH [symbol] CH [symbol] | CH [symbol] | Pr | Bu | (1-2-19b-14) |
| A-14 | N(CH3) | CH [symbol] CH [symbol] CH [symbol] | Et | Et | Et | (1-2-20b-14) |
| A-14 | N(CH3) | CH [symbol] CH [symbol] CH [symbol] | [symbol] Pr | [symbol] Pr | [symbol] Pr | (1-2-21b-14) |
| A-14 | N(CH3) | CH [symbol] CH [symbol] CH [symbol] | Bu | Bu | Bu | (1-2-22b-14) |
| A-14 | N(CH3) | CH [symbol] (CH [symbol] ) [symbol] CH [symbol] | CH [symbol] | CH [symbol] | CH [symbol] | (1-2-23b-14) |
| A-14 | N(CH3) | CH [symbol] (CH [symbol] ) [symbol] CH [symbol] | CH [symbol] | CH [symbol] | Et | (1-2-24b-14) |
| A-14 | N(CH3) | CH [symbol] (CH [symbol] ) [symbol] CH [symbol] | CH [symbol] | CH [symbol] | [symbol] Pr | (1-2-25b-14) |
| A-14 | N(CH3) | CH [symbol] (CH [symbol] ) [symbol] CH [symbol] | CH [symbol] | CH [symbol] | Bu | (1-2-26b-14) |
| A-14 | N(CH3) | CH [symbol] (CH [symbol] ) [symbol] CH [symbol] | CH [symbol] | CH [symbol] | [symbol] | (1-2-27b-14) |
| A-14 | N(CH3) | CH [symbol] (CH [symbol] ) [symbol] CH [symbol] | CH [symbol] | Et | Et | (1-2-28b-14) |
| A-14 | N(CH3) | CH [symbol] (CH [symbol] ) [symbol] CH [symbol] | CH [symbol] | CH [symbol] CH [symbol] OCH [symbol] | CH [symbol] CH [symbol] OCH [symbol] | (1-2-29b-14) |
| A-14 | N(CH3) | CH [symbol] (CH [symbol] ) [symbol] CH [symbol] | CH [symbol] | Pr | Bu | (1-2-30b-14) |
| A-14 | N(CH3) | CH [symbol] (CH [symbol] ) [symbol] CH [symbol] | Et | Et | Et | (1-2-31b-14) |
| A-14 | N(CH3) | CH [symbol] (CH [symbol] ) [symbol] CH [symbol] | [symbol] Pr | [symbol] Pr | [symbol] Pr | (1-2-32b-14) |
| A-14 | N(CH3) | CH [symbol] (CH [symbol] ) [symbol] CH [symbol] | Bu | Bu | Bu | (1-2-33b-14) |

[symbol]

indicates data missing or illegible when filed

| A | X (R [symbol] ) [symbol] | R [symbol] | R [symbol] | R [symbol] | R [symbol] | |
|---|---|---|---|---|---|---|
| A-14 | 0 | CH [symbol] | CH [symbol] | CH [symbol] | CH [symbol] | (1-2-1c-14) |
| A-14 | 0 | CH [symbol] | CH [symbol] | CH [symbol] | Et | (1-2-2c-14) |
| A-14 | 0 | CH [symbol] | CH [symbol] | CH [symbol] | [symbol] Pr | (1-2-3c-14) |
| A-14 | 0 | CH [symbol] | CH [symbol] | CH [symbol] | Bu | (1-2-4c-14) |
| A-14 | 0 | CH [symbol] | CH [symbol] | CH [symbol] | [symbol] | (1-2-5c-14) |
| A-14 | 0 | CH [symbol] | CH [symbol] | Et | Et | (1-2-6c-14) |
| A-14 | 0 | CH [symbol] | CH [symbol] | CH [symbol] CH [symbol] OCH [symbol] | CH [symbol] CH [symbol] OCH [symbol] | (1-2-7c-14) |
| A-14 | 0 | CH [symbol] | CH [symbol] | Pr | Bu | (1-2-8c-14) |
| A-14 | 0 | CH [symbol] | Et | Et | Et | (1-2-9c-14) |
| A-14 | 0 | CH [symbol] | [symbol] Pr | [symbol] Pr | [symbol] Pr | (1-2-10c-14) |
| A-14 | 0 | CH [symbol] | Bu | Bu | Bu | (1-2-11c-14) |
| A-14 | 0 | CH [symbol] CH [symbol] CH [symbol] | CH [symbol] | CH [symbol] | CH [symbol] | (1-2-12c-14) |
| A-14 | 0 | CH [symbol] CH [symbol] CH [symbol] | CH [symbol] | CH [symbol] | Et | (1-2-13c-14) |
| A-14 | 0 | CH [symbol] CH [symbol] CH [symbol] | CH [symbol] | CH [symbol] | [symbol] Pr | (1-2-14c-14) |
| A-14 | 0 | CH [symbol] CH [symbol] CH [symbol] | CH [symbol] | CH [symbol] | Bu | (1-2-15c-14) |
| A-14 | 0 | CH [symbol] CH [symbol] CH [symbol] | CH [symbol] | CH [symbol] | [symbol] | (1-2-16c-14) |
| A-14 | 0 | CH [symbol] CH [symbol] CH [symbol] | CH [symbol] | Et | Et | (1-2-17c-14) |
| A-14 | 0 | CH [symbol] CH [symbol] CH [symbol] | CH [symbol] | CH [symbol] CH [symbol] OCH [symbol] | CH [symbol] CH [symbol] OCH [symbol] | (1-2-18c-14) |
| A-14 | 0 | CH [symbol] CH [symbol] CH [symbol] | CH [symbol] | Pr | Bu | (1-2-19c-14) |
| A-14 | 0 | CH [symbol] CH [symbol] CH [symbol] | Et | Et | Et | (1-2-20c-14) |
| A-14 | 0 | CH [symbol] CH [symbol] CH [symbol] | [symbol] Pr | [symbol] Pr | [symbol] Pr | (1-2-21c-14) |
| A-14 | 0 | CH [symbol] CH [symbol] CH [symbol] | Bu | Bu | Bu | (1-2-22c-14) |
| A-14 | 0 | CH [symbol] (CH [symbol] ) [symbol] CH [symbol] | CH [symbol] | CH [symbol] | CH [symbol] | (1-2-23c-14) |
| A-14 | 0 | CH [symbol] (CH [symbol] ) [symbol] CH [symbol] | CH [symbol] | CH [symbol] | Et | (1-2-24c-14) |
| A-14 | 0 | CH [symbol] (CH [symbol] ) [symbol] CH [symbol] | CH [symbol] | CH [symbol] | [symbol] Pr | (1-2-25c-14) |
| A-14 | 0 | CH [symbol] (CH [symbol] ) [symbol] CH [symbol] | CH [symbol] | CH [symbol] | Bu | (1-2-26c-14) |
| A-14 | 0 | CH [symbol] (CH [symbol] ) [symbol] CH [symbol] | CH [symbol] | CH [symbol] | [symbol] | (1-2-27c-14) |
| A-14 | 0 | CH [symbol] (CH [symbol] ) [symbol] CH [symbol] | CH [symbol] | Et | Et | (1-2-28c-14) |
| A-14 | 0 | CH [symbol] (CH [symbol] ) [symbol] CH [symbol] | CH [symbol] | CH [symbol] CH [symbol] OCH [symbol] | CH [symbol] CH [symbol] OCH [symbol] | (1-2-29c-14) |
| A-14 | 0 | CH [symbol] (CH [symbol] ) [symbol] CH [symbol] | CH [symbol] | Pr | Bu | (1-2-30c-14) |
| A-14 | 0 | CH [symbol] (CH [symbol] ) [symbol] CH [symbol] | Et | Et | Et | (1-2-31c-14) |
| A-14 | 0 | CH [symbol] (CH [symbol] ) [symbol] CH [symbol] | [symbol] Pr | [symbol] Pr | [symbol] Pr | (1-2-32c-14) |
| A-14 | 0 | CH [symbol] (CH [symbol] ) [symbol] CH [symbol] | Bu | Bu | Bu | (1-2-32c-14) |

[symbol]

indicates data missing or illegible when filed

The zwitterionic compound (1) is preferably a compound represented by any one of (1-2-1a-1) to (1-2-33a-1), (1-2-1b-1) to (1-2-33b-1), (1-2-1c-1) to (1-2-33c-1), (1-2-1a-2) to (1-2-33a-2), (1-2-1b-2) to (1-2-33b-2), (1-2-1c-2) to (1-2-33c-2), (1-2-1a-6) to (1-2-33a-6), (1-2-1b-6) to (1-2-33b-6), (1-2-1c-6) to (1-2-33c-6), (1-2-1a-14) to (1-2-33a-14), (1-2-1b-14) to (1-2-33b-14), and (1-2-1c-14) to (1-2-33c-14), and particularly preferably a compound represented by (1-2-12a-1) or (1-2-12a-2).

The method for producing the zwitterionic compound (1) may be, for example, a method comprising stirring a compound represented by formula (8) in a solvent (Production Method 1).

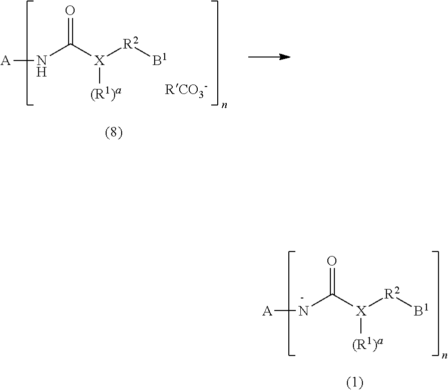

(In the formulas, R′ represents a hydrogen atom or a substituted or unsubstituted hydrocarbon group, and R¹, R², A, a, X, and B¹ are as defined above.)

Production Method 1 is described below.

The reaction can usually be carried out at a temperature of 0° C. to the boiling temperature of the solvent. The reaction temperature is preferably 20° C. to 150° C., more preferably 40° C. to 150° C., and even more preferably 40° C. to 120° C. When X=an oxygen atom in the compound represented by formula (8), the reaction temperature is preferably 20 to 60° C.

The reaction time is usually 1 to 20 hours, and preferably 1 to 10 hours.

Examples of the solvent include tetrahydrofuran, ethyl acetate, acetonitrile, toluene, acetone, methanol, and the like. The amount of the solvent for use is usually 100 parts by mass or less, and preferably 0.1 to 50 parts by mass, per part by mass of the compound represented by formula (8). Two or more different solvents may be used in combination, if necessary.

In Production Method 1 of the present invention, the reaction may be performed, if necessary, in an inert gas atmosphere, such as nitrogen, argon, or helium, which do not affect the reaction.

The obtained zwitterionic compound (1) can be purified according to common methods, such as concentration and recrystallization.

The compound represented by formula (8) for use in Production Method 1 may be produced, for example, according to the following methods.

Method A: A method comprising step 1 and step 2.

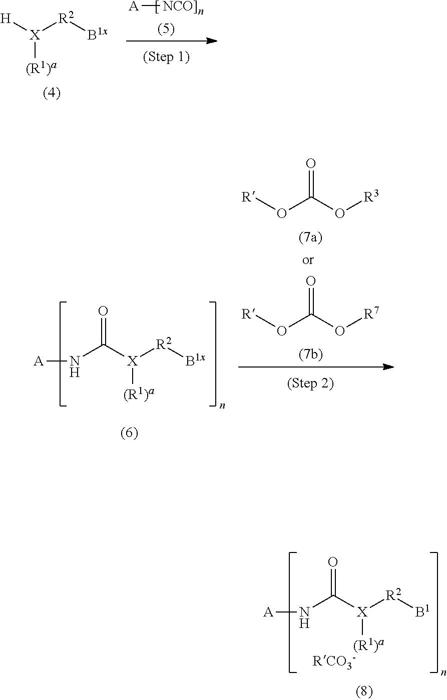

(In the formulas, B^{1x} represents a group represented by the following formula (10) or formula (11), wherein when B^{1x} represents a group represented by the following formula (10), an ester carbonate compound represented by formula (7a) is used, and when B^{1x} represents a group represented by the following formula (11), an ester carbonate compound represented by formula (7b) is used. R′ represents a hydrogen atom, a substituted or unsubstituted hydrocarbon group, R³, or R⁷, and R¹, R², R³, R⁷, B¹, A, a, X, and n are as defined above.)
Step 1: A compound represented by formula (4) is reacted with an isocyanate compound represented by formula (5) to obtain a compound represented by formula (6).
Step 2: The compound represented by formula (6) is reacted with an ester carbonate compound represented by formula (7a) or formula (7b) to thus obtain a compound that is represented by formula (8) and in which R′ represents R³ or R⁷ in formula (8) (hereinafter referred to as “the compound (8b′)”). The compound (8b′) is a compound represented by formula (8) and is included in compounds in which R′ represents a substituted or unsubstituted hydrocarbon group in formula (8) (hereinafter referred to as “the compound (8b)”).

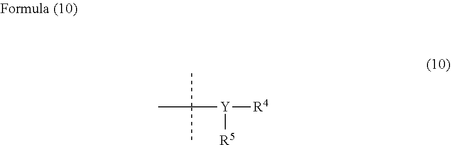

(In the formula, Y represents a nitrogen atom or a phosphorus atom, and R⁴ and R⁵ are as defined above.)

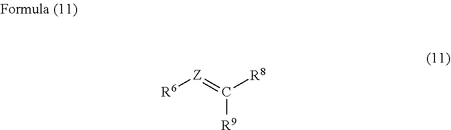

(In the formula, Z represents a nitrogen atom or a phosphorus atom, and R⁶, R⁸, and R⁹ are as defined above.)

Step 1 is described below.

A compound represented by formula (4) is reacted with an isocyanate compound represented by formula (5) to obtain a urethane compound or a urea compound represented by formula (6).

Specific examples of the compound represented by formula (4) are shown below. However, the present invention is not limited thereto. In the following specific examples, Et represents an ethyl group, Pr represents a propyl group, ⁱPr represents an isopropyl group, Bu represents a butyl group, and Bnz represents a benzyl group.

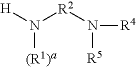

| X(R [symbol] ) [symbol] | R [symbol] | R [symbol] | R [symbol] | |
|---|---|---|---|---|
| NH | CH [symbol] | CH [symbol] | CH [symbol] | (4-2-1a) |
| NH | CH [symbol] | CH [symbol] | Et | (4-2-2a) |
| NH | CH [symbol] | CH [symbol] | ⁱPr | (4-2-3a) |
| NH | CH [symbol] | CH [symbol] | Bu | (4-2-4a) |
| NH | CH [symbol] | CH [symbol] | Bnz | (4-2-5a) |
| NH | CH [symbol] | Et | Et | (4-2-6a) |
| NH | CH [symbol] | CH [symbol] CH [symbol] OCH [symbol] | CH [symbol] CH [symbol] OCH [symbol] | (4-2-7a) |
| NH | CH [symbol] | Pr | Bu | (4-2-8a) |
| NH | CH [symbol] | Et | Et | (4-2-9a) |
| NH | CH [symbol] | ⁱPr | ⁱPr | (4-2-10a) |
| NH | CH [symbol] | Bu | Bu | (4-2-11a) |
| NH | CH [symbol] CH [symbol] CH [symbol] | CH [symbol] | CH [symbol] | (4-2-12a) |
| NH | CH [symbol] CH [symbol] CH [symbol] | CH [symbol] | Et | (4-2-13a) |
| NH | CH [symbol] CH [symbol] CH [symbol] | CH [symbol] | ⁱPr | (4-2-14a) |
| NH | CH [symbol] CH [symbol] CH [symbol] | CH [symbol] | Bu | (4-2-15a) |
| NH | CH [symbol] CH [symbol] CH [symbol] | CH [symbol] | Bnz | (4-2-16a) |
| NH | CH [symbol] CH [symbol] CH [symbol] | Et | Et | (4-2-17a) |
| NH | CH [symbol] CH [symbol] CH [symbol] | CH [symbol] CH [symbol] OCH [symbol] | CH [symbol] CH [symbol] OCH [symbol] | (4-2-18a) |
| NH | CH [symbol] CH [symbol] CH [symbol] | Pr | Bu | (4-2-19a) |
| NH | CH [symbol] CH [symbol] CH [symbol] | Et | Et | (4-2-20a) |
| NH | CH [symbol] CH [symbol] CH [symbol] | ⁱPr | ⁱPr | (4-2-21a) |
| NH | CH [symbol] CH [symbol] CH [symbol] | Bu | Bu | (4-2-22a) |
| NH | CH [symbol] (CH [symbol] ) [symbol] CH [symbol] | CH [symbol] | CH [symbol] | (4-2-23a) |
| NH | CH [symbol] (CH [symbol] ) [symbol] CH [symbol] | CH [symbol] | Et | (4-2-24a) |
| NH | CH [symbol] (CH [symbol] ) [symbol] CH [symbol] | CH [symbol] | ⁱPr | (4-2-25a) |
| NH | CH [symbol] (CH [symbol] ) [symbol] CH [symbol] | CH [symbol] | Bu | (4-2-26a) |
| NH | CH [symbol] (CH [symbol] ) [symbol] CH [symbol] | CH [symbol] | Bnz | (4-2-27a) |
| NH | CH [symbol] (CH [symbol] ) [symbol] CH [symbol] | Et | Et | (4-2-28a) |
| NH | CH [symbol] (CH [symbol] ) [symbol] CH [symbol] | CH [symbol] CH [symbol] OCH [symbol] | CH [symbol] CH [symbol] OCH [symbol] | (4-2-29a) |
| NH | CH [symbol] (CH [symbol] ) [symbol] CH [symbol] | Pr | Bu | (4-2-30a) |
| NH | CH [symbol] (CH [symbol] ) [symbol] CH [symbol] | Et | Et | (4-2-31a) |
| NH | CH [symbol] (CH [symbol] ) [symbol] CH [symbol] | ⁱPr | ⁱPr | (4-2-32a) |
| NH | CH [symbol] (CH [symbol] ) [symbol] CH [symbol] | Bu | Bu | (4-2-33a) |

[symbol]

indicates data missing or illegible when filed

| X(R [symbol] ) [symbol] | R [symbol] | R [symbol] | R [symbol] | |
|---|---|---|---|---|
| N(CH [symbol] ) | CH [symbol] | CH [symbol] | CH [symbol] | (4-2-1b) |
| N(CH [symbol] ) | CH [symbol] | CH [symbol] | Et | (4-2-2b) |
| N(CH [symbol] ) | CH [symbol] | CH [symbol] | ⁱPr | (4-2-3b) |
| N(CH [symbol] ) | CH [symbol] | CH [symbol] | Bu | (4-2-4b) |
| N(CH [symbol] ) | CH [symbol] | CH [symbol] | Bnz | (4-2-5b) |
| N(CH [symbol] ) | CH [symbol] | Et | Et | (4-2-6b) |
| N(CH [symbol] ) | CH [symbol] | CH [symbol] CH [symbol] OCH [symbol] | CH [symbol] CH [symbol] OCH [symbol] | (4-2-7b) |
| N(CH [symbol] ) | CH [symbol] | Pr | Bu | (4-2-8b) |
| N(CH [symbol] ) | CH [symbol] | Et | Et | (4-2-9b) |
| N(CH [symbol] ) | CH [symbol] | ⁱPr | ⁱPr | (4-2-10b) |
| N(CH [symbol] ) | CH [symbol] | Bu | Bu | (4-2-11b) |
| N(CH [symbol] ) | CH [symbol] CH [symbol] CH [symbol] | CH [symbol] | CH [symbol] | (4-2-12b) |
| N(CH [symbol] ) | CH [symbol] CH [symbol] CH [symbol] | CH [symbol] | Et | (4-2-13b) |
| N(CH [symbol] ) | CH [symbol] CH [symbol] CH [symbol] | CH [symbol] | ⁱPr | (4-2-14b) |
| N(CH [symbol] ) | CH [symbol] CH [symbol] CH [symbol] | CH [symbol] | Bu | (4-2-15b) |
| N(CH [symbol] ) | CH [symbol] CH [symbol] CH [symbol] | CH [symbol] | Bnz | (4-2-16b) |
| N(CH [symbol] ) | CH [symbol] CH [symbol] CH [symbol] | Et | Et | (4-2-17b) |
| N(CH [symbol] ) | CH [symbol] CH [symbol] CH [symbol] | CH [symbol] CH [symbol] OCH [symbol] | CH [symbol] CH [symbol] OCH [symbol] | (1-2-18b) |
| N(CH [symbol] ) | CH [symbol] CH [symbol] CH [symbol] | Pr | Bu | (4-8-19b) |
| N(CH [symbol] ) | CH [symbol] CH [symbol] CH [symbol] | Et | Et | (4-2-20b) |
| N(CH [symbol] ) | CH [symbol] CH [symbol] CH [symbol] | ⁱPr | ⁱPr | (4-2-21b) |
| N(CH [symbol] ) | CH [symbol] CH [symbol] CH [symbol] | Bu | Bu | (4-2-22b) |
| N(CH [symbol] ) | CH [symbol] (CH [symbol] ) [symbol] CH [symbol] | CH [symbol] | CH [symbol] | (4-2-23b) |
| N(CH [symbol] ) | CH [symbol] (CH [symbol] ) [symbol] CH [symbol] | CH [symbol] | Et | (4-2-24b) |
| N(CH [symbol] ) | CH [symbol] (CH [symbol] ) [symbol] CH [symbol] | CH [symbol] | ⁱPr | (4-2-25b) |
| N(CH [symbol] ) | CH [symbol] (CH [symbol] ) [symbol] CH [symbol] | CH [symbol] | Bu | (4-2-26b) |
| N(CH [symbol] ) | CH [symbol] (CH [symbol] ) [symbol] CH [symbol] | CH [symbol] | Bnz | (4-2-276) |
| N(CH [symbol] ) | CH [symbol] (CH [symbol] ) [symbol] CH [symbol] | Et | Et | (4-2-28b) |
| N(CH [symbol] ) | CH [symbol] (CH [symbol] ) [symbol] CH [symbol] | CH [symbol] CH [symbol] OCH [symbol] | CH [symbol] CH [symbol] OCH [symbol] | (4-2-29b) |
| N(CH [symbol] ) | CH [symbol] (CH [symbol] ) [symbol] CH [symbol] | Pr | Bu | (4-2-30b) |
| N(CH [symbol] ) | CH [symbol] (CH [symbol] ) [symbol] CH [symbol] | Et | Et | (4-2-31b) |
| N(CH [symbol] ) | CH [symbol] (CH [symbol] ) [symbol] CH [symbol] | ⁱPr | ⁱPr | (4-2-32b) |
| N(CH [symbol] ) | CH [symbol] (CH [symbol] ) [symbol] CH [symbol] | Bu | Bu | (4-2-33b) |

[symbol]

indicates data missing or illegible when filed

| X(R [symbol] ) [symbol] | R [symbol] | R [symbol] | R [symbol] | |
|---|---|---|---|---|
| O | CH [symbol] | CH [symbol] | CH [symbol] | (4-2-1c) |
| O | CH [symbol] | CH [symbol] | Et | (4-2-2c) |
| O | CH [symbol] | CH [symbol] | ⁱPr | (4-2-3c) |
| O | CH [symbol] | CH [symbol] | Bu | (4-2-4c) |
| O | CH [symbol] | CH [symbol] | Bnz | (4-2-5c) |
| O | CH [symbol] | Et | Et | (4-2-6c) |
| O | CH [symbol] | CH [symbol] CH [symbol] OCH [symbol] | CH [symbol] CH [symbol] OCH [symbol] | (4-2-7c) |
| O | CH [symbol] | Pr | Bu | (4-2-8c) |
| O | CH [symbol] | Et | Et | (4-2-9c) |
| O | CH [symbol] | ⁱPr | ⁱPr | (4-2-10c) |
| O | CH [symbol] | Bu | Bu | (4-2-11c) |
| O | CH [symbol] CH [symbol] CH [symbol] | CH [symbol] | CH [symbol] | (4-2-12c) |
| O | CH [symbol] CH [symbol] CH [symbol] | CH [symbol] | Et | (4-2-13c) |
| O | CH [symbol] CH [symbol] CH [symbol] | CH [symbol] | ⁱPr | (4-2-14c) |
| O | CH [symbol] CH [symbol] CH [symbol] | CH [symbol] | Bu | (4-2-15c) |
| O | CH [symbol] CH [symbol] CH [symbol] | CH [symbol] | Bnz | (4-2-16c) |
| O | CH [symbol] CH [symbol] CH [symbol] | Et | Et | (4-8-17c) |
| O | CH [symbol] CH [symbol] CH [symbol] | CH [symbol] CH [symbol] OCH [symbol] | CH [symbol] CH [symbol] OCH [symbol] | (4-2-18c) |
| O | CH [symbol] CH [symbol] CH [symbol] | Pr | Bu | (4-2-19c) |
| O | CH [symbol] CH [symbol] CH [symbol] | Et | Et | (4-2-20c) |
| O | CH [symbol] CH [symbol] CH [symbol] | ⁱPr | ⁱPr | (4-2-21c) |
| O | CH [symbol] CH [symbol] CH [symbol] | Bu | Bu | (4-2-22c) |
| O | CH [symbol] (CH [symbol] ) [symbol] CH [symbol] | CH [symbol] | CH [symbol] | (4-2-23c) |
| O | CH [symbol] (CH [symbol] ) [symbol] CH [symbol] | CH [symbol] | Et | (4-2-24c) |
| O | CH [symbol] (CH [symbol] ) [symbol] CH [symbol] | CH [symbol] | ⁱPr | (4-2-25c) |
| O | CH [symbol] (CH [symbol] ) [symbol] CH [symbol] | CH [symbol] | Bu | (4-2-26c) |
| O | CH [symbol] (CH [symbol] ) [symbol] CH [symbol] | CH [symbol] | Bnz | (4-2-27c) |
| O | CH [symbol] (CH [symbol] ) [symbol] CH [symbol] | Et | Et | (4-2-28c) |
| O | CH [symbol] (CH [symbol] ) [symbol] CH [symbol] | CH [symbol] CH [symbol] OCH [symbol] | CH [symbol] CH [symbol] OCH [symbol] | (4-2-29c) |
| O | CH [symbol] (CH [symbol] ) [symbol] CH [symbol] | Pr | Bu | (4-2-30c) |
| O | CH [symbol] (CH [symbol] ) [symbol] CH [symbol] | Et | Et | (4-2-31c) |
| O | CH [symbol] (CH [symbol] ) [symbol] CH [symbol] | ⁱPr | ⁱPr | (4-2-32c) |
| O | CH [symbol] (CH [symbol] ) [symbol] CH [symbol] | Bu | Bu | (4-2-33c) |

[symbol]

indicates data missing or illegible when filed

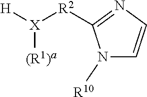

X, a, R¹, R², and R¹⁰ are as defined above. Preferably, X(R¹)^{a} represents NH or N(CH₃), R² represents CH₂, CH₂CH₂, CH₂(CH₂)₄, or CH₂, and R¹⁰ represents CH₃, Et, ⁱPr, Bu, Bnz, or CH₂CH₂OCH₃.

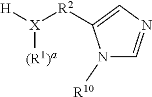

X, a, R¹, R², R¹⁰ are as defined above. Preferably, X(R¹)^{a} represents NH or N(CH₃), R² represents CH₂, CH₂CH₂, or CH₂(CH₂)₄CH₂, and R¹⁰ represents CH₃, Et, ⁱPr, Bu, Bnz, or CH₂CH₂OCH₃.

The compound represented by formula (4) is preferably a compound represented by any one of (4-2-1a) to (4-2-33a), (4-2-1b) to (4-2-33b), and (4-2-1c) to (4-2-33c), and particularly preferably (4-2-12a).

Specific examples of the isocyanate compound represented by formula (5) are shown below. However, the present invention is not limited thereto.

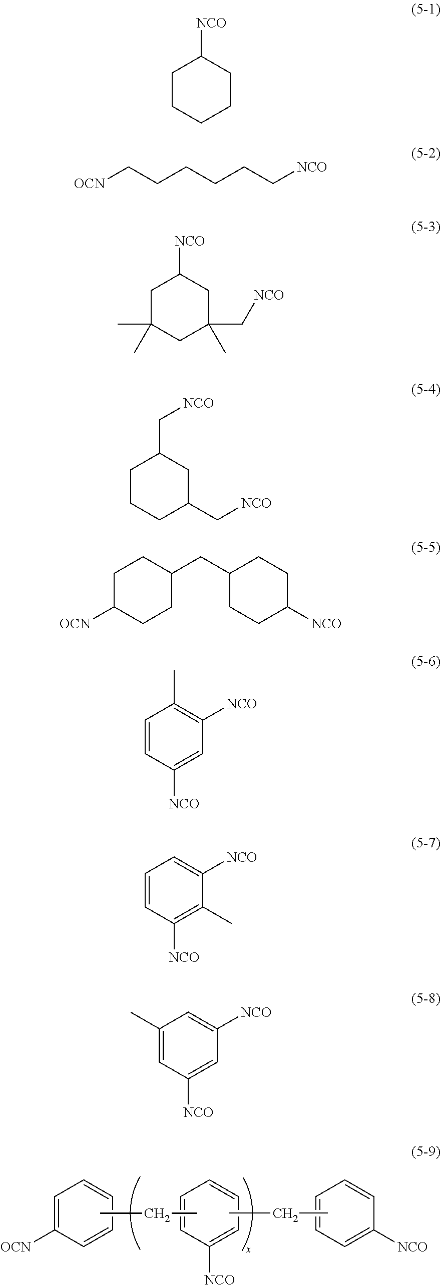

(In formula (5-9), x represents an integer of 0 or more and 20 or less, and preferably an integer of 1 or more and 20 or less.)

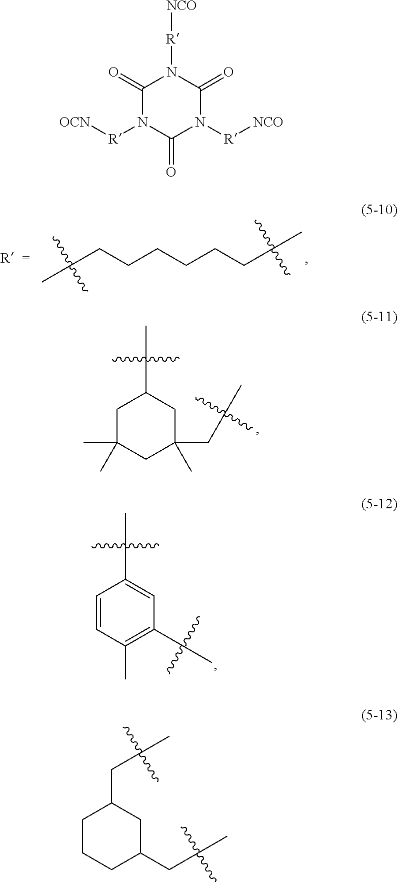

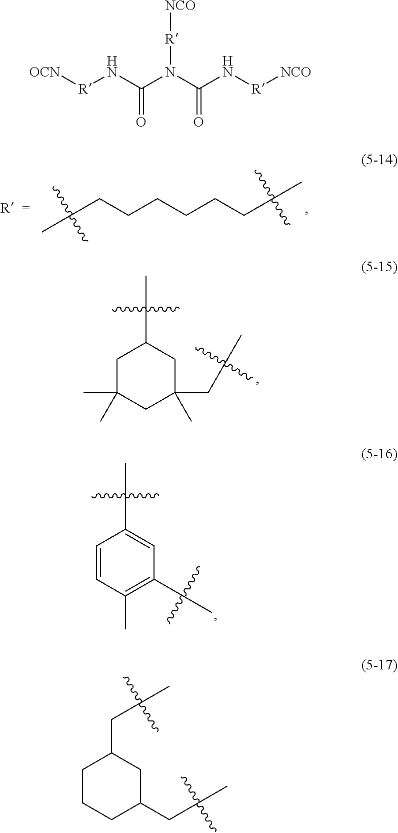

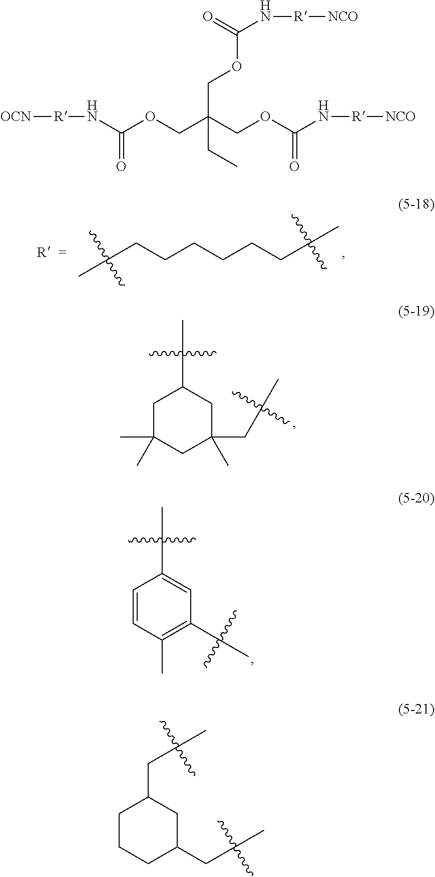

The isocyanate compound represented by formula (5) is preferably a compound represented by (5-1), (5-2), (5-6), or (5-14), and particularly preferably (5-1), (5-2), or (5-14).

Specific examples of the compound represented by formula (6) are shown below. However, the present invention is not limited thereto. In the following specific examples, Et represents an ethyl group, Pr represents a propyl group, iPr represents an isopropyl group, Bu represents a butyl group, and Bnz represents a benzyl group.

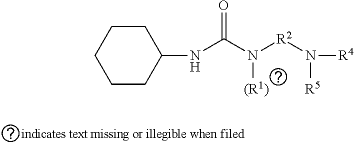

| [symbol] | R [symbol] | R [symbol] | R [symbol] | |
|---|---|---|---|---|
| NH | CH [symbol] | CH [symbol] | CH [symbol] | (6-2-1a-1) |
| NH | CH [symbol] | CH [symbol] | [symbol] | (6-2-2a-1) |
| NH | CH [symbol] | CH [symbol] | [symbol] | (6-2-3a-1) |
| NH | CH [symbol] | CH [symbol] | Bu | (6-2-4a-1) |
| NH | CH [symbol] | CH [symbol] | [symbol] | (6-2-5a-1) |
| NH | CH [symbol] | Et | Et | (6-2-6a-1) |
| NH | CH [symbol] | CH [symbol] CH [symbol] OCH [symbol] | CH [symbol] CH [symbol] OCH [symbol] | (6-2-7a-1) |
| NH | CH [symbol] | Pr | Bu | (6-2-8a-1) |
| NH | CH [symbol] | Et | Et | (6-2-9a-1) |
| NH | CH [symbol] | ⁱPr | ⁱPr | (6-2-10a-1) |
| NH | CH [symbol] | Bu | Bu | (6-2-11a-1) |
| NH | CH [symbol] CH [symbol] CH [symbol] | CH [symbol] | CH [symbol] | (6-2-12a-1) |
| NH | CH [symbol] CH [symbol] CH [symbol] | CH [symbol] | Et | (6-2-13a-1) |
| NH | CH [symbol] CH [symbol] CH [symbol] | CH [symbol] | ⁱPr | (6-2-14a-1) |
| NH | CH [symbol] CH [symbol] CH [symbol] | CH [symbol] | Bu | (6-2-15a-1) |
| NH | CH [symbol] CH [symbol] CH [symbol] | CH [symbol] | Bnz | (6-2-16a-1) |
| NH | CH [symbol] CH [symbol] CH [symbol] | Et | Et | (6-2-17a-1) |
| NH | CH [symbol] CH [symbol] CH [symbol] | CH [symbol] CH [symbol] OCH [symbol] | CH [symbol] CH [symbol] OCH [symbol] | (6-2-18a-1) |
| NH | CH [symbol] CH [symbol] CH [symbol] | [symbol] | Bu | (6-2-19a-1) |
| NH | CH [symbol] CH [symbol] CH [symbol] | Et | Et | (6-2-20a-1) |
| NH | CH [symbol] CH [symbol] CH [symbol] | ⁱPr | ⁱPr | (6-2-21a-1) |
| NH | CH [symbol] CH [symbol] CH [symbol] | Bu | Bu | (6-2-22a-1) |
| NH | CH [symbol] (CH [symbol] ) [symbol] CH [symbol] | CH [symbol] | CH [symbol] | (6-2-23a-1) |
| NH | CH [symbol] (CH [symbol] ) [symbol] CH [symbol] | CH [symbol] | [symbol] | (6-2-24a-1) |
| NH | CH [symbol] (CH [symbol] ) [symbol] CH [symbol] | CH [symbol] | ⁱPr | (6-2-25a-1) |
| NH | CH [symbol] (CH [symbol] ) [symbol] CH [symbol] | CH [symbol] | Bu | (6-2-26a-1) |
| NH | CH [symbol] (CH [symbol] ) [symbol] CH [symbol] | CH [symbol] | Bnz | (6-2-27a-1) |
| NH | CH [symbol] (CH [symbol] ) [symbol] CH [symbol] | Et | Et | (6-2-28a-1) |
| NH | CH [symbol] (CH [symbol] ) [symbol] CH [symbol] | CH [symbol] CH [symbol] OCH [symbol] | CH [symbol] CH [symbol] OCH [symbol] | (6-2-29a-1) |
| NH | CH [symbol] (CH [symbol] ) [symbol] CH [symbol] | Pr | Bu | (6-2-30a-1) |
| NH | CH [symbol] (CH [symbol] ) [symbol] CH [symbol] | Et | Et | (6-2-31a-1) |
| NH | CH [symbol] (CH [symbol] ) [symbol] CH [symbol] | ⁱPr | ⁱPr | (6-2-32a-1) |
| NH | CH [symbol] (CH [symbol] ) [symbol] CH [symbol] | Bu | Bu | (6-2-33a-1) |

[symbol]

indicates data missing or illegible when filed

| [symbol] | R [symbol] | R [symbol] | R [symbol] | |
|---|---|---|---|---|
| N(CH [symbol] ) | CH [symbol] | CH [symbol] | CH [symbol] | (6-2-1b-1) |
| N(CH [symbol] ) | CH [symbol] | CH [symbol] | Et | (6-2-2b-1) |
| N(CH [symbol] ) | CH [symbol] | CH [symbol] | ⁱPr | (6-2-3b-1) |
| N(CH [symbol] ) | CH [symbol] | CH [symbol] | Bu | (6-2-4b-1) |
| N(CH [symbol] ) | CH [symbol] | CH [symbol] | Bnz | (6-2-5b-1) |
| N(CH [symbol] ) | CH [symbol] | Et | Et | (6-2-6b-1) |
| N(CH [symbol] ) | CH [symbol] | CH [symbol] CH [symbol] OCH [symbol] | CH [symbol] CH [symbol] OCH [symbol] | (6-2-7b-1) |
| N(CH [symbol] ) | CH [symbol] | Pr | Bu | (6-2-8b-1) |
| N(CH [symbol] ) | CH [symbol] | Et | Et | (8-3-9b-1) |
| N(CH [symbol] ) | CH [symbol] | ⁱPr | ⁱPr | (6-2-10b-1) |
| N(CH [symbol] ) | CH [symbol] | Bu | Bu | (6-2-11b-1) |
| N(CH [symbol] ) | CH [symbol] CH [symbol] CH [symbol] | CH [symbol] | CH [symbol] | (6-2-12b-1) |
| N(CH [symbol] ) | CH [symbol] CH [symbol] CH [symbol] | CH [symbol] | Et | (6-2-13b-1) |
| N(CH [symbol] ) | CH [symbol] CH [symbol] CH [symbol] | CH [symbol] | ⁱPr | (6-2-14b-1) |
| N(CH [symbol] ) | CH [symbol] CH [symbol] CH [symbol] | CH [symbol] | Bu | (6-2-15b-1) |
| N(CH [symbol] ) | CH [symbol] CH [symbol] CH [symbol] | CH [symbol] | Bnz | (6-2-16b-1) |
| N(CH [symbol] ) | CH [symbol] CH [symbol] CH [symbol] | Et | Et | (6-2-17b-1) |
| N(CH [symbol] ) | CH [symbol] CH [symbol] CH [symbol] | CH [symbol] CH [symbol] OCH [symbol] | CH [symbol] CH [symbol] OCH [symbol] | (6-2-18b-1) |
| N(CH [symbol] ) | CH [symbol] CH [symbol] CH [symbol] | Pr | Bu | (6-2-19b-1) |
| N(CH [symbol] ) | CH [symbol] CH [symbol] CH [symbol] | Et | Et | (6-2-20b-1) |
| N(CH [symbol] ) | CH [symbol] CH [symbol] CH [symbol] | ⁱPr | ⁱPr | (6-2-21b-1) |
| N(CH [symbol] ) | CH [symbol] CH [symbol] CH [symbol] | Bu | Bu | (6-2-22b-1) |
| N(CH [symbol] ) | CH [symbol] (CH [symbol] ) [symbol] CH [symbol] | CH [symbol] | CH [symbol] | (6-2-23b-1) |
| N(CH [symbol] ) | CH [symbol] (CH [symbol] ) [symbol] CH [symbol] | CH [symbol] | Et | (6-2-24b-1) |
| N(CH [symbol] ) | CH [symbol] (CH [symbol] ) [symbol] CH [symbol] | CH [symbol] | ⁱPr | (6-2-25b-1) |
| N(CH [symbol] ) | CH [symbol] (CH [symbol] ) [symbol] CH [symbol] | CH [symbol] | Bu | (6-2-26b-1) |
| N(CH [symbol] ) | CH [symbol] (CH [symbol] ) [symbol] CH [symbol] | CH [symbol] | Bnz | (6-2-27b-1) |
| N(CH [symbol] ) | CH [symbol] (CH [symbol] ) [symbol] CH [symbol] | Et | Et | (6-2-28b-1) |
| N(CH [symbol] ) | CH [symbol] (CH [symbol] ) [symbol] CH [symbol] | CH [symbol] CH [symbol] OCH [symbol] | CH [symbol] CH [symbol] OCH [symbol] | (6-2-29b-1) |
| N(CH [symbol] ) | CH [symbol] (CH [symbol] ) [symbol] CH [symbol] | Pr | Bu | (6-2-30b-1) |
| N(CH [symbol] ) | CH [symbol] (CH [symbol] ) [symbol] CH [symbol] | Et | Et | (6-2-31b-1) |
| N(CH [symbol] ) | CH [symbol] (CH [symbol] ) [symbol] CH [symbol] | ⁱPr | ⁱPr | (6-2-32b-1) |
| N(CH [symbol] ) | CH [symbol] (CH [symbol] ) [symbol] CH [symbol] | Bu | Bu | (6-2-33b-1) |

[symbol]

indicates data missing or illegible when filed

| [symbol] | R [symbol] | R [symbol] | R [symbol] | |
|---|---|---|---|---|
| O | CH [symbol] | CH [symbol] | CH [symbol] | (6-2-1c-1) |
| O | CH [symbol] | CH [symbol] | Et | (6-2-2c-1) |
| O | CH [symbol] | CH [symbol] | ⁱPr | (6-2-3c-1) |
| O | CH [symbol] | CH [symbol] | Bu | (6-2-4c-1) |
| O | CH [symbol] | CH [symbol] | Bnz | (6-2-5c-1) |
| O | CH [symbol] | Et | Et | (6-2-6c-1) |
| O | CH [symbol] | CH [symbol] CH [symbol] OCH [symbol] | CH [symbol] CH [symbol] OCH [symbol] | (6-2-7c-1) |
| O | CH [symbol] | Pr | Bu | (6-2-8c-1) |
| O | CH [symbol] | Et | Et | (6-2-9c-1) |
| O | CH [symbol] | ⁱPr | ⁱPr | (6-2-10c-1) |
| O | CH [symbol] | Bu | Bu | (6-2-11c-1) |
| O | CH [symbol] CH [symbol] CH [symbol] | CH [symbol] | CH [symbol] | (6-2-12c-1) |
| O | CH [symbol] CH [symbol] CH [symbol] | CH [symbol] | Et | (6-2-13c-1) |
| O | CH [symbol] CH [symbol] CH [symbol] | CH [symbol] | ⁱPr | (8-2-14c-1) |
| O | CH [symbol] CH [symbol] CH [symbol] | CH [symbol] | Bu | (6-2-15c-1) |
| O | CH [symbol] CH [symbol] CH [symbol] | CH [symbol] | Bnz | (6-2-16c-1) |
| O | CH [symbol] CH [symbol] CH [symbol] | Et | Et | (6-2-17c-1) |
| O | CH [symbol] CH [symbol] CH [symbol] | CH [symbol] CH [symbol] OCH [symbol] | CH [symbol] CH [symbol] OCH [symbol] | (6-2-18c-1) |
| O | CH [symbol] CH [symbol] CH [symbol] | Pr | Bu | (6-2-19c-1) |
| O | CH [symbol] CH [symbol] CH [symbol] | Et | Et | (6-2-20c-1) |
| O | CH [symbol] CH [symbol] CH [symbol] | ⁱPr | ⁱPr | (6-2-21c-1) |
| O | CH [symbol] CH [symbol] CH [symbol] | Bu | Bu | (6-2-22c-1) |
| O | CH [symbol] (CH [symbol] ) [symbol] CH [symbol] | CH [symbol] | CH [symbol] | (6-2-23c-1) |
| O | CH [symbol] (CH [symbol] ) [symbol] CH [symbol] | CH [symbol] | Et | (6-2-24c-1) |
| O | CH [symbol] (CH [symbol] ) [symbol] CH [symbol] | CH [symbol] | ⁱPr | (6-2-25c-1) |
| O | CH [symbol] (CH [symbol] ) [symbol] CH [symbol] | CH [symbol] | Bu | (6-2-26c-1) |
| O | CH [symbol] (CH [symbol] ) [symbol] CH [symbol] | CH [symbol] | Bnz | (6-2-27c-1) |
| O | CH [symbol] (CH [symbol] ) [symbol] CH [symbol] | Et | Et | (6-2-28c-1) |
| O | CH [symbol] (CH [symbol] ) [symbol] CH [symbol] | CH [symbol] CH [symbol] OCH [symbol] | CH [symbol] CH [symbol] OCH [symbol] | (6-2-29c-1) |
| O | CH [symbol] (CH [symbol] ) [symbol] CH [symbol] | Pr | Bu | (6-2-30c-1) |
| O | CH [symbol] (CH [symbol] ) [symbol] CH [symbol] | Et | Et | (6-2-31c-1) |
| O | CH [symbol] (CH [symbol] ) [symbol] CH [symbol] | ⁱPr | ⁱPr | (6-2-32c-1) |
| O | CH [symbol] (CH [symbol] ) [symbol] CH [symbol] | Bu | Bu | (6-2-33c-1) |

[symbol]

indicates data missing or illegible when filed

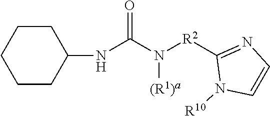

X, a, R¹, R², and R¹⁰ are as defined above. Preferably, X(R¹)^{a} represents NH or N(CH₃), R² represents CH₂, CH₂CH₂, or CH₂(CH₂)₄CH₂, and R¹⁰ represents CH₃, Et, ⁱPr, Bu, Bnz, or CH₂CH₂OCH₃.

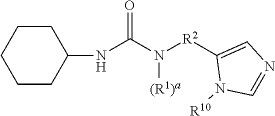

X, a, R¹, R², and R¹⁰ are as defined above. Preferably, X(R¹)^{a} represents NH, N(CH₃), or O, R² represents CH₂, CH₂CH₂, or CH₂(CH₂)₄CH₂, and R¹⁰ represents CH₃, Et, ⁱPr, Bu, Bnz, or CH₂CH₂OCH₃.

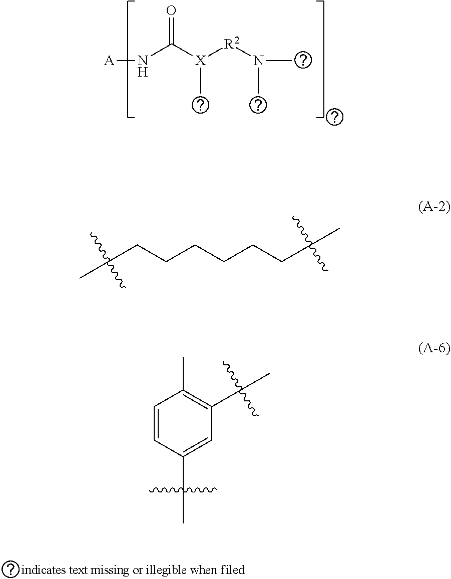

| A | [symbol] | R [symbol] | R [symbol] | R [symbol] | |
|---|---|---|---|---|---|
| A-2 | NH | CH [symbol] | CH [symbol] | CH [symbol] | (6-2-2a-2) |
| A-2 | NH | CH [symbol] | CH [symbol] | Et | (6-2-2a-2) |
| A-2 | NH | CH [symbol] | CH [symbol] | ⁱPr | (6-2-3a-2) |
| A-2 | NH | CH [symbol] | CH [symbol] | Bu | (6-2-4a-2) |
| A-2 | NH | CH [symbol] | CH [symbol] | Bnz | (6-2-5a-2) |
| A-2 | NH | CH [symbol] | Et | Et | (6-2-6a-2) |
| A-2 | NH | CH [symbol] | CH [symbol] CH [symbol] OCH [symbol] | CH [symbol] CH [symbol] OCH [symbol] | (6-2-7a-2) |
| A-2 | NH | CH [symbol] | Pr | Bu | (6-2-8a-2) |
| A-2 | NH | CH [symbol] | Et | Et | (6-2-9a-2) |
| A-2 | NH | CH [symbol] | ⁱPr | ⁱPr | (6-2-10a-2) |
| A-2 | NH | CH [symbol] | Bu | Bu | (6-2-11a-2) |
| A-2 | NH | CH [symbol] CH [symbol] CH [symbol] | CH [symbol] | CH [symbol] | (6-2-12a-2) |
| A-2 | NH | CH [symbol] CH [symbol] CH [symbol] | CH [symbol] | Et | (6-2-13a-2) |
| A-2 | NH | CH [symbol] CH [symbol] CH [symbol] | CH [symbol] | ⁱPr | (6-2-14a-2) |
| A-2 | NH | CH [symbol] CH [symbol] CH [symbol] | CH [symbol] | Bu | (6-2-15a-2) |
| A-2 | NH | CH [symbol] CH [symbol] CH [symbol] | CH [symbol] | Bnz | (6-2-16a-2) |
| A-2 | NH | CH [symbol] CH [symbol] CH [symbol] | Et | Et | (6-2-17a-2) |
| A-2 | NH | CH [symbol] CH [symbol] CH [symbol] | CH [symbol] CH [symbol] OCH [symbol] | CH [symbol] CH [symbol] OCH [symbol] | (6-2-18a-2) |
| A-2 | NH | CH [symbol] CH [symbol] CH [symbol] | Pr | Bu | (6-2-19a-2) |
| A-2 | NH | CH [symbol] CH [symbol] CH [symbol] | Et | Et | (6-2-20a-2) |
| A-2 | NH | CH [symbol] CH [symbol] CH [symbol] | ⁱPr | ⁱPr | (6-2-21a-2) |
| A-2 | NH | CH [symbol] CH [symbol] CH [symbol] | Bu | Bu | (6-2-22a-2) |
| A-2 | NH | CH [symbol] (CH [symbol] ) [symbol] CH [symbol] | CH [symbol] | CH [symbol] | (6-2-23a-2) |
| A-2 | NH | CH [symbol] (CH [symbol] ) [symbol] CH [symbol] | CH [symbol] | Et | (6-2-24a-2) |
| A-2 | NH | CH [symbol] (CH [symbol] ) [symbol] CH [symbol] | CH [symbol] | ⁱPr | (6-2-25a-2) |
| A-2 | NH | CH [symbol] (CH [symbol] ) [symbol] CH [symbol] | CH [symbol] | Bu | (6-2-26a-2) |
| A-2 | NH | CH [symbol] (CH [symbol] ) [symbol] CH [symbol] | CH [symbol] | Bnz | (6-2-27a-2) |
| A-2 | NH | CH [symbol] (CH [symbol] ) [symbol] CH [symbol] | Et | Et | (6-2-28a-2) |
| A-2 | NH | CH [symbol] (CH [symbol] ) [symbol] CH [symbol] | CH [symbol] CH [symbol] OCH [symbol] | CH [symbol] CH [symbol] OCH [symbol] | (6-2-29a-2) |
| A-2 | NH | CH [symbol] (CH [symbol] ) [symbol] CH [symbol] | Pr | Bu | (6-2-30a-2) |
| A-2 | NH | CH₂(CH₂)₄CH₂ | Et | Et | (6-2-31a-2) |
| A-2 | NH | CH₂(CH₂)₄CH₂ | ⁱPr | ⁱPr | (6-2-32a-2) |
| A-2 | NH | CH₂(CH₂)₄CH₂ | Bu | Bu | (6-2-33a-2) |

[symbol]

indicates data missing or illegible when filed

| A | [symbol] | R [symbol] | R [symbol] | R [symbol] | |
|---|---|---|---|---|---|
| A-2 | N(CH [symbol] ) | CH [symbol] | CH [symbol] | CH [symbol] | (6-2-1b-2) |
| A-2 | N(CH [symbol] ) | CH [symbol] | CH [symbol] | Et | (6-2-2b-2) |
| A-2 | N(CH [symbol] ) | CH [symbol] | CH [symbol] | ⁱPr | (6-2-3b-2) |
| A-2 | N(CH [symbol] ) | CH [symbol] | CH [symbol] | Bu | (6-2-4b-2) |
| A-2 | N(CH [symbol] ) | CH [symbol] | CH [symbol] | Bnz | (6-2-5b-2) |
| A-2 | N(CH [symbol] ) | CH [symbol] | Et | Et | (6-2-6b-2) |
| A-2 | N(CH [symbol] ) | CH [symbol] | CH [symbol] CH [symbol] OCH [symbol] | CH [symbol] CH [symbol] OCH [symbol] | (6-2-7b-2) |
| A-2 | N(CH [symbol] ) | CH [symbol] | Pr | Bu | (6-2-8b-2) |
| A-2 | N(CH [symbol] ) | CH [symbol] | Et | Et | (6-2-9b-2) |
| A-2 | N(CH [symbol] ) | CH [symbol] | ⁱPr | ⁱPr | (6-2-10b-2) |
| A-2 | N(CH [symbol] ) | CH [symbol] | Bu | Bu | (6-2-11b-2) |
| A-2 | N(CH [symbol] ) | CH [symbol] CH [symbol] CH [symbol] | CH [symbol] | CH [symbol] | (6-2-12b-2) |
| A-2 | N(CH [symbol] ) | CH [symbol] CH [symbol] CH [symbol] | CH [symbol] | Et | (6-2-13b-2) |
| A-2 | N(CH [symbol] ) | CH [symbol] CH [symbol] CH [symbol] | CH [symbol] | ⁱPr | (8-2-14b-2) |
| A-2 | N(CH [symbol] ) | CH [symbol] CH [symbol] CH [symbol] | CH [symbol] | Bu | (6-2-15b-2) |
| A-2 | N(CH [symbol] ) | CH [symbol] CH [symbol] CH [symbol] | CH [symbol] | Bnz | (6-2-16b-2) |
| A-2 | N(CH [symbol] ) | CH [symbol] CH [symbol] CH [symbol] | Et | Et | (6-2-17b-2) |
| A-2 | N(CH [symbol] ) | CH [symbol] CH [symbol] CH [symbol] | CH [symbol] CH [symbol] OCH [symbol] | CH [symbol] CH [symbol] OCH [symbol] | (6-2-18b-2) |
| A-2 | N(CH [symbol] ) | CH [symbol] CH [symbol] CH [symbol] | Pr | Bu | (6-2-19b-2) |
| A-2 | N(CH [symbol] ) | CH [symbol] CH [symbol] CH [symbol] | Et | Et | (6-2-20b-2) |
| A-2 | N(CH [symbol] ) | CH [symbol] CH [symbol] CH [symbol] | ⁱPr | ⁱPr | (6-2-21b-2) |
| A-2 | N(CH [symbol] ) | CH [symbol] CH [symbol] CH [symbol] | Bu | Bu | (6-2-22b-2) |
| A-2 | N(CH [symbol] ) | CH [symbol] (CH [symbol] ) [symbol] CH [symbol] | CH [symbol] | CH [symbol] | (6-2-23b-2) |
| A-2 | N(CH [symbol] ) | CH [symbol] (CH [symbol] ) [symbol] CH [symbol] | CH [symbol] | Et | (6-2-24b-2) |
| A-2 | N(CH [symbol] ) | CH [symbol] (CH [symbol] ) [symbol] CH [symbol] | CH [symbol] | ⁱPr | (6-2-25b-2) |
| A-2 | N(CH [symbol] ) | CH [symbol] (CH [symbol] ) [symbol] CH [symbol] | CH [symbol] | Bu | (6-2-26b-2) |
| A-2 | N(CH [symbol] ) | CH [symbol] (CH [symbol] ) [symbol] CH [symbol] | CH [symbol] | Bnz | (6-2-27b-2) |
| A-2 | N(CH [symbol] ) | CH [symbol] (CH [symbol] ) [symbol] CH [symbol] | Et | Et | (6-2-28b-2) |
| A-2 | N(CH [symbol] ) | CH [symbol] (CH [symbol] ) [symbol] CH [symbol] | CH [symbol] CH [symbol] OCH [symbol] | CH [symbol] CH [symbol] OCH [symbol] | (6-2-29b-2) |
| A-2 | N(CH [symbol] ) | CH [symbol] (CH [symbol] ) [symbol] CH [symbol] | Pr | Bu | (6-2-30b-2) |
| A-2 | N(CH [symbol] ) | CH [symbol] (CH [symbol] ) [symbol] CH [symbol] | Et | Et | (6-2-31b-2) |
| A-2 | N(CH [symbol] ) | CH [symbol] (CH [symbol] ) [symbol] CH [symbol] | ⁱPr | ⁱPr | (6-2-32b-2) |
| A-2 | N(CH [symbol] ) | CH [symbol] (CH [symbol] ) [symbol] CH [symbol] | Bu | Bu | (6-2-33b-2) |

[symbol]

indicates data missing or illegible when filed

| A | [symbol] | R [symbol] | R [symbol] | R [symbol] | |
|---|---|---|---|---|---|
| A-2 | O | CH [symbol] | CH [symbol] | CH [symbol] | (6-2-1c-2) |
| A-2 | O | CH [symbol] | CH [symbol] | Et | (6-2-2c-2) |
| A-2 | O | CH [symbol] | CH [symbol] | ⁱPr | (6-2-3c-2) |
| A-2 | O | CH [symbol] | CH [symbol] | Bu | (6-2-4c-2) |
| A-2 | O | CH [symbol] | CH [symbol] | Bnz | (6-2-5c-2) |
| A-2 | O | CH [symbol] | Et | Et | (6-2-6c-2) |
| A-2 | O | CH [symbol] | CH [symbol] CH [symbol] OCH [symbol] | CH [symbol] CH [symbol] OCH [symbol] | (6-2-7c-2) |
| A-2 | O | CH [symbol] | Pr | Bu | (6-2-8c-2) |
| A-2 | O | CH [symbol] | Et | Et | (6-2-9c-2) |
| A-2 | O | CH [symbol] | ⁱPr | ⁱPr | (6-2-10c-2) |
| A-2 | O | CH [symbol] | Bu | Bu | (6-2-11c-2) |
| A-2 | O | CH [symbol] CH [symbol] CH [symbol] | CH [symbol] | CH [symbol] | (6-2-12c-2) |
| A-2 | O | CH [symbol] CH [symbol] CH [symbol] | CH [symbol] | Et | (6-2-13c-2) |
| A-2 | O | CH [symbol] CH [symbol] CH [symbol] | CH [symbol] | ⁱPr | (6-2-14c-2) |
| A-2 | O | CH [symbol] CH [symbol] CH [symbol] | CH [symbol] | Bu | (6-2-15c-2) |
| A-2 | O | CH [symbol] CH [symbol] CH [symbol] | CH [symbol] | Bnz | (6-2-16c-2) |
| A-2 | O | CH [symbol] CH [symbol] CH [symbol] | Et | Et | (6-2-17c-2) |
| A-2 | O | CH [symbol] CH [symbol] CH [symbol] | CH [symbol] CH [symbol] OCH [symbol] | CH [symbol] CH [symbol] OCH [symbol] | (6-2-18c-2) |
| A-2 | O | CH [symbol] CH [symbol] CH [symbol] | Pr | Bu | (6-2-19c-2) |
| A-2 | O | CH [symbol] CH [symbol] CH [symbol] | Et | Et | (6-2-20c-2) |
| A-2 | O | CH [symbol] CH [symbol] CH [symbol] | ⁱPr | ⁱPr | (6-2-21c-2) |
| A-2 | O | CH [symbol] CH [symbol] CH [symbol] | Bu | Bu | (6-2-22c-2) |
| A-2 | O | CH [symbol] (CH [symbol] ) [symbol] CH [symbol] | CH [symbol] | CH [symbol] | (6-2-23c-2) |
| A-2 | O | CH [symbol] (CH [symbol] ) [symbol] CH [symbol] | CH [symbol] | Et | (6-2-24c-2) |
| A-2 | O | CH [symbol] (CH [symbol] ) [symbol] CH [symbol] | CH [symbol] | ⁱPr | (6-2-25c-2) |
| A-2 | O | CH [symbol] (CH [symbol] ) [symbol] CH [symbol] | CH [symbol] | Bu | (6-2-26c-2) |
| A-2 | O | CH [symbol] (CH [symbol] ) [symbol] CH [symbol] | CH [symbol] | Bnz | (6-2-27c-2) |
| A-2 | O | CH [symbol] (CH [symbol] ) [symbol] CH [symbol] | Et | Et | (6-2-28c-2) |
| A-2 | O | CH [symbol] (CH [symbol] ) [symbol] CH [symbol] | CH [symbol] CH [symbol] OCH [symbol] | CH [symbol] CH [symbol] OCH [symbol] | (6-2-29c-2) |
| A-2 | O | CH [symbol] (CH [symbol] ) [symbol] CH [symbol] | Pr | Bu | (6-2-30c-2) |
| A-2 | O | CH [symbol] (CH [symbol] ) [symbol] CH [symbol] | Et | Et | (6-2-31c-2) |
| A-2 | O | CH [symbol] (CH [symbol] ) [symbol] CH [symbol] | ⁱPr | ⁱPr | (6-2-32c-2) |
| A-2 | O | CH [symbol] (CH [symbol] ) [symbol] CH [symbol] | Bu | Bu | (6-2-33c-2) |

[symbol]

indicates data missing or illegible when filed

| A | [symbol] | R [symbol] | R [symbol] | R [symbol] | |
|---|---|---|---|---|---|
| A-6 | NH | CH [symbol] | CH [symbol] | CH [symbol] | (6-2-1a-6) |
| A-6 | NH | CH [symbol] | CH [symbol] | Et | (6-2-2a-6) |
| A-6 | NH | CH [symbol] | CH [symbol] | ⁱPr | (6-2-3a-6) |
| A-6 | NH | CH [symbol] | CH [symbol] | Bu | (6-2-4a-6) |
| A-6 | NH | CH [symbol] | CH [symbol] | Bnz | (6-2-5a-6) |
| A-6 | NH | CH [symbol] | Et | Et | (6-2-6a-6) |
| A-6 | NH | CH [symbol] | CH [symbol] CH [symbol] OCH [symbol] | CH [symbol] CH [symbol] OCH [symbol] | (6-2-7a-6) |
| A-6 | NH | CH [symbol] | Pr | Bu | (6-2-8a-6) |
| A-6 | NH | CH [symbol] | Et | Et | (6-2-9a-6) |
| A-6 | NH | CH [symbol] | ⁱPr | ⁱPr | (6-2-10a-6) |
| A-6 | NH | CH [symbol] | Bu | Bu | (6-2-11a-6) |
| A-6 | NH | CH [symbol] CH [symbol] CH [symbol] | CH [symbol] | CH [symbol] | (6-2-12a-6) |
| A-6 | NH | CH [symbol] CH [symbol] CH [symbol] | CH [symbol] | Et | (6-2-13a-6) |
| A-6 | NH | CH [symbol] CH [symbol] CH [symbol] | CH [symbol] | ⁱPr | (6-2-14a-6) |
| A-6 | NH | CH [symbol] CH [symbol] CH [symbol] | CH [symbol] | Bu | (6-2-15a-6) |
| A-6 | NH | CH [symbol] CH [symbol] CH [symbol] | CH [symbol] | Bnz | (6-2-16a-6) |
| A-6 | NH | CH [symbol] CH [symbol] CH [symbol] | Et | Et | (6-2-17a-6) |
| A-6 | NH | CH [symbol] CH [symbol] CH [symbol] | CH [symbol] CH [symbol] OCH [symbol] | CH [symbol] CH [symbol] OCH [symbol] | (6-2-18a-6) |
| A-6 | NH | CH [symbol] CH [symbol] CH [symbol] | Pr | Bu | (6-2-19a-6) |
| A-6 | NH | CH [symbol] CH [symbol] CH [symbol] | Et | Et | (6-2-20a-6) |
| A-6 | NH | CH [symbol] CH [symbol] CH [symbol] | ⁱPr | ⁱPr | (6-2-21a-6) |
| A-6 | NH | CH [symbol] CH [symbol] CH [symbol] | Bu | Bu | (6-2-22a-6) |
| A-6 | NH | CH [symbol] (CH [symbol] ) [symbol] CH [symbol] | CH [symbol] | CH [symbol] | (6-2-23a-6) |
| A-6 | NH | CH [symbol] (CH [symbol] ) [symbol] CH [symbol] | CH [symbol] | Et | (6-2-24a-6) |
| A-6 | NH | CH [symbol] (CH [symbol] ) [symbol] CH [symbol] | CH [symbol] | ⁱPr | (6-2-25a-6) |
| A-6 | NH | CH [symbol] (CH [symbol] ) [symbol] CH [symbol] | CH [symbol] | Bu | (6-2-26a-6) |
| A-6 | NH | CH [symbol] (CH [symbol] ) [symbol] CH [symbol] | CH [symbol] | Bnz | (6-2-27a-6) |
| A-6 | NH | CH [symbol] (CH [symbol] ) [symbol] CH [symbol] | Et | Et | (6-2-28a-6) |
| A-6 | NH | CH [symbol] (CH [symbol] ) [symbol] CH [symbol] | CH [symbol] CH [symbol] OCH [symbol] | CH [symbol] CH [symbol] OCH [symbol] | (6-2-29a-6) |
| A-6 | NH | CH [symbol] (CH [symbol] ) [symbol] CH [symbol] | Pr | Bu | (6-2-30a-6) |
| A-6 | NH | CH [symbol] (CH [symbol] ) [symbol] CH [symbol] | Et | Et | (6-2-31a-6) |
| A-6 | NH | CH [symbol] (CH [symbol] ) [symbol] CH [symbol] | ⁱPr | ⁱPr | (6-2-32a-6) |
| A-6 | NH | CH [symbol] (CH [symbol] ) [symbol] CH [symbol] | Bu | Bu | (6-2-33a-6) |

[symbol]

indicates data missing or illegible when filed

| A | [symbol] | R [symbol] | R [symbol] | R [symbol] | |
|---|---|---|---|---|---|
| A-6 | N(CH [symbol] ) | CH [symbol] | CH [symbol] | CH [symbol] | (6-2-1b-6) |
| A-6 | N(CH [symbol] ) | CH [symbol] | CH [symbol] | Et | (6-2-2b-6) |
| A-6 | N(CH [symbol] ) | CH [symbol] | CH [symbol] | ⁱPr | (6-2-3b-6) |
| A-6 | N(CH [symbol] ) | CH [symbol] | CH [symbol] | Bu | (6-2-4b-6) |
| A-6 | N(CH [symbol] ) | CH [symbol] | CH [symbol] | Bnz | (6-2-5b-6) |
| A-6 | N(CH [symbol] ) | CH [symbol] | Et | Et | (6-2-6b-6) |
| A-6 | N(CH [symbol] ) | CH [symbol] | CH [symbol] CH [symbol] OCH [symbol] | CH [symbol] CH [symbol] OCH [symbol] | (6-2-7b-6) |
| A-6 | N(CH [symbol] ) | CH [symbol] | Pr | Bu | (6-2-8b-6) |
| A-6 | N(CH [symbol] ) | CH [symbol] | Et | Et | (6-3-9b-6) |
| A-6 | N(CH [symbol] ) | CH [symbol] | ⁱPr | ⁱPr | (6-2-10b-6) |
| A-6 | N(CH [symbol] ) | CH [symbol] | Bu | Bu | (6-2-11b-6) |
| A-6 | N(CH [symbol] ) | CH [symbol] CH [symbol] CH [symbol] | CH [symbol] | CH [symbol] | (6-2-12b-6) |
| A-6 | N(CH [symbol] ) | CH [symbol] CH [symbol] CH [symbol] | CH [symbol] | Et | (6-2-13b-6) |
| A-6 | N(CH [symbol] ) | CH [symbol] CH [symbol] CH [symbol] | CH [symbol] | ⁱPr | (6-2-14b-6) |
| A-6 | N(CH [symbol] ) | CH [symbol] CH [symbol] CH [symbol] | CH [symbol] | Bu | (6-2-15b-6) |
| A-6 | N(CH [symbol] ) | CH [symbol] CH [symbol] CH [symbol] | CH [symbol] | Bnz | (6-2-16b-6) |
| A-6 | N(CH [symbol] ) | CH [symbol] CH [symbol] CH [symbol] | Et | Et | (6-2-176-6) |
| A-6 | N(CH [symbol] ) | CH [symbol] CH [symbol] CH [symbol] | CH [symbol] CH [symbol] OCH [symbol] | CH [symbol] CH [symbol] OCH [symbol] | (6-2-18b-6) |
| A-6 | N(CH [symbol] ) | CH [symbol] CH [symbol] CH [symbol] | Pr | Bu | (6-2-19b-6) |
| A-6 | N(CH [symbol] ) | CH [symbol] CH [symbol] CH [symbol] | Et | Et | (6-2-20b-6) |
| A-6 | N(CH [symbol] ) | CH [symbol] CH [symbol] CH [symbol] | ⁱPr | ⁱPr | (6-2-21b-6) |
| A-6 | N(CH [symbol] ) | CH [symbol] CH [symbol] CH [symbol] | Bu | Bu | (6-2-22b-6) |
| A-6 | N(CH [symbol] ) | CH [symbol] (CH [symbol] ) [symbol] CH [symbol] | CH [symbol] | CH [symbol] | (6-2-23b-6) |
| A-6 | N(CH [symbol] ) | CH [symbol] (CH [symbol] ) [symbol] CH [symbol] | CH [symbol] | Et | (6-2-24b-6) |
| A-6 | N(CH [symbol] ) | CH [symbol] (CH [symbol] ) [symbol] CH [symbol] | CH [symbol] | ⁱPr | (6-2-25b-6) |
| A-6 | N(CH [symbol] ) | CH [symbol] (CH [symbol] ) [symbol] CH [symbol] | CH [symbol] | Bu | (6-2-26b-6) |
| A-6 | N(CH [symbol] ) | CH [symbol] (CH [symbol] ) [symbol] CH [symbol] | CH [symbol] | Bnz | (6-2-27b-6) |
| A-6 | N(CH [symbol] ) | CH [symbol] (CH [symbol] ) [symbol] CH [symbol] | Et | Et | (6-2-28b-6) |
| A-6 | N(CH [symbol] ) | CH [symbol] (CH [symbol] ) [symbol] CH [symbol] | CH [symbol] CH [symbol] OCH [symbol] | CH [symbol] CH [symbol] OCH [symbol] | (6-2-29b-6) |
| A-6 | N(CH [symbol] ) | CH [symbol] (CH [symbol] ) [symbol] CH [symbol] | Pr | Bu | (6-2-30b-6) |
| A-6 | N(CH [symbol] ) | CH [symbol] (CH [symbol] ) [symbol] CH [symbol] | Et | Et | (6-2-31b-6) |
| A-6 | N(CH [symbol] ) | CH [symbol] (CH [symbol] ) [symbol] CH [symbol] | ⁱPr | ⁱPr | (6-2-32b-6) |
| A-6 | N(CH [symbol] ) | CH [symbol] (CH [symbol] ) [symbol] CH [symbol] | Bu | Bu | (6-2-33b-6) |

[symbol]

indicates data missing or illegible when filed

| A | [symbol] | R [symbol] | R [symbol] | R [symbol] | |
|---|---|---|---|---|---|
| A-6 | O | CH [symbol] | CH [symbol] | CH [symbol] | (6-2-1c-6) |
| A-6 | O | CH [symbol] | CH [symbol] | Et | (6-2-2c-6) |
| A-6 | O | CH [symbol] | CH [symbol] | ⁱPr | (6-2-3c-6) |
| A-6 | O | CH [symbol] | CH [symbol] | Bu | (6-2-4c-6) |
| A-6 | O | CH [symbol] | CH [symbol] | Bnz | (6-2-5c-6) |
| A-6 | O | CH [symbol] | Et | Et | (6-2-6c-6) |
| A-6 | O | CH [symbol] | CH [symbol] CH [symbol] OCH [symbol] | CH [symbol] CH [symbol] OCH [symbol] | (6-2-7c-6) |
| A-6 | O | CH [symbol] | Pr | Bu | (6-2-8c-6) |
| A-6 | O | CH [symbol] | Et | Et | (6-2-9c-6) |
| A-6 | O | CH [symbol] | ⁱPr | ⁱPr | (6-2-10c-6) |
| A-6 | O | CH [symbol] | Bu | Bu | (6-2-11c-6) |
| A-6 | O | CH [symbol] CH [symbol] CH [symbol] | CH [symbol] | CH [symbol] | (6-2-12c-6) |
| A-6 | O | CH [symbol] CH [symbol] CH [symbol] | CH [symbol] | Et | (6-2-13c-6) |
| A-6 | O | CH [symbol] CH [symbol] CH [symbol] | CH [symbol] | ⁱPr | (6-2-14c-6) |
| A-6 | O | CH [symbol] CH [symbol] CH [symbol] | CH [symbol] | Bu | (6-2-15c-6) |
| A-6 | O | CH [symbol] CH [symbol] CH [symbol] | CH [symbol] | Bnz | (6-2-16c-6) |
| A-6 | O | CH [symbol] CH [symbol] CH [symbol] | Et | Et | (6-2-17c-6) |
| A-6 | O | CH [symbol] CH [symbol] CH [symbol] | CH [symbol] CH [symbol] OCH [symbol] | CH [symbol] CH [symbol] OCH [symbol] | (6-2-18c-6) |
| A-6 | O | CH [symbol] CH [symbol] CH [symbol] | Pr | Bu | (6-2-19c-6) |
| A-6 | O | CH [symbol] CH [symbol] CH [symbol] | Et | Et | (6-2-20c-6) |
| A-6 | O | CH [symbol] CH [symbol] CH [symbol] | ⁱPr | ⁱPr | (6-2-21c-6) |
| A-6 | O | CH [symbol] CH [symbol] CH [symbol] | Bu | Bu | (6-2-22c-6) |
| A-6 | O | CH [symbol] (CH [symbol] ) [symbol] CH [symbol] | CH [symbol] | CH [symbol] | (6-2-23c-6) |
| A-6 | O | CH [symbol] (CH [symbol] ) [symbol] CH [symbol] | CH [symbol] | Et | (6-2-24c-6) |
| A-6 | O | CH [symbol] (CH [symbol] ) [symbol] CH [symbol] | CH [symbol] | ⁱPr | (6-2-25c-6) |
| A-6 | O | CH [symbol] (CH [symbol] ) [symbol] CH [symbol] | CH [symbol] | Bu | (6-2-26c-6) |
| A-6 | O | CH [symbol] (CH [symbol] ) [symbol] CH [symbol] | CH [symbol] | Bnz | (6-2-27c-6) |
| A-6 | O | CH [symbol] (CH [symbol] ) [symbol] CH [symbol] | Et | Et | (6-2-28c-6) |
| A-6 | O | CH [symbol] (CH [symbol] ) [symbol] CH [symbol] | CH [symbol] CH [symbol] OCH [symbol] | CH [symbol] CH [symbol] OCH [symbol] | (6-2-29c-6) |
| A-6 | O | CH [symbol] (CH [symbol] ) [symbol] CH [symbol] | Pr | Bu | (6-2-30c-6) |
| A-6 | O | CH [symbol] (CH [symbol] ) [symbol] CH [symbol] | Et | Et | (6-2-31c-6) |
| A-6 | O | CH [symbol] (CH [symbol] ) [symbol] CH [symbol] | ⁱPr | ⁱPr | (6-2-32c-6) |
| A-6 | O | CH [symbol] (CH [symbol] ) [symbol] CH [symbol] | Bu | Bu | (6-2-33c-6) |

[symbol]

indicates data missing or illegible when filed

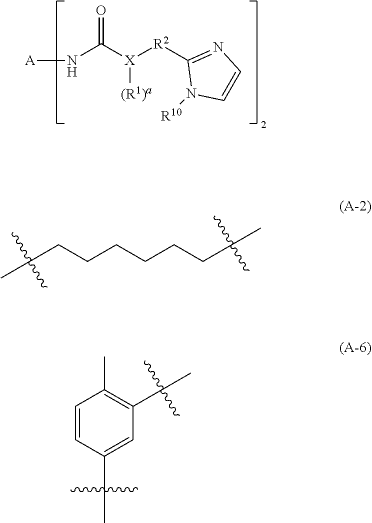

A, X, a, R¹, R², and R¹⁰ are as defined above. Preferably, A represents a group represented by formula (A-2) or a group represented by formula (A-6), X(R¹)^{a} represents NH, N(CH₃), or O, R² represents CH₂, CH₂CH₂, or CH₂(CH₂)₄CH₂, and R¹⁰ represents CH₃, Et, ⁱPr, Bu, Bnz, or CH₂CH₂OCH₃.

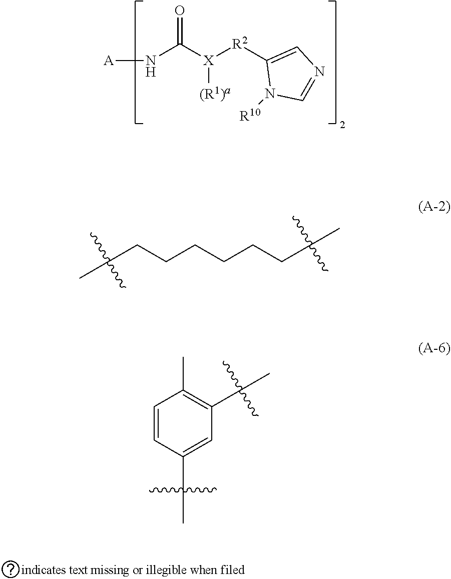

A, X, a, R¹, R², and R¹⁰ are as defined above. Preferably, A represents a group represented by formula (A-2) or a group represented by formula (A-6), X(R¹)^{a} represents NH, N(CH₃), or O, R² represents CH₂, CH₂CH₂, or CH₂(CH₂)₄CH₂, and R¹⁰ represents CH₃, Et, ⁱPr, Bu, Bnz, or CH₂CH₂OCH₃.

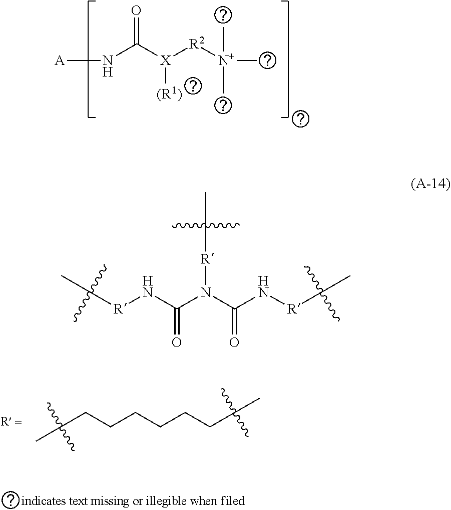

| A | [symbol] | R [symbol] | R [symbol] | R [symbol] | |
|---|---|---|---|---|---|
| A-14 | NH | CH [symbol] | CH [symbol] | CH [symbol] | (6-2-1a-14) |
| A-14 | NH | CH [symbol] | CH [symbol] | Et | (6-2-2a-14) |
| A-14 | NH | CH [symbol] | CH [symbol] | ⁱPr | (6-2-3a-14) |
| A-14 | NH | CH [symbol] | CH [symbol] | Bu | (6-2-4a-14) |
| A-14 | NH | CH [symbol] | CH [symbol] | Bnz | (6-2-5a-14) |
| A-14 | NH | CH [symbol] | Et | Et | (6-2-6a-14) |
| A-14 | NH | CH [symbol] | CH [symbol] CH [symbol] OCH [symbol] | CH [symbol] CH [symbol] OCH [symbol] | (6-2-7a-14) |
| A-14 | NH | CH [symbol] | Pr | Bu | (6-2-8a-14) |
| A-14 | NH | CH [symbol] | Et | Et | (6-2-9a-14) |
| A-14 | NH | CH [symbol] | ⁱPr | ⁱPr | (6-2-10a-14) |
| A-14 | NH | CH [symbol] | Bu | Bu | (6-2-11a-14) |
| A-14 | NH | CH [symbol] CH [symbol] CH [symbol] | CH [symbol] | CH [symbol] | (6-2-12a-14) |
| A-14 | NH | CH [symbol] CH [symbol] CH [symbol] | CH [symbol] | Et | (6-2-13a-14) |
| A-14 | NH | CH [symbol] CH [symbol] CH [symbol] | CH [symbol] | ⁱPr | (6-2-14a-14) |
| A-14 | NH | CH [symbol] CH [symbol] CH [symbol] | CH [symbol] | Bu | (6-2-15a-14) |
| A-14 | NH | CH [symbol] CH [symbol] CH [symbol] | CH [symbol] | Bnz | (6-2-16a-14) |
| A-14 | NH | CH [symbol] CH [symbol] CH [symbol] | Et | Et | (6-2-17a-14) |
| A-14 | NH | CH [symbol] CH [symbol] CH [symbol] | CH [symbol] CH [symbol] OCH [symbol] | CH [symbol] CH [symbol] OCH [symbol] | (6-2-18a-14) |
| A-14 | NH | CH [symbol] CH [symbol] CH [symbol] | Pr | Bu | (6-2-19a-14) |
| A-14 | NH | CH [symbol] CH [symbol] CH [symbol] | Et | Et | (6-2-20a-14) |
| A-14 | NH | CH [symbol] CH [symbol] CH [symbol] | ⁱPr | ⁱPr | (6-2-21a-14) |
| A-14 | NH | CH [symbol] CH [symbol] CH [symbol] | Bu | Bu | (6-2-22a-14) |
| A-14 | NH | CH [symbol] (CH [symbol] ) [symbol] CH [symbol] | CH [symbol] | CH [symbol] | (6-2-23a-14) |
| A-14 | NH | CH [symbol] (CH [symbol] ) [symbol] CH [symbol] | CH [symbol] | Et | (6-2-24a-14) |
| A-14 | NH | CH [symbol] (CH [symbol] ) [symbol] CH [symbol] | CH [symbol] | ⁱPr | (6-2-25a-14) |
| A-14 | NH | CH [symbol] (CH [symbol] ) [symbol] CH [symbol] | CH [symbol] | Bu | (6-2-26a-14) |
| A-14 | NH | CH [symbol] (CH [symbol] ) [symbol] CH [symbol] | CH [symbol] | Bnz | (6-2-27a-14) |
| A-14 | NH | CH [symbol] (CH [symbol] ) [symbol] CH [symbol] | Et | Et | (6-2-28a-14) |
| A-14 | NH | CH [symbol] (CH [symbol] ) [symbol] CH [symbol] | CH [symbol] CH [symbol] OCH [symbol] | CH [symbol] CH [symbol] OCH [symbol] | (6-2-29a-14) |
| A-14 | NH | CH [symbol] (CH [symbol] ) [symbol] CH [symbol] | Pr | Bu | (6-2-30a-14) |
| A-14 | NH | CH₂(CH₂)₄CH₂ | Et | Et | (6-2-31a-14) |
| A-14 | NH | CH₂(CH₂)₄CH₂ | ⁱPr | ⁱPr | (6-2-32a-14) |
| A-14 | NH | CH₂(CH₂)₄CH₂ | Bu | Bu | (6-2-33a-14) |

[symbol]

indicates data missing or illegible when filed

| A | [symbol] | R [symbol] | R [symbol] | R [symbol] | |
|---|---|---|---|---|---|
| A-14 | N(CH [symbol] ) | CH [symbol] | CH [symbol] | CH [symbol] | (6-2-1b-14) |
| A-14 | N(CH [symbol] ) | CH [symbol] | CH [symbol] | Et | (6-2-2b-14) |
| A-14 | N(CH [symbol] ) | CH [symbol] | CH [symbol] | ⁱPr | (6-2-3b-14) |
| A-14 | N(CH [symbol] ) | CH [symbol] | CH [symbol] | Bu | (6-2-4b-14) |
| A-14 | N(CH [symbol] ) | CH [symbol] | CH [symbol] | Bnz | (6-2-5b-14) |
| A-14 | N(CH [symbol] ) | CH [symbol] | Et | Et | (6-2-6b-14) |
| A-14 | N(CH [symbol] ) | CH [symbol] | CH [symbol] CH [symbol] OCH [symbol] | CH [symbol] CH [symbol] OCH [symbol] | (6-2-7b-14) |
| A-14 | N(CH [symbol] ) | CH [symbol] | Pr | Bu | (6-2-8b-14) |
| A-14 | N(CH [symbol] ) | CH [symbol] | Et | Et | (6-2-9b-14) |
| A-14 | N(CH [symbol] ) | CH [symbol] | ⁱPr | ⁱPr | (6-2-10b-14) |
| A-14 | N(CH [symbol] ) | CH [symbol] | Bu | Bu | (6-2-11b-14) |
| A-14 | N(CH [symbol] ) | CH [symbol] CH [symbol] CH [symbol] | CH [symbol] | CH [symbol] | (6-2-12b-14) |
| A-14 | N(CH [symbol] ) | CH [symbol] CH [symbol] CH [symbol] | CH [symbol] | Et | (6-2-13b-14) |
| A-14 | N(CH [symbol] ) | CH [symbol] CH [symbol] CH [symbol] | CH [symbol] | ⁱPr | (6-2-14b-14) |
| A-14 | N(CH [symbol] ) | CH [symbol] CH [symbol] CH [symbol] | CH [symbol] | Bu | (6-2-15b-14) |
| A-14 | N(CH [symbol] ) | CH [symbol] CH [symbol] CH [symbol] | CH [symbol] | Bnz | (6-2-16b-14) |
| A-14 | N(CH [symbol] ) | CH [symbol] CH [symbol] CH [symbol] | Et | Et | (6-2-17b-14) |
| A-14 | N(CH [symbol] ) | CH [symbol] CH [symbol] CH [symbol] | CH [symbol] CH [symbol] OCH [symbol] | CH [symbol] CH [symbol] OCH [symbol] | (6-2-18b-14) |
| A-14 | N(CH [symbol] ) | CH [symbol] CH [symbol] CH [symbol] | Pr | Bu | (6-2-19b-14) |
| A-14 | N(CH [symbol] ) | CH [symbol] CH [symbol] CH [symbol] | Et | Et | (6-2-20b-14) |
| A-14 | N(CH [symbol] ) | CH [symbol] CH [symbol] CH [symbol] | ⁱPr | ⁱPr | (6-2-21b-14) |
| A-14 | N(CH [symbol] ) | CH [symbol] CH [symbol] CH [symbol] | Bu | Bu | (6-2-22b-14) |
| A-14 | N(CH [symbol] ) | CH [symbol] (CH [symbol] ) [symbol] CH [symbol] | CH [symbol] | CH [symbol] | (6-2-23b-14) |
| A-14 | N(CH [symbol] ) | CH [symbol] (CH [symbol] ) [symbol] CH [symbol] | CH [symbol] | Et | (6-2-24b-14) |
| A-14 | N(CH [symbol] ) | CH [symbol] (CH [symbol] ) [symbol] CH [symbol] | CH [symbol] | ⁱPr | (6-2-25b-14) |
| A-14 | N(CH [symbol] ) | CH [symbol] (CH [symbol] ) [symbol] CH [symbol] | CH [symbol] | Bu | (6-2-26b-14) |
| A-14 | N(CH [symbol] ) | CH [symbol] (CH [symbol] ) [symbol] CH [symbol] | CH [symbol] | Bnz | (6-2-27b-14) |
| A-14 | N(CH [symbol] ) | CH [symbol] (CH [symbol] ) [symbol] CH [symbol] | Et | Et | (6-2-28b-14) |
| A-14 | N(CH [symbol] ) | CH [symbol] (CH [symbol] ) [symbol] CH [symbol] | CH [symbol] CH [symbol] OCH [symbol] | CH [symbol] CH [symbol] OCH [symbol] | (6-2-29b-14) |
| A-14 | N(CH [symbol] ) | CH [symbol] (CH [symbol] ) [symbol] CH [symbol] | Pr | Bu | (6-2-30b-14) |
| A-14 | N(CH [symbol] ) | CH [symbol] (CH [symbol] ) [symbol] CH [symbol] | Et | Et | (6-2-31b-14) |
| A-14 | N(CH [symbol] ) | CH [symbol] (CH [symbol] ) [symbol] CH [symbol] | ⁱPr | ⁱPr | (6-2-32b-14) |
| A-14 | N(CH [symbol] ) | CH [symbol] (CH [symbol] ) [symbol] CH [symbol] | Bu | Bu | (6-2-33b-14) |

[symbol]

indicates data missing or illegible when filed

| A | [symbol] | R [symbol] | R [symbol] | R [symbol] | |
|---|---|---|---|---|---|
| A-14 | O | CH [symbol] | CH [symbol] | CH [symbol] | (6-2-1c-14) |
| A-14 | O | CH [symbol] | CH [symbol] | Et | (6-2-2c-14) |
| A-14 | O | CH [symbol] | CH [symbol] | ⁱPr | (6-2-3c-14) |
| A-14 | O | CH [symbol] | CH [symbol] | Bu | (6-2-4c-14) |
| A-14 | O | CH [symbol] | CH [symbol] | Bnz | (6-2-5c-14) |
| A-14 | O | CH [symbol] | Et | Et | (6-2-6c-14) |
| A-14 | O | CH [symbol] | CH [symbol] CH [symbol] OCH [symbol] | CH [symbol] CH [symbol] OCH [symbol] | (6-2-7c-14) |
| A-14 | O | CH [symbol] | Pr | Bu | (6-2-8c-14) |
| A-14 | O | CH [symbol] | Et | Et | (6-2-9c-14) |
| A-14 | O | CH [symbol] | ⁱPr | ⁱPr | (6-2-10c-14) |
| A-14 | O | CH [symbol] | Bu | Bu | (6-2-11c-14) |
| A-14 | O | CH [symbol] CH [symbol] CH [symbol] | CH [symbol] | CH [symbol] | (6-2-12c-14) |
| A-14 | O | CH [symbol] CH [symbol] CH [symbol] | CH [symbol] | Et | (6-2-13c-14) |
| A-14 | O | CH [symbol] CH [symbol] CH [symbol] | CH [symbol] | ⁱPr | (6-2-14c-14) |
| A-14 | O | CH [symbol] CH [symbol] CH [symbol] | CH [symbol] | Bu | (6-2-15c-14) |
| A-14 | O | CH [symbol] CH [symbol] CH [symbol] | CH [symbol] | Bnz | (6-2-16c-14) |
| A-14 | O | CH [symbol] CH [symbol] CH [symbol] | Et | Et | (6-2-17c-14) |
| A-14 | O | CH [symbol] CH [symbol] CH [symbol] | CH [symbol] CH [symbol] OCH [symbol] | CH [symbol] CH [symbol] OCH [symbol] | (6-2-18c-14) |
| A-14 | O | CH [symbol] CH [symbol] CH [symbol] | Pr | Bu | (6-2-19c-14) |
| A-14 | O | CH [symbol] CH [symbol] CH [symbol] | Et | Et | (6-2-20c-14) |
| A-14 | O | CH [symbol] CH [symbol] CH [symbol] | ⁱPr | ⁱPr | (6-2-21c-14) |
| A-14 | O | CH [symbol] CH [symbol] CH [symbol] | Bu | Bu | (6-2-22c-14) |
| A-14 | O | CH [symbol] (CH [symbol] ) [symbol] CH [symbol] | CH [symbol] | CH [symbol] | (6-2-23c-14) |
| A-14 | O | CH [symbol] (CH [symbol] ) [symbol] CH [symbol] | CH [symbol] | Et | (6-2-24c-14) |
| A-14 | O | CH [symbol] (CH [symbol] ) [symbol] CH [symbol] | CH [symbol] | ⁱPr | (6-2-25c-14) |
| A-14 | O | CH [symbol] (CH [symbol] ) [symbol] CH [symbol] | CH [symbol] | Bu | (6-2-26c-14) |
| A-14 | O | CH [symbol] (CH [symbol] ) [symbol] CH [symbol] | CH [symbol] | Bnz | (6-2-27c-14) |
| A-14 | O | CH [symbol] (CH [symbol] ) [symbol] CH [symbol] | Et | Et | (6-2-28c-14) |
| A-14 | O | CH [symbol] (CH [symbol] ) [symbol] CH [symbol] | CH [symbol] CH [symbol] OCH [symbol] | CH [symbol] CH [symbol] OCH [symbol] | (6-2-29c-14) |
| A-14 | O | CH [symbol] (CH [symbol] ) [symbol] CH [symbol] | Pr | Bu | (6-2-30c-14) |
| A-14 | O | CH [symbol] (CH [symbol] ) [symbol] CH [symbol] | Et | Et | (6-2-31c-14) |
| A-14 | O | CH [symbol] (CH [symbol] ) [symbol] CH [symbol] | ⁱPr | ⁱPr | (6-2-32c-14) |
| A-14 | O | CH [symbol] (CH [symbol] ) [symbol] CH [symbol] | Bu | Bu | (6-2-33c-14) |

[symbol]

indicates data missing or illegible when filed

The compound represented by formula (6) is preferably a compound represented by any one of (6-2-1a-1) to (6-2-33a-1), (6-2-1b-1) to (6-2-33b-1), (6-2-1c-1) to (6-2-33c-1), (6-2-1a-2) to (6-2-33a-2), (6-2-1b-2) to (6-2-33b-2), (6-2-1c-2) to (6-2-33c-2), (6-2-1a-6) to (6-2-33a-6), (6-2-1b-6) to (6-2-33b-6), (6-2-1c-6) to (6-2-33c-6), (6-2-1a-14) to (6-2-33a-14), (6-2-1b-14) to (6-2-33b-14), and (6-2-1c-14) to (6-2-33c-14), and particularly preferably a compound represented by (6-2-12a-1) or (6-2-12a-2).

The isocyanate compound represented by formula (5) is usually used in an amount of 0.5 to 10 mol, and preferably 0.8 to 3 mol, per n mol of the compound represented by formula (4).

The reaction temperature is usually 10 to 200° C. or the boiling temperature of the solvent. The reaction temperature is preferably 20 to 150° C.

The reaction time is usually 1 to 20 hours, and preferably 1 to 10 hours.

Solvents may or may not be used. Examples of solvents include tetrahydrofuran, ethyl acetate, acetonitrile, toluene, acetone, and the like. When a solvent is used, the amount of the solvent for use is usually 100 parts by mass or less, and preferably 0.1 to 50 parts by mass, per part by mass of the compound represented by formula (4).

In step 1, the reaction may be performed, if necessary, in an inert gas atmosphere, such as nitrogen, argon, or helium, which do not affect the reaction.

The obtained compound represented by formula (6) can be purified according to common methods, such as concentration and recrystallization, or can be used as a raw material for step 2 without purification.

Step 2 is described below.

In formula (8), R′ represents a hydrogen atom, a substituted or unsubstituted hydrocarbon group, R³, or R⁷. When R′ represents a substituted or unsubstituted hydrocarbon group, in one embodiment, R′ preferably represents a substituted or unsubstituted C₁-C₁₀₀ hydrocarbon group, more preferably a substituted or unsubstituted C₁-C₅₀ hydrocarbon group, and particularly preferably a substituted or unsubstituted C₁-C₃₀ hydrocarbon group. Among these, R′ preferably represents a C₁-C₃₀ alkyl group, and more preferably a C₁-C₆ alkyl group.

In another embodiment, R′ represents a substituted or unsubstituted aliphatic hydrocarbon group, alicyclic hydrocarbon group, aromatic hydrocarbon group, or aromatic aliphatic hydrocarbon group,
preferably a substituted or unsubstituted C₁-C₁₀₀ aliphatic hydrocarbon group, alicyclic hydrocarbon group, aromatic hydrocarbon group, or aromatic aliphatic hydrocarbon group, more preferably a substituted or unsubstituted C₁-C₅₀ aliphatic hydrocarbon group, alicyclic hydrocarbon group, aromatic hydrocarbon group, or aromatic aliphatic hydrocarbon group, and
particularly preferably a substituted or unsubstituted C₁-C₃₀ aliphatic hydrocarbon group, alicyclic hydrocarbon group, aromatic hydrocarbon group, or aromatic aliphatic hydrocarbon group. Among these, R′ preferably represents a C₁-C₃₀ alkyl group, and more preferably a C₁-C₆ alkyl group.

The unsubstituted hydrocarbon group is, for example, a methyl, ethyl, propyl, isopropyl, butyl, sec-butyl, tert-butyl, pentyl, hexyl, heptyl, decyl, dodecyl, octadecyl, cyclopropyl, cyclopentyl, cyclohexyl, phenyl, naphthyl, benzyl, phenethyl, tolyl, or allyl group.

The unsubstituted aliphatic hydrocarbon group is, for example, a methyl, ethyl, propyl, isopropyl, butyl, sec-butyl, tert-butyl, pentyl, hexyl, heptyl, decyl, dodecyl, octadecyl, or allyl group.

The unsubstituted alicyclic hydrocarbon group is, for example, a cyclopropyl, cyclopentyl, or cyclohexyl group.

The unsubstituted aromatic hydrocarbon group is, for example, a phenyl, naphthyl, or tolyl group.

The unsubstituted aromatic aliphatic hydrocarbon group is, for example, a benzyl or phenethyl group.

Specific examples of the ester carbonate compound represented by formula (7a) or formula (7b) (hereinafter referred to as “the ester carbonate (7a) or (7b)”) include dialkyl carbonates, such as dimethyl carbonate, diethyl carbonate, dipropyl carbonate, dibutyl carbonate, dipentyl carbonate, and dihexyl carbonate; and alkylene carbonates, such as ethylene carbonate, propylene carbonate, and butylene carbonate. Preferred are dimethyl carbonate, diethyl carbonate, dipropyl carbonate, and dibutyl carbonate, and particularly preferred is dimethyl carbonate.

Specific examples of the compound represented by formula (8) are shown below. However, the present invention is not limited thereto. In the following specific examples, Et represents an ethyl group, Pr represents a propyl group, ⁱPr represents an isopropyl group, Bu represents a butyl group, and Bnz represents a benzyl group.

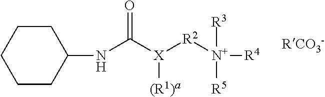

| X (R [symbol] ) [symbol] | R [symbol] | R [symbol] | R [symbol] | R [symbol] | R [symbol] | |
|---|---|---|---|---|---|---|
| NH | CH [symbol] | CH [symbol] | CH [symbol] | CH [symbol] | H | (8a-2-1a-1) |
| NH | CH [symbol] | CH [symbol] | CH [symbol] | Et | H | (8a-2-2a-1) |
| NH | CH [symbol] | CH [symbol] | CH [symbol] | [symbol] Pr | H | (8a-2-3a-1) |
| NH | CH [symbol] | CH [symbol] | CH [symbol] | Bu | H | (8a-2-4a-1) |
| NH | CH [symbol] | CH [symbol] | CH [symbol] | [symbol] | H | (8a-2-5a-1) |
| NH | CH [symbol] | CH [symbol] | Et | Et | H | (8a-2-6a-1) |
| NH | CH [symbol] | CH [symbol] | CH [symbol] CH [symbol] OCH [symbol] | CH [symbol] OCH [symbol] | H | (8a-2-7a-1) |
| NH | CH [symbol] | CH [symbol] | Pr | Bu | H | (8a-2-8a-1) |
| NH | CH [symbol] | Et | Et | Et | H | (8a-2-9a-1) |
| NH | CH [symbol] | [symbol] Pr | [symbol] Pr | [symbol] Pr | H | (8a-2-10a-1) |
| NH | CH [symbol] | Bu | Bu | Bu | H | (8a-2-11a-1) |
| NH | CH [symbol] CH [symbol] CH [symbol] | CH [symbol] | CH [symbol] | CH [symbol] | H | (8a-2-12a-1) |
| NH | CH [symbol] CH [symbol] CH [symbol] | CH [symbol] | CH [symbol] | Et | H | (8a-2-13a-1) |
| NH | CH [symbol] CH [symbol] CH [symbol] | CH [symbol] | CH [symbol] | [symbol] Pr | H | (8a-2-14a-1) |
| NH | CH [symbol] CH [symbol] CH [symbol] | CH [symbol] | CH [symbol] | Bu | H | (8a-2-15a-1) |
| NH | CH [symbol] CH [symbol] CH [symbol] | CH [symbol] | CH [symbol] | [symbol] | H | (8a-2-16a-1) |
| NH | CH [symbol] CH [symbol] CH [symbol] | CH [symbol] | Et | Et | H | (8a-2-17a-1) |
| NH | CH [symbol] CH [symbol] CH [symbol] | CH [symbol] | CH [symbol] CH [symbol] OCH [symbol] | CH [symbol] CH [symbol] OCH [symbol] | H | (8a-2-18a-1) |
| NH | CH [symbol] CH [symbol] CH [symbol] | CH [symbol] | Pr | Bu | H | (8a-2-19a-1) |
| NH | CH [symbol] CH [symbol] CH [symbol] | Et | Et | Et | H | (8a-2-20a-1) |
| NH | CH [symbol] CH [symbol] CH [symbol] | [symbol] Pr | [symbol] Pr | [symbol] Pr | H | (8a-2-21a-1) |
| NH | CH [symbol] CH [symbol] CH [symbol] | Bu | Bu | Bu | H | (8a-2-22a-1) |

[symbol]

indicates data missing or illegible when filed

| X (R [symbol] ) [symbol] | R [symbol] | R [symbol] | R [symbol] | R [symbol] | R [symbol] | |
|---|---|---|---|---|---|---|
| NH | CH [symbol] (CH [symbol] ) [symbol] CH [symbol] | CH [symbol] | CH [symbol] | CH [symbol] | H | (8 [symbol] -2-23a-1) |
| NH | CH [symbol] (CH [symbol] ) [symbol] CH [symbol] | CH [symbol] | CH [symbol] | Et | H | (8 [symbol] -2-24a-1) |
| NH | CH [symbol] (CH [symbol] ) [symbol] CH [symbol] | CH [symbol] | CH [symbol] | [symbol] Pr | H | (8 [symbol] -2-25a-1) |
| NH | CH [symbol] (CH [symbol] ) [symbol] CH [symbol] | CH [symbol] | CH [symbol] | Bu | H | (8 [symbol] -2-26a-1) |
| NH | CH [symbol] (CH [symbol] ) [symbol] CH [symbol] | CH [symbol] | CH [symbol] | [symbol] | H | (8 [symbol] -2-27a-1) |
| NH | CH [symbol] (CH [symbol] ) [symbol] CH [symbol] | CH [symbol] | Et | Et | H | (8 [symbol] -2-28a-1) |
| NH | CH [symbol] (CH [symbol] ) [symbol] CH [symbol] | CH [symbol] | CH [symbol] CH [symbol] OCH [symbol] | CH [symbol] CH [symbol] OCH [symbol] | H | (8 [symbol] -2-29a-1) |
| NH | CH [symbol] (CH [symbol] ) [symbol] CH [symbol] | CH [symbol] | Pr | Bu | H | (8 [symbol] -2-30a-1) |
| NH | CH [symbol] (CH [symbol] ) [symbol] CH [symbol] | Et | Et | Et | H | (8 [symbol] -2-31a-1) |
| NH | CH [symbol] (CH [symbol] ) [symbol] CH [symbol] | [symbol] Pr | [symbol] Pr | [symbol] Pr | H | (8 [symbol] -2-32a-1) |
| NH | CH [symbol] (CH [symbol] ) [symbol] CH [symbol] | Bu | Bu | Bu | H | (8 [symbol] -2-33a-1) |
| NH | CH [symbol] | CH [symbol] | CH [symbol] | CH [symbol] | CH [symbol] | (8b-2-1a-1) |
| NH | CH [symbol] | CH [symbol] | CH [symbol] | Et | CH [symbol] | (8b-2-2a-1) |
| NH | CH [symbol] | CH [symbol] | CH [symbol] | [symbol] Pr | CH [symbol] | (8b-2-3a-1) |
| NH | CH [symbol] | CH [symbol] | CH [symbol] | Bu | CH [symbol] | (8b-2-4a-1) |
| NH | CH [symbol] | CH [symbol] | CH [symbol] | [symbol] | CH [symbol] | (8b-2-5a-1) |
| NH | CH [symbol] | CH [symbol] | Et | Et | CH [symbol] | (8b-2-6a-1) |
| NH | CH [symbol] | CH [symbol] | CH [symbol] CH [symbol] OCH [symbol] | CH [symbol] CH [symbol] OCH [symbol] | CH [symbol] | (8b-2-7a-1) |
| NH | CH [symbol] | CH [symbol] | Pr | Bu | CH [symbol] | (8b-2-8a-1) |
| NH | CH [symbol] | CH [symbol] | CH [symbol] | Et | Et | (8b-2-9a-1) |
| NH | CH [symbol] CH [symbol] CH [symbol] | CH [symbol] | [symbol] Pr | [symbol] Pr | CH [symbol] | (8b-2-10a-1) |
| NH | CH [symbol] CH [symbol] CH [symbol] | CH [symbol] | CH [symbol] | Et | CH [symbol] | (8b-2-11a-1) |
| NH | CH [symbol] CH [symbol] CH [symbol] | CH [symbol] | CH [symbol] | [symbol] Pr | CH [symbol] | (8b-2-12a-1) |
| NH | CH [symbol] CH [symbol] CH [symbol] | CH [symbol] | CH [symbol] | Bu | CH [symbol] | (8b-2-13a-1) |
| NH | CH [symbol] CH [symbol] CH [symbol] | CH [symbol] | CH [symbol] | [symbol] | CH [symbol] | (8b-2-14a-1) |
| NH | CH [symbol] CH [symbol] CH [symbol] | CH [symbol] | Et | Et | CH [symbol] | (8b-2-15a-1) |
| NH | CH [symbol] CH [symbol] CH [symbol] | CH [symbol] | CH [symbol] CH [symbol] OCH [symbol] | CH [symbol] CH [symbol] OCH [symbol] | CH [symbol] | (8b-2-16a-1) |
| NH | CH [symbol] CH [symbol] CH [symbol] | CH [symbol] | Pr | Bu | CH [symbol] | (8b-2-17a-1) |
| NH | CH [symbol] CH [symbol] CH [symbol] | CH [symbol] | CH [symbol] | Et | Et | (8b-2-18a-1) |
| NH | CH [symbol] (CH [symbol] ) [symbol] CH [symbol] | CH [symbol] | CH [symbol] | CH [symbol] | CH [symbol] | (8b-2-19a-1) |
| NH | CH [symbol] (CH [symbol] ) [symbol] CH [symbol] | CH [symbol] | CH [symbol] | Et | CH [symbol] | (8b-2-20a-1) |
| NH | CH [symbol] (CH [symbol] ) [symbol] CH [symbol] | CH [symbol] | CH [symbol] | [symbol] Pr | CH [symbol] | (8b-2-21a-1) |
| NH | CH [symbol] (CH [symbol] ) [symbol] CH [symbol] | CH [symbol] | CH [symbol] | Bu | CH [symbol] | (8b-2-22a-1) |
| NH | CH [symbol] (CH [symbol] ) [symbol] CH [symbol] | CH [symbol] | CH [symbol] | [symbol] | CH [symbol] | (8b-2-23a-1) |
| NH | CH [symbol] (CH [symbol] ) [symbol] CH [symbol] | CH [symbol] | Et | Et | CH [symbol] | (8b-2-24a-1) |
| NH | CH [symbol] (CH [symbol] ) [symbol] CH [symbol] | CH [symbol] | CH [symbol] CH [symbol] OCH [symbol] | CH [symbol] CH [symbol] OCH [symbol] | CH [symbol] | (8b-2-25a-1) |
| NH | CH [symbol] (CH [symbol] ) [symbol] CH [symbol] | CH [symbol] | Pr | Bu | CH [symbol] | (8b-2-26a-1) |

[symbol]

indicates data missing or illegible when filed

| | | | | | | |
|---|---|---|---|---|---|---|
| NH | CH₂(CH₂) ₄CH₂ | CH₃ | CH₃ | Et | Et | (8b-2-27a-1) |

| X (R [symbol] ) [symbol] | R [symbol] | R [symbol] | R [symbol] | R [symbol] | R [symbol] | |
|---|---|---|---|---|---|---|
| N(CH₃) | CH [symbol] | CH [symbol] | CH [symbol] | CH [symbol] | H | (8a-2-1a-1) |
| N(CH₃) | CH [symbol] | CH [symbol] | CH [symbol] | Et | H | (8a-2-2a-1) |
| N(CH₃) | CH [symbol] | CH [symbol] | CH [symbol] | [symbol] Pr | H | (8a-2-3a-1) |
| N(CH₃) | CH [symbol] | CH [symbol] | CH [symbol] | Bu | H | (8a-2-4a-1) |
| N(CH₃) | CH [symbol] | CH [symbol] | CH [symbol] | [symbol] | H | (8a-2-5a-1) |
| N(CH₃) | CH [symbol] | CH [symbol] | Et | Et | H | (8a-2-6a-1) |
| N(CH₃) | CH [symbol] | CH [symbol] | CH [symbol] CH [symbol] OCH [symbol] | CH [symbol] CH [symbol] OCH [symbol] | H | (8a-2-7a-1) |
| N(CH₃) | CH [symbol] | CH [symbol] | Pr | Bu | H | (8a-2-8a-1) |
| N(CH₃) | CH [symbol] | Et | Et | Et | H | (8a-2-9a-1) |
| N(CH₃) | CH [symbol] | [symbol] Pr | [symbol] Pr | [symbol] Pr | H | (8a-2-10a-1) |
| N(CH₃) | CH [symbol] | Bu | Bu | Bu | H | (8a-2-11a-1) |
| N(CH₃) | CH [symbol] CH [symbol] CH [symbol] | CH [symbol] | CH [symbol] | CH [symbol] | H | (8a-2-12a-1) |
| N(CH₃) | CH [symbol] CH [symbol] CH [symbol] | CH [symbol] | CH [symbol] | Et | H | (8a-2-13a-1) |
| N(CH₃) | CH [symbol] CH [symbol] CH [symbol] | CH [symbol] | CH [symbol] | [symbol] Pr | H | (8a-2-14a-1) |
| N(CH₃) | CH [symbol] CH [symbol] CH [symbol] | CH [symbol] | CH [symbol] | Bu | H | (8a-2-15a-1) |
| N(CH₃) | CH [symbol] CH [symbol] CH [symbol] | CH [symbol] | CH [symbol] | [symbol] | H | (8a-2-16a-1) |
| N(CH₃) | CH [symbol] CH [symbol] CH [symbol] | CH [symbol] | Et | Et | H | (8a-2-17a-1) |
| N(CH₃) | CH [symbol] CH [symbol] CH [symbol] | CH [symbol] | CH [symbol] CH [symbol] OCH [symbol] | CH [symbol] CH [symbol] OCH [symbol] | H | (8a-2-18a-1) |
| N(CH₃) | CH [symbol] CH [symbol] CH [symbol] | CH [symbol] | Pr | Bu | H | (8a-2-19a-1) |
| N(CH₃) | CH [symbol] CH [symbol] CH [symbol] | Et | Et | Et | H | (8a-2-20a-1) |
| N(CH₃) | CH [symbol] CH [symbol] CH [symbol] | [symbol] Pr | [symbol] Pr | [symbol] Pr | H | (8a-2-21a-1) |
| N(CH₃) | CH [symbol] CH [symbol] CH [symbol] | Bu | Bu | Bu | H | (8a-2-22a-1) |
| N(CH₃) | CH [symbol] (CH [symbol] ) [symbol] CH [symbol] | CH [symbol] | CH [symbol] | CH [symbol] | H | (8a-2-23a-1) |
| N(CH₃) | CH [symbol] (CH [symbol] ) [symbol] CH [symbol] | CH [symbol] | CH [symbol] | Et | H | (8a-2-24a-1) |
| N(CH₃) | CH [symbol] (CH [symbol] ) [symbol] CH [symbol] | CH [symbol] | CH [symbol] | [symbol] Pr | H | (8a-2-25a-1) |
| N(CH₃) | CH [symbol] (CH [symbol] ) [symbol] CH [symbol] | CH [symbol] | CH [symbol] | Bu | H | (8a-2-26a-1) |
| N(CH₃) | CH [symbol] (CH [symbol] ) [symbol] CH [symbol] | CH [symbol] | CH [symbol] | [symbol] | H | (8a-2-27a-1) |
| N(CH₃) | CH [symbol] (CH [symbol] ) [symbol] CH [symbol] | CH [symbol] | Et | Et | H | (8a-2-28a-1) |
| N(CH₃) | CH [symbol] (CH [symbol] ) [symbol] CH [symbol] | CH [symbol] | CH [symbol] CH [symbol] OCH [symbol] | CH [symbol] CH [symbol] OCH [symbol] | H | (8a-2-29a-1) |
| N(CH₃) | CH [symbol] (CH [symbol] ) [symbol] CH [symbol] | CH [symbol] | Pr | Bu | H | (8a-2-30a-1) |
| N(CH₃) | CH [symbol] (CH [symbol] ) [symbol] CH [symbol] | Et | Et | Et | H | (8a-2-31a-1) |
| N(CH₃) | CH [symbol] (CH [symbol] ) [symbol] CH [symbol] | [symbol] Pr | [symbol] Pr | [symbol] | H | (8a-2-32a-1) |
| N(CH₃) | CH [symbol] (CH [symbol] ) [symbol] CH [symbol] | Bu | Bu | Bu | H | (8a-2-33a-1) |

[symbol]

indicates data missing or illegible when filed

| χ(R¹)ⁿ | R² | R³ | R⁴ | R⁵ | R′ | |
|---|---|---|---|---|---|---|
| N(CH₃) | CH₃ | CH₃ | CH₃ | CH₃ | CH₃ | (8b-2-1b-1) |
| N(CH₃) | CH₃ | CH₃ | CH₃ | Et | CH₃ | (8b-2-2b-1) |
| N(CH₃) | CH₃ | CH₃ | CH₃ | ⁱPr | CH₃ | (8b-2-3b-1) |
| N(CH₃) | CH₃ | CH₃ | CH₃ | Bu | CH₃ | (8b-2-4b-1) |
| N(CH₃) | CH₃ | CH₃ | CH₃ | Bnz | CH₃ | (8b-2-5b-3) |
| N(CH₃) | CH₃ | CH₃ | Et | Et | CH₃ | (8b-2-6b-1) |
| N(CH₃) | CH₃ | CH₃ | CH₃CH₃OCH₃ | CH₃CH₃OCH₃ | CH₃ | (8b-2-7b-1) |
| N(CH₃) | CH₃ | CH₃ | Pr | Bu | CH₃ | (8b-2-8b-1) |
| N(CH₃) | CH₃ | CH₃ | CH₃ | Et | Et | (8b-2-9b-1) |
| N(CH₃) | CH₃CH₃CH₃ | CH₃ | CH₃ | CH₃ | CH₃ | (8b-2-10b-1) |
| N(CH₃) | CH₃CH₃CH₃ | CH₃ | CH₃ | Et | CH₃ | (8b-2-11b-1) |
| N(CH₃) | CH₃CH₃CH₃ | CH₃ | CH₃ | ⁱPr | CH₃ | (8b-2-12b-1) |
| N(CH₃) | CH₃CH₃CH₃ | CH₃ | CH₃ | Bu | CH₃ | (8b-2-13b-1) |
| N(CH₃) | CH₃CH₃CH₃ | CH₃ | CH₃ | Bnz | CH₃ | (8b-2-14b-1) |
| N(CH₃) | CH₃CH₃CH₃ | CH₃ | Et | Et | CH₃ | (8b-2-16b-1) |
| N(CH₃) | CH₃CH₃CH₃ | CH₃ | CH₃CH₃OCH₃ | CH₃CH₃OCH₃ | CH₃ | (8b-2-16b-1) |
| N(CH₃) | CH₃CH₃CH₃ | CH₃ | Pr | Bu | CH₃ | (8b-2-17b-1) |
| N(CH₃) | CH₃CH₃CH₃ | CH₃ | CH₃ | Et | Et | (8b-2-18b-1) |
| N(CH₃) | CH₃(CH₃)₄CH₃ | CH₃ | CH₃ | CH₃ | CH₃ | (8b-2-19b-1) |
| N(CH₃) | CH₃(CH₃)₄CH₃ | CH₃ | CH₃ | Et | CH₃ | (8b-2-20b-1) |
| N(CH₃) | CH₃(CH₃)₄CH₃ | CH₃ | CH₃ | ⁱPr | CH₃ | (8b-2-21b-1) |
| N(CH₃) | CH₃(CH₃)₄CH₃ | CH₃ | CH₃ | Bu | CH₃ | (8b-2-22b-1) |
| N(CH₃) | CH₃(CH₃)₄CH₃ | CH₃ | CH₃ | Bnz | CH₃ | (8b-2-23b-1) |
| N(CH₃) | CH₃(CH₃)₄CH₃ | CH₃ | Et | Et | CH₃ | (8b-2-24b-1) |
| N(CH₃) | CH₃(CH₃)₄CH₃ | CH₃ | CH₃CH₃OCH₃ | CH₃CH₃OCH₃ | CH₃ | (8b-2-25b-1) |
| N(CH₃) | CH₃(CH₃)₄CH₃ | CH₃ | Pr | Bu | CH₃ | (8b-2-26b-1) |
| N(CH₃) | CH₃(CH₃)₄CH₃ | CH₃ | CH₃ | Et | Et | (8b-2-27b-1) |

| χ(R¹)ⁿ | R² | R³ | R⁴ | R⁵ | R′ | |
|---|---|---|---|---|---|---|
| O | CH₃ | CH₃ | CH₃ | CH₃ | H | (8a-2-1c-1) |
| O | CH₃ | CH₃ | CH₃ | Et | H | (8a-2-2c-1) |
| O | CH₃ | CH₃ | CH₃ | ⁱPr | H | (8a-2-3c-1) |
| O | CH₃ | CH₃ | CH₃ | Bu | H | (8a-2-4c-1) |
| O | CH₃ | CH₃ | CH₃ | Bnz | H | (8a-2-5c-1) |
| O | CH₃ | CH₃ | CH₃ | Et | H | (8a-2-6c-1) |
| O | CH₃ | CH₃ | CH₃CH₃OCH₃ | CH₃CH₃OCH₃ | H | (8a-2-7c-1) |
| O | CH₃ | CH₃ | Pr | Bu | H | (8a-2-8c-1) |
| O | CH₃ | Et | Et | Et | H | (8a-2-9c-1) |
| O | CH₃ | ⁱPr | ⁱPr | ⁱPr | H | (8a-2-10c-1) |
| O | CH₃ | Bu | Bu | Bu | H | (8a-2-11c-1) |
| O | CH₃CH₃CH₃ | CH₃ | CH₃ | CH₃ | H | (8a-2-12c-1) |
| O | CH₃CH₃CH₃ | CH₃ | CH₃ | Et | H | (8a-2-13c-1) |
| O | CH₃CH₃CH₃ | CH₃ | CH₃ | ⁱPr | H | (8a-2-14c-1) |
| O | CH₃CH₃CH₃ | CH₃ | CH₃ | Bu | H | (8a-2-15c-1) |
| O | CH₃CH₃CH₃ | CH₃ | CH₃ | Bnz | H | (8a-2-16c-1) |
| O | CH₃CH₃CH₃ | CH₃ | Et | Et | H | (8a-2-17c-1) |
| O | CH₃CH₃CH₃ | CH₃ | CH₃CH₃OCH₃ | CH₃CH₃OCH₃ | H | (8a-2-18c-1) |
| O | CH₃CH₃CH₃ | CH₃ | Pr | Bu | H | (8a-2-19c-1) |
| O | CH₃CH₃CH₃ | Et | Et | Et | H | (8a-2-20c-1) |
| O | CH₃CH₃CH₃ | ⁱPr | ⁱPr | ⁱPr | H | (8a-2-21c-1) |
| O | CH₃CH₃CH₃ | Bu | Bu | Bu | H | (8a-2-22c-1) |
| O | CH₃(CH₃)₄CH₃ | CH₃ | CH₃ | CH₃ | H | (8a-2-23c-1) |
| O | CH₃(CH₃)₄CH₃ | CH₃ | CH₃ | Et | H | (8a-2-24c-1) |
| O | CH₃(CH₃)₄CH₃ | CH₃ | CH₃ | ⁱPr | H | (8a-2-25c-1) |
| O | CH₃(CH₃)₄CH₃ | CH₃ | CH₃ | Bu | H | (8a-2-26c-1) |
| O | CH₃(CH₃)₄CH₃ | CH₃ | CH₃ | Bnz | H | (8a-2-27c-1) |
| O | CH₃(CH₃)₄CH₃ | CH₃ | Et | Et | H | (8a-2-28c-1) |
| O | CH₃(CH₃)₄CH₃ | CH₃ | CH₃CH₃OCH₃ | CH₃CH₃OCH₃ | H | (8a-2-29c-1) |
| O | CH₃(CH₃)₄CH₃ | CH₃ | Pr | Bu | H | (8a-2-30c-1) |
| O | CH₃(CH₃)₄CH₃ | Et | Et | Et | H | (8a-2-31c-1) |
| O | CH₃(CH₃)₄CH₃ | ⁱPr | ⁱPr | ⁱPr | H | (8a-2-32c-1) |
| O | CH₃(CH₃)₄CH₃ | Bu | Bu | Bu | H | (8a-2-33c-1) |

| χ(R¹)ⁿ | R² | R³ | R⁴ | R⁵ | R′ | |
|---|---|---|---|---|---|---|
| O | CH₃ | CH₃ | CH₃ | CH₃ | CH₃ | (8b-2-1c-1) |
| O | CH₃ | CH₃ | CH₃ | Et | CH₃ | (8b-2-2c-1) |
| O | CH₃ | CH₃ | CH₃ | ⁱPr | CH₃ | (8b-2-3c-1) |
| O | CH₃ | CH₃ | CH₃ | Bu | CH₃ | (8b-2-4c-1) |
| O | CH₃ | CH₃ | CH₃ | Bnz | CH₃ | (8b-2-5c-1) |
| O | CH₃ | CH₃ | Et | Et | CH₃ | (8b-2-6c-1) |
| O | CH₃ | CH₃ | CH₃CH₃OCH₃ | CH₃CH₃OCH₃ | CH₃ | (8b-2-7c-1) |
| O | CH₃ | CH₃ | Pr | Bu | CH₃ | (8b-2-8c-1) |
| O | CH₃ | CH₃ | CH₃ | Et | Et | (8b-2-9c-1) |
| O | CH₃CH₃CH₃ | CH₃ | CH₃ | CH₃ | CH₃ | (8b-2-10c-1) |
| O | CH₃CH₃CH₃ | CH₃ | CH₃ | Et | CH₃ | (8b-2-11c-1) |
| O | CH₃CH₃CH₃ | CH₃ | CH₃ | ⁱPr | CH₃ | (8b-2-12c-1) |
| O | CH₃CH₃CH₃ | CH₃ | CH₃ | Bu | CH₃ | (8b-2-13c-1) |
| O | CH₃CH₃CH₃ | CH₃ | CH₃ | Bnz | CH₃ | (8b-2-14c-1) |
| O | CH₃CH₃CH₃ | CH₃ | Et | Et | CH₃ | (8b-2-15c-1) |
| O | CH₃CH₃CH₃ | CH₃ | CH₃CH₃OCH₃ | CH₃CH₃OCH₃ | CH₃ | (8b-2-16c-1) |
| O | CH₃CH₃CH₃ | CH₃ | Pr | Bu | CH₃ | (8b-2-17c-1) |
| O | CH₃CH₃CH₃ | CH₃ | CH₃ | Et | Et | (8b-2-18c-1) |
| O | CH₃(CH₃)₄CH₃ | CH₃ | CH₃ | CH₃ | CH₃ | (8b-2-19c-1) |
| O | CH₃(CH₃)₄CH₃ | CH₃ | CH₃ | Et | CH₃ | (8b-2-20c-1) |
| O | CH₃(CH₃)₄CH₃ | CH₃ | CH₃ | ⁱPr | CH₃ | (8b-2-21c-1) |
| O | CH₃(CH₃)₄CH₃ | CH₃ | CH₃ | Bu | CH₃ | (8b-2-22c-1) |
| O | CH₃(CH₃)₄CH₃ | CH₃ | CH₃ | Bnz | CH₃ | (8b-2-23c-1) |
| O | CH₃(CH₃)₄CH₃ | CH₃ | Et | Et | CH₃ | (8b-2-24c-1) |
| O | CH₃(CH₃)₄CH₃ | CH₃ | CH₃CH₃OCH₃ | CH₃CH₃OCH₃ | CH₃ | (8b-2-25c-1) |
| O | CH₃(CH₃)₄CH₃ | CH₃ | Pr | Bu | CH₃ | (8b-2-26c-1) |
| O | CH₃(CH₃)₄CH₃ | CH₃ | CH₃ | Et | Et | (8b-2-27c-1) |

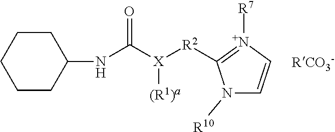

X, a, R¹, R², R⁷, R¹⁰, and R′ are as defined above. Preferably, X(R¹)^{a} represents NH, N(CH₃), or O, R² represents CH₂, CH₂CH₂, or CH₂(CH₂)₄CH₂, R⁷ represents CH₃ or Et, R¹⁰ represents CH₃, Et, ⁱPr, Bu, Bnz, or CH₂CH₂OCH₃, and R′ represents H, CH₃, or Et.

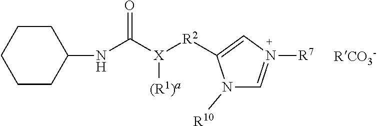

X, a, R¹, R², R⁷, R¹⁰, and R′ are as defined above. Preferably, X(R¹)^{a} represents NH, N(CH₃), or O, R² represents CH₂, CH₂CH₂, or CH₂(CH₂)₄CH₂, R⁷ represents CH₃ or Et, R¹⁰ represents CH₃, Et, ⁱPr, Bu, Bnz, or CH₂CH₂OCH₃, and R′ represents H, CH₃, or Et.

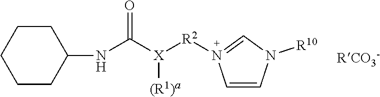

X, a, R¹, R², R⁷, R¹⁰, and R′ are as defined above. Preferably, X(R¹)^{a} represents NH, N(CH₃), or O, R² represents CH₂, CH₂CH₂, or CH₂(CH₂)₄CH₂, R⁷ represents CH₃ or Et, R¹⁰ represents CH₃, Et, ⁱPr, Bu, Bnz, or CH₂CH₂OCH₃, and R′ represents H, CH₃, or Et.

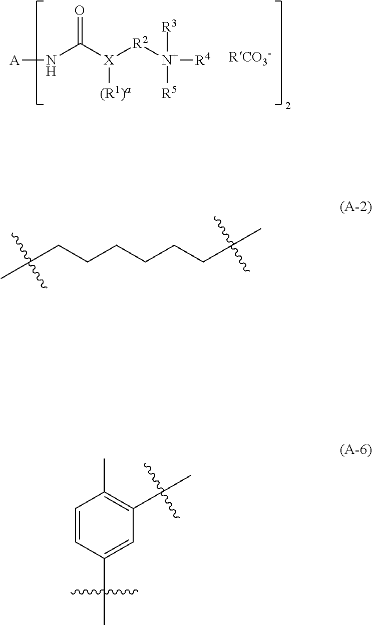

| A | χ(R¹)ⁿ | R² | R³ | R⁴ | R⁵ | R′ | |
|---|---|---|---|---|---|---|---|
| A-2 | NH | CH₃ | CH₃ | CH₃ | CH₃ | H | (8a-2-1a-2) |
| A-2 | NH | CH₃ | CH₃ | CH₃ | Et | H | (8a-2-2a-2) |
| A-2 | NH | CH₃ | CH₃ | CH₃ | ⁱPr | H | (8a-2-3a-2) |
| A-2 | NH | CH₃ | CH₃ | CH₃ | Bu | H | (8a-2-4a-2) |
| A-2 | NH | CH₃ | CH₃ | CH₃ | Bnz | H | (8a-2-5a-2) |
| A-2 | NH | CH₃ | CH₃ | Et | Et | H | (8a-2-6a-2) |
| A-2 | NH | CH₃ | CH₃ | CH₃CH₃OCH₃ | CH₃CH₃OCH₃ | H | (8a-2-7a-2) |
| A-2 | NH | CH₃ | CH₃ | Pr | Bu | H | (8a-2-8a-2) |
| A-2 | NH | CH₃ | Et | Et | Et | H | (8a-2-9a-2) |
| A-2 | NH | CH₃ | ⁱPr | ⁱPr | ⁱPr | H | (8a-2-10a-2) |
| A-2 | NH | CH₃ | Bu | Bu | Bu | H | (8a-2-11a-2) |
| A-2 | NH | CH₃CH₃CH₃ | CH₃ | CH₃ | CH₃ | H | (8a-2-12a-2) |
| A-2 | NH | CH₃CH₃CH₃ | CH₃ | CH₃ | Et | H | (8a-2-13a-2) |
| A-2 | NH | CH₃CH₃CH₃ | CH₃ | CH₃ | ⁱPr | H | (8a-2-14a-2) |
| A-2 | NH | CH₃CH₃CH₃ | CH₃ | CH₃ | Bu | H | (8a-2-15a-2) |
| A-2 | NH | CH₃CH₃CH₃ | CH₃ | CH₃ | Bnz | H | (8a-2-16a-2) |
| A-2 | NH | CH₃CH₃CH₃ | CH₃ | Et | Et | H | (8a-2-17a-2) |
| A-2 | NH | CH₃CH₃CH₃ | CH₃ | CH₃CH₃OCH₃ | CH₃CH₃OCH₃ | H | (8a-2-18a-2) |
| A-2 | NH | CH₃CH₃CH₃ | CH₃ | Pr | Bu | H | (8a-2-19a-2) |
| A-2 | NH | CH₃CH₃CH₃ | Et | Et | Et | H | (8a-2-20a-2) |
| A-2 | NH | CH₃CH₃CH₃ | ⁱPr | ⁱPr | ⁱPr | H | (8a-2-21a-2) |
| A-2 | NH | CH₃CH₃CH₃ | Bu | Bu | Bu | H | (8a-2-22a-2) |
| A-2 | NH | CH₃(CH₃)₄CH₃ | CH₃ | CH₃ | CH₃ | H | (8a-2-23a-2) |
| A-2 | NH | CH₃(CH₃)₄CH₃ | CH₃ | CH₃ | Et | H | (8a-2-24a-2) |
| A-2 | NH | CH₃(CH₃)₄CH₃ | CH₃ | CH₃ | ⁱPr | H | (8a-2-25a-2) |
| A-2 | NH | CH₃(CH₃)₄CH₃ | CH₃ | CH₃ | Bu | H | (8a-2-26a-2) |
| A-2 | NH | CH₃(CH₃)₄CH₃ | CH₃ | CH₃ | Bnz | H | (8a-2-27a-2) |
| A-2 | NH | CH₃(CH₃)₄CH₃ | CH₃ | Et | Et | H | (8a-2-28a-2) |
| A-2 | NH | CH₃(CH₃)₄CH₃ | CH₃ | CH₃CH₃OCH₃ | CH₃CH₃OCH₃ | H | (8a-2-29a-2) |
| A-2 | NH | CH₃(CH₃)₄CH₃ | CH₃ | Pr | Bu | H | (8a-2-30a-2) |

| | | | | | | | |
|---|---|---|---|---|---|---|---|
| A-2 | NH | CH₃(CH₃)₄CH₃ | Et | Et | Et | H | (8a-2-31a-2) |
| A-2 | NH | CH₃(CH₃)₄CH₃ | ⁱPr | ⁱPr | ⁱPr | H | (8a-2-32a-2) |
| A-2 | NH | CH₃(CH₃)₄CH₃ | Bu | Bu | Bu | H | (8a-2-33a-2) |

| A | χ(R¹)ⁿ | R² | R³ | R⁴ | R⁵ | R′ | |
|---|---|---|---|---|---|---|---|
| A-2 | N(CH₃) | CH₃ | CH₃ | CH₃ | CH₃ | H | (8a-2-1b-2) |
| A-2 | N(CH₃) | CH₃ | CH₃ | CH₃ | Et | H | (8a-2-2b-2) |
| A-2 | N(CH₃) | CH₃ | CH₃ | CH₃ | ⁱPr | H | (8a-2-3b-2) |
| A-2 | N(CH₃) | CH₃ | CH₃ | CH₃ | Bu | H | (8a-2-4b-2) |
| A-2 | N(CH₃) | CH₃ | CH₃ | CH₃ | Bnz | H | (8a-2-5b-2) |
| A-2 | N(CH₃) | CH₃ | CH₃ | Et | Et | H | (8a-2-6b-2) |
| A-2 | N(CH₃) | CH₃ | CH₃ | CH₃CH₃OCH₃ | CH₃CH₃OCH₃ | H | (8a-2-7b-2) |
| A-2 | N(CH₃) | CH₃ | CH₃ | Pr | Bu | H | (8a-2-8b-2) |
| A-2 | N(CH₃) | CH₃ | Et | Et | Et | H | (8a-2-9b-2) |
| A-2 | N(CH₃) | CH₃ | ⁱPr | ⁱPr | ⁱPr | H | (8a-2-10b-2) |
| A-2 | N(CH₃) | CH₃ | Bu | Bu | Bu | H | (8a-2-11b-2) |
| A-2 | N(CH₃) | CH₃CH₃CH₃ | CH₃ | CH₃ | CH₃ | H | (8a-2-12b-2) |
| A-2 | N(CH₃) | CH₃CH₃CH₃ | CH₃ | CH₃ | Et | H | (8a-2-13b-2) |
| A-2 | N(CH₃) | CH₃CH₃CH₃ | CH₃ | CH₃ | ⁱPr | H | (8a-2-14b-2) |
| A-2 | N(CH₃) | CH₃CH₃CH₃ | CH₃ | CH₃ | Bu | H | (8a-2-15b-2) |
| A-2 | N(CH₃) | CH₃CH₃CH₃ | CH₃ | CH₃ | Bnz | H | (8a-2-16b-2) |
| A-2 | N(CH₃) | CH₃CH₃CH₃ | CH₃ | Et | Et | H | (8a-2-17b-2) |
| A-2 | N(CH₃) | CH₃CH₃CH₃ | CH₃ | CH₃CH₃OCH₃ | CH₃CH₃OCH₃ | H | (8a-2-18b-2) |
| A-2 | N(CH₃) | CH₃CH₃CH₃ | CH₃ | Pr | Bu | H | (8a-2-19b-2) |
| A-2 | N(CH₃) | CH₃CH₃CH₃ | Et | Et | Et | H | (8a-2-20b-2) |
| A-2 | N(CH₃) | CH₃CH₃CH₃ | ⁱPr | ⁱPr | ⁱPr | H | (8a-2-21b-2) |
| A-2 | N(CH₃) | CH₃CH₃CH₃ | Bu | Bu | Bu | H | (8a-2-22b-2) |
| A-2 | N(CH₃) | CH₃(CH₃)₄CH₃ | CH₃ | CH₃ | CH₃ | H | (8a-2-23b-2) |
| A-2 | N(CH₃) | CH₃(CH₃)₄CH₃ | CH₃ | CH₃ | Et | H | (8a-2-24b-2) |
| A-2 | N(CH₃) | CH₃(CH₃)₄CH₃ | CH₃ | CH₃ | ⁱPr | H | (8a-2-25b-2) |
| A-2 | N(CH₃) | CH₃(CH₃)₄CH₃ | CH₃ | CH₃ | Bu | H | (8a-2-26b-2) |
| A-2 | N(CH₃) | CH₃(CH₃)₄CH₃ | CH₃ | CH₃ | Bnz | H | (8a-2-27b-2) |
| A-2 | N(CH₃) | CH₃(CH₃)₄CH₃ | CH₃ | Et | Et | H | (8a-2-28b-2) |
| A-2 | N(CH₃) | CH₃(CH₃)₄CH₃ | CH₃ | CH₃CH₃OCH₃ | CH₃CH₃OCH₃ | H | (8a-2-29b-2) |
| A-2 | N(CH₃) | CH₃(CH₃)₄CH₃ | CH₃ | Pr | Bu | H | (8a-2-30b-2) |
| A-2 | N(CH₃) | CH₃(CH₃)₄CH₃ | Et | Et | Et | H | (8a-2-31b-2) |
| A-2 | N(CH₃) | CH₃(CH₃)₄CH₃ | ⁱPr | ⁱPr | ⁱPr | H | (8a-2-32b-2) |
| A-2 | N(CH₃) | CH₃(CH₃)₄CH₃ | Bu | Bu | Bu | H | (8a-2-33b-2) |

| A | χ(R¹)ⁿ | R² | R³ | R⁴ | R⁵ | R′ | |
|---|---|---|---|---|---|---|---|
| A-2 | O | CH₃ | CH₃ | CH₃ | CH₃ | H | (8a-2-1c-2) |
| A-2 | O | CH₃ | CH₃ | CH₃ | Et | H | (8a-2-2c-2) |
| A-2 | O | CH₃ | CH₃ | CH₃ | ⁱPr | H | (8a-2-3c-2) |
| A-2 | O | CH₃ | CH₃ | CH₃ | Bu | H | (8a-2-4c-2) |
| A-2 | O | CH₃ | CH₃ | CH₃ | Bnz | H | (8a-2-5c-2) |
| A-2 | O | CH₃ | CH₃ | Et | Et | H | (8a-2-6c-2) |
| A-2 | O | CH₃ | CH₃ | CH₃CH₃OCH₃ | CH₃CH₃OCH₃ | H | (8a-2-7c-2) |
| A-2 | O | CH₃ | CH₃ | Pr | Bu | H | (8a-2-8c-2) |
| A-2 | O | CH₃ | Et | Et | Et | H | (8a-2-9c-2) |
| A-2 | O | CH₃ | ⁱPr | ⁱPr | ⁱPr | H | (8a-2-10c-2) |
| A-2 | O | CH₃ | Bu | Bu | Bu | H | (8a-2-11c-2) |
| A-2 | O | CH₃CH₃CH₃ | CH₃ | CH₃ | CH₃ | H | (8a-2-12c-2) |
| A-2 | O | CH₃CH₃CH₃ | CH₃ | CH₃ | Et | H | (8a-2-13c-2) |
| A-2 | O | CH₃CH₃CH₃ | CH₃ | CH₃ | ⁱPr | H | (8a-2-14c-2) |
| A-2 | O | CH₃CH₃CH₃ | CH₃ | CH₃ | Bu | H | (8a-2-15c-2) |
| A-2 | O | CH₃CH₃CH₃ | CH₃ | CH₃ | Bnz | H | (8a-2-16c-2) |
| A-2 | O | CH₃CH₃CH₃ | CH₃ | Et | Et | H | (8a-2-17c-2) |
| A-2 | O | CH₃CH₃CH₃ | CH₃ | CH₃CH₃OCH₃ | CH₃CH₃OCH₃ | H | (8a-2-18c-2) |
| A-2 | O | CH₃CH₃CH₃ | CH₃ | Pr | Bu | H | (8a-2-19c-2) |
| A-2 | O | CH₃CH₃CH₃ | Et | Et | Et | H | (8a-2-20c-2) |
| A-2 | O | CH₃CH₃CH₃ | ⁱPr | ⁱPr | ⁱPr | H | (8a-2-21c-2) |
| A-2 | O | CH₃CH₃CH₃ | Bu | Bu | Bu | H | (8a-2-22c-2) |
| A-2 | O | CH₃(CH₃)₄CH₃ | CH₃ | CH₃ | CH₃ | H | (8a-2-23c-2) |
| A-2 | O | CH₃(CH₃)₄CH₃ | CH₃ | CH₃ | Et | H | (8a-2-24c-2) |
| A-2 | O | CH₃(CH₃)₄CH₃ | CH₃ | CH₃ | ⁱPr | H | (8a-2-25c-2) |
| A-2 | O | CH₃(CH₃)₄CH₃ | CH₃ | CH₃ | Bu | H | (8a-2-26c-2) |
| A-2 | O | CH₃(CH₃)₄CH₃ | CH₃ | CH₃ | Bnz | H | (8a-2-27c-2) |
| A-2 | O | CH₃(CH₃)₄CH₃ | CH₃ | Et | Et | H | (8a-2-28c-2) |
| A-2 | O | CH₃(CH₃)₄CH₃ | CH₃ | CH₃CH₃OCH₃ | CH₃CH₃OCH₃ | H | (8a-2-29c-2) |
| A-2 | O | CH₃(CH₃)₄CH₃ | CH₃ | Pr | Bu | H | (8a-2-30c-2) |
| A-2 | O | CH₃(CH₃)₄CH₃ | Et | Et | Et | H | (8a-2-31c-2) |
| A-2 | O | CH₃(CH₃)₄CH₃ | ⁱPr | ⁱPr | ⁱPr | H | (8a-2-32c-2) |
| A-2 | O | CH₃(CH₃)₄CH₃ | Bu | Bu | Bu | H | (8a-2-33c-2) |

| A | χ(R¹)ⁿ | R² | R³ | R⁴ | R⁵ | R′ | |
|---|---|---|---|---|---|---|---|
| A-6 | NH | CH₃ | CH₃ | CH₃ | CH₃ | H | (8a-2-1a-6) |
| A-6 | NH | CH₃ | CH₃ | CH₃ | Et | H | (8a-2-2a-6) |
| A-6 | NH | CH₃ | CH₃ | CH₃ | ⁱPr | H | (8a-2-3a-6) |
| A-6 | NH | CH₃ | CH₃ | CH₃ | Bu | H | (8a-2-4a-6) |
| A-6 | NH | CH₃ | CH₃ | CH₃ | Bnz | H | (8a-2-5a-6) |
| A-6 | NH | CH₃ | CH₃ | Et | Et | H | (8a-2-6a-6) |
| A-6 | NH | CH₃ | CH₃ | CH₃CH₃OCH₃ | CH₃CH₃OCH₃ | H | (8a-2-7a-6) |
| A-6 | NH | CH₃ | CH₃ | Pr | Bu | H | (8a-2-8a-6) |
| A-6 | NH | CH₃ | Et | Et | Et | H | (8a-2-9a-6) |
| A-6 | NH | CH₃ | ⁱPr | ⁱPr | ⁱPr | H | (8a-2-10a-6) |
| A-6 | NH | CH₃ | Bu | Bu | Bu | H | (8a-2-11a-6) |
| A-6 | NH | CH₃CH₃CH₃ | CH₃ | CH₃ | CH₃ | H | (8a-2-12a-6) |
| A-6 | NH | CH₃CH₃CH₃ | CH₃ | CH₃ | Et | H | (8a-2-13a-6) |
| A-6 | NH | CH₃CH₃CH₃ | CH₃ | CH₃ | ⁱPr | H | (8a-2-14a-6) |
| A-6 | NH | CH₃CH₃CH₃ | CH₃ | CH₃ | Bu | H | (8a-2-15a-6) |
| A-6 | NH | CH₃CH₃CH₃ | CH₃ | CH₃ | Bnz | H | (8a-2-16a-6) |
| A-6 | NH | CH₃CH₃CH₃ | CH₃ | Et | Et | H | (8a-2-17a-6) |
| A-6 | NH | CH₃CH₃CH₃ | CH₃ | CH₃CH₃OCH₃ | CH₃CH₃OCH₃ | H | (8a-2-18a-6) |
| A-6 | NH | CH₃CH₃CH₃ | CH₃ | Pr | Bu | H | (8a-2-19a-6) |
| A-6 | NH | CH₃CH₃CH₃ | Et | Et | Et | H | (8a-2-20a-6) |
| A-6 | NH | CH₃CH₃CH₃ | ⁱPr | ⁱPr | ⁱPr | H | (8a-2-21a-6) |
| A-6 | NH | CH₃CH₃CH₃ | Bu | Bu | Bu | H | (8a-2-22a-6) |
| A-6 | NH | CH₃(CH₃)₄CH₃ | CH₃ | CH₃ | CH₃ | H | (8a-2-23a-6) |
| A-6 | NH | CH₃(CH₃)₄CH₃ | CH₃ | CH₃ | Et | H | (8a-2-24a-6) |
| A-6 | NH | CH₃(CH₃)₄CH₃ | CH₃ | CH₃ | ⁱPr | H | (8a-2-25a-6) |
| A-6 | NH | CH₃(CH₃)₄CH₃ | CH₃ | CH₃ | Bu | H | (8a-2-26a-6) |
| A-6 | NH | CH₃(CH₃)₄CH₃ | CH₃ | CH₃ | Bnz | H | (8a-2-27a-6) |
| A-6 | NH | CH₃(CH₃)₄CH₃ | CH₃ | Et | Et | H | (8a-2-28a-6) |
| A-6 | NH | CH₃(CH₃)₄CH₃ | CH₃ | CH₃CH₃OCH₃ | CH₃CH₃OCH₃ | H | (8a-2-29a-6) |
| A-6 | NH | CH₃(CH₃)₄CH₃ | CH₃ | Pr | Bu | H | (8a-2-30a-6) |
| A-6 | NH | CH₃(CH₃)₄CH₃ | Et | Et | Et | H | (8a-2-31a-6) |
| A-6 | NH | CH₃(CH₃)₄CH₃ | ⁱPr | ⁱPr | ⁱPr | H | (8a-2-32a-6) |
| A-6 | NH | CH₃(CH₃)₄CH₃ | Bu | Bu | Bu | H | (8a-2-33a-6) |

| A | χ(R¹)ⁿ | R² | R³ | R⁴ | R⁵ | R′ | |
|---|---|---|---|---|---|---|---|
| A-6 | N(CH₃) | CH₃ | CH₃ | CH₃ | CH₃ | H | (8a-2-1b-6) |
| A-6 | N(CH₃) | CH₃ | CH₃ | CH₃ | Et | H | (8a-2-2b-6) |
| A-6 | N(CH₃) | CH₃ | CH₃ | CH₃ | ⁱPr | H | (8a-2-3b-6) |
| A-6 | N(CH₃) | CH₃ | CH₃ | CH₃ | Bu | H | (8a-2-4b-6) |
| A-6 | N(CH₃) | CH₃ | CH₃ | CH₃ | Bnz | H | (8a-2-5b-6) |
| A-6 | N(CH₃) | CH₃ | CH₃ | Et | Et | H | (8a-2-6b-6) |
| A-6 | N(CH₃) | CH₃ | CH₃ | CH₃CH₃OCH₃ | CH₃CH₃OCH₃ | H | (8a-2-7b-6) |
| A-6 | N(CH₃) | CH₃ | CH₃ | Pr | Bu | H | (8a-2-8b-6) |
| A-6 | N(CH₃) | CH₃ | Et | Et | Et | H | (8a-2-9b-6) |
| A-6 | N(CH₃) | CH₃ | ⁱPr | ⁱPr | ⁱPr | H | (8a-2-10b-6) |
| A-6 | N(CH₃) | CH₃ | Bu | Bu | Bu | H | (8a-2-11b-6) |
| A-6 | N(CH₃) | CH₃CH₃CH₃ | CH₃ | CH₃ | CH₃ | H | (8a-2-12b-6) |
| A-6 | N(CH₃) | CH₃CH₃CH₃ | CH₃ | CH₃ | Et | H | (8a-2-13b-6) |
| A-6 | N(CH₃) | CH₃CH₃CH₃ | CH₃ | CH₃ | ⁱPr | H | (8a-2-14b-6) |
| A-6 | N(CH₃) | CH₃CH₃CH₃ | CH₃ | CH₃ | Bu | H | (8a-2-15b-6) |
| A-6 | N(CH₃) | CH₃CH₃CH₃ | CH₃ | CH₃ | Bnz | H | (8a-2-16b-6) |
| A-6 | N(CH₃) | CH₃CH₃CH₃ | CH₃ | Et | Et | H | (8a-2-17b-6) |
| A-6 | N(CH₃) | CH₃CH₃CH₃ | CH₃ | CH₃CH₃OCH₃ | CH₃CH₃OCH₃ | H | (8a-2-18b-6) |
| A-6 | N(CH₃) | CH₃CH₃CH₃ | CH₃ | Pr | Bu | H | (8a-2-19b-6) |
| A-6 | N(CH₃) | CH₃CH₃CH₃ | Et | Et | Et | H | (8a-2-20b-6) |
| A-6 | N(CH₃) | CH₃CH₃CH₃ | ⁱPr | ⁱPr | ⁱPr | H | (8a-2-21b-6) |
| A-6 | N(CH₃) | CH₃CH₃CH₃ | Bu | Bu | Bu | H | (8a-2-22b-6) |
| A-6 | N(CH₃) | CH₃(CH₃)₄CH₃ | CH₃ | CH₃ | CH₃ | H | (8a-2-23b-6) |
| A-6 | N(CH₃) | CH₃(CH₃)₄CH₃ | CH₃ | CH₃ | Et | H | (8a-2-24b-6) |
| A-6 | N(CH₃) | CH₃(CH₃)₄CH₃ | CH₃ | CH₃ | ⁱPr | H | (8a-2-25b-6) |
| A-6 | N(CH₃) | CH₃(CH₃)₄CH₃ | CH₃ | CH₃ | Bu | H | (8a-2-26b-6) |
| A-6 | N(CH₃) | CH₃(CH₃)₄CH₃ | CH₃ | CH₃ | Bnz | H | (8a-2-27b-6) |
| A-6 | N(CH₃) | CH₃(CH₃)₄CH₃ | CH₃ | Et | Et | H | (8a-2-28b-6) |
| A-6 | N(CH₃) | CH₃(CH₃)₄CH₃ | CH₃ | CH₃CH₃OCH₃ | CH₃CH₃OCH₃ | H | (8a-2-29b-6) |
| A-6 | N(CH₃) | CH₃(CH₃)₄CH₃ | CH₃ | Pr | Bu | H | (8a-2-30b-6) |
| A-6 | N(CH₃) | CH₃(CH₃)₄CH₃ | Et | Et | Et | H | (8a-2-31b-6) |
| A-6 | N(CH₃) | CH₃(CH₃)₄CH₃ | ⁱPr | ⁱPr | ⁱPr | H | (8a-2-32b-6) |
| A-6 | N(CH₃) | CH₃(CH₃)₄CH₃ | Bu | Bu | Bu | H | (8a-2-33b-6) |

| A | χ(R¹)ⁿ | R² | R³ | R⁴ | R⁵ | R′ | |
|---|---|---|---|---|---|---|---|
| A-6 | O | CH₃ | CH₃ | CH₃ | CH₃ | H | (8a-2-1c-6) |
| A-6 | O | CH₃ | CH₃ | CH₃ | Et | H | (8a-2-2c-6) |
| A-6 | O | CH₃ | CH₃ | CH₃ | ⁱPr | H | (8a-2-3c-6) |
| A-6 | O | CH₃ | CH₃ | CH₃ | Bu | H | (8a-2-4c-6) |
| A-6 | O | CH₃ | CH₃ | CH₃ | Bnz | H | (8a-2-5c-6) |
| A-6 | O | CH₃ | CH₃ | Et | Et | H | (8a-2-6c-6) |
| A-6 | O | CH₃ | CH₃ | CH₃CH₃OCH₃ | CH₃CH₃OCH₃ | H | (8a-2-7c-6) |
| A-6 | O | CH₃ | CH₃ | Pr | Bu | H | (8a-2-8c-6) |
| A-6 | O | CH₃ | Et | Et | Et | H | (8a-2-9c-6) |
| A-6 | O | CH₃ | ⁱPr | ⁱPr | ⁱPr | H | (8a-2-10c-6) |
| A-6 | O | CH₃ | Bu | Bu | Bu | H | (8a-2-11c-6) |
| A-6 | O | CH₃CH₃CH₃ | CH₃ | CH₃ | CH₃ | H | (8a-2-12c-6) |
| A-6 | O | CH₃CH₃CH₃ | CH₃ | CH₃ | Et | H | (8a-2-13c-6) |
| A-6 | O | CH₃CH₃CH₃ | CH₃ | CH₃ | ⁱPr | H | (8a-2-14c-6) |
| A-6 | O | CH₃CH₃CH₃ | CH₃ | CH₃ | Bu | H | (8a-2-15c-6) |
| A-6 | O | CH₃CH₃CH₃ | CH₃ | CH₃ | Bnz | H | (8a-2-16c-6) |
| A-6 | O | CH₃CH₃CH₃ | CH₃ | Et | Et | H | (8a-2-17c-6) |
| A-6 | O | CH₃CH₃CH₃ | CH₃ | CH₃CH₃OCH₃ | CH₃CH₃OCH₃ | H | (8a-2-18c-6) |
| A-6 | O | CH₃CH₃CH₃ | CH₃ | Pr | Bu | H | (8a-2-19c-6) |
| A-6 | O | CH₃CH₃CH₃ | Et | Et | Et | H | (8a-2-20c-6) |
| A-6 | O | CH₃CH₃CH₃ | ⁱPr | ⁱPr | ⁱPr | H | (8a-2-21c-6) |
| A-6 | O | CH₃CH₃CH₃ | Bu | Bu | Bu | H | (8a-2-22c-6) |
| A-6 | O | CH₃(CH₃)₄CH₃ | CH₃ | CH₃ | CH₃ | H | (8a-2-23c-6) |
| A-6 | O | CH₃(CH₃)₄CH₃ | CH₃ | CH₃ | Et | H | (8a-2-24c-6) |
| A-6 | O | CH₃(CH₃)₄CH₃ | CH₃ | CH₃ | ⁱPr | H | (8a-2-25c-6) |
| A-6 | O | CH₃(CH₃)₄CH₃ | CH₃ | CH₃ | Bu | H | (8a-2-26c-6) |
| A-6 | O | CH₃(CH₃)₄CH₃ | CH₃ | CH₃ | Bnz | H | (8a-2-27c-6) |
| A-6 | O | CH₃(CH₃)₄CH₃ | CH₃ | Et | Et | H | (8a-2-28c-6) |
| A-6 | O | CH₃(CH₃)₄CH₃ | CH₃ | CH₃CH₃OCH₃ | CH₃CH₃OCH₃ | H | (8a-2-29c-6) |
| A-6 | O | CH₃(CH₃)₄CH₃ | CH₃ | Pr | Bu | H | (8a-2-30c-6) |
| A-6 | O | CH₃(CH₃)₄CH₃ | Et | Et | Et | H | (8a-2-31c-6) |
| A-6 | O | CH₃(CH₃)₄CH₃ | ⁱPr | ⁱPr | ⁱPr | H | (8a-2-32c-6) |
| A-6 | O | CH₃(CH₃)₄CH₃ | Bu | Bu | Bu | H | (8a-2-33c-6) |

| A | χ(R¹)ⁿ | R² | R³ | R⁴ | R⁵ | R′ | |
|---|---|---|---|---|---|---|---|
| A-2 | NH | CH₃ | CH₃ | CH₃ | CH₃ | CH₃ | (8b-2-1a-2) |
| A-2 | NH | CH₃ | CH₃ | CH₃ | Et | CH₃ | (8b-2-2a-2) |
| A-2 | NH | CH₃ | CH₃ | CH₃ | ⁱPr | CH₃ | (8b-2-3a-2) |
| A-2 | NH | CH₃ | CH₃ | CH₃ | Bu | CH₃ | (8b-2-4a-2) |
| A-2 | NH | CH₃ | CH₃ | CH₃ | Bnz | CH₃ | (8b-2-5a-2) |
| A-2 | NH | CH₃ | CH₃ | Et | Et | CH₃ | (8b-2-6a-2) |
| A-2 | NH | CH₃ | CH₃ | CH₃CH₃OCH₃ | CH₃CH₃OCH₃ | CH₃ | (8b-2-7a-2) |
| A-2 | NH | CH₃ | CH₃ | Pr | Bu | CH₃ | (8b-2-8a-2) |
| A-2 | NH | CH₃ | CH₃ | CH₃ | Et | Et | (8b-2-9a-2) |
| A-2 | NH | CH₃CH₃CH₃ | CH₃ | CH₃ | CH₃ | CH₃ | (8b-2-10a-2) |
| A-2 | NH | CH₃CH₃CH₃ | CH₃ | CH₃ | Et | CH₃ | (8b-2-11a-2) |
| A-2 | NH | CH₃CH₃CH₃ | CH₃ | CH₃ | ⁱPr | CH₃ | (8b-2-12a-2) |
| A-2 | NH | CH₃CH₃CH₃ | CH₃ | CH₃ | Bu | CH₃ | (8b-2-13a-2) |
| A-2 | NH | CH₃CH₃CH₃ | CH₃ | CH₃ | Bnz | CH₃ | (8b-2-14a-2) |
| A-2 | NH | CH₃CH₃CH₃ | CH₃ | Et | Et | CH₃ | (8b-2-15a-2) |
| A-2 | NH | CH₃CH₃CH₃ | CH₃ | CH₃CH₃OCH₃ | CH₃CH₃OCH₃ | CH₃ | (8b-2-16a-2) |
| A-2 | NH | CH₃CH₃CH₃ | CH₃ | Pr | Bu | CH₃ | (8b-2-17a-2) |
| A-2 | NH | CH₃CH₃CH₃ | CH₃ | CH₃ | Et | Et | (8b-2-18a-2) |
| A-2 | NH | CH₃(CH₃)₄CH₃ | CH₃ | CH₃ | CH₃ | CH₃ | (8b-2-19a-2) |
| A-2 | NH | CH₃(CH₃)₄CH₃ | CH₃ | CH₃ | Et | CH₃ | (8b-2-20a-2) |
| A-2 | NH | CH₃(CH₃)₄CH₃ | CH₃ | CH₃ | ⁱPr | CH₃ | (8b-2-21a-2) |
| A-2 | NH | CH₃(CH₃)₄CH₃ | CH₃ | CH₃ | Bu | CH₃ | (8b-2-22a-2) |
| A-2 | NH | CH₃(CH₃)₄CH₃ | CH₃ | CH₃ | Bnz | CH₃ | (8b-2-23a-2) |
| A-2 | NH | CH₃(CH₃)₄CH₃ | CH₃ | Et | Et | CH₃ | (8b-2-24a-2) |
| A-2 | NH | CH₃(CH₃)₄CH₃ | CH₃ | CH₃CH₃OCH₃ | CH₃CH₃OCH₃ | CH₃ | (8b-2-25a-2) |
| A-2 | NH | CH₃(CH₃)₄CH₃ | CH₃ | Pr | Bu | CH₃ | (8b-2-26a-2) |
| A-2 | NH | CH₃(CH₃)₄CH₃ | CH₃ | CH₃ | Et | Et | (8b-2-27a-2) |

| A | χ(R¹)ⁿ | R² | R³ | R⁴ | R⁵ | R′ | |
|---|---|---|---|---|---|---|---|
| A-2 | N(CH₃) | CH₃ | CH₃ | CH₃ | CH₃ | CH₃ | (8b-2-1b-2) |
| A-2 | N(CH₃) | CH₃ | CH₃ | CH₃ | Et | CH₃ | (8b-2-2b-2) |
| A-2 | N(CH₃) | CH₃ | CH₃ | CH₃ | ⁱPr | CH₃ | (8b-2-3b-2) |
| A-2 | N(CH₃) | CH₃ | CH₃ | CH₃ | Bu | CH₃ | (8b-2-4b-2) |
| A-2 | N(CH₃) | CH₃ | CH₃ | CH₃ | Bnz | CH₃ | (8b-2-5b-2) |
| A-2 | N(CH₃) | CH₃ | CH₃ | Et | Et | CH₃ | (8b-2-6b-2) |
| A-2 | N(CH₃) | CH₃ | CH₃ | CH₃CH₃OCH₃ | CH₃CH₃OCH₃ | CH₃ | (8b-2-7b-2) |
| A-2 | N(CH₃) | CH₃ | CH₃ | Pr | Bu | CH₃ | (8b-2-8b-2) |
| A-2 | N(CH₃) | CH₃ | CH₃ | CH₃ | Et | Et | (8b-2-9b-2) |
| A-2 | N(CH₃) | CH₃CH₃CH₃ | CH₃ | CH₃ | CH₃ | CH₃ | (8b-2-10b-2) |
| A-2 | N(CH₃) | CH₃CH₃CH₃ | CH₃ | CH₃ | Et | CH₃ | (8b-2-11b-2) |
| A-2 | N(CH₃) | CH₃CH₃CH₃ | CH₃ | CH₃ | ⁱPr | CH₃ | (8b-2-12b-2) |
| A-2 | N(CH₃) | CH₃CH₃CH₃ | CH₃ | CH₃ | Bu | CH₃ | (8b-2-13b-2) |
| A-2 | N(CH₃) | CH₃CH₃CH₃ | CH₃ | CH₃ | Bnz | CH₃ | (8b-2-14b-2) |
| A-2 | N(CH₃) | CH₃CH₃CH₃ | CH₃ | Et | Et | CH₃ | (8b-2-15b-2) |
| A-2 | N(CH₃) | CH₃CH₃CH₃ | CH₃ | CH₃CH₃OCH₃ | CH₃CH₃OCH₃ | CH₃ | (8b-2-16b-2) |
| A-2 | N(CH₃) | CH₃CH₃CH₃ | CH₃ | Pr | Bu | CH₃ | (8b-2-17b-2) |
| A-2 | N(CH₃) | CH₃CH₃CH₃ | CH₃ | CH₃ | Et | Et | (8b-2-18b-2) |
| A-2 | N(CH₃) | CH₃(CH₃)₄CH₃ | CH₃ | CH₃ | CH₃ | CH₃ | (8b-2-19b-2) |
| A-2 | N(CH₃) | CH₃(CH₃)₄CH₃ | CH₃ | CH₃ | Et | CH₃ | (8b-2-20b-2) |
| A-2 | N(CH₃) | CH₃(CH₃)₄CH₃ | CH₃ | CH₃ | ⁱPr | CH₃ | (8b-2-21b-2) |
| A-2 | N(CH₃) | CH₃(CH₃)₄CH₃ | CH₃ | CH₃ | Bu | CH₃ | (8b-2-22b-2) |
| A-2 | N(CH₃) | CH₃(CH₃)₄CH₃ | CH₃ | CH₃ | Bnz | CH₃ | (8b-2-23b-2) |
| A-2 | N(CH₃) | CH₃(CH₃)₄CH₃ | CH₃ | Et | Et | CH₃ | (8b-2-24b-2) |
| A-2 | N(CH₃) | CH₃(CH₃)₄CH₃ | CH₃ | CH₃CH₃OCH₃ | CH₃CH₃OCH₃ | CH₃ | (8b-2-25b-2) |
| A-2 | N(CH₃) | CH₃(CH₃)₄CH₃ | CH₃ | Pr | Bu | CH₃ | (8b-2-26b-2) |
| A-2 | N(CH₃) | CH₃(CH₃)₄CH₃ | CH₃ | CH₃ | Et | Et | (8b-2-27b-2) |

| A | χ(R¹)ⁿ | R² | R³ | R⁴ | R⁵ | R′ | |
|---|---|---|---|---|---|---|---|
| A-2 | O | CH₃ | CH₃ | CH₃ | CH₃ | CH₃ | (8b-2-1c-2) |
| A-2 | O | CH₃ | CH₃ | CH₃ | Et | CH₃ | (8b-2-2c-2) |
| A-2 | O | CH₃ | CH₃ | CH₃ | ⁱPr | CH₃ | (8b-2-3c-2) |
| A-2 | O | CH₃ | CH₃ | CH₃ | Bu | CH₃ | (8b-2-4c-2) |
| A-2 | O | CH₃ | CH₃ | CH₃ | Bnz | CH₃ | (8b-2-5c-2) |
| A-2 | O | CH₃ | CH₃ | Et | Et | CH₃ | (8b-2-6c-2) |
| A-2 | O | CH₃ | CH₃ | CH₃CH₃OCH₃ | CH₃CH₃OCH₃ | CH₃ | (8b-2-7c-2) |
| A-2 | O | CH₃ | CH₃ | Pr | Bu | CH₃ | (8b-2-8c-2) |
| A-2 | O | CH₃ | CH₃ | CH₃ | Et | Et | (8b-2-9c-2) |
| A-2 | O | CH₃CH₃CH₃ | CH₃ | CH₃ | CH₃ | CH₃ | (8b-2-10c-2) |
| A-2 | O | CH₃CH₃CH₃ | CH₃ | CH₃ | Et | CH₃ | (8b-2-11c-2) |
| A-2 | O | CH₃CH₃CH₃ | CH₃ | CH₃ | ⁱPr | CH₃ | (8b-2-12c-2) |
| A-2 | O | CH₃CH₃CH₃ | CH₃ | CH₃ | Bu | CH₃ | (8b-2-13c-2) |
| A-2 | O | CH₃CH₃CH₃ | CH₃ | CH₃ | Bnz | CH₃ | (8b-2-14c-2) |
| A-2 | O | CH₃CH₃CH₃ | CH₃ | Et | Et | CH₃ | (8b-2-15c-2) |
| A-2 | O | CH₃CH₃CH₃ | CH₃ | CH₃CH₃OCH₃ | CH₃CH₃OCH₃ | CH₃ | (8b-2-16c-2) |
| A-2 | O | CH₃CH₃CH₃ | CH₃ | Pr | Bu | CH₃ | (8b-2-17c-2) |
| A-2 | O | CH₃CH₃CH₃ | CH₃ | CH₃ | Et | Et | (8b-2-18c-2) |
| A-2 | O | CH₃(CH₃)₄CH₃ | CH₃ | CH₃ | CH₃ | CH₃ | (8b-2-19c-2) |
| A-2 | O | CH₃(CH₃)₄CH₃ | CH₃ | CH₃ | Et | CH₃ | (8b-2-20c-2) |
| A-2 | O | CH₃(CH₃)₄CH₃ | CH₃ | CH₃ | ⁱPr | CH₃ | (8b-2-21c-2) |
| A-2 | O | CH₃(CH₃)₄CH₃ | CH₃ | CH₃ | Bu | CH₃ | (8b-2-22c-2) |
| A-2 | O | CH₃(CH₃)₄CH₃ | CH₃ | CH₃ | Bnz | CH₃ | (8b-2-23c-2) |
| A-2 | O | CH₃(CH₃)₄CH₃ | CH₃ | Et | Et | CH₃ | (8b-2-24c-2) |
| A-2 | O | CH₃(CH₃)₄CH₃ | CH₃ | CH₃CH₃OCH₃ | CH₃CH₃OCH₃ | CH₃ | (8b-2-25c-2) |
| A-2 | O | CH₃(CH₃)₄CH₃ | CH₃ | Pr | Bu | CH₃ | (8b-2-26c-2) |
| A-2 | O | CH₃(CH₃)₄CH₃ | CH₃ | CH₃ | Et | Et | (8b-2-27c-2) |

| A | χ(R¹)ⁿ | R² | R³ | R⁴ | R⁵ | R′ | |
|---|---|---|---|---|---|---|---|
| A-6 | NH | CH₃ | CH₃ | CH₃ | CH₃ | CH₃ | (8b-2-1a-6) |
| A-6 | NH | CH₃ | CH₃ | CH₃ | Et | CH₃ | (86-3-2a-6) |
| A-6 | NH | CH₃ | CH₃ | CH₃ | ⁱPr | CH₃ | (8b-2-3a-6) |
| A-6 | NH | CH₃ | CH₃ | CH₃ | Bu | CH₃ | (8b-2-4a-6) |
| A-6 | NH | CH₃ | CH₃ | CH₃ | Bnz | CH₃ | (8b-2-5a-6) |
| A-6 | NH | CH₃ | CH₃ | Et | Et | CH₃ | (8b-2-6a-6) |
| A-6 | NH | CH₃ | CH₃ | CH₃CH₃OCH₃ | CH₃CH₃OCH₃ | CH₃ | (8b-2-7a-6) |
| A-6 | NH | CH₃ | CH₃ | Pr | Bu | CH₃ | (8b-2-8a-6) |
| A-6 | NH | CH₃ | CH₃ | CH₃ | Et | Et | (8b-2-9a-6) |
| A-6 | NH | CH₃CH₃CH₃ | CH₃ | CH₃ | CH₃ | CH₃ | (8b-2-10a-6) |
| A-6 | NH | CH₃CH₃CH₃ | CH₃ | CH₃ | Et | CH₃ | (8b-2-11a-6) |
| A-6 | NH | CH₃CH₃CH₃ | CH₃ | CH₃ | ⁱPr | CH₃ | (8b-2-12a-6) |
| A-6 | NH | CH₃CH₃CH₃ | CH₃ | CH₃ | Bu | CH₃ | (8b-2-13a-6) |
| A-6 | NH | CH₃CH₃CH₃ | CH₃ | CH₃ | Bnz | CH₃ | (8b-2-14a-6) |
| A-6 | NH | CH₃CH₃CH₃ | CH₃ | Et | Et | CH₃ | (8b-2-15a-6) |
| A-6 | NH | CH₃CH₃CH₃ | CH₃ | CH₃CH₃OCH₃ | CH₃CH₃OCH₃ | CH₃ | (8b-2-16a-6) |
| A-6 | NH | CH₃CH₃CH₃ | CH₃ | Pr | Bu | CH₃ | (8b-2-17a-6) |
| A-6 | NH | CH₃CH₃CH₃ | CH₃ | CH₃ | Et | Et | (8b-2-18a-6) |
| A-6 | NH | CH₃(CH₃)₄CH₃ | CH₃ | CH₃ | CH₃ | CH₃ | (8b-2-19a-6) |
| A-6 | NH | CH₃(CH₃)₄CH₃ | CH₃ | CH₃ | Et | CH₃ | (8b-2-20a-6) |
| A-6 | NH | CH₃(CH₃)₄CH₃ | CH₃ | CH₃ | ⁱPr | CH₃ | (8b-2-21a-6) |
| A-6 | NH | CH₃(CH₃)₄CH₃ | CH₃ | CH₃ | Bu | CH₃ | (8b-2-22a-6) |
| A-6 | NH | CH₃(CH₃)₄CH₃ | CH₃ | CH₃ | Bnz | CH₃ | (8b-2-23a-6) |
| A-6 | NH | CH₃(CH₃)₄CH₃ | CH₃ | Et | Et | CH₃ | (8b-2-24a-6) |
| A-6 | NH | CH₃(CH₃)₄CH₃ | CH₃ | CH₃CH₃OCH₃ | CH₃CH₃OCH₃ | CH₃ | (8b-2-25a-6) |
| A-6 | NH | CH₃(CH₃)₄CH₃ | CH₃ | Pr | Bu | CH₃ | (8b-2-26a-6) |
| A-6 | NH | CH₃(CH₃)₄CH₃ | CH₃ | CH₃ | Et | Et | (86-2-27a-6) |

| A | χ(R¹)ⁿ | R² | R³ | R⁴ | R⁵ | R′ | |
|---|---|---|---|---|---|---|---|
| A-6 | N(CH₃) | CH₃ | CH₃ | CH₃ | CH₃ | CH₃ | (8b-2-1b-6) |
| A-6 | N(CH₃) | CH₃ | CH₃ | CH₃ | Et | CH₃ | (8b-2-2b-6) |
| A-6 | N(CH₃) | CH₃ | CH₃ | CH₃ | ⁱPr | CH₃ | (8b-2-3b-6) |
| A-6 | N(CH₃) | CH₃ | CH₃ | CH₃ | Bu | CH₃ | (8b-2-4b-6) |
| A-6 | N(CH₃) | CH₃ | CH₃ | CH₃ | Bnz | CH₃ | (8b-2-5b-6) |
| A-6 | N(CH₃) | CH₃ | CH₃ | Et | Et | CH₃ | (8b-2-6b-6) |
| A-6 | N(CH₃) | CH₃ | CH₃ | CH₃CH₃OCH₃ | CH₃CH₃OCH₃ | CH₃ | (8b-2-7b-6) |
| A-6 | N(CH₃) | CH₃ | CH₃ | Pr | Bu | CH₃ | (8b-2-8b-6) |
| A-6 | N(CH₃) | CH₃ | CH₃ | CH₃ | Et | Et | (8b-2-9b-6) |
| A-6 | N(CH₃) | CH₃CH₃CH₃ | CH₃ | CH₃ | CH₃ | CH₃ | (8b-2-10b-6) |
| A-6 | N(CH₃) | CH₃CH₃CH₃ | CH₃ | CH₃ | Et | CH₃ | (86-2-11b-6) |
| A-6 | N(CH₃) | CH₃CH₃CH₃ | CH₃ | CH₃ | ⁱPr | CH₃ | (8b-2-12b-6) |
| A-6 | N(CH₃) | CH₃CH₃CH₃ | CH₃ | CH₃ | Bu | CH₃ | (8b-2-12b-6) |
| A-6 | N(CH₃) | CH₃CH₃CH₃ | CH₃ | CH₃ | Bnz | CH₃ | (86-2-14b-6) |
| A-6 | N(CH₃) | CH₃CH₃CH₃ | CH₃ | Et | Et | CH₃ | (8b-2-15b-6) |
| A-6 | N(CH₃) | CH₃CH₃CH₃ | CH₃ | CH₃CH₃OCH₃ | CH₃CH₃OCH₃ | CH₃ | (8b-2-16b-6) |
| A-6 | N(CH₃) | CH₃CH₃CH₃ | CH₃ | Pr | Bu | CH₃ | (8b-2-17b-6) |
| A-6 | N(CH₃) | CH₃CH₃CH₃ | CH₃ | CH₃ | Et | Et | (8b-2-18b-6) |
| A-6 | N(CH₃) | CH₃(CH₃)₄CH₃ | CH₃ | CH₃ | CH₃ | CH₃ | (8b-2-19b-6) |
| A-6 | N(CH₃) | CH₃(CH₃)₄CH₃ | CH₃ | CH₃ | Et | CH₃ | (8b-2-20b-6) |
| A-6 | N(CH₃) | CH₃(CH₃)₄CH₃ | CH₃ | CH₃ | ⁱPr | CH₃ | (8b-2-21b-6) |
| A-6 | N(CH₃) | CH₃(CH₃)₄CH₃ | CH₃ | CH₃ | Bu | CH₃ | (8b-2-22b-6) |
| A-6 | N(CH₃) | CH₃(CH₃)₄CH₃ | CH₃ | CH₃ | Bnz | CH₃ | (8b-2-23b-6) |
| A-6 | N(CH₃) | CH₃(CH₃)₄CH₃ | CH₃ | Et | Et | CH₃ | (86-2-24b-6) |
| A-6 | N(CH₃) | CH₃(CH₃)₄CH₃ | CH₃ | CH₃CH₃OCH₃ | CH₃CH₃OCH₃ | CH₃ | (8b-2-25b-6) |
| A-6 | N(CH₃) | CH₃(CH₃)₄CH₃ | CH₃ | Pr | Bu | CH₃ | (8b-2-26b-6) |
| A-6 | N(CH₃) | CH₃(CH₃)₄CH₃ | CH₃ | CH₃ | Et | Et | (8b-2-27b-6) |

| A | X(R [symbol] ) [symbol] | R [symbol] | R [symbol] | R [symbol] | R [symbol] | R [symbol] | |
|---|---|---|---|---|---|---|---|
| A-6 | 0 | CH [symbol] | CH [symbol] | CH [symbol] | CH [symbol] | CH [symbol] | (8b-2-1c-b) |
| A-6 | 0 | CH [symbol] | CH [symbol] | CH [symbol] | Et | CH [symbol] | (8b-2-2c-b) |
| A-6 | 0 | CH [symbol] | CH [symbol] | CH [symbol] | ⁱPr | CH [symbol] | (8b-2-3c-b) |
| A-6 | 0 | CH [symbol] | CH [symbol] | CH [symbol] | Bu | CH [symbol] | (8b-2-4c-b) |
| A-6 | 0 | CH [symbol] | CH [symbol] | CH [symbol] | Bnz | CH [symbol] | (8b-2-5c-b) |
| A-6 | 0 | CH [symbol] | CH [symbol] | Et | Et | CH [symbol] | (8b-2-6c-b) |
| A-6 | 0 | CH [symbol] | CH [symbol] | CH [symbol] CH [symbol] OCH [symbol] | CH [symbol] CH [symbol] OCH [symbol] | CH [symbol] | (8b-2-7c-b) |
| A-6 | 0 | CH [symbol] | CH [symbol] | Pr | Bu | CH [symbol] | (8b-2-8c-b) |
| A-6 | 0 | CH [symbol] | CH [symbol] | CH [symbol] | Et | Et | (8b-2-9c-b) |
| A-6 | 0 | CH [symbol] CH [symbol] CH [symbol] | CH [symbol] | CH [symbol] | CH [symbol] | CH [symbol] | (8b-2-10c-b) |
| A-6 | 0 | CH [symbol] CH [symbol] CH [symbol] | CH [symbol] | CH [symbol] | Et | CH [symbol] | (8b-2-11c-b) |
| A-6 | 0 | CH [symbol] CH [symbol] CH [symbol] | CH [symbol] | CH [symbol] | ⁱPr | CH [symbol] | (8b-2-12c-b) |
| A-6 | 0 | CH [symbol] CH [symbol] CH [symbol] | CH [symbol] | CH [symbol] | Bu | CH [symbol] | (8b-2-13c-b) |
| A-6 | 0 | CH [symbol] CH [symbol] CH [symbol] | CH [symbol] | CH [symbol] | Bnz | CH [symbol] | (8b-2-14c-b) |
| A-6 | 0 | CH [symbol] CH [symbol] CH [symbol] | CH [symbol] | Et | Et | CH [symbol] | (8b-2-15c-b) |
| A-6 | 0 | CH [symbol] CH [symbol] CH [symbol] | CH [symbol] | CH [symbol] CH [symbol] OCH [symbol] | CH [symbol] CH [symbol] OCH [symbol] | CH [symbol] | (8b-2-16c-b) |
| A-6 | 0 | CH [symbol] CH [symbol] CH [symbol] | CH [symbol] | Pr | Bu | CH [symbol] | (8b-2-17c-b) |
| A-6 | 0 | CH [symbol] CH [symbol] CH [symbol] | CH [symbol] | CH [symbol] | Et | Et | (8b-2-18c-b) |
| A-6 | 0 | CH [symbol] (CH [symbol] ) [symbol] CH [symbol] | CH [symbol] | CH [symbol] | CH [symbol] | CH [symbol] | (8b-2-19c-b) |
| A-6 | 0 | CH [symbol] (CH [symbol] ) [symbol] CH [symbol] | CH [symbol] | CH [symbol] | Et | CH [symbol] | (8b-2-20c-b) |
| A-6 | 0 | CH [symbol] (CH [symbol] ) [symbol] CH [symbol] | CH [symbol] | CH [symbol] | ⁱPr | CH [symbol] | (8b-2-21c-b) |
| A-6 | 0 | CH [symbol] (CH [symbol] ) [symbol] CH [symbol] | CH [symbol] | CH [symbol] | Bu | CH [symbol] | (8b-2-22c-b) |
| A-6 | 0 | CH [symbol] (CH [symbol] ) [symbol] CH [symbol] | CH [symbol] | CH [symbol] | Bnz | CH [symbol] | (8b-2-23c-b) |
| A-6 | 0 | CH [symbol] (CH [symbol] ) [symbol] CH [symbol] | CH [symbol] | Et | Et | CH [symbol] | (8b-2-24c-b) |
| A-6 | 0 | CH [symbol] (CH [symbol] ) [symbol] CH [symbol] | CH [symbol] | CH [symbol] CH [symbol] OCH [symbol] | CH [symbol] CH [symbol] OCH [symbol] | CH [symbol] | (8b-2-25c-b) |
| A-6 | 0 | CH [symbol] (CH [symbol] ) [symbol] CH [symbol] | CH [symbol] | Pr | Bu | CH [symbol] | (8b-2-26c-b) |
| A-6 | 0 | CH [symbol] (CH [symbol] ) [symbol] CH [symbol] | CH [symbol] | CH [symbol] | Et | Et | (8b-2-27c-b) |

[symbol]

indicates data missing or illegible when filed

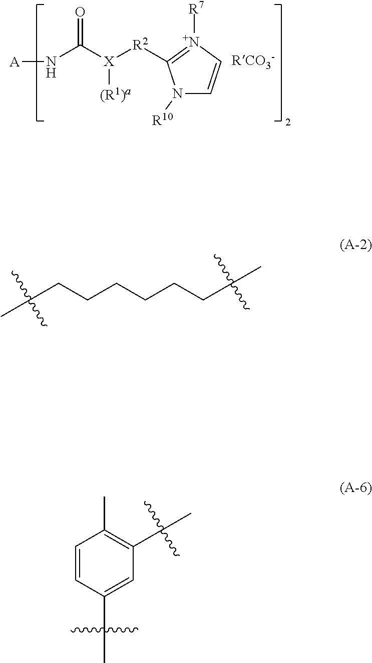

A, X, a, R¹, R², R⁷, R¹⁰, and R′ are as defined above. Preferably, A represents a group represented by formula (A-2) or a group represented by formula (A-6), X(R¹)^{a} represents NH, N(CH₃), or O, R² represents CH₂, CH₂CH₂, or CH₂(CH₂)₄CH₂, R⁷ represents CH₃ or Et, R¹⁰ represents CH₃, Et, ⁱPr, Bu, Bnz, or CH₂CH₂OCH₃, and R′ represents H, CH₃, or Et.

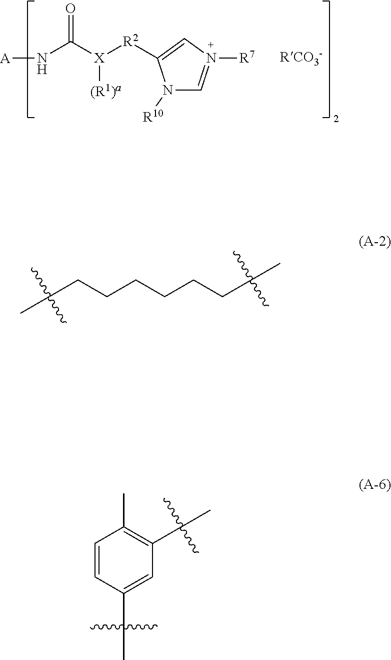

A, X, a, R¹, R², R⁷, R¹⁰, and R⁹ are as defined above. Preferably, A represents a group represented by formula (A-2) or a group represented by formula (A-6), X(R¹)^{a} represents NH, N(CH₃), or O, R² represents CH₂, CH₂CH₂, or CH₂(CH₂)₄CH₂, R⁷ represents CH₃ or Et, R¹⁰ represents CH₃, Et, ⁱPr, Bu, Bnz, or CH₂CH₂OCH₃, and R′ represents H, CH₃, or Et.

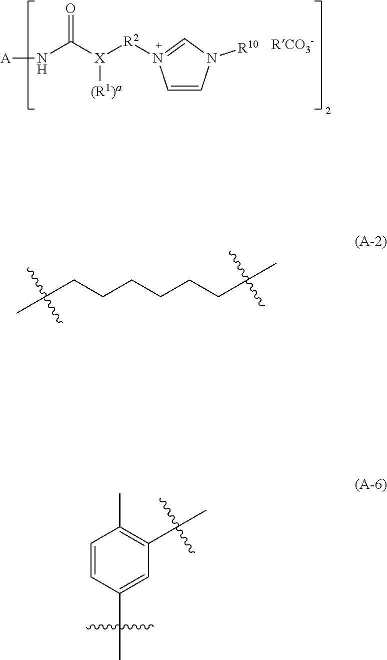

A, X, a, R¹, R², R¹⁰, and R′ are as defined above. Preferably, A represents a group represented by formula (A-2) or a group represented by formula (A-6), X(R¹)^{a} represents NH, N(CH₃), or O, R² represents CH₂, CH₂CH₂, or CH₂(CH₂)₄CH₂, R¹⁰ represents CH₃, Et, ⁱPr, Bu, Bnz, or CH₂CH₂OCH₃, and R′ represents H, CH₃, or Et.

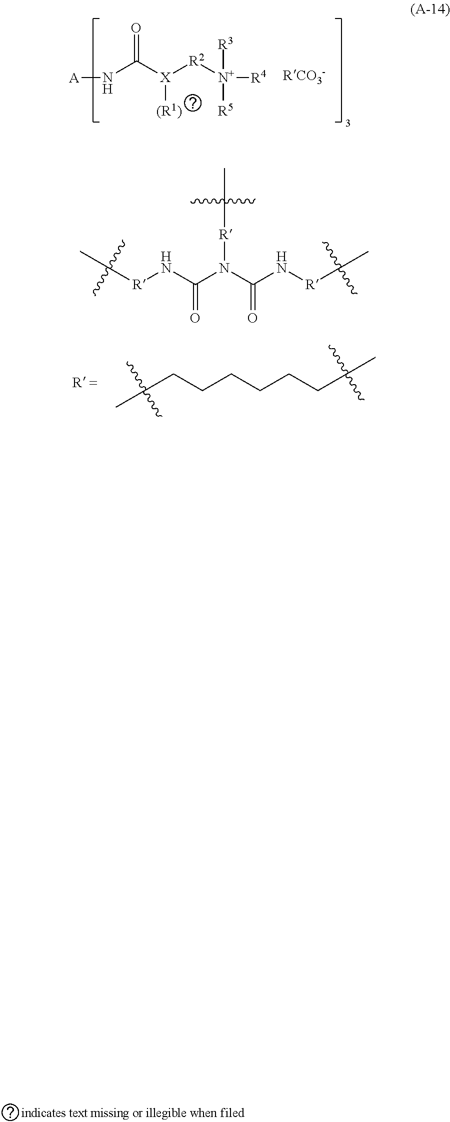

| A | X(R [symbol] ) [symbol] R [symbol] | R [symbol] | R [symbol] | R [symbol] | R [symbol] | R [symbol] | |
|---|---|---|---|---|---|---|---|
| A-14 | NH | CH [symbol] | CH [symbol] | CH [symbol] | CH [symbol] | H | (8a-2-1a-14) |
| A-14 | NH | CH [symbol] | CH [symbol] | CH [symbol] | Et | H | (8a-2-2a-14) |
| A-14 | NH | CH [symbol] | CH [symbol] | CH [symbol] | ⁱPr | H | (8a-2-3a-14) |
| A-14 | NH | CH [symbol] | CH [symbol] | CH [symbol] | Bu | H | (8a-2-4a-14) |
| A-14 | NH | CH [symbol] | CH [symbol] | CH [symbol] | Bnz | H | (8a-2-5a-14) |
| A-14 | NH | CH [symbol] | CH [symbol] | Et | Et | H | (8a-2-6a-14) |
| A-14 | NH | CH [symbol] | CH [symbol] | CH [symbol] CH [symbol] OCH [symbol] | CH [symbol] CH [symbol] OCH [symbol] | H | (8a-2-7a-14) |
| A-14 | NH | CH [symbol] | CH [symbol] | Pr | Bu | H | (8a-2-8a-14) |
| A-14 | NH | CH [symbol] | Et | Et | Et | H | (8a-2-9a-14) |
| A-14 | NH | CH [symbol] | ⁱPr | ⁱPr | ⁱPr | H | (8a-2-10a-14) |
| A-14 | NH | CH [symbol] | Bu | Bu | Bu | H | (8a-2-11a-14) |
| A-14 | NH | CH [symbol] CH [symbol] CH [symbol] | CH [symbol] | CH [symbol] | CH [symbol] | H | (8a-2-12a-14) |
| A-14 | NH | CH [symbol] CH [symbol] CH [symbol] | CH [symbol] | CH [symbol] | Et | H | (8a-2-13a-14) |
| A-14 | NH | CH [symbol] CH [symbol] CH [symbol] | CH [symbol] | CH [symbol] | ⁱPr | H | (8a-2-14a-14) |
| A-14 | NH | CH [symbol] CH [symbol] CH [symbol] | CH [symbol] | CH [symbol] | Bu | H | (8a-2-15a-14) |
| A-14 | NH | CH [symbol] CH [symbol] CH [symbol] | CH [symbol] | CH [symbol] | Bnz | H | (8a-2-16a-14) |
| A-14 | NH | CH [symbol] CH [symbol] CH [symbol] | CH [symbol] | Et | Et | H | (8a-2-17a-14) |
| A-14 | NH | CH [symbol] CH [symbol] CH [symbol] | CH [symbol] | CH [symbol] CH [symbol] OCH [symbol] | CH [symbol] CH [symbol] OCH [symbol] | H | (8a-2-18a-14) |
| A-14 | NH | CH [symbol] CH [symbol] CH [symbol] | CH [symbol] | Pr | Bu | H | (8a-2-19a-14) |
| A-14 | NH | CH [symbol] CH [symbol] CH [symbol] | Et | Et | Et | H | (8a-2-20a-14) |
| A-14 | NH | CH [symbol] CH [symbol] CH [symbol] | ⁱPr | ⁱPr | ⁱPr | H | (8a-2-21a-14) |
| A-14 | NH | CH [symbol] CH [symbol] CH [symbol] | Bu | Bu | Bu | H | (8a-2-22a-14) |
| A-14 | NH | CH [symbol] (CH [symbol] ) [symbol] CH [symbol] | CH [symbol] | CH [symbol] | CH [symbol] | H | (8a-2-23a-14) |
| A-14 | NH | CH [symbol] (CH [symbol] ) [symbol] CH [symbol] | CH [symbol] | CH [symbol] | Et | H | (8a-2-24a-14) |
| A-14 | NH | CH [symbol] (CH [symbol] ) [symbol] CH [symbol] | CH [symbol] | CH [symbol] | ⁱPr | H | (8a-2-25a-14) |
| A-14 | NH | CH [symbol] (CH [symbol] ) [symbol] CH [symbol] | CH [symbol] | CH [symbol] | Bu | H | (8a-2-26a-14) |
| A-14 | NH | CH [symbol] (CH [symbol] ) [symbol] CH [symbol] | CH [symbol] | CH [symbol] | Bnz | H | (8a-2-27a-14) |
| A-14 | NH | CH [symbol] (CH [symbol] ) [symbol] CH [symbol] | CH [symbol] | Et | Et | H | (8a-2-28a-14) |
| A-14 | NH | CH [symbol] (CH [symbol] ) [symbol] CH [symbol] | CH [symbol] | CH [symbol] CH [symbol] OCH [symbol] | CH [symbol] CH [symbol] OCH [symbol] | H | (8a-2-29a-14) |
| A-14 | NH | CH [symbol] (CH [symbol] ) [symbol] CH [symbol] | CH [symbol] | Pr | Bu | H | (8a-2-30a-14) |

[symbol]

indicates data missing or illegible when filed

| | | | | | | | |
|---|---|---|---|---|---|---|---|
| A-14 | NH | CH₂(CH₂)₄CH₂ | Et | Et | Et | H | (8a-2-31a-14) |
| A-14 | NH | CH₂(CH₂)₄CH₂ | ⁱPr | ⁱPr | ⁱPr | H | (8a-2-32a-14) |
| A-14 | NH | CH₂(CH₂)₄CH₂ | Bu | Bu | Bu | H | (8a-2-33a-14) |

| A | X(R [symbol] ) [symbol] | R [symbol] | R [symbol] | R [symbol] | R [symbol] | R [symbol] | |
|---|---|---|---|---|---|---|---|
| A-14 | N(CH [symbol] ) | CH [symbol] | CH [symbol] | CH [symbol] | CH [symbol] | H | (8a-2-1b-14) |
| A-14 | N(CH [symbol] ) | CH [symbol] | CH [symbol] | CH [symbol] | Et | H | (8a-2-2b-14) |
| A-14 | N(CH [symbol] ) | CH [symbol] | CH [symbol] | CH [symbol] | ⁱPr | H | (8a-2-3b-14) |
| A-14 | N(CH [symbol] ) | CH [symbol] | CH [symbol] | CH [symbol] | Bu | H | (8a-2-4b-14) |
| A-14 | N(CH [symbol] ) | CH [symbol] | CH [symbol] | CH [symbol] | Bnz | H | (8a-2-5b-14) |
| A-14 | N(CH [symbol] ) | CH [symbol] | CH [symbol] | Et | Et | H | (8a-2-6b-14) |
| A-14 | N(CH [symbol] ) | CH [symbol] | CH [symbol] | CH [symbol] CH [symbol] OCH [symbol] | CH [symbol] CH [symbol] OCH [symbol] | H | (8a-2-7b-14) |
| A-14 | N(CH [symbol] ) | CH [symbol] | CH [symbol] | Pr | Bu | H | (8a-2-8b-14) |
| A-14 | N(CH [symbol] ) | CH [symbol] | Et | Et | Et | H | (8a-2-9b-14) |
| A-14 | N(CH [symbol] ) | CH [symbol] | ⁱPr | ⁱPr | ⁱPr | H | (8a-2-10b-14) |
| A-14 | N(CH [symbol] ) | CH [symbol] | Bu | Bu | Bu | H | (8a-2-11b-14) |
| A-14 | N(CH [symbol] ) | CH [symbol] CH [symbol] CH [symbol] | CH [symbol] | CH [symbol] | CH [symbol] | H | (8a-2-12b-14) |
| A-14 | N(CH [symbol] ) | CH [symbol] CH [symbol] CH [symbol] | CH [symbol] | CH [symbol] | Et | H | (8a-2-13b-14) |
| A-14 | N(CH [symbol] ) | CH [symbol] CH [symbol] CH [symbol] | CH [symbol] | CH [symbol] | ⁱPr | H | (8a-2-14b-14) |
| A-14 | N(CH [symbol] ) | CH [symbol] CH [symbol] CH [symbol] | CH [symbol] | CH [symbol] | Bu | H | (8a-2-15b-14) |
| A-14 | N(CH [symbol] ) | CH [symbol] CH [symbol] CH [symbol] | CH [symbol] | CH [symbol] | Bnz | H | (8a-2-16b-14) |
| A-14 | N(CH [symbol] ) | CH [symbol] CH [symbol] CH [symbol] | CH [symbol] | Et | Et | H | (8a-2-17b-14) |
| A-14 | N(CH [symbol] ) | CH [symbol] CH [symbol] CH [symbol] | CH [symbol] | CH [symbol] CH [symbol] OCH [symbol] | CH [symbol] CH [symbol] OCH [symbol] | H | (8a-2-18b-14) |
| A-14 | N(CH [symbol] ) | CH [symbol] CH [symbol] CH [symbol] | CH [symbol] | Pr | Bu | H | (8a-2-19b-14) |
| A-14 | N(CH [symbol] ) | CH [symbol] CH [symbol] CH [symbol] | Et | Et | Et | H | (8a-2-20b-14) |
| A-14 | N(CH [symbol] ) | CH [symbol] CH [symbol] CH [symbol] | ⁱPr | ⁱPr | ⁱPr | H | (8a-2-21b-14) |
| A-14 | N(CH [symbol] ) | CH [symbol] CH [symbol] CH [symbol] | Bu | Bu | Bu | H | (8a-2-22b-14) |
| A-14 | N(CH [symbol] ) | CH [symbol] (CH [symbol] ) [symbol] CH [symbol] | CH [symbol] | CH [symbol] | CH [symbol] | H | (8a-2-23b-14) |
| A-14 | N(CH [symbol] ) | CH [symbol] (CH [symbol] ) [symbol] CH [symbol] | CH [symbol] | CH [symbol] | Et | H | (8a-2-24b-14) |
| A-14 | N(CH [symbol] ) | CH [symbol] (CH [symbol] ) [symbol] CH [symbol] | CH [symbol] | CH [symbol] | ⁱPr | H | (8a-2-25b-14) |
| A-14 | N(CH [symbol] ) | CH [symbol] (CH [symbol] ) [symbol] CH [symbol] | CH [symbol] | CH [symbol] | Bu | H | (8a-2-26b-14) |
| A-14 | N(CH [symbol] ) | CH [symbol] (CH [symbol] ) [symbol] CH [symbol] | CH [symbol] | CH [symbol] | Bnz | H | (8a-2-27b-14) |
| A-14 | N(CH [symbol] ) | CH [symbol] (CH [symbol] ) [symbol] CH [symbol] | CH [symbol] | Et | Et | H | (8a-2-28b-14) |
| A-14 | N(CH [symbol] ) | CH [symbol] (CH [symbol] ) [symbol] CH [symbol] | CH [symbol] | CH [symbol] CH [symbol] OCH [symbol] | CH [symbol] CH [symbol] OCH [symbol] | H | (8a-2-29b-14) |
| A-14 | N(CH [symbol] ) | CH [symbol] (CH [symbol] ) [symbol] CH [symbol] | CH [symbol] | Pr | Bu | H | (8a-2-30b-14) |
| A-14 | N(CH [symbol] ) | CH [symbol] (CH [symbol] ) [symbol] CH [symbol] | Et | Et | Et | H | (8a-2-31b-14) |
| A-14 | N(CH [symbol] ) | CH [symbol] (CH [symbol] ) [symbol] CH [symbol] | ⁱPr | ⁱPr | ⁱPr | H | (8a-2-32b-14) |
| A-14 | N(CH [symbol] ) | CH [symbol] (CH [symbol] ) [symbol] CH [symbol] | Bu | Bu | Bu | H | (8a-2-33b-14) |

[symbol]

indicates data missing or illegible when filed

| A | X(R [symbol] ) [symbol] | R [symbol] | R [symbol] | R [symbol] | R [symbol] | R [symbol] | |
|---|---|---|---|---|---|---|---|
| A-14 | 0 | CH [symbol] | CH [symbol] | CH [symbol] | CH [symbol] | H | (8a-2-1c-14) |
| A-14 | 0 | CH [symbol] | CH [symbol] | CH [symbol] | Et | H | (8a-2-2c-14) |
| A-14 | 0 | CH [symbol] | CH [symbol] | CH [symbol] | ⁱPr | H | (8a-2-3c-14) |
| A-14 | 0 | CH [symbol] | CH [symbol] | CH [symbol] | Bu | H | (8a-2-4c-14) |
| A-14 | 0 | CH [symbol] | CH [symbol] | CH [symbol] | Bnz | H | (8a-2-5c-14) |
| A-14 | 0 | CH [symbol] | CH [symbol] | Et | Et | H | (8a-2-6c-14) |
| A-14 | 0 | CH [symbol] | CH [symbol] | CH [symbol] CH [symbol] OCH [symbol] | CH [symbol] CH [symbol] OCH [symbol] | H | (8a-2-7c-14) |
| A-14 | 0 | CH [symbol] | CH [symbol] | Pr | Bu | H | (8a-2-8c-14) |
| A-14 | 0 | CH [symbol] | Et | Et | Et | H | (8a-2-9c-14) |
| A-14 | 0 | CH [symbol] | ⁱPr | ⁱPr | ⁱPr | H | (8a-2-10c-14) |
| A-14 | 0 | CH [symbol] | Bu | Bu | Bu | H | (8a-2-11c-14) |
| A-14 | 0 | CH [symbol] CH [symbol] CH [symbol] | CH [symbol] | CH [symbol] | CH [symbol] | H | (8a-2-12c-14) |
| A-14 | 0 | CH [symbol] CH [symbol] CH [symbol] | CH [symbol] | CH [symbol] | Et | H | (8a-2-13c-14) |
| A-14 | 0 | CH [symbol] CH [symbol] CH [symbol] | CH [symbol] | CH [symbol] | ⁱPr | H | (8a-2-14c-14) |
| A-14 | 0 | CH [symbol] CH [symbol] CH [symbol] | CH [symbol] | CH [symbol] | Bu | H | (8a-2-15c-14) |
| A-14 | 0 | CH [symbol] CH [symbol] CH [symbol] | CH [symbol] | CH [symbol] | Bnz | H | (8a-2-16c-14) |
| A-14 | 0 | CH [symbol] CH [symbol] CH [symbol] | CH [symbol] | Et | Et | H | (8a-2-17c-14) |
| A-14 | 0 | CH [symbol] CH [symbol] CH [symbol] | CH [symbol] | CH [symbol] CH [symbol] OCH [symbol] | CH [symbol] CH [symbol] OCH [symbol] | H | (8a-2-18c-14) |
| A-14 | 0 | CH [symbol] CH [symbol] CH [symbol] | CH [symbol] | Pr | Bu | H | (8a-2-19c-14) |
| A-14 | 0 | CH [symbol] CH [symbol] CH [symbol] | Et | Et | Et | H | (8a-2-20c-14) |
| A-14 | 0 | CH [symbol] CH [symbol] CH [symbol] | ⁱPr | ⁱPr | ⁱPr | H | (8a-2-21c-14) |
| A-14 | 0 | CH [symbol] CH [symbol] CH [symbol] | Bu | Bu | Bu | H | (8a-2-22c-14) |
| A-14 | 0 | CH [symbol] (CH [symbol] ) [symbol] CH [symbol] | CH [symbol] | CH [symbol] | CH [symbol] | H | (8a-2-23c-14) |
| A-14 | 0 | CH [symbol] (CH [symbol] ) [symbol] CH [symbol] | CH [symbol] | CH [symbol] | Et | H | (8a-2-24c-14) |
| A-14 | 0 | CH [symbol] (CH [symbol] ) [symbol] CH [symbol] | CH [symbol] | CH [symbol] | ⁱPr | H | (8a-2-25c-14) |
| A-14 | 0 | CH [symbol] (CH [symbol] ) [symbol] CH [symbol] | CH [symbol] | CH [symbol] | Bu | H | (8a-2-26c-14) |
| A-14 | 0 | CH [symbol] (CH [symbol] ) [symbol] CH [symbol] | CH [symbol] | CH [symbol] | Bnz | H | (8a-2-27c-14) |
| A-14 | 0 | CH [symbol] (CH [symbol] ) [symbol] CH [symbol] | CH [symbol] | Et | Et | H | (8a-2-28c-14) |
| A-14 | 0 | CH [symbol] (CH [symbol] ) [symbol] CH [symbol] | CH [symbol] | CH [symbol] CH [symbol] OCH [symbol] | CH [symbol] CH [symbol] OCH [symbol] | H | (8a-2-29c-14) |
| A-14 | 0 | CH [symbol] (CH [symbol] ) [symbol] CH [symbol] | CH [symbol] | Pr | Bu | H | (8a-2-30c-14) |
| A-14 | 0 | CH [symbol] (CH [symbol] ) [symbol] CH [symbol] | Et | Et | Et | H | (8a-2-31c-14) |
| A-14 | 0 | CH [symbol] (CH [symbol] ) [symbol] CH [symbol] | ⁱPr | ⁱPr | ⁱPr | H | (8a-2-32c-14) |
| A-14 | 0 | CH [symbol] (CH [symbol] ) [symbol] CH [symbol] | Bu | Bu | Bu | H | (8a-2-33c-14) |

[symbol]

indicates data missing or illegible when filed

| A | X(R [symbol] ) [symbol] | R [symbol] | R [symbol] | R [symbol] | R [symbol] | R [symbol] | |
|---|---|---|---|---|---|---|---|
| A-14 | NH | CH [symbol] | CH [symbol] | CH [symbol] | CH [symbol] | CH [symbol] | (8b-2-1a-14) |
| A-14 | NH | CH [symbol] | CH [symbol] | CH [symbol] | Et | CH [symbol] | (8b-2-2a-14) |
| A-14 | NH | CH [symbol] | CH [symbol] | CH [symbol] | ⁱPr | CH [symbol] | (8b-2-3a-14) |
| A-14 | NH | CH [symbol] | CH [symbol] | CH [symbol] | Bu | CH [symbol] | (8b-2-4a-14) |
| A-14 | NH | CH [symbol] | CH [symbol] | CH [symbol] | Bnz | CH [symbol] | (8b-2-5a-14) |
| A-14 | NH | CH [symbol] | CH [symbol] | Et | Et | CH [symbol] | (8b-2-6a-14) |
| A-14 | NH | CH [symbol] | CH [symbol] | CH [symbol] CH [symbol] OCH [symbol] | CH [symbol] CH [symbol] OCH [symbol] | CH [symbol] | (8b-2-7a-14) |
| A-14 | NH | CH [symbol] | CH [symbol] | Pr | Bu | CH [symbol] | (8b-2-8a-14) |
| A-14 | NH | CH [symbol] | CH [symbol] | CH [symbol] | Et | Et | (8b-2-9a-14) |
| A-14 | NH | CH [symbol] CH [symbol] CH [symbol] | CH [symbol] | CH [symbol] | CH [symbol] | CH [symbol] | (8b-2-10a-14) |
| A-14 | NH | CH [symbol] CH [symbol] CH [symbol] | CH [symbol] | CH [symbol] | Et | CH [symbol] | (8b-2-11a-14) |
| A-14 | NH | CH [symbol] CH [symbol] CH [symbol] | CH [symbol] | CH [symbol] | ⁱPr | CH [symbol] | (8b-2-12a-14) |
| A-14 | NH | CH [symbol] CH [symbol] CH [symbol] | CH [symbol] | CH [symbol] | Bu | CH [symbol] | (8b-2-13a-14) |
| A-14 | NH | CH [symbol] CH [symbol] CH [symbol] | CH [symbol] | CH [symbol] | Bnz | CH [symbol] | (8b-2-14a-14) |
| A-14 | NH | CH [symbol] CH [symbol] CH [symbol] | CH [symbol] | Et | Et | CH [symbol] | (8b-2-15a-14) |
| A-14 | NH | CH [symbol] CH [symbol] CH [symbol] | CH [symbol] | CH [symbol] CH [symbol] OCH [symbol] | CH [symbol] CH [symbol] OCH [symbol] | CH [symbol] | (8b-2-16a-14) |
| A-14 | NH | CH [symbol] CH [symbol] CH [symbol] | CH [symbol] | Pr | Bu | CH [symbol] | (8b-2-17a-14) |
| A-14 | NH | CH [symbol] CH [symbol] CH [symbol] | CH [symbol] | CH [symbol] | Et | Et | (8b-2-18a-14) |
| A-14 | NH | CH [symbol] (CH [symbol] ) [symbol] CH [symbol] | CH [symbol] | CH [symbol] | CH [symbol] | CH [symbol] | (8b-2-19a-14) |
| A-14 | NH | CH [symbol] (CH [symbol] ) [symbol] CH [symbol] | CH [symbol] | CH [symbol] | Et | CH [symbol] | (8b-2-20a-14) |
| A-14 | NH | CH [symbol] (CH [symbol] ) [symbol] CH [symbol] | CH [symbol] | CH [symbol] | ⁱPr | CH [symbol] | (8b-2-21a-14) |
| A-14 | NH | CH [symbol] (CH [symbol] ) [symbol] CH [symbol] | CH [symbol] | CH [symbol] | Bu | CH [symbol] | (8b-2-22a-14) |
| A-14 | NH | CH [symbol] (CH [symbol] ) [symbol] CH [symbol] | CH [symbol] | CH [symbol] | Bnz | CH [symbol] | (8b-2-23a-14) |
| A-14 | NH | CH [symbol] (CH [symbol] ) [symbol] CH [symbol] | CH [symbol] | Et | Et | CH [symbol] | (8b-2-24a-14) |
| A-14 | NH | CH [symbol] (CH [symbol] ) [symbol] CH [symbol] | CH [symbol] | CH [symbol] CH [symbol] OCH [symbol] | CH [symbol] CH [symbol] OCH [symbol] | CH [symbol] | (8b-2-25a-14) |
| A-14 | NH | CH [symbol] (CH [symbol] ) [symbol] CH [symbol] | CH [symbol] | Pr | Bu | CH [symbol] | (8b-2-26a-14) |
| A-14 | NH | CH [symbol] (CH [symbol] ) [symbol] CH [symbol] | CH [symbol] | CH [symbol] | Et | Et | (8b-2-27a-14) |
| A-14 | N(CH [symbol] ) | CH [symbol] | CH [symbol] | CH [symbol] | CH [symbol] | CH [symbol] | (8b-2-1b-14) |
| A-14 | N(CH [symbol] ) | CH [symbol] | CH [symbol] | CH [symbol] | Et | CH [symbol] | (8b-2-2b-14) |
| A-14 | N(CH [symbol] ) | CH [symbol] | CH [symbol] | CH [symbol] | ⁱPr | CH [symbol] | (8b-2-3b-14) |
| A-14 | N(CH [symbol] ) | CH [symbol] | CH [symbol] | CH [symbol] | Bu | CH [symbol] | (8b-2-4b-14) |
| A-14 | N(CH [symbol] ) | CH [symbol] | CH [symbol] | CH [symbol] | Bnz | CH [symbol] | (8b-2-5b-14) |
| A-14 | N(CH [symbol] ) | CH [symbol] | CH [symbol] | Et | Et | CH [symbol] | (8b-2-6b-14) |
| A-14 | N(CH [symbol] ) | CH [symbol] | CH [symbol] | CH [symbol] CH [symbol] OCH [symbol] | CH [symbol] CH [symbol] OCH [symbol] | CH [symbol] | (8b-2-7b-14) |
| A-14 | N(CH [symbol] ) | CH [symbol] | CH [symbol] | Pr | Bu | CH [symbol] | (8b-2-8b-14) |
| A-14 | N(CH [symbol] ) | CH [symbol] | CH [symbol] | CH [symbol] | Et | Et | (8b-2-9b-14) |

[symbol]

indicates data missing or illegible when filed

| A | X(R [symbol] ) [symbol] | R [symbol] | R [symbol] | R [symbol] | R [symbol] | R [symbol] | |
|---|---|---|---|---|---|---|---|
| A-14 | N(CH [symbol] ) | CH [symbol] CH [symbol] CH [symbol] | CH [symbol] | CH [symbol] | CH [symbol] | CH [symbol] | (8b-2-10b-14) |
| A-14 | N(CH [symbol] ) | CH [symbol] CH [symbol] CH [symbol] | CH [symbol] | CH [symbol] | Et | CH [symbol] | (8b-2-11b-14) |
| A-14 | N(CH [symbol] ) | CH [symbol] CH [symbol] CH [symbol] | CH [symbol] | CH [symbol] | ⁱPr | CH [symbol] | (8b-2-12b-14) |
| A-14 | N(CH [symbol] ) | CH [symbol] CH [symbol] CH [symbol] | CH [symbol] | CH [symbol] | Bu | CH [symbol] | (8b-2-13b-14) |
| A-14 | N(CH [symbol] ) | CH [symbol] CH [symbol] CH [symbol] | CH [symbol] | CH [symbol] | Bnz | CH [symbol] | (8b-2-14b-14) |
| A-14 | N(CH [symbol] ) | CH [symbol] CH [symbol] CH [symbol] | CH [symbol] | Et | Et | CH [symbol] | (8b-2-15b-14) |
| A-14 | N(CH [symbol] ) | CH [symbol] CH [symbol] CH [symbol] | CH [symbol] | CH [symbol] CH [symbol] OCH [symbol] | CH [symbol] CH [symbol] OCH [symbol] | CH [symbol] | (8b-2-16b-14) |
| A-14 | N(CH [symbol] ) | CH [symbol] CH [symbol] CH [symbol] | CH [symbol] | Pr | Bu | CH [symbol] | (8b-2-17b-14) |
| A-14 | N(CH [symbol] ) | CH [symbol] CH [symbol] CH [symbol] | CH [symbol] | CH [symbol] | Et | Et | (8b-2-18b-14) |
| A-14 | N(CH [symbol] ) | CH [symbol] (CH [symbol] ) [symbol] CH [symbol] | CH [symbol] | CH [symbol] | CH [symbol] | CH [symbol] | (8b-2-19b-14) |
| A-14 | N(CH [symbol] ) | CH [symbol] (CH [symbol] ) [symbol] CH [symbol] | CH [symbol] | CH [symbol] | Et | CH [symbol] | (8b-2-20b-14) |
| A-14 | N(CH [symbol] ) | CH [symbol] (CH [symbol] ) [symbol] CH [symbol] | CH [symbol] | CH [symbol] | ⁱPr | CH [symbol] | (8b-2-21b-14) |
| A-14 | N(CH [symbol] ) | CH [symbol] (CH [symbol] ) [symbol] CH [symbol] | CH [symbol] | CH [symbol] | Bu | CH [symbol] | (8b-2-22b-14) |
| A-14 | N(CH [symbol] ) | CH [symbol] (CH [symbol] ) [symbol] CH [symbol] | CH [symbol] | CH [symbol] | Bnz | CH [symbol] | (8b-2-23b-14) |
| A-14 | N(CH [symbol] ) | CH [symbol] (CH [symbol] ) [symbol] CH [symbol] | CH [symbol] | Et | Et | CH [symbol] | (8b-2-24b-14) |
| A-14 | N(CH [symbol] ) | CH [symbol] (CH [symbol] ) [symbol] CH [symbol] | CH [symbol] | CH [symbol] CH [symbol] OCH [symbol] | CH [symbol] CH [symbol] OCH [symbol] | CH [symbol] | (8b-2-25b-14) |
| A-14 | N(CH [symbol] ) | CH [symbol] (CH [symbol] ) [symbol] CH [symbol] | CH [symbol] | Pr | Bu | CH [symbol] | (8b-2-26b-14) |
| A-14 | N(CH [symbol] ) | CH [symbol] (CH [symbol] ) [symbol] CH [symbol] | CH [symbol] | CH [symbol] | Et | Et | (8b-2-27b-14) |
| A-14 | 0 | CH [symbol] | CH [symbol] | CH [symbol] | CH [symbol] | CH [symbol] | (8b-2-1c-14) |
| A-14 | 0 | CH [symbol] | CH [symbol] | CH [symbol] | Et | CH [symbol] | (8b-2-2c-14) |
| A-14 | 0 | CH [symbol] | CH [symbol] | CH [symbol] | ⁱPr | CH [symbol] | (8b-2-3c-14) |
| A-14 | 0 | CH [symbol] | CH [symbol] | CH [symbol] | Bu | CH [symbol] | (8b-2-4c-14) |
| A-14 | 0 | CH [symbol] | CH [symbol] | CH [symbol] | Bnz | CH [symbol] | (8b-2-5c-14) |
| A-14 | 0 | CH [symbol] | CH [symbol] | Et | Et | CH [symbol] | (8b-2-6c-14) |
| A-14 | 0 | CH [symbol] | CH [symbol] | CH [symbol] CH [symbol] OCH [symbol] | CH [symbol] CH [symbol] OCH [symbol] | CH [symbol] | (8b-2-7c-14) |
| A-14 | 0 | CH [symbol] | CH [symbol] | Pr | Bu | CH [symbol] | (8b-2-8c-14) |
| A-14 | 0 | CH [symbol] | CH [symbol] | CH [symbol] | Et | Et | (8b-2-9c-14) |
| A-14 | 0 | CH [symbol] CH [symbol] CH [symbol] | CH [symbol] | CH [symbol] | CH [symbol] | CH [symbol] | (8b-2-10c-14) |
| A-14 | 0 | CH [symbol] CH [symbol] CH [symbol] | CH [symbol] | CH [symbol] | Et | CH [symbol] | (8b-2-11c-14) |
| A-14 | 0 | CH [symbol] CH [symbol] CH [symbol] | CH [symbol] | CH [symbol] | ⁱPr | CH [symbol] | (8b-2-12c-14) |
| A-14 | 0 | CH [symbol] CH [symbol] CH [symbol] | CH [symbol] | CH [symbol] | Bu | CH [symbol] | (8b-2-13c-14) |
| A-14 | 0 | CH [symbol] CH [symbol] CH [symbol] | CH [symbol] | CH [symbol] | Bnz | CH [symbol] | (8b-2-14c-14) |
| A-14 | 0 | CH [symbol] CH [symbol] CH [symbol] | CH [symbol] | Et | Et | CH [symbol] | (8b-2-15c-14) |
| A-14 | 0 | CH [symbol] CH [symbol] CH [symbol] | CH [symbol] | CH [symbol] CH [symbol] OCH [symbol] | CH [symbol] CH [symbol] OCH [symbol] | CH [symbol] | (8b-2-16c-14) |
| A-14 | 0 | CH [symbol] CH [symbol] CH [symbol] | CH [symbol] | Pr | Bu | CH [symbol] | (8b-2-17c-14) |
| A-14 | 0 | CH [symbol] CH [symbol] CH [symbol] | CH [symbol] | CH [symbol] | Et | Et | (8b-2-18c-14) |
| A-14 | 0 | CH [symbol] (CH [symbol] ) [symbol] CH [symbol] | CH [symbol] | CH [symbol] | CH [symbol] | CH [symbol] | (8b-2-19c-14) |

[symbol]

indicates data missing or illegible when filed

| | | | | | | | |
|---|---|---|---|---|---|---|---|
| A-14 | 0 | CH₂(CH₂)₄CH₂ | CH₃ | CH₃ | Et | CH₃ | (8b-2-20c-14) |
| A-14 | 0 | CH₂(CH₂)₄CH₂ | CH₃ | CH₃ | ⁱPr | CH₃ | (8b-2-21c-14) |
| A-14 | 0 | CH₂(CH₂)₄CH₂ | CH₃ | CH₃ | Bu | CH₃ | (8b-2-22c-14) |
| A-14 | 0 | CH₂(CH₂)₄CH₂ | CH₃ | CH₃ | Bnz | CH₃ | (8b-2-23c-14) |
| A-14 | 0 | CH₂(CH₂)₄CH₂ | CH₃ | Et | Et | CH₃ | (8b-2-24c-14) |
| A-14 | 0 | CH₂(CH₂)₄CH₂ | CH₃ | CH₂CH₂OCH₃ | CH₂CH₂OCH₃ | CH₃ | (8b-2-25c-14) |
| A-14 | 0 | CH₂(CH₂)₄CH₂ | CH₃ | Pr | Bu | CH₃ | (8b-2-26c-14) |
| A-14 | 0 | CH₂(CH₂)₄CH₂ | CH₃ | CH₃ | Et | Et | (8b-2-27c-14) |

Formula (8) is preferably any one of (8a-2-1a-1) to (8a-2-33a-1), (8b-2-1a-1) to (8b-2-27a-1), (8a-2-1b-1) to (8a-2-33b-1), (8b-2-1b-1) to (8b-2-27b-1), (8a-2-1c-1) to (8a-2-33c-1), (8b-2-1c-1) to (8b-2-27c-1), (8a-2-1a-2) to (8a-2-33a-2), (8b-2-1a-2) to (8b-2-27a-2), (8a-2-1b-2) to (8a-2-33b-2), (8b-2-1b-2) to (8b-2-27b-2), (8a-2-1c-2) to (8a-2-33c-2), (8b-2-1c-2) to (8b-2-27c-2), (8a-2-1a-6) to (8a-2-33a-6), (8b-2-1a-6) to (8b-2-27a-6), (8a-2-1b-6) to (8a-2-33b-6), (8b-2-1b-6) to (8b-2-27b-6), (8a-2-1c-6) to (8a-2-33c-6), (8b-2-1c-6) to (8b-2-27c-6), (8a-2-1a-14) to (8a-2-33a-14), (8b-2-1a-14) to (8b-2-27a-14), (8a-2-1b-14) to (8a-2-33b-14), (8b-2-1b-14) to (8b-2-27b-14), (8a-2-1c-14) to (8a-2-33c-14), and (8b-2-1c-14) to (8b-2-27c-14), and particularly preferably (8a-2-12a-1), (8b-2-10a-1), (8a-2-12a-2), or (8b-2-10a-2).

For the reaction, the ester carbonate compound represented by formula (7) is usually used in an amount of 0.5 to 10 mol, and preferably 0.8 to 3 mol, per 1/n mol of the compound represented by formula (6).

The reaction temperature is usually 10° C. to 120° C. or the boiling temperature of the solvent.

The reaction time is usually 1 to 20 hours, and preferably 1 to 10 hours.

The ester carbonate compound (7a) or (7b) may be used in an excessive amount for use as a solvent; however, it is also possible to use other solvents. Examples of solvents include tetrahydrofuran, ethyl acetate, acetonitrile, toluene, acetone, and the like. When other solvents are used, the amount for use is usually 100 parts by mass or less, and preferably 0.1 to 50 parts by mass, per part by mass of the compound represented by formula (6).

In step 2, the reaction may be performed, if necessary, in an inert gas atmosphere, such as nitrogen, argon, or helium, which do not affect the reaction.

The obtained compound (8b) can be purified according to common methods, such as concentration and recrystallization, or can be used as a raw material for Production Method 1 or step 3 described below without purification.

After the completion of step 2, the obtained compound (8b) can be used as a raw material for Production Method 1. Alternatively, the compound (8b) may be reacted with water to produce a compound in which R′ represents a hydrogen atom in formula (8) (hereinafter referred to as “the compound (8a)”) (hereinafter referred to as “step 3”) for use as a raw material for Production Method 1.

In Production Method 1, the reaction proceeds more easily with the compound (8a) than with the compound (8b). Therefore, it is preferable to carry out step 3 and use the compound (8a) in Production Method 1.

Step 3 is described below.

The amount of water for use is usually 1 to 500 mol, and preferably 3 to 250 mol, per mole of the compound (8b). In step 3, when the reaction liquid obtained in step 2 is used as is as the compound (8b), the ester carbonate compound (7) may remain in the reaction liquid. In this case, water is preferably used in an excessive amount in step 3. Water can also be used as a solvent. When water is used as a solvent, in addition to the amount mentioned above, water can be used in an amount of usually 100 parts by mass or less, and preferably 0.1 to 50 parts by mass, per part by mass of the compound (8b).

The reaction temperature for the reaction of the compound (8b) with water is usually 10° C. or more, preferably 10° C. to 100° C., and more preferably 10° C. to 80° C.

The reaction time is usually 0.1 to 10 hours, and preferably 0.1 to 5 hours.

Solvents may or may not be used. Water can be used as a solvent. When a solvent other than water is used, examples of the solvent include tetrahydrofuran, ethyl acetate, acetonitrile, toluene, acetone, methanol, and the like. When a solvent other than water is used, the amount for use is usually 100 parts by mass or less, and preferably 0.1 to 50 parts by mass, per part by mass of the compound (8b). Two or more different solvents may be used in combination, if necessary.

In step 3, the reaction may be performed, if necessary, in an inert gas atmosphere, such as nitrogen, argon, or helium, which do not affect the reaction.

The obtained compound (8a) can be purified according to common methods, such as concentration and recrystallization, or can be used as a raw material for Production Method 1 without purification.

When a compound represented by the following formula (9) is used as a raw material, the compound represented by formula (8) can also be produced by the following method instead of step 2.

A compound represented by the following formula (9) is reacted with an isocyanate compound represented by formula (5) to produce a compound represented by formula (8) (Method B). Formula (9):

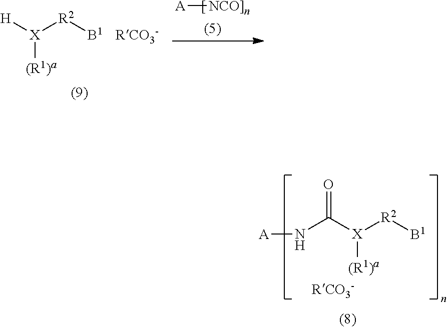

(In the formulas, A, R′, n, a, R¹, R², and B¹ are as defined above.)

Specific examples of the compound represented by formula (9) are shown below. However, the present invention is not limited thereto. In the following specific examples below, Et represents an ethyl group, Pr represents a propyl group, ⁱPr represents an isopropyl group, Bu represents a butyl group, and Bnz represents a benzyl group.

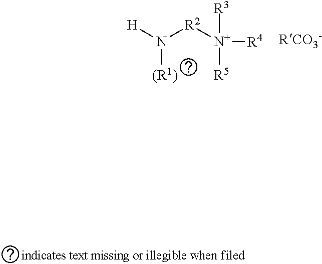

| X(R [symbol] ) [symbol] | R [symbol] | R [symbol] | R [symbol] | R [symbol] | R [symbol] | |
|---|---|---|---|---|---|---|
| NH | CH [symbol] | CH [symbol] | CH [symbol] | CH [symbol] | H | (9a-2-1a) |
| NH | CH [symbol] | CH [symbol] | CH [symbol] | Et | H | (9a-2-2a) |
| NH | CH [symbol] | CH [symbol] | CH [symbol] | ⁱPr | H | (9a-2-3a) |
| NH | CH [symbol] | CH [symbol] | CH [symbol] | Bu | H | (9a-2-4a) |
| NH | CH [symbol] | CH [symbol] | CH [symbol] | Bnz | H | (9a-2-5a) |
| NH | CH [symbol] | CH [symbol] | Et | Et | H | (9a-2-6a) |
| NH | CH [symbol] | CH [symbol] | CH [symbol] CH [symbol] OCH [symbol] | CH [symbol] CH [symbol] OCH [symbol] | H | (9a-2-7a) |
| NH | CH [symbol] | CH [symbol] | Pr | Bu | H | (9a-2-8a) |
| NH | CH [symbol] | Et | Et | Et | H | (9a-2-9a) |
| NH | CH [symbol] | ⁱPr | ⁱPr | ⁱPr | H | (9a-2-10a) |
| NH | CH [symbol] | Bu | Bu | Bu | H | (9a-2-11a) |
| NH | CH [symbol] CH [symbol] CH [symbol] | CH [symbol] | CH [symbol] | CH [symbol] | H | (9a-2-12a) |
| NH | CH [symbol] CH [symbol] CH [symbol] | CH [symbol] | CH [symbol] | Et | H | (9a-2-13a) |
| NH | CH [symbol] CH [symbol] CH [symbol] | CH [symbol] | CH [symbol] | ⁱPr | H | (9a-2-14a) |
| NH | CH [symbol] CH [symbol] CH [symbol] | CH [symbol] | CH [symbol] | Bu | H | (9a-2-15a) |
| NH | CH [symbol] CH [symbol] CH [symbol] | CH [symbol] | CH [symbol] | Bnz | H | (9a-2-16a) |
| NH | CH [symbol] CH [symbol] CH [symbol] | CH [symbol] | Et | Et | H | (9a-2-17a) |
| NH | CH [symbol] CH [symbol] CH [symbol] | CH [symbol] | CH [symbol] CH [symbol] OCH [symbol] | CH [symbol] CH [symbol] OCH [symbol] | H | (9a-2-18a) |
| NH | CH [symbol] CH [symbol] CH [symbol] | CH [symbol] | Pr | Bu | H | (9a-2-19a) |
| NH | CH [symbol] CH [symbol] CH [symbol] | Et | Et | Et | H | (9a-2-20a) |
| NH | CH [symbol] CH [symbol] CH [symbol] | ⁱPr | ⁱPr | ⁱPr | H | (9a-2-21a) |
| NH | CH [symbol] CH [symbol] CH [symbol] | Bu | Bu | Bu | H | (9a-2-22a) |
| NH | CH [symbol] (CH [symbol] ) [symbol] CH [symbol] | CH [symbol] | CH [symbol] | CH [symbol] | H | (9a-2-23a) |
| NH | CH [symbol] (CH [symbol] ) [symbol] CH [symbol] | CH [symbol] | CH [symbol] | Et | H | (9a-2-24a) |
| NH | CH [symbol] (CH [symbol] ) [symbol] CH [symbol] | CH [symbol] | CH [symbol] | ⁱPr | H | (9a-2-25a) |
| NH | CH [symbol] (CH [symbol] ) [symbol] CH [symbol] | CH [symbol] | CH [symbol] | Bu | H | (9a-2-26a) |
| NH | CH [symbol] (CH [symbol] ) [symbol] CH [symbol] | CH [symbol] | CH [symbol] | Bnz | H | (9a-2-27a) |
| NH | CH [symbol] (CH [symbol] ) [symbol] CH [symbol] | CH [symbol] | Et | Et | H | (9a-2-28a) |
| NH | CH [symbol] (CH [symbol] ) [symbol] CH [symbol] | CH [symbol] | CH [symbol] CH [symbol] OCH [symbol] | CH [symbol] CH [symbol] OCH [symbol] | H | (9a-2-29a) |
| NH | CH [symbol] (CH [symbol] ) [symbol] CH [symbol] | CH [symbol] | Pr | Bu | H | (9a-2-30a) |
| NH | CH [symbol] (CH [symbol] ) [symbol] CH [symbol] | Et | Et | Et | H | (9a-2-31a) |
| NH | CH [symbol] (CH [symbol] ) [symbol] CH [symbol] | ⁱPr | ⁱPr | ⁱPr | H | (9a-2-32a) |
| NH | CH [symbol] (CH [symbol] ) [symbol] CH [symbol] | Bu | Bu | Bu | H | (9a-2-33a) |
| NH | CH [symbol] | CH [symbol] | CH [symbol] | CH [symbol] | CH [symbol] | (9b-2-1a) |

[symbol]

indicates data missing or illegible when filed

| | | | | | | |
|---|---|---|---|---|---|---|
| NH | CH₂ | CH₃ | CH₃ | Et | CH₃ | (9b-2-2a) |
| NH | CH₂ | CH₃ | CH₃ | ⁱPr | CH₃ | (9b-2-3a) |
| NH | CH₂ | CH₃ | CH₃ | Bu | CH₃ | (9b-2-4a) |
| NH | CH₂ | CH₃ | CH₃ | Bnz | CH₃ | (9b-2-5a) |
| NH | CH₂ | CH₃ | Et | Et | CH₃ | (9b-2-6a) |
| NH | CH₂ | CH₃ | CH₂CH₂OCH₃ | CH₂CH₂OCH₃ | CH₃ | (9b-2-7a) |
| NH | CH₂ | CH₃ | Pr | Bu | CH₃ | (9b-2-8a) |
| NH | CH₂ | CH₃ | CH₃ | Et | Et | (9b-2-9a) |

| X(R¹) [symbol] | R² | R³ | R⁴ | R⁵ | R′ | |
|---|---|---|---|---|---|---|
| NH | CH2CH2CH2 | CH₃ | CH₃ | CH₃ | CH₃ | (9b-2-10a) |
| NH | CH2CH2CH2 | CH₃ | CH₃ | Et | CH₃ | (9b-2-11a) |
| NH | CH2CH2CH2 | CH₃ | CH₃ | ⁱPr | CH₃ | (9b-2-12a) |
| NH | CH2CH2CH2 | CH₃ | CH₃ | Bu | CH₃ | (9b-2-13a) |
| NH | CH2CH2CH2 | CH₃ | CH₃ | Bnz | CH₃ | (9b-2-14a) |
| NH | CH2CH2CH2 | CH₃ | Et | Et | CH₃ | (9b-2-15a) |
| NH | CH2CH2CH2 | CH₃ | CH₂CH₂OCH₃ | CH₂CH₂OCH₃ | CH₃ | (9b-2-16a) |
| NH | CH2CH2CH2 | CH₃ | Pr | Bu | CH₃ | (9b-2-17a) |
| NH | CH2CH2CH2 | CH₃ | CH₃ | Et | Et | (9b-2-18a) |
| NH | CH₂(CH₂)₄CH₂ | CH₃ | CH₃ | CH₃ | CH₃ | (9b-2-19a) |
| NH | CH₂(CH₂)₄CH₂ | CH₃ | CH₃ | Et | CH₃ | (9b-2-20a) |
| NH | CH₂(CH₂)₄CH₂ | CH₃ | CH₃ | ⁱPr | CH₃ | (9b-2-21a) |
| NH | CH₂(CH₂)₄CH₂ | CH₃ | CH₃ | Bu | CH₃ | (9b-2-22a) |
| NH | CH₂(CH₂)₄CH₂ | CH₃ | CH₃ | Bnz | CH₃ | (9b-2-23a) |
| NH | CH₂(CH₂)₄CH₂ | CH₃ | Et | Et | CH₃ | (9b-2-24a) |
| NH | CH₂(CH₂)₄CH₂ | CH₃ | CH₂CH₂OCH₃ | CH₂CH₂OCH₃ | CH₃ | (9b-2-25a) |
| NH | CH₂(CH₂)₄CH₂ | CH₃ | Pr | Bu | CH₃ | (9b-2-26a) |
| NH | CH₂(CH₂)₄CH₂ | CH₃ | CH₃ | Et | Et | (9b-2-27a) |

[symbol]

indicates data missing or illegible when filed

| X(R [symbol] ) [symbol] | R [symbol] | R [symbol] | R [symbol] | R [symbol] | R [symbol] | |
|---|---|---|---|---|---|---|
| N(CH [symbol] ) | CH [symbol] | CH [symbol] | CH [symbol] | CH [symbol] | H | (9a-2-1b) |
| N(CH [symbol] ) | CH [symbol] | CH [symbol] | CH [symbol] | Et | H | (9a-2-2b) |
| N(CH [symbol] ) | CH [symbol] | CH [symbol] | CH [symbol] | ⁱPr | H | (9a-2-3b) |
| N(CH [symbol] ) | CH [symbol] | CH [symbol] | CH [symbol] | Bu | H | (9a-2-4b) |
| N(CH [symbol] ) | CH [symbol] | CH [symbol] | CH [symbol] | Bnz | H | (9a-2-5b) |
| N(CH [symbol] ) | CH [symbol] | CH [symbol] | Et | Et | H | (9a-2-6b) |
| N(CH [symbol] ) | CH [symbol] | CH [symbol] | CH [symbol] CH [symbol] OCH [symbol] | CH [symbol] CH [symbol] OCH [symbol] | H | (9a-2-7b) |
| N(CH [symbol] ) | CH [symbol] | CH [symbol] | Pr | Bu | H | (9a-2-8b) |
| N(CH [symbol] ) | CH [symbol] | Et | Et | Et | H | (9a-2-9b) |
| N(CH [symbol] ) | CH [symbol] | ⁱPr | ⁱPr | ⁱPr | H | (9a-2-10b) |
| N(CH [symbol] ) | CH [symbol] | Bu | Bu | Bu | H | (9a-2-11b) |
| N(CH [symbol] ) | CH [symbol] CH [symbol] CH [symbol] | CH [symbol] | CH [symbol] | CH [symbol] | H | (9a-2-12b) |
| N(CH [symbol] ) | CH [symbol] CH [symbol] CH [symbol] | CH [symbol] | CH [symbol] | Et | H | (9a-2-13b) |
| N(CH [symbol] ) | CH [symbol] CH [symbol] CH [symbol] | CH [symbol] | CH [symbol] | ⁱPr | H | (9a-2-14b) |
| N(CH [symbol] ) | CH [symbol] CH [symbol] CH [symbol] | CH [symbol] | CH [symbol] | Bu | H | (9a-2-15b) |
| N(CH [symbol] ) | CH [symbol] CH [symbol] CH [symbol] | CH [symbol] | CH [symbol] | Bnz | H | (9a-2-16b) |
| N(CH [symbol] ) | CH [symbol] CH [symbol] CH [symbol] | CH [symbol] | Et | Et | H | (9a-2-17b) |
| N(CH [symbol] ) | CH [symbol] CH [symbol] CH [symbol] | CH [symbol] | CH [symbol] CH [symbol] OCH [symbol] | CH [symbol] CH [symbol] OCH [symbol] | H | (9a-2-18b) |
| N(CH [symbol] ) | CH [symbol] CH [symbol] CH [symbol] | CH [symbol] | Pr | Bu | H | (9a-2-19b) |
| N(CH [symbol] ) | CH [symbol] CH [symbol] CH [symbol] | Et | Et | Et | H | (9a-2-20b) |
| N(CH [symbol] ) | CH [symbol] CH [symbol] CH [symbol] | ⁱPr | ⁱPr | ⁱPr | H | (9a-2-21b) |
| N(CH [symbol] ) | CH [symbol] CH [symbol] CH [symbol] | Bu | Bu | Bu | H | (9a-2-22b) |
| N(CH [symbol] ) | CH [symbol] (CH [symbol] ) [symbol] CH [symbol] | CH [symbol] | CH [symbol] | CH [symbol] | H | (9a-2-23b) |
| N(CH [symbol] ) | CH [symbol] (CH [symbol] ) [symbol] CH [symbol] | CH [symbol] | CH [symbol] | Et | H | (9a-2-24b) |
| N(CH [symbol] ) | CH [symbol] (CH [symbol] ) [symbol] CH [symbol] | CH [symbol] | CH [symbol] | ⁱPr | H | (9a-2-25b) |
| N(CH [symbol] ) | CH [symbol] (CH [symbol] ) [symbol] CH [symbol] | CH [symbol] | CH [symbol] | Bu | H | (9a-2-26b) |
| N(CH [symbol] ) | CH [symbol] (CH [symbol] ) [symbol] CH [symbol] | CH [symbol] | CH [symbol] | Bnz | H | (9a-2-27b) |
| N(CH [symbol] ) | CH [symbol] (CH [symbol] ) [symbol] CH [symbol] | CH [symbol] | Et | Et | H | (9a-2-28b) |
| N(CH [symbol] ) | CH [symbol] (CH [symbol] ) [symbol] CH [symbol] | CH [symbol] | CH [symbol] CH [symbol] OCH [symbol] | CH [symbol] CH [symbol] OCH [symbol] | H | (9a-2-29b) |
| N(CH [symbol] ) | CH [symbol] (CH [symbol] ) [symbol] CH [symbol] | CH [symbol] | Pr | Bu | H | (9a-2-30b) |
| N(CH [symbol] ) | CH [symbol] (CH [symbol] ) [symbol] CH [symbol] | Et | Et | Et | H | (9a-2-31b) |
| N(CH [symbol] ) | CH [symbol] (CH [symbol] ) [symbol] CH [symbol] | ⁱPr | ⁱPr | ⁱPr | H | (9a-2-32b) |
| N(CH [symbol] ) | CH [symbol] (CH [symbol] ) [symbol] CH [symbol] | Bu | Bu | Bu | H | (9a-2-33b) |

[symbol]

indicates data missing or illegible when filed

| X(R [symbol] ) [symbol] | R [symbol] | R [symbol] | R [symbol] | R [symbol] | R [symbol] | |
|---|---|---|---|---|---|---|
| N(CH [symbol] ) | CH [symbol] | CH [symbol] | CH [symbol] | CH [symbol] | CH [symbol] | (9b-2-1b) |
| N(CH [symbol] ) | CH [symbol] | CH [symbol] | CH [symbol] | Et | CH [symbol] | (9b-2-2b) |
| N(CH [symbol] ) | CH [symbol] | CH [symbol] | CH [symbol] | ⁱPr | CH [symbol] | (9b-2-3b) |
| N(CH [symbol] ) | CH [symbol] | CH [symbol] | CH [symbol] | Bu | CH [symbol] | (9b-2-4b) |
| N(CH [symbol] ) | CH [symbol] | CH [symbol] | CH [symbol] | Bnz | CH [symbol] | (9b-2-5b) |
| N(CH [symbol] ) | CH [symbol] | CH [symbol] | Et | Et | CH [symbol] | (9b-2-6b) |
| N(CH [symbol] ) | CH [symbol] | CH [symbol] | CH [symbol] CH [symbol] OCH [symbol] | CH [symbol] CH [symbol] OCH [symbol] | CH [symbol] | (9b-2-7b) |
| N(CH [symbol] ) | CH [symbol] | CH [symbol] | Pr | Bu | CH [symbol] | (9b-2-8b) |
| N(CH [symbol] ) | CH [symbol] | CH [symbol] | CH [symbol] | Et | Et | (9b-2-9b) |
| N(CH [symbol] ) | CH [symbol] CH [symbol] CH [symbol] | CH [symbol] | CH [symbol] | CH [symbol] | CH [symbol] | (9b-2-10b) |
| N(CH [symbol] ) | CH [symbol] CH [symbol] CH [symbol] | CH [symbol] | CH [symbol] | Et | CH [symbol] | (9b-2-11b) |
| N(CH [symbol] ) | CH [symbol] CH [symbol] CH [symbol] | CH [symbol] | CH [symbol] | ⁱPr | CH [symbol] | (9b-2-12b) |
| N(CH [symbol] ) | CH [symbol] CH [symbol] CH [symbol] | CH [symbol] | CH [symbol] | Bu | CH [symbol] | (9b-2-13b) |
| N(CH [symbol] ) | CH [symbol] CH [symbol] CH [symbol] | CH [symbol] | CH [symbol] | Bnz | CH [symbol] | (9b-2-14b) |
| N(CH [symbol] ) | CH [symbol] CH [symbol] CH [symbol] | CH [symbol] | Et | Et | CH [symbol] | (9b-2-15b) |
| N(CH [symbol] ) | CH [symbol] CH [symbol] CH [symbol] | CH [symbol] | CH [symbol] CH [symbol] OCH [symbol] | CH [symbol] CH [symbol] OCH [symbol] | CH [symbol] | (9b-2-16b) |
| N(CH [symbol] ) | CH [symbol] CH [symbol] CH [symbol] | CH [symbol] | Pr | Bu | CH [symbol] | (9b-2-17b) |
| N(CH [symbol] ) | CH [symbol] CH [symbol] CH [symbol] | CH [symbol] | CH [symbol] | Et | Et | (9b-2-18b) |
| N(CH [symbol] ) | CH [symbol] (CH [symbol] ) [symbol] CH [symbol] | CH [symbol] | CH [symbol] | CH [symbol] | CH [symbol] | (9b-2-19b) |
| N(CH [symbol] ) | CH [symbol] (CH [symbol] ) [symbol] CH [symbol] | CH [symbol] | CH [symbol] | Et | CH [symbol] | (9b-2-20b) |
| N(CH [symbol] ) | CH [symbol] (CH [symbol] ) [symbol] CH [symbol] | CH [symbol] | CH [symbol] | ⁱPr | CH [symbol] | (9b-2-21b) |
| N(CH [symbol] ) | CH [symbol] (CH [symbol] ) [symbol] CH [symbol] | CH [symbol] | CH [symbol] | Bu | CH [symbol] | (9b-2-22b) |
| N(CH [symbol] ) | CH [symbol] (CH [symbol] ) [symbol] CH [symbol] | CH [symbol] | CH [symbol] | Bnz | CH [symbol] | (9b-2-23b) |
| N(CH [symbol] ) | CH [symbol] (CH [symbol] ) [symbol] CH [symbol] | CH [symbol] | Et | Et | CH [symbol] | (9b-2-24b) |
| N(CH [symbol] ) | CH [symbol] (CH [symbol] ) [symbol] CH [symbol] | CH [symbol] | CH [symbol] CH [symbol] OCH [symbol] | CH [symbol] CH [symbol] OCH [symbol] | CH [symbol] | (9b-2-25b) |
| N(CH [symbol] ) | CH [symbol] (CH [symbol] ) [symbol] CH [symbol] | CH [symbol] | Pr | Bu | CH [symbol] | (9b-2-26b) |
| N(CH [symbol] ) | CH [symbol] (CH [symbol] ) [symbol] CH [symbol] | CH [symbol] | CH [symbol] | Et | Et | (9b-2-27b) |

[symbol]

indicates data missing or illegible when filed

| X(R [symbol] ) [symbol] | R [symbol] | R [symbol] | R [symbol] | R [symbol] | R [symbol] | |
|---|---|---|---|---|---|---|
| 0 | CH [symbol] | CH [symbol] | CH [symbol] | CH [symbol] | H | (9a-2-1c) |
| 0 | CH [symbol] | CH [symbol] | CH [symbol] | Et | H | (9a-2-2c) |
| 0 | CH [symbol] | CH [symbol] | CH [symbol] | ⁱPr | H | (9a-2-3c) |
| 0 | CH [symbol] | CH [symbol] | CH [symbol] | Bu | H | (9a-2-4c) |
| 0 | CH [symbol] | CH [symbol] | CH [symbol] | Bnz | H | (9a-2-5c) |
| 0 | CH [symbol] | CH [symbol] | Et | Et | H | (9a-2-6c) |
| 0 | CH [symbol] | CH [symbol] | CH [symbol] CH [symbol] OCH [symbol] | CH [symbol] CH [symbol] OCH [symbol] | H | (9a-2-7c) |
| 0 | CH [symbol] | CH [symbol] | Pr | Bu | H | (9a-2-8c) |
| 0 | CH [symbol] | Et | Et | Et | H | (9a-2-9c) |
| 0 | CH [symbol] | ⁱPr | ⁱPr | ⁱPr | H | (9a-2-10c) |
| 0 | CH [symbol] | Bu | Bu | Bu | H | (9a-2-11c) |
| 0 | CH [symbol] CH [symbol] CH [symbol] | CH [symbol] | CH [symbol] | CH [symbol] | H | (9a-2-12c) |
| 0 | CH [symbol] CH [symbol] CH [symbol] | CH [symbol] | CH [symbol] | Et | H | (9a-2-13c) |
| 0 | CH [symbol] CH [symbol] CH [symbol] | CH [symbol] | CH [symbol] | ⁱPr | H | (9a-2-14c) |
| 0 | CH [symbol] CH [symbol] CH [symbol] | CH [symbol] | CH [symbol] | Bu | H | (9a-2-15c) |
| 0 | CH [symbol] CH [symbol] CH [symbol] | CH [symbol] | CH [symbol] | Bnz | H | (9a-2-16c) |
| 0 | CH [symbol] CH [symbol] CH [symbol] | CH [symbol] | Et | Et | H | (9a-2-17c) |
| 0 | CH [symbol] CH [symbol] CH [symbol] | CH [symbol] | CH [symbol] CH [symbol] OCH [symbol] | CH [symbol] CH [symbol] OCH [symbol] | H | (9a-2-18c) |
| 0 | CH [symbol] CH [symbol] CH [symbol] | CH [symbol] | Pr | Bu | H | (9a-2-19c) |
| 0 | CH [symbol] CH [symbol] CH [symbol] | Et | Et | Et | H | (9a-2-20c) |
| 0 | CH [symbol] CH [symbol] CH [symbol] | ⁱPr | ⁱPr | ⁱPr | H | (9a-2-21c) |
| 0 | CH [symbol] CH [symbol] CH [symbol] | Bu | Bu | Bu | H | (9a-2-22c) |
| 0 | CH [symbol] (CH [symbol] ) [symbol] CH [symbol] | CH [symbol] | CH [symbol] | CH [symbol] | H | (9a-2-23c) |
| 0 | CH [symbol] (CH [symbol] ) [symbol] CH [symbol] | CH [symbol] | CH [symbol] | Et | H | (9a-2-24c) |
| 0 | CH [symbol] (CH [symbol] ) [symbol] CH [symbol] | CH [symbol] | CH [symbol] | ⁱPr | H | (9a-2-25c) |
| 0 | CH [symbol] (CH [symbol] ) [symbol] CH [symbol] | CH [symbol] | CH [symbol] | Bu | H | (9a-2-26c) |
| 0 | CH [symbol] (CH [symbol] ) [symbol] CH [symbol] | CH [symbol] | CH [symbol] | Bnz | H | (9a-2-27c) |
| 0 | CH [symbol] (CH [symbol] ) [symbol] CH [symbol] | CH [symbol] | Et | Et | H | (9a-2-28c) |
| 0 | CH [symbol] (CH [symbol] ) [symbol] CH [symbol] | CH [symbol] | CH [symbol] CH [symbol] OCH [symbol] | CH [symbol] CH [symbol] OCH [symbol] | H | (9a-2-29c) |
| 0 | CH [symbol] (CH [symbol] ) [symbol] CH [symbol] | CH [symbol] | Pr | Bu | H | (9a-2-30c) |
| 0 | CH [symbol] (CH [symbol] ) [symbol] CH [symbol] | Et | Et | Et | H | (9a-2-31c) |
| 0 | CH [symbol] (CH [symbol] ) [symbol] CH [symbol] | ⁱPr | ⁱPr | ⁱPr | H | (9a-2-32c) |
| 0 | CH [symbol] (CH [symbol] ) [symbol] CH [symbol] | Bu | Bu | Bu | H | (9a-2-33c) |

[symbol]

indicates data missing or illegible when filed

| X(R [symbol] ) [symbol] | R [symbol] | R [symbol] | R [symbol] | R [symbol] | R [symbol] | |
|---|---|---|---|---|---|---|
| 0 | CH [symbol] | CH [symbol] | CH [symbol] | CH [symbol] | CH [symbol] | (9b-2-1c) |
| 0 | CH [symbol] | CH [symbol] | CH [symbol] | Et | CH [symbol] | (9b-2-2c) |
| 0 | CH [symbol] | CH [symbol] | CH [symbol] | ⁱPr | CH [symbol] | (9b-2-3c) |
| 0 | CH [symbol] | CH [symbol] | CH [symbol] | Bu | CH [symbol] | (9b-2-4c) |
| 0 | CH [symbol] | CH [symbol] | CH [symbol] | Bnz | CH [symbol] | (9b-2-5c) |
| 0 | CH [symbol] | CH [symbol] | Et | Et | CH [symbol] | (9b-2-6c) |
| 0 | CH [symbol] | CH [symbol] | CH [symbol] CH [symbol] OCH [symbol] | CH [symbol] CH [symbol] OCH [symbol] | CH [symbol] | (9b-2-7c) |
| 0 | CH [symbol] | CH [symbol] | Pr | Bu | CH [symbol] | (9b-2-8c) |
| 0 | CH [symbol] | CH [symbol] | CH [symbol] | Et | Et | (9b-2-9c) |
| 0 | CH [symbol] CH [symbol] CH [symbol] | CH [symbol] | CH [symbol] | CH [symbol] | CH [symbol] | (9b-2-10c) |
| 0 | CH [symbol] CH [symbol] CH [symbol] | CH [symbol] | CH [symbol] | Et | CH [symbol] | (9b-2-11c) |
| 0 | CH [symbol] CH [symbol] CH [symbol] | CH [symbol] | CH [symbol] | ⁱPr | CH [symbol] | (9b-2-12c) |
| 0 | CH [symbol] CH [symbol] CH [symbol] | CH [symbol] | CH [symbol] | Bu | CH [symbol] | (9b-2-13c) |
| 0 | CH [symbol] CH [symbol] CH [symbol] | CH [symbol] | CH [symbol] | Bnz | CH [symbol] | (9b-2-14c) |
| 0 | CH [symbol] CH [symbol] CH [symbol] | CH [symbol] | Et | Et | CH [symbol] | (9b-2-15c) |
| 0 | CH [symbol] CH [symbol] CH [symbol] | CH [symbol] | CH [symbol] CH [symbol] OCH [symbol] | CH [symbol] CH [symbol] OCH [symbol] | CH [symbol] | (9b-2-16c) |
| 0 | CH [symbol] CH [symbol] CH [symbol] | CH [symbol] | Pr | Bu | CH [symbol] | (9b-2-17c) |
| 0 | CH [symbol] CH [symbol] CH [symbol] | CH [symbol] | CH [symbol] | Et | Et | (9b-2-18c) |
| 0 | CH [symbol] (CH [symbol] ) [symbol] CH [symbol] | CH [symbol] | CH [symbol] | CH [symbol] | CH [symbol] | (9b-2-19c) |
| 0 | CH [symbol] (CH [symbol] ) [symbol] CH [symbol] | CH [symbol] | CH [symbol] | Et | CH [symbol] | (9b-2-20c) |
| 0 | CH [symbol] (CH [symbol] ) [symbol] CH [symbol] | CH [symbol] | CH [symbol] | ⁱPr | CH [symbol] | (9b-2-21c) |
| 0 | CH [symbol] (CH [symbol] ) [symbol] CH [symbol] | CH [symbol] | CH [symbol] | Bu | CH [symbol] | (9b-2-22c) |
| 0 | CH [symbol] (CH [symbol] ) [symbol] CH [symbol] | CH [symbol] | CH [symbol] | Bnz | CH [symbol] | (9b-2-23c) |
| 0 | CH [symbol] (CH [symbol] ) [symbol] CH [symbol] | CH [symbol] | Et | Et | CH [symbol] | (9b-2-24c) |
| 0 | CH [symbol] (CH [symbol] ) [symbol] CH [symbol] | CH [symbol] | CH [symbol] CH [symbol] OCH [symbol] | CH [symbol] CH [symbol] OCH [symbol] | CH [symbol] | (9b-2-25c) |
| 0 | CH [symbol] (CH [symbol] ) [symbol] CH [symbol] | CH [symbol] | Pr | Bu | CH [symbol] | (9b-2-26c) |
| 0 | CH [symbol] (CH [symbol] ) [symbol] CH [symbol] | CH [symbol] | CH [symbol] | Et | Et | (9b-2-27c) |

[symbol]

indicates data missing or illegible when filed

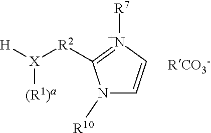

X, a, R¹, R², R⁷, R¹⁰, and R′ are as defined above. Preferably, X(R¹)ₐ represents NH, N(CH³), or O, R² represents CH₂, CH₂CH₂, or CH₂(CH₂)₄CH₂, R⁷ represents CH₃ or Et, R¹⁰ represents CH₃, Et, ⁱPr, Bu, Bnz, or CH₂CH₂OCH₃, and R′ represents H, CH₃, or Et.

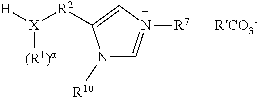

X, a, R¹, R², R⁷, R¹⁰, and R′ are as defined above. Preferably, X(R¹)^{a} represents NH, N(CH₃), or O, R² represents CH₂, CH₂CH₂, or CH₂(CH₂)₄CH₂, R⁷ represents CH₃ or Et, R¹⁰ represents CH₃, Et, ⁱPr, Bu, Bnz, or CH₂CH₂OCH₃, and R′ represents H, CH₃, or Et.

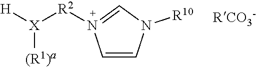

X, a, R¹, R², R¹⁰ and R′ are as defined above. Preferably, X(R¹) a represents NH, N(CH₃), or O, R² represents CH₂, CH₂CH₂, or CH₂(CH₂)₄CH₂, R⁷ represents CH₃ or Et, R¹⁰ represents CH₃, Et, ⁱPr, Bu, Bnz, or CH₂CH₂OCH₃, and R′ represents H, CH₃ or Et.

The compound represented by formula (9) is preferably any one of (9a-2-1a) to (9a-2-33a), (9b-2-1a) to (9b-2-27a), (9a-2-1b) to (9a-2-33b), (9b-2-1b) to (9b-2-27b), (9a-2-1c) to (9a-2-33c), and (9b-2-1c) to (9b-2-27c), and particularly preferably (9a-2-12a) or (9b-2-10a).

For the reaction, the isocyanate compound represented by formula (5) is usually used in an amount of 0.5 to 10 mol, and preferably 0.8 to 3 mol, per 1/n mol of the compound represented by formula (9).

The reaction temperature is usually 10° C. to 100° C. or the boiling temperature of the solvent. The reaction temperature is preferably 20 to 150° C.

The reaction time is usually 1 to 20 hours, and preferably 1 to 10 hours.

Solvents may or may not be used. Examples of solvents include tetrahydrofuran, ethyl acetate, acetonitrile, toluene, acetone, and the like. The amount of solvents when used is usually 100 parts by mass or less, and preferably 0.1 to 50 parts by mass, per part by mass of the compound represented by formula (9). Two or more different solvents may be used in combination, if necessary.

### Blocked Isocyanate Composition Comprising Blocked Isocyanate Compound and Zwitterionic Compound Represented by Formula (1)

The blocked isocyanate composition of the present invention comprises a blocked isocyanate compound and a zwitterionic compound represented by formula (1).

The blocked isocyanate compound is explained.

Examples of the blocked isocyanate compound include compounds obtained by reacting isocyanates and blocking agents to block the isocyanate groups in the isocyanates with the blocking agents. The blocked isocyanate compounds may be used singly or as a mixture of two or more.

The isocyanate that constitutes the blocked isocyanate compound is not particularly limited, as long as it is a compound having two or more isocyanate groups. Examples of isocyanates include the following:
(i) aliphatic isocyanates,
(ii) alicyclic isocyanates,
(iii) aromatic isocyanates,
(iv) aromatic aliphatic isocyanates, and
(v) modified isocyanates formed from at least one member selected from the group consisting of aliphatic isocyanates, alicyclic isocyanates, aromatic isocyanates, and aromatic aliphatic isocyanates.

Preferred among these are the aliphatic isocyanates (i), alicyclic isocyanates (ii), and modified isocyanates (v) formed from at least one member selected from the group consisting of aliphatic isocyanates, alicyclic isocyanates, aromatic isocyanates, and aromatic aliphatic isocyanates.

These isocyanates may be used singly or as a mixture of two or more.

Examples of aliphatic isocyanates include 1,4-tetramethylene diisocyanate, 1,6-hexamethylene diisocyanate, 2,2,4-trimethylhexamethylene diisocyanate, 2,4,4-trimethylhexamethylene diisocyanate, lysine diisocyanate, dimer acid diisocyanate, and the like.

Examples of alicyclic isocyanates include 1,3-bis(isocyanatomethyl)cyclohexane, 1,4-bis(isocyanatomethyl)cyclohexane, 3-isocyanatomethyl-3,3,5-trimethylcyclohexane (isophorone diisocyanate (IPDI)), bis-(4-isocyanatocyclohexyl)methane, norbornane diisocyanate, and the like.

Examples of aromatic isocyanates include 2,4′-diphenylmethane diisocyanate, 4,4′-diphenylmethane diisocyanate, crude diphenylmethane diisocyanate, 1,4-phenylene diisocyanate, 2,4-tolylene diisocyanate, 2,6-tolylene diisocyanate, 3,3′-dimethyl-4,4′-diisocyanatobiphenyl, 3,3′-dimethyl-4,4′-diisocyanatodiphenylmethane, 1,5-naphthylene diisocyanate, and the like.

Examples of aromatic aliphatic isocyanates include 1,3-xylylene diisocyanate, 1,4-xylylene diisocyanate, α,α,α′,α′-tetramethylxylylene diisocyanate, and the like.

Examples of modified isocyanates include isocyanate-terminated compounds obtained by the reaction of the above isocyanate compounds with compounds having an active hydrogen group, and reaction products of the isocyanate compounds and/or the isocyanate-terminated compounds (e.g., adduct-type isocyanates, and modified isocyanates obtained by allophanatization reaction, carbodiimidization reaction, uretodionization reaction, isocyanuration reaction, uretoniminization reaction, biuretization reaction, or the like); and preferably adduct-type isocyanates, isocyanates modified by isocyanuration reaction, and isocyanates modified by biuretization reaction (isocyanates having a biuret bond).

An isocyanate having a biuret bond is obtained by reacting a so-called biuretizing agent, such as water, tert-butanol, or urea, with an isocyanate at a molar ratio of the biuretizing agent/isocyanate groups in the isocyanate of about ½ to about 1/100, followed by purification by removing the unreacted isocyanate. An isocyanate having an isocyanurate bond is obtained, for example, by performing the cyclic trimerization reaction using a catalyst etc., stopping the reaction when the conversion rate reaches about 5 to about 80 mass %, and removing the unreacted isocyanate for purification. In this case, a mono- to hexavalent alcohol compound can be used in combination.

Examples of isocyanates having a biuret bond include a biuret modified product of 1,6-hexamethylene diisocyanate (HDI), a biuret modified product of isophorone diisocyanate (IPDI), and a biuret modified product of toluene diisocyanate (TDI) shown below. Commercial products include Desmodur N75, Desmodur N100, and Desmodur N3200 (all produced by Sumika Covestro Urethane Co., Ltd.); Duranate 24A-100, Duranate 22A-75P, and Duranate 21S-75E (all produced by Asahi Kasei Corporation); and the like.

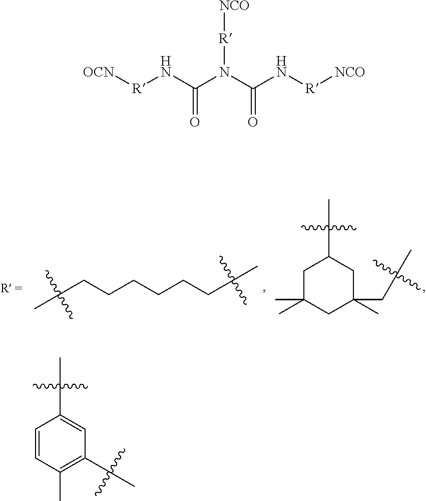

An isocyanate having an isocyanurate bond is obtained, for example, by performing an isocyanuration reaction using a catalyst etc., stopping the reaction when the conversion rate reaches about 5 to about 80 mass %, and removing the unreacted isocyanate for purification. In this case, a mono- to hexavalent alcohol compound can be used in combination.

The catalyst for the above isocyanuration reaction is generally preferably a basic catalyst.

Examples of the catalyst include the following:
(1) hydroxides of tetraalkylammonium, such as tetramethylammonium, tetraethylammonium, and trimethylbenzylammonium; and organic weak acid salts, such as acetic acid and capric acid;
(2) hydroxides of hydroxyalkylammonium, such as trimethylhydroxypropylammonium, trimethylhydroxyethylammonium, triethylhydroxypropylammonium, and triethylhydroxyethylammonium; and organic weak acid salts, such as acetic acid and capric acid;
(3) metal salts of alkyl carboxylic acids with, for example, tin, zinc, and lead;
(4) metal alcoholates of sodium, potassium, etc.;
(5) aminosilyl group-containing compounds, such as hexamethyldisilazane;
(6) Mannich bases;
(7) combination of tertiary amines and epoxy compounds; and
(8) phosphorus compounds, such as tributylphosphine.
These can be used in combination of two or more.

If the catalyst may adversely affect paints or coating film properties, the catalyst may be neutralized with an acidic compound. Examples of acidic compounds include inorganic acids, such as hydrochloric acid, phosphorous acid, and phosphoric acid; sulfonic acids or derivatives thereof, such as methanesulfonic acid, p-toluenesulfonic acid, p-toluenesulfonic acid methyl ester, and p-toluenesulfonic acid ethyl ester; ethyl phosphate, diethyl phosphate, isopropyl phosphate, diisopropyl phosphate, butyl phosphate, dibutyl phosphate, 2-ethylhexyl phosphate, di(2-ethylhexyl)phosphate, isodecyl phosphate, diisodecyl phosphate, oleyl acid phosphate, tetracosyl acid phosphate, ethyl glycol acid phosphate, butyl pyrophosphate, butyl phosphite, and the like. These may be used in combination of two or more.

Examples of isocyanates having an isocyanurate bond include isocyanurate-modified HDI, isocyanurate-modified IPDI, and isocyanurate-modified TDI, each having the structure shown below. Commercial products include Sumidur N3300, Desmodur 3900, Desmodur Z4470BA, Desmodur XP2763, Desmodur IL1351BA, and Desmodur HLBA (all produced by Sumika Covestro Urethane Co., Ltd.); Duranate TPA-100, Duranate MFA-75B, Duranate TUL-100, and Duranate TSA-100 (all produced by Asahi Kasei Corporation); and the like.

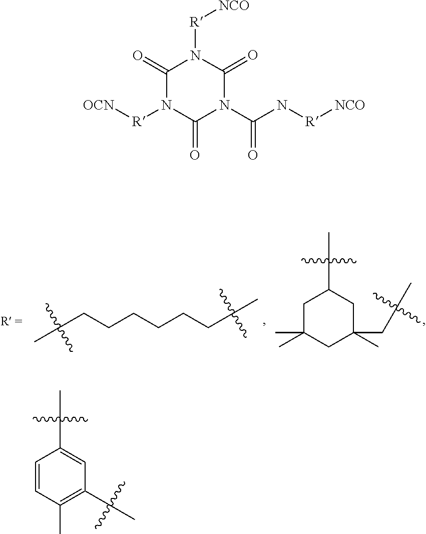

An isocyanate having a urethane bond is obtained, for example, by reacting a di- to hexavalent alcohol compound, such as trimethylolpropane (hereinafter referred to as TMP), with a diisocyanate at a molar ratio of hydroxyl groups in the alcohol compound/isocyanate groups in the isocyanate of about ½ to about 1/100, and then removing the unreacted isocyanate for purification. Removal of the unreacted isocyanate for purification is not necessarily required.

Examples of isocyanates having a urethane bond include a reaction product of HDI and TMP, a reaction product of IPDI and TMP, and a reaction product of TDI and TMP, each having the structure shown below. Commercial products include Sumidur HT, Desmodur L75 (C), Desmodur Ultra L75, and Desmodur L67BA (all produced by Sumika Covestro Urethane Co., Ltd.); Duranate P301-75E, Duranate AE700-100, Duranate E402-80B, and Duranate E405-70B (all produced by Asahi Kasei Corporation); and the like.

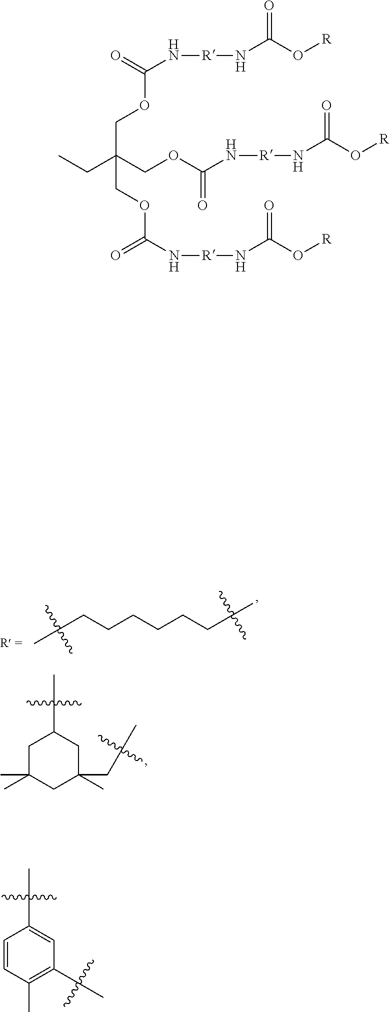

Examples of known blocking agents for isocyanates in which some isocyanate groups of the above isocyanates or modified isocyanates are blocked with known blocking agents include phenols, such as phenol, thiophenol, methylthiophenol, xylenol, cresol, resorcinol, nitrophenol, and chlorophenol; oximes, such as acetone oxime, methyl ethyl ketone oxime, and cyclohexanone oxime; alcohols, such as methanol, ethanol, n-propyl alcohol, isopropyl alcohol, n-butyl alcohol, isobutyl alcohol, t-butyl alcohol, t-pentanol, ethylene glycol monoethyl ether, ethylene glycol monobutyl ether, diethylene glycol monoethyl ether, propylene glycol monomethyl ether, and benzyl alcohol; pyrazoles, such as 3,5-dimethyl pyrazole and 1,2-pyrazole; triazoles, such as 1,2,4-triazole; halogen-substituted alcohols, such as ethylene chlorohydrin and 1,3-dichloro-2-propanol; lactams, such as ε-caprolactam, δ-valerolactam, γ-butyrolactam, and β-propyllactam; active methylene compounds, such as methyl acetoacetate, ethyl acetoacetate, acetylacetone, methyl malonate, and ethyl malonate; and the like. Other examples include amines, imides, mercaptans, imines, ureas, diaryls, and the like.

Examples of blocking agents include alcohol compounds, phenol compounds, amine compounds, lactam compounds, oxime compounds, ketoenol compounds, active methylene compounds, pyrazole compounds, triazole compounds, imide compounds, mercaptan compounds, imine compounds, urea compounds, and diaryl compounds; preferably alcohol compounds, lactam compounds, oxime compounds, and pyrazole compounds; preferably alcohol compounds because when they are combined with the zwitterionic compound represented by formula (1), the blocking agent can be dissociated in a short period of time even at 120° C.; and particularly preferably fluorinated alcohol compounds.

Examples of alcohol compounds include methanol, ethanol, n-propyl alcohol, isopropyl alcohol, n-butyl alcohol, isobutyl alcohol, t-butyl alcohol, t-pentanol, ethylene glycol monoethyl ether, ethylene glycol monobutyl ether, diethylene glycol monoethyl ether, propylene glycol monomethyl ether, benzyl alcohol, and the like. Alcohol compounds include fluorinated alcohol compounds, such as 2,2,2-trifluoroethanol and 1,1,1,3,3,3-hexafluoro-2-propanol.

The alcohol compound is preferably a fluorinated alcohol compound, and particularly preferably 2,2,2-trifluoroethanol.

Examples of phenol compounds include phenol, thiophenol, methylthiophenol, xylenol, cresol, resorcinol, nitrophenol, chlorophenol, 2-hydroxypyridine, and the like.

Examples of amine compounds include diisopropylamine and the like.

Examples of lactam compounds include ε-caprolactam, δ-valerolactam, γ-butyrolactam, and the like; and preferably ε-caprolactam.

Preferred examples of oxime compounds include compounds represented by the following formula (L).

Formula (L):

HO—N═R″ (L)

wherein R″ is a hydrogen atom or a C₁-C₂₀ alkyl group. R is a hydrogen atom or a C₁-C₂₀ alkyl group, preferably a C₁-C₂₀ alkyl group, more preferably a C₁-C₆ alkyl group, and particularly preferably a C₁-C₄ alkyl group.

Examples of C₁-C₂₀ alkyl groups include methyl, ethyl, propyl, isopropyl, butyl, sec-butyl, tert-butyl, 1-methylpropyl, pentyl, hexyl, heptyl, octyl, 1-ethylpentyl, nonyl, 2-ethylhexyl, undecyl, tridecyl, pentadecyl, and heptadecyl groups.

Examples of oxime compounds include formaldehyde oxime, acetaldehyde oxime, acetone oxime, methyl ethyl ketoxime, methyl isobutyl ketoxime, and the like; and preferably methyl ethyl ketoxime.

Examples of pyrazole compounds include 1,2-pyrazole, 3,5-dimethylpyrazole, and the like. Examples of triazole compounds include 1,2,4-triazole and the like, and preferably 3,5-dimethylpyrazole.

Examples of active methylene compounds include methyl acetoacetate, ethyl acetoacetate, acetylacetone, methyl malonate, ethyl malonate, and the like.

In the blocked isocyanate composition of the present invention, known catalysts for polyurethane production, additives, pigments, solvents, and the like that are commonly used in this technical field can be used, if necessary.

Known catalysts for polyurethane production are not particularly limited. Examples include tin compounds, such as dibutyltin dilaurate, dibutyltin di-2-ethylhexanate, dioctyltin dilaurate, dibutyltin diacetate, dibutyltin dioxide, dioctyltin dioxide, tin acetylacetonate, tin acetate, tin octylate, and tin laurate; bismuth compounds, such as bismuth octylate, bismuth naphthenate, and bismuth acetylacetonate; titanium compounds, such as tetra-n-butyl titanate, tetraisopropyl titanate, and titanium terephthalate; tertiary amine compounds, such as triethylamine, N,N,N′,N′-tetramethylethylenediamine, N,N,N′,N′-tetramethylpropylenediamine, N,N,N′,N″,N″-pentamethyldiethylenetriamine, N,N,N′,N″,N″-pentamethyldipropylenetriamine, N,N,N′,N′-tetramethylguanidine, 1,3,5-tris(N, N-dimethylaminopropyl)hexahydro-S-triazine, 1,4-diazabicyclo[2.2.2]octane (DABCO), 1,8-diazabicyclo[5.4.0]undecene-7, triethylenediamine, N,N,N′,N′-tetramethylhexamethylenediamine, N-methyl-N′-(2-dimethylaminoethyl) piperazine, N,N′-dimethylpiperazine, dimethylcyclohexylamine, N-methylmorpholine, N-ethylmorpholine, bis(2-dimethylaminoethyl)ether, 1-methylimidazole, 1,2-dimethylimidazole, 1-isobutyl-2-methylimidazole, and 1-dimethylaminopropylimidazole; and quaternary ammonium salt compounds, such as tetraalkylammonium halides (e.g., tetramethylammonium chloride), tetraalkylammonium hydroxides (e.g., tetramethylammonium hydroxide salts), tetraalkylammonium organic acid salts (e.g., tetramethylammonium-2-ethylhexanoate, 2-hydroxypropyl trimethylammonium formate, and 2-hydroxypropyl trimethylammonium-2-ethylhexanoate).

Additives are not particularly limited. Examples include hindered amine-based, benzotriazole-based, and benzophenone-based UV absorbers; perchlorate-based and hydroxylamine-based coloration inhibitors; hindered phenol-based, phosphorus-based, sulfur-based, and hydrazide-based antioxidants; tin-based, zinc-based, and amine-based urethanization catalysts; leveling agents, rheology control agents, pigment dispersants, and the like.

Pigments are not particularly limited. Examples include organic pigments, such as quinacridone-based, azo-based, and phthalocyanine-based pigments; inorganic pigments, such as titanium oxide, barium sulfate, calcium carbonate, and silica; and other pigments, such as carbon-based pigments, metal foil pigments, and rust-preventive pigments.

Solvents are not particularly limited. Examples include hydrocarbons, such as benzene, toluene, xylene, cyclohexane, mineral spirit, and naphtha; ketones, such as acetone, methyl ethyl ketone, and methyl isobutyl ketone; esters, such as ethyl acetate, butyl acetate, and cellosolve acetate; alcohols, such as methanol, ethanol, 2-propanol, butanol, 2-methoxyethanol, 2-ethoxyethanol, and 2-butoxyethanol; polyhydric alcohols, such as ethylene glycol, propylene glycol, diethylene glycol, polyethylene glycol, and glycerol; water; and the like. These solvents may be used singly or in combination of two or more.

The thermosetting resin composition of the present invention is explained.

The thermosetting resin composition of the present invention comprises the blocked isocyanate composition of the present invention and a compound having an isocyanate-reactive group.

Examples of the compound having an isocyanate-reactive group include compounds having two or more active hydrogen groups, such as polyols, polyamines, and alkanolamines; and preferably polyols. These compounds having an isocyanate-reactive group may be a mixture of two or more.

In the present invention, polyols are compounds having two or more hydroxyl groups. Examples include polyester polyols, polyether polyols, acrylic polyols, polyolefin polyols, fluorine polyols, and the like. Preferred polyols among these are acrylic polyols in terms of weather resistance, chemical resistance, and hardness. Alternatively, polyols preferred in terms of mechanical strength and oil resistance are polyester polyols. These polyols may be a mixture of two or more.

Polyester polyols can be obtained, for example, by the condensation reaction of a single dibasic acid or a mixture of two or more dibasic acids with a single polyhydric alcohol or a mixture of two or more polyhydric alcohols.

Examples of dibasic acids include carboxylic acids, such as succinic acid, adipic acid, dimer acid, maleic anhydride, phthalic anhydride, isophthalic acid, terephthalic acid, and 1,4-cyclohexanedicarboxylic acid; and the like.

Examples of polyhydric alcohols include ethylene glycol, propylene glycol, diethylene glycol, 1,4-butanediol, neopentyl glycol, 1,6-hexanediol, trimethylpentanediol, cyclohexanediol, trimethylolpropane, glycerol, pentaerythritol, 2-methylolpropanediol, ethoxylated trimethylolpropane, and the like.

As a specific method for producing polyester polyols, for example, the condensation reaction can be carried out by mixing the above components, and heating the mixture at about 160 to 220° C. Alternatively, for example, polycaprolactones obtained by the ring-opening polymerization of lactones, such as ε-caprolactone, with polyhydric alcohols can also be used as polyester polyols.

These polyester polyols can be modified by using, for example, aromatic diisocyanates, aliphatic diisocyanates, alicyclic diisocyanates, and isocyanates obtained from them. Among these, in terms of weather resistance, yellowing resistance, etc., polyester polyols are preferably modified by using aliphatic diisocyanates, alicyclic diisocyanates, and isocyanates obtained from them.

When the thermosetting resin composition of the present embodiment is used as an aqueous-based paint, some carboxylic acids derived from the dibasic acid etc. in the polyester polyol can be allowed to remain and neutralized with a base, such as amine or ammonia, thereby forming the polyester polyol into a water-soluble or water-dispersible resin.

Examples of polyether polyols include active hydrogen compounds, such as aliphatic amine polyols, aromatic amine polyols, Mannich polyols, polyhydric alcohols, polyhydric phenols, and bisphenols; compounds obtained by adding alkylene oxides to these active hydrogen compounds; and the like. These polyether polyols may be a mixture of two or more.

Examples of aliphatic amine polyols include alkylenediamine-based polyols and alkanolamine-based polyols. These polyol compounds are polyfunctional polyol compounds having terminal hydroxyl groups obtained by the ring-opening addition of at least one cyclic ether, such as ethylene oxide or propylene oxide, using alkylenediamine or alkanolamine as an initiator. As the alkylenediamine, known compounds can be used without limitation. Specifically, C₂₋₈ alkylenediamines, such as ethylenediamine, propylenediamine, butylenediamine, hexamethylenediamine, and neopentyldiamine, are preferably used. These aliphatic amine polyols may be a mixture of two or more.

Aromatic amine polyols are polyfunctional polyether polyol compounds having terminal hydroxyl groups obtained by the ring-opening addition of at least one cyclic ether, such as ethylene oxide or propylene oxide, using an aromatic diamine as an initiator. As the initiator, a known aromatic diamine can be used without limitation. Specific examples include 2,4-toluenediamine, 2,6-toluenediamine, diethyltoluenediamine, 4,4′-diaminodiphenylmethane, p-phenylenediamine, o-phenylenediamine, naphthalenediamine, and the like. Among these, toluenediamine (2,4-toluenediamine, 2,6-toluenediamine, or a mixture thereof) is particularly preferably used. These aromatic amine polyols may be a mixture of two or more.

Mannich polyols are active hydrogen compounds obtained by the Mannich reaction of phenol and/or an alkyl-substituted derivative thereof, formaldehyde, and alkanolamine, or polyol compounds obtained by the ring-opening addition polymerization of the active hydrogen compounds with at least one of ethylene oxide and propylene oxide. These Mannich polyols may be a mixture of two or more.

Examples of polyhydric alcohols include dihydric alcohols (e.g., ethylene glycol, propylene glycol, 1,4-butanediol, 1,6-hexanediol, diethylene glycol, triethylene glycol, dipropylene glycol, and neopentyl glycol), trihydric or higher alcohols (e.g., glycerol, trimethylolpropane, pentaerythritol, methylglucoside, sorbitol, and sucrose), and the like. These polyhydric alcohols may be a mixture of two or more.

Examples of polyhydric phenols include pyrogallol, hydroquinone, and the like. These polyhydric phenols may be a mixture of two or more.

Examples of bisphenols include bisphenol A, bisphenol S, bisphenol F, low-condensates of phenols and formaldehyde, and the like. These bisphenols may be a mixture of two or more.

Polyether polyols can be obtained, for example, by any of the following methods (1) to (3).

(1) A method of performing random or block addition of an alkylene oxide alone or a mixture of alkylene oxides to a polyhydroxy compound alone or a mixture of polyhydroxy compounds using a catalyst to obtain polyether polyols.

Examples of catalysts include hydroxides (of lithium, sodium, potassium, etc.), strong base catalysts (alcoholates, alkylamines, etc.), composite metal cyanide compound complexes (metal porphyrins, zinc hexacyanocobaltate complexes, etc.), and the like.

Examples of alkylene oxides include ethylene oxide, propylene oxide, butylene oxide, cyclohexene oxide, styrene oxide, and the like.

(2) A method of reacting polyamines with alkylene oxides to obtain polyethers polyols.

Examples of polyamines include ethylene diamines and the like described below.

Examples of alkylene oxides include those mentioned in (1).

(3) A method of polymerizing acrylamide etc. using the polyether polyols obtained in (1) or (2) as media to obtain so-called polymer polyols.

Examples of polyhydroxy compounds include the following (i) to (vi).

(i) diglycol, ditrimethylolpropane, pentaerythritol, dipentaerythritol, etc.

(ii) sugar alcohol compounds, such as erythritol, D-threitol, L-arabinitol, ribitol, xylitol, sorbitol, mannitol, galactitol, and rhamnitol

(iii) monosaccharides, such as arabinose, ribose, xylose, glucose, mannose, galactose, fructose, sorbose, rhamnose, fucose, and ribodesose

(iv) disaccharides, such as trehalose, sucrose, maltose, cellobiose, gentiobiose, lactose, and melibiose

(v) trisaccharides, such as raffinose, gentianose, and melezitose

(vi) tetrasaccharides, such as stachyose Acrylic polyols can be obtained, for example, by polymerizing polymerizable monomers having one or more active hydrogens per molecule, or by copolymerizing polymerizable monomers having one or more active hydrogens per molecule with other monomers copolymerizable with the polymerizable monomers, if necessary.

Examples of polymerizable monomers having one or more active hydrogens per molecule include the following (i) to (vi). These may be used singly or in combination of two or more.
(i) acrylic acid esters having active hydrogen, such as 2-hydroxyethyl acrylate, 2-hydroxypropyl acrylate, and 2-hydroxybutyl acrylate
(ii) methacrylic acid esters having active hydrogen, such as 2-hydroxyethyl methacrylate, 2-hydroxypropyl methacrylate, 2-hydroxybutyl methacrylate, 3-hydroxypropyl methacrylate, and 4-hydroxybutyl methacrylate
(iii) (meth)acrylic acid esters having polyvalent active hydrogen, such as (meth)acrylic acid monoesters of triols, such as glycerol and trimethylolpropane
(iv) monoethers of polyether polyols (e.g., polyethylene glycol, polypropylene glycol, and polybutylene glycol) with the above (meth)acrylic acid esters having active hydrogen
(v) adducts of glycidyl (meth)acrylate with monobasic acids (e.g., acetic acid, propionic acid, and p-tert-butyl benzoic acid)
(vi) adducts obtained by the ring-opening polymerization of lactones (e.g., ε-caprolactam and γ-valerolactone) with the active hydrogen of the above (meta)acrylic acid esters having active hydrogen

Examples of monomers copolymerizable with the above polymerizable monomers include the following (i) to (iv). These may be used singly or in combination of two or more.
(i) (meth)acrylic acid esters, such as methyl acrylate, ethyl acrylate, isopropyl acrylate, n-butyl acrylate, 2-ethylhexyl acrylate, methyl methacrylate, ethyl methacrylate, isopropyl methacrylate, n-butyl methacrylate, isobutyl methacrylate, n-hexyl methacrylate, cyclohexyl methacrylate, lauryl methacrylate, and glycidyl methacrylate
(ii) unsaturated carboxylic acids, such as acrylic acid, methacrylic acid, maleic acid, and itaconic acid; and unsaturated amides, such as acrylamide, N-methylolacrylamide, and diacetoneacrylamide
(iii) vinyl monomers having a hydrolyzable silyl group, such as vinyl trimethoxysilane, vinyl methyl dimethoxysilane, and γ-(meth)acrylopropyltrimethoxysilane
(iv) other polymerizable monomers, such as styrene, vinyl toluene, vinyl acetate, acrylonitrile, and dibutyl fumarate

As a specific method for producing acrylic polyols, for example, the above monomer components are subjected to solution polymerization in the presence of a known radical polymerization initiator, such as a peroxide or an azo compound, optionally followed by dilution with an organic solvent etc., thereby obtaining acrylic polyols.

When the thermosetting resin composition of the present embodiment is used as an aqueous-based paint, aqueous-based acrylic polyols can be produced by solution polymerization of the above monomer components, and conversion to an aqueous layer, or by using a known method, such as emulsion polymerization. In that case, the acidic portions of carboxylic acid-containing monomers and sulfonic acid-containing monomers, such as acrylic acid and methacrylic acid, can be neutralized with amine or ammonia to make acrylic polyols water-soluble or water-dispersible.

Examples of polyolefin polyols include polybutadiene having two or more hydroxyl groups, hydrogenated polybutadiene having two or more hydroxyl groups, hydrogenated polyisoprene having two or more hydroxyl groups, and the like.

In the polyolefin polyol, the number of hydroxyl groups is preferably three because higher coating film strength can be obtained.

In the present specification, “fluorine polyols” refer to polyols containing fluorine in the molecule. Specific examples of fluorine polyols include the copolymers of fluoroolefin, cyclovinyl ether, hydroxyalkyl vinyl ether, and vinyl monocarboxylate disclosed in JPS57-34107A, JPS61-275311A, etc. JPS57-34107A and JPS61-275311A are incorporated herein by reference in their entirety.

The lower limit of the hydroxyl value of the polyol is preferably 10 mgKOH/g or more, more preferably 20 mgKOH/g or more, and even more preferably 30 mgKOH/g or more.

On the other hand, the upper limit of the hydroxyl value of the polyol is not particularly limited, and may be, for example, 200 mgKOH/g or less.

Specifically, the hydroxyl value of the polyol is preferably 10 mgKOH/g or more and 200 mgKOH/g or less, more preferably 20 mgKOH/g or more and 200 mgKOH/g or less, and even more preferably 30 mgKOH/g or more and 200 mgKOH/g or less.

Further, the acid value of the polyol is preferably 0 mgKOH/g or more and 30 mgKOH/g or less.

The hydroxyl value and acid value can be measured according to JIS K1557.

The molar equivalent ratio (NCO/OH) of isocyanate groups in the blocked isocyanate composition to hydroxyl groups in the polyol is preferably 0.2 or more and 5.0 or less, more preferably 0.4 or more and 3.0 or less, and even more preferably 0.5 or more and 2.0 or less.

Usable polyamines are those having two or more primary amino groups or secondary amino groups per molecule. Preferred among these are those having three or more such amino groups per molecule.

Specific examples of polyamines include diamines, such as ethylenediamine, propylenediamine, butylenediamine, triethylenediamine, hexamethylenediamine, 4,4′-diaminodicyclohexylmethane, piperazine, 2-methylpiperazine, and isophoronediamine; chain polyamines having three or more amino groups, such as bishexamethylenetriamine, diethylenetriamine, triethylenetetramine, tetraethylenepentamine, pentamethylenehexamine, and tetrapropylenepentamine; and cyclic polyamines, such as 1,4,7,10,13,16-hexaazacyclooctadecane, 1,4,7,10-tetraazacyclodecane, 1,4,8,12-tetraazacyclopentadecane, and 1,4,8,11-tetraazacyclotetradecane.

Alkanolamines refer to compounds having an amino group and a hydroxyl group per molecule. Examples of alkanolamines include monoethanolamine, diethanolamine, aminoethylethanolamine, N-(2-hydroxypropyl)ethylenediamine, mono-, di-(n- or iso-) propanolamine, ethylene glycol-bis-propylamine, neopentanolamine, methylethanolamine, and the like.

The thermosetting resin composition of the present embodiment may contain, if necessary, melamine-based curing agents, such as complete alkyl type, methylol type, and alkylamino group type alkyl.

The thermosetting resin composition of the present embodiment may contain an organic solvent.

Further, the compound having an isocyanate-reactive group and the blocked isocyanate composition described above may contain an organic solvent.

Preferable organic solvents are those that are compatible with the blocked isocyanate composition.

Specific examples of organic solvents include hydrocarbons, such as benzene, toluene, xylene, cyclohexane, mineral spirit, and naphtha; ketones, such as acetone, methyl ethyl ketone, and methyl isobutyl ketone; esters, such as ethyl acetate, butyl acetate, and cellosolve acetate; alcohols, such as methanol, ethanol, 2-propanol, butanol, 2-methoxyethanol, 2-ethoxyethanol, and 2-butoxyethanol; polyhydric alcohols, such as ethylene glycol, propylene glycol, diethylene glycol, polyethylene glycol, and glycerol; water; and the like. These solvents may be used singly or in combination of two or more.

Further, the thermosetting resin composition of the present embodiment can be used as an aqueous thermosetting resin composition dissolved or dispersed in water. When the thermosetting resin composition of the present invention is used as an aqueous thermosetting resin composition, in order to improve the compatibility of the thermosetting resin composition, surfactants, solvents that tend to be miscible with water, etc. may be used for the blocked isocyanate composition of the present invention. Examples of surfactants include anionic surfactants, such as aliphatic soaps, rosin acid soaps, alkyl sulfonates, dialkylaryl sulfonates, alkyl sulfosuccinates, polyoxyethylene alkyl sulfates, and polyoxyethylene alkylaryl sulfates; and nonionic surfactants, such as polyoxyethylene alkyl ethers, polyoxyethylene alkyl aryl ethers, and polyoxyethylene oxypropylene block copolymers. Examples of solvents that tend to be miscible with water include diethylene glycol dimethyl ether, diethylene glycol diethyl ether, propylene glycol monomethyl ether acetate, propylene glycol monomethyl ether, propylene glycol dimethyl ether, dipropylene glycol dimethyl ether, isobutanol, butyl glycol, N-methylpyrrolidone, butyl diglycol, butyl diglycol acetate, and the like.

Preferred among the above solvents are diethylene glycol dimethyl ether, diethylene glycol diethyl ether, propylene glycol monomethyl ether, propylene glycol dimethyl ether, dipropylene glycol dimethyl ether, isobutanol, butyl glycol, N-methylpyrrolidone, and butyl diglycol; and more preferred are diethylene glycol dimethyl ether, diethylene glycol diethyl ether, propylene glycol dimethyl ether, and dipropylene glycol dimethyl ether. These solvents may be used singly or in combination of two or more. Ester solvents, such as ethyl acetate, n-butyl acetate, and cellosolve acetate, are not preferred because the solvents themselves may hydrolyze during storage.

In the thermosetting resin composition of the present invention, the mixing ratio of the blocked isocyanate composition and the compound having an isocyanate-reactive group is determined by the required physical properties, and is not particularly limited. The mixing ratio is generally within the following range: [the amount of effective isocyanate groups (mol) in the blocked isocyanate compound in the blocked isocyanate composition]/[the amount of active hydrogen groups (mol) in the compound having an isocyanate-reactive group]=0.2 to 5, and preferably 0.5 to 3. The effective isocyanate groups in the blocked isocyanate compound refer to isocyanate groups that are regenerated when the blocking agent is dissociated from the blocked isocyanate compound.

In the thermosetting resin composition of the present invention, known catalysts for polyurethane production, additives, pigments, and the like that are commonly used in this technical field can be used, if necessary. These may be used as a mixture with known blocked isocyanates.

Known catalysts for polyurethane production are not particularly limited. Examples include tin compounds, such as dibutyltin dilaurate, dibutyltin di-2-ethylhexanate, dioctyltin dilaurate, dibutyltin diacetate, dibutyltin dioxide, dioctyltin dioxide, tin acetylacetonate, tin acetate, tin octylate, and tin laurate; bismuth compounds, such as bismuth octylate, bismuth naphthenate, and bismuth acetylacetonate; titanium compounds, such as tetra-n-butyl titanate, tetraisopropyl titanate, and titanium terephthalate; tertiary amine compounds, such as triethylamine, N,N,N′,N′-tetramethylethylenediamine, N,N,N′,N′-tetramethylpropylenediamine, N,N,N′,N″,N″-pentamethyldiethylenetriamine, N,N,N′,N″,N″-pentamethyldipropylenetriamine, N,N,N′,N′-tetramethylguanidine, 1,3,5-tris(N, N-dimethylaminopropyl)hexahydro-S-triazine, 1,4-diazabicyclo[2.2.2]octane (DABCO), 1,8-diazabicyclo[5.4.0]undecene-7, triethylenediamine, N,N,N′,N′-tetramethylhexamethylenediamine, N-methyl-N′-(2-dimethylaminoethyl) piperazine, N,N′-dimethylpiperazine, dimethylcyclohexylamine, N-methylmorpholine, N-ethylmorpholine, bis(2-dimethylaminoethyl)ether, 1-methylimidazole, 1,2-dimethylimidazole, 1-isobutyl-2-methylimidazole, and 1-dimethylaminopropylimidazole; and quaternary ammonium salt compounds, such as tetraalkylammonium halides (e.g., tetramethylammonium chloride), tetraalkylammonium hydroxides (e.g., tetramethylammonium hydroxide salts), tetraalkylammonium organic acid salts (e.g., tetramethylammonium-2-ethylhexanoate, 2-hydroxypropyl trimethylammonium formate, and 2-hydroxypropyl trimethylammonium-2-ethylhexanoate).

Additives are not particularly limited. Examples include hindered amine-based, benzotriazole-based, and benzophenone-based UV absorbers; perchlorate-based and hydroxylamine-based coloration inhibitors; hindered phenol-based, phosphorus-based, sulfur-based, and hydrazide-based antioxidants; tin-based, zinc-based, and amine-based urethanization catalysts; leveling agents, antifoaming agents, rheology control agents, thixotropy-imparting agents, thickeners, light stabilizers, plasticizers, surfactants, coupling agents, flame retardants, rust inhibitors, fluorescent whitening agents, pigment dispersants, and the like that are commonly used in this technical field.

Pigments are not particularly limited. Examples include organic pigments, such as quinacridone-based, azo-based, and phthalocyanine-based pigments; inorganic pigments, such as titanium oxide, barium sulfate, calcium carbonate, and silica; and other pigments, such as carbon-based pigments, metal foil pigments, and rust-preventive pigments.

Examples of known blocked isocyanates include blocked isocyanates obtained by reacting isocyanates and known blocking agents. Examples of known blocking agents include phenol compounds, such as phenol, thiophenol, methylthiophenol, xylenol, cresol, resorcinol, nitrophenol, and chlorophenol; oxime compounds, such as acetone oxime, methyl ethyl ketone oxime, and cyclohexanone oxime; alcohol compounds, such as methanol, ethanol, n-propyl alcohol, isopropyl alcohol, n-butyl alcohol, isobutyl alcohol, t-butyl alcohol, t-pentanol, ethylene glycol monoethyl ether, ethylene glycol monobutyl ether, diethylene glycol monoethyl ether, propylene glycol monomethyl ether, and benzyl alcohol; pyrazole compounds, such as 3,5-dimethyl pyrazole and 1,2-pyrazole; triazole compounds, such as 1,2,4-triazole; halogen-substituted alcohol compounds, such as ethylene chlorohydrin and 1,3-dichloro-2-propanol; lactam compounds, such as ε-caprolactam, δ-valerolactam, γ-butyrolactam, and β-propyllactam; active methylene compounds, such as methyl acetoacetate, ethyl acetoacetate, acetylacetone, methyl malonate, and ethyl malonate; and the like. Other examples include amine compounds, imide compounds, mercaptan compounds, imine compounds, urea compounds, diaryl compounds, and the like.

The thermosetting resin composition of the present invention can be used as paints for automobiles, for buildings, for metal products such as steel furniture, for wooden products such as musical instruments, for mechanical vehicles such as construction machinery, for building materials such as sashes, and for electrical appliances such as office equipment; coating materials for artificial leather, rubber rolls, etc.; inks, adhesives, pressure-sensitive adhesives, sealing materials for electronic components, sealing materials for automobiles, buildings, etc., molding materials for 3D printers, and the like.

Next, the method for curing the thermosetting resin composition of the present invention is explained.

In the method of the present invention, the thermosetting resin composition, which is a mixture of the blocked isocyanate composition and the compound having an isocyanate-reactive group described above, is heated.

The reaction temperature varies depending on the blocked isocyanate compound and the zwitterionic compound (1) in the blocked isocyanate composition used, but is generally about 60 to 250° C., and preferably about 80 to 200° C. The reaction time is about 30 seconds to 5 hours, and preferably about 1 minute to 60 minutes.

The cured product of the present invention can be produced through the above method for curing the thermosetting resin composition of the present invention.

### EXAMPLES

The present invention is described in more detail below with reference to Production Examples and Examples; however, the present invention is not limited to these Examples.

### (1) Conditions of Infrared Spectroscopy (IR Analysis)

Device: FT/IR-6600, produced by JASCO Corporation
Measurement method: total reflection measurement method (crystal: germanium)
Cumulative number: 16

### (2) ¹H-NMR Analysis Conditions

Device: AV400 produced by Bruker Corporation
Frequency: 400 MHZ

### (3) Measurement Conditions of Curing Temperature and Time

Device: Madoka automatic curing time measuring device produced by Cyber Co., Ltd.
Stirring rod: Model number 3JC-5060W
Stirring rate: rotation 100 rpm, revolutions 25 rpm

### (4) Calculation of Solids Content

About 1.5 g of a sample was heated at 110° C. for 3 hours, and the solids content (%) in the sample was calculated from the mass before and after heating.

### (5) Formulation of Thermosetting Resin Composition

A blocked isocyanate compound, a compound having an isocyanate-reactive group, and a zwitterionic compound were added such that effective NCO group (mol):hydroxyl group (mol):amidate group (mol)=1.00:0.95:0.05, and methyl isobutyl ketone was added such that solids content in the blocked isocyanate compound (g):solvent (g)=1.0:1.0. The solvent referred to here includes the solvent used for diluting the blocked isocyanate compound. The effective NCO group (mol) and hydroxyl group (mol) were calculated according to the following formulas.

Effective NC group (mol)=amount of the blocked isocyanate used (g)/effective NCO group content (%) in the blocked isocyanate/4.202

Hydroxyl group (mol)=amount of the polyol user (g)×hydroxyl value of the polyol (mgKOH/g)/56.1

### Production Example A-1

### Synthesis of [TMPACyHU]

### Reaction 1: Production of [TMPACyHU][MeCO₃]

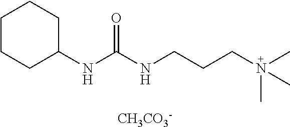

(3-Aminopropyl)dimethylamine (10 g, 97.8 mmol) (produced by Tokyo Chemical Industry Co., Ltd.) was placed in a 200-ml autoclave purged with nitrogen, and methanol (50.0 g) was added and stirred for dissolution. Cyclohexyl isocyanate (12.25 g, 97.4 mmol) (produced by Tokyo Chemical Industry Co., Ltd.) was added dropwise at 25° C. to the dissolved mixed solution. Dimethyl carbonate (13.22 g, 146 mmol) (produced by Tokyo Chemical Industry Co., Ltd.) was added to the mixed solution after dropwise addition, followed by heating at 120° C. for 8 hours. The heated reaction liquid was cooled to 25° C. and then concentrated under reduced pressure at 60° C. for 1 hour to give [TMPACyHU][MeCO₃] represented by the above formula as the concentrated residue.

The ¹H-NMR analysis results of the compound represented by the above formula are shown below.

¹H-NMR (DMSO-d⁶) δ (ppm)=3.68 (s, 3H), 3.38-3.30 (m, 3H), 3.08-3.00 (m, 11H), 1.87-1.43 (m, 7H), 1.23-1.04 (m, 5H)

### Reaction 2: Production of [TMPACyHU][HCO₃]

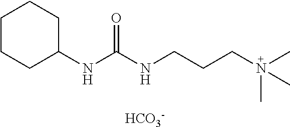

Water (100 g) was added to the concentrated residue obtained in Reaction 1, followed by stirring. Then, the reaction liquid was concentrated to give [TMPACyHU][HCO₃] as the concentrated residue.

The H-NMR analysis results of the compound represented by the above formula are shown below.

¹H-NMR (DMSO-d⁶) δ (ppm)=3.39-3.30 (m, 3H), 3.07-2.99 (m, 11H), 1.82-1.49 (m, 7H), 1.24-1.04 (m, 5H)

### Reaction 3: Production of [TMPACyHU]

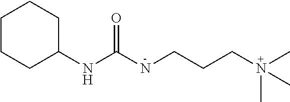

Methanol (50 g) was added to the concentrated residue obtained in Reaction 2, followed by heating at 60° C. for 16 hours to obtain a mixture. The heated mixture was cooled to 25° C. and concentrated under reduced pressure at 60° C. for 1 hour to give 28.2 g of [TMPACyHU] represented by the above formula (yield: quant.). The ¹H-NMR analysis results of the compound represented by the above formula are shown below.

¹H-NMR (MeOD) δ (ppm)=3.43-3.00 (m, 13H), 1.88 (br, 3H), 1.77 (br, 2H), 1.63 (br, 2H), 1.51 (br, 1H), 1.25 (br, 2H), 1.10 (br, 3H)

### Production Example A-2: Synthesis of [DTMPAHDU]

### Reaction 1: Production of [DTMPAHDU][MeCO₃]

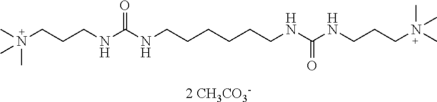

(3-Aminopropyl)dimethylamine (5.03 g, 49.2 mmol) (produced by Tokyo Chemical Industry Co., Ltd.) was placed in a 200-ml autoclave purged with nitrogen, and toluene (50.0 g) was added and stirred for dissolution. Hexamethylene diisocyanate (4.12 g, 24.4 mmol) (produced by Tokyo Chemical Industry Co., Ltd.) was added dropwise at 25° C. to the dissolved mixture, followed by stirring. Dimethyl carbonate (13.2 g, 147 mmol) (produced by Tokyo Chemical Industry Co., Ltd.) was added to the stirred mixture, followed by heating at 120° C. for 8 hours. The heated reaction liquid was cooled to 25° C. and then concentrated under reduced pressure at 60° C. for 1 hour to give [DTMPAHDU][MeCO₃] as the concentrated residue.

### Reaction 2: Production of [DTMPAHDU][HCO₃]

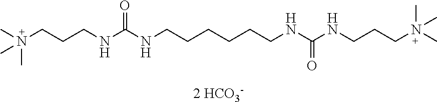

Water (100 g) was added to the reaction residue obtained in Reaction 1, followed by stirring for 1 hour to give a mixture. Then, the mixture was concentrated under reduced pressure at 60° C. for 1 hour to give [DTMPAHDU][HCO₃] represented by the above formula as the concentrated residue.

### Reaction 3: Production of [DTMPAHDU]

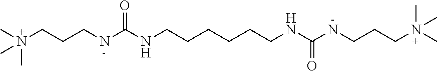

Toluene (30 g) was added to the concentrated residue obtained in Reaction 2, followed by heating at 60° C. for 3 hours to give a mixture. The heated mixture was cooled to 25° C. and concentrated under reduced pressure at 60° C. for 1 hour to give 10.5 g of [DTMPAHDU] represented by the above formula (yield: quant.). The ¹H-NMR analysis results of the compound represented by the above formula are shown below.

¹H-NMR (MeOD) δ (ppm)=3.36-3.28 (m, 4H), 3.04-3.03 (m, 22H), 2.94-2.92 (t, J=6.4 Hz, 4H), 1.82-1.78 (m, 4H), 1.36-1.22 (m, 8H)

### Production Example A-3: Method for Producing [TMPADBHDIU]

### Reaction 1: Production of [TMPADBHDIU][MeCO₃]

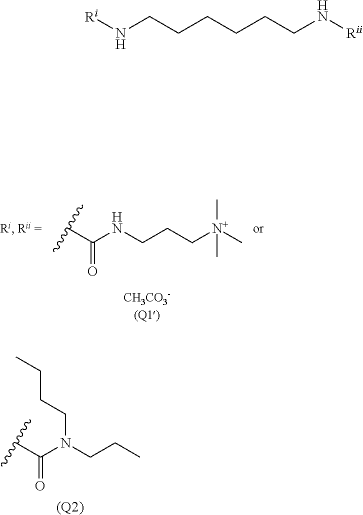

wherein at least one of Rⁱ and Rⁱⁱ is substituted with a group represented by (Q1′), and the other one is substituted with a group represented by (Q2). The reaction mixture may contain a compound wherein Rⁱ and Rⁱⁱ are both substituted with a group represented by (Q1′), or a compound wherein Rⁱ and Rⁱⁱ are both substituted with a group represented by (Q2).

Methanol (50.1 g) was added to dissolve hexamethylene diisocyanate (50.0 g, 297 mmol) (produced by Tokyo Chemical Industry Co., Ltd.) placed in a 200-ml cylindrical flask. To the obtained mixture, dibutylamine (38.3 g, 296 mmol) (produced by Tokyo Chemical Industry Co., Ltd.) was added dropwise at 25 to 75° C. over 1 hour. Next, (3-aminopropyl)dimethylamine (30.3 g, 297 mmol) (produced by Tokyo Chemical Industry Co., Ltd.) was added dropwise at 25 to 75° C. over 1 hour. The reaction liquid was stirred for 1 hour at 25 to 75° C. The obtained reaction liquid was added to a 500-ml autoclave. Dimethyl carbonate (40.0 g, 445 mmol) (produced by Tokyo Chemical Industry Co., Ltd.) was added. The mixture was reacted at 120° C. for 6 hours. The obtained reaction liquid was concentrated under reduced pressure at 60° C. for 1 hour to give [TMPADBHDIU][MeCO₃], which was assumed to be a mixture of the compounds represented by the above formulas, as the concentrated residue.

### Reaction 2: Production of [TMPADBHDIU]

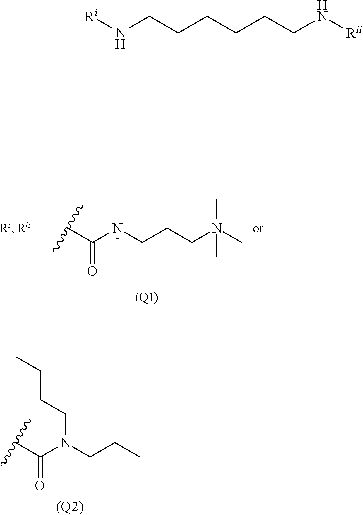

wherein at least one of Rⁱ and Rⁱⁱ is substituted with a group represented by (Q1), and the other one is substituted with a group represented by (Q2). The reaction mixture may contain a compound wherein Rⁱ and Rⁱⁱ are both substituted with a group represented by (Q1), or a compound wherein Rⁱ and Rⁱⁱ are both substituted with a group represented by (Q2).

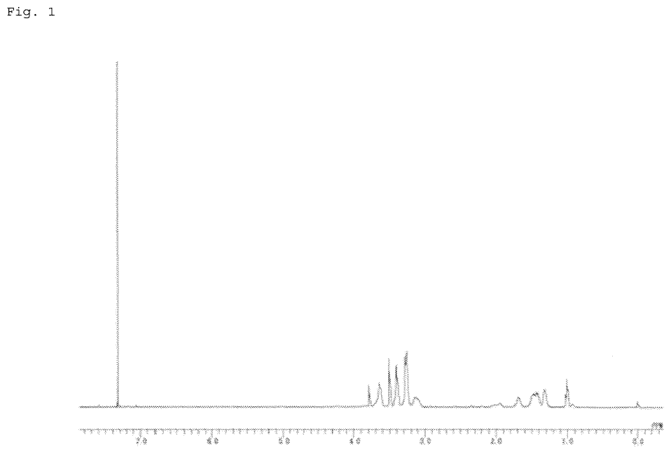

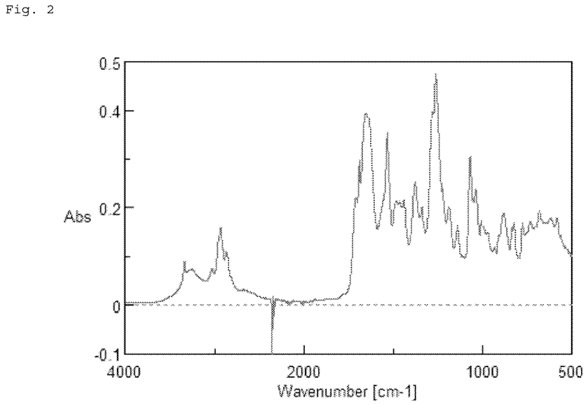

The concentrated residue obtained in Reaction 1 was dissolved in toluene (100 g) and reacted for 3 hours under reflux conditions. After concentration under reduced pressure at 60° C., 134.2 g (yield: quant.) of a mixture [TMPADBHDIU] represented by the above formula was obtained. The abundance ratio of (Q1) and (Q2) groups in the mixture is 1:1. FIG. 1 shows the ¹H-NMR (DMSO-d₆) analysis results of [TMPADBHDIU], which is assumed to be a mixture of the compounds represented by the above formulas, and FIG. 2 shows the IR analysis results.

### Production Example A-4: Method for Producing [TMPADBcrMDIU]

### Reaction 1: Production of [TMPADBcrMDIU][MeCO₃]

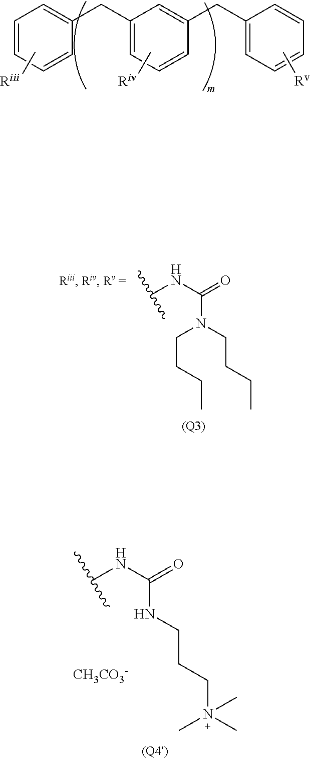

wherein at least one of Rⁱⁱⁱ, R^{iv}, and R^{v} is substituted with a group represented by (Q3), and the others are substituted with a group represented by (Q4′). The reaction mixture may contain a compound wherein Rⁱⁱⁱ, R^{iv}, and R^{v} are all substituted with a group represented by (Q3), or a compound wherein Rⁱⁱⁱ, R^{iv}, and R^{v} are all substituted with a group represented by (Q4′). m is an integer of 0 to 4.

Toluene (50 g) was added to dissolve crMDI (25.0 g, NCO group content: 31.0 (%)) (Sumidur 44V20L, produced by Sumika Covestro Urethane Co., Ltd.) placed in a 200-ml cylindrical flask purged with nitrogen. To the obtained mixture, dibutylamine (14.2 g, 109 mmol) (produced by Tokyo Chemical Industry Co., Ltd.) was added dropwise at 25 to 45° C. over 1 hour, and then (3-aminopropyl)dimethylamine (7.68 g, 75.1 mmol) (produced by Tokyo Chemical Industry Co., Ltd.) was added dropwise at 25° C. to 45° C. over 1 hour. The reaction liquid was stirred for 1 hour at 25° C. to 45° C. The obtained reaction liquid was concentrated under reduced pressure at 60° C. for 1 hour. The obtained concentrated residue was added to a 180-ml autoclave and dissolved in methanol (20.3 g), followed by addition of dimethyl carbonate (40.77 g, 451 mmol) (produced by Tokyo Chemical Industry Co., Ltd.). The mixture was reacted at 125° C. for 8 hours. The obtained reaction liquid was concentrated under reduced pressure at 60° C. for 1 hour to give [TMPADBcrMDIU][MeCO₃] as the concentrated residue.

### Reaction 2: Production of [TMPADBcrMDIU]

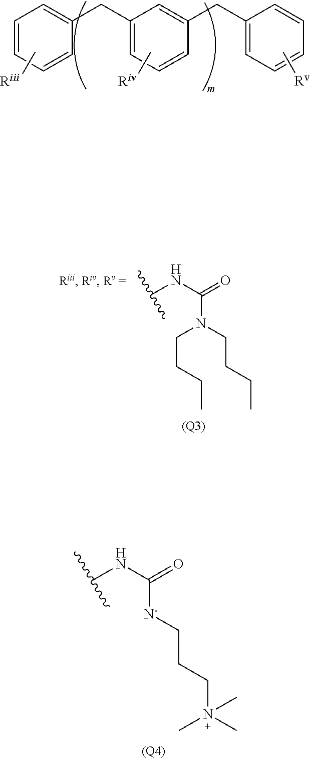

wherein at least one of Rⁱⁱⁱ, R^{iv}, and R^{v} is substituted with a group represented by (Q3), and the others are substituted with a group represented by (Q4). The reaction mixture may contain a compound wherein Rⁱⁱⁱ, R^{iv}, and R^{v} are all substituted with a group represented by (Q3), or a compound wherein Rⁱⁱⁱ, R^{iv}, and R^{v} are all substituted with a group represented by (Q4). m is an integer of 0 to 4.

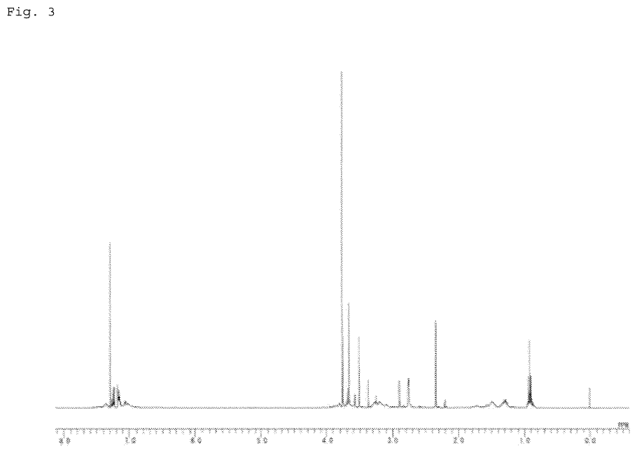

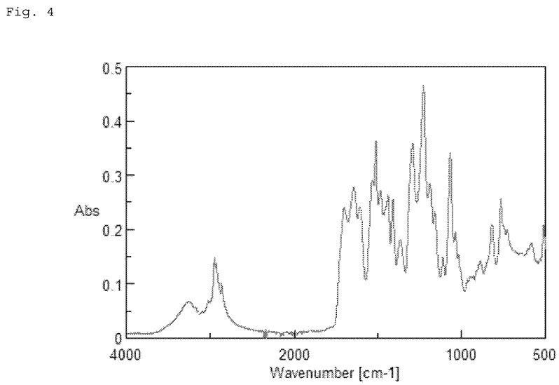

The concentrated residue obtained in Reaction 1 was dissolved in methanol (60 g) and reacted for 5 hours under reflux conditions. After concentration under reduced pressure at 60° C., 55.96 g (yield: quant.) of [TMPADBcrMDIU] represented by the above formula was obtained. The abundance ratio of (C) and (D) groups in the mixture is 3:2. The mixture is assumed to be a mixture of the compounds represented by the above formulas. FIG. 3 shows the ¹H-NMR (DMSO-d₆) analysis results of [TMPADBcrMDIU], and FIG. 4 shows the IR analysis results.

### Production Example B-1: Synthesis of Trifluoroethanol-Blocked HDI Biuret

150.0 g (NCO group: 0.81 mol) of HDI biuret (Desmodur N3200A, NCO group content: 22.8 (%), produced by Sumika Covestro Urethane Co., Ltd.) and 73.3 g of methyl isobutyl ketone (“MIBK” below) were placed in a 200-mL three-necked reactor purged with nitrogen and heated to 65° C., followed by addition of 1.4 g of triethylamine (“TEA” below). Thereafter, 27.0 g (0.99 mol) of 2,2,2-trifluoroethanol (“TFE” below) and 79.4 g of MIBK were added dropwise to the reactor and stirred at 65° C. for 2 hours. Then, the disappearance of the infrared absorption peak of isocyanate group near 2270 cm⁻¹ was confirmed by infrared spectroscopic analysis. The obtained reaction solution was concentrated under reduced pressure to remove TEA and most of MIBK, and 59.9 g of MIBK was added, thus obtaining 306.1 g of a MIBK solution of TFE-blocked HDI biuret. The obtained TFE-blocked HDI biuret had a solids content of 76.1% and an effective NCO group content of 11.2%.

### Example 1

The TFE-blocked HDI biuret obtained in Production Example B-1, a polyester polyol (P-510, produced by Kuraray Co., Ltd.), and the blocking agent dissociation catalyst [TMPACyHU] obtained in Production Example A-1 were added such that the formulation of a thermosetting composition satisfied that effective NCO group (mol):hydroxyl group (mol):curing catalyst (mol)=1.00:0.95:0.05. The mixture was then stirred for 30 minutes, thus preparing a thermosetting resin composition.

About 0.6 mL of the prepared thermosetting resin composition was poured onto the hot plate of the automatic curing time measuring device that had been heated to 120° C. in advance, and stirring was performed. During this procedure, the curing time was measured and evaluated, taking the time between the stirring torque immediately after the start of stirring of less than 1% (0.04 mN·m) and the stirring torque exceeding 50% (0.86 mN·m) as the curing time. Table 1 shows the results.

### Examples 2, 3, and 4, and Comparative Example 1

Thermosetting compositions were prepared in the same manner as in Example 1, except that the blocking agent dissociation catalysts shown in Table 1 were used. The curing time was measured and evaluated. Table 1 shows the results.

TABLE 1

| | | | | | Comparative |
|---|---|---|---|---|---|
| | Example 1 | Example 2 | Example 3 | Example 4 | Example 1 |
| Blocking agent | TMPACyHU | DTMPAHDU | [TMPADBHDIU] | [TMPADBcrMDIU] | Dibutyltin |
| dissociation | | | | | dilaurate |
| catalyst | | | | | |
| Curing time | 20 | 20 | 6 | 8 | Not cured |
| (min) | | | | | even after 90 |
| | | | | | minutes |

1. A blocking agent dissociation catalyst for blocked isocyanates comprising a zwitterionic compound represented by the following formula (1): 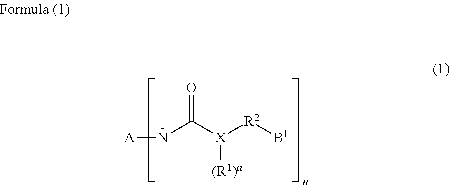
wherein
R¹ represents a hydrogen atom or a substituted or unsubstituted hydrocarbon group,
X represents a nitrogen atom or an oxygen atom,
a represents 0 or 1, wherein a represents 1 when X represents a nitrogen atom, and a represents 0 when X represents an oxygen atom,
R² represents a substituted or unsubstituted divalent hydrocarbon group,
B¹ represents a cationic group represented by formula (2) or formula (3),
n represents an integer of 1 or more, and
A represents an n-valent substituted or unsubstituted hydrocarbon group; 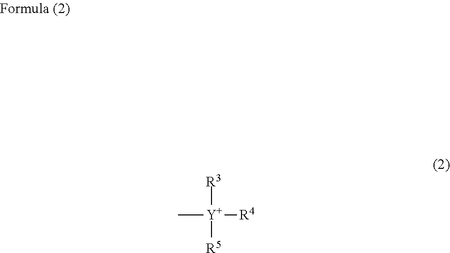
wherein
Y⁺ represents a nitrogen cation or a phosphorus cation, and
R³, R⁴, and R⁵ are the same or different, and each represents a substituted or unsubstituted hydrocarbon group, wherein some or all of R³, R⁴, and R⁵ may be bonded together to form one or more ring structures; 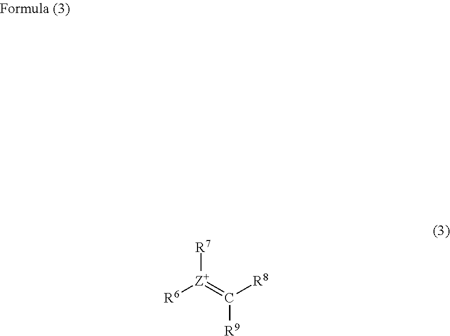
wherein
R⁸ represents a bonding hand with R², a hydrogen atom, a substituted or unsubstituted hydrocarbon group, or a substituted or unsubstituted hydrocarbon group with a bonding hand with R²,
R⁶, R⁷, and R⁹ are the same or different, and each represents a bonding hand with R², a substituted or unsubstituted hydrocarbon group, or a substituted or unsubstituted hydrocarbon group with a bonding hand with R², wherein any one of R⁶, R⁷, R⁸, and R⁹ represents a bonding hand with R² or a substituted or unsubstituted hydrocarbon group with a bonding hand with R², wherein some or all of R⁶, R⁷, R⁸, and R⁹ may be bonded together to form one or more ring structures, and wherein when some or all of R⁶, R⁷, R⁸, and R⁹ are bonded together to form one or more ring structures, and when a group or groups that do not form the ring structures from among R⁶, R⁷, R⁸, and R⁹ do not represent a bonding hand with R² or a substituted or unsubstituted hydrocarbon group with a bonding hand with R², then one of the ring structures has a bonding hand for binding to R², and
Z⁺ represents a nitrogen cation or a phosphorus cation.

2. The blocking agent dissociation catalyst for blocked isocyanates according to claim 1,
wherein
Y⁺ represents a nitrogen cation, or
the cationic group represented by formula (3) is a group represented by formula (3-1), formula (3-2), formula (3-1a), or formula (3-2a); 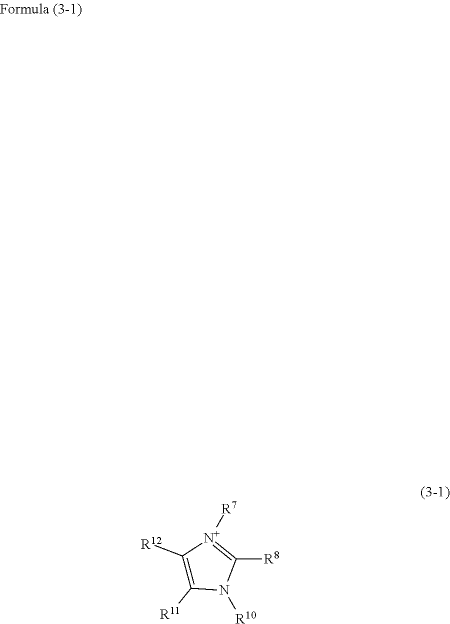
wherein
R⁷ represents a bonding hand with R², a substituted or unsubstituted hydrocarbon group, or a substituted or unsubstituted hydrocarbon group with a bonding hand with R², and
R⁸, R¹⁰, R¹¹, and R¹² are the same or different, and each represents a bonding hand with R², a hydrogen atom, a substituted or unsubstituted hydrocarbon group, or a substituted or unsubstituted hydrocarbon group with a bonding hand with R², wherein any one of R⁷, R⁸, R¹⁰, R¹¹, and R¹² represents a bonding hand for binding to R² or a substituted or unsubstituted hydrocarbon group with a bonding hand with R², and wherein when R⁷, R⁸, R¹⁰, R¹¹, and R¹² each represent a substituted or unsubstituted hydrocarbon group, some or all of R⁷, R⁸, R¹⁰, R¹¹, and R¹² may be bonded together to form one or more ring structures; 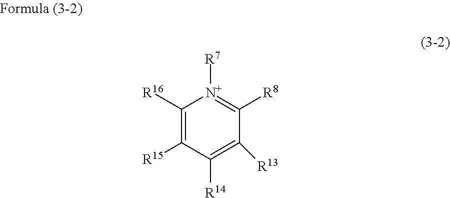
wherein
R⁷ represents a bonding hand with R², a substituted or unsubstituted hydrocarbon group, or a substituted or unsubstituted hydrocarbon group with a bonding hand with R², and
R⁸, R¹³, R¹⁴, R¹⁵, and R¹⁶ are the same or different, and each represents a bonding hand with R², a hydrogen atom, a substituted or unsubstituted hydrocarbon group, or a substituted or unsubstituted hydrocarbon group with a bonding hand with R², wherein any one of R⁷, R⁸, R¹³, R¹⁴, R¹⁵, and R¹⁶ represents a bonding hand for binding to R² or a substituted or unsubstituted hydrocarbon group with a bonding hand with R², and wherein when R⁷, R⁸, R¹³, R¹⁴, R¹⁵, and R¹⁶ each represent a substituted or unsubstituted hydrocarbon group, some or all of R⁷, R⁸, R¹³, R¹⁴, R¹⁵, and R¹⁶ may be bonded together to form one or more ring structures; 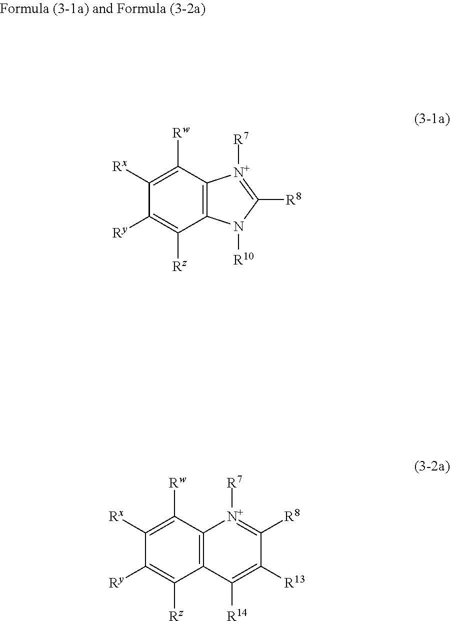
wherein
R⁷, R⁸, R¹⁰, R¹³, and R¹⁴ are as defined above, and
R^{w}, R^{x}, R^{y}, and R^{z} each represent a bonding hand with R², a hydrogen atom, or a C₁-C₂₀ hydrocarbon group.

3. The blocking agent dissociation catalyst for blocked isocyanates according to claim 1, wherein A represents an n-valent substituted or unsubstituted aliphatic hydrocarbon group, an n-valent substituted or unsubstituted alicyclic hydrocarbon group, an n-valent substituted or unsubstituted aromatic hydrocarbon group, or an n-valent substituted or unsubstituted aromatic aliphatic hydrocarbon group.

4. The blocking agent dissociation catalyst for blocked isocyanates according to claim 1, wherein n represents 1 to 20.

5. The blocking agent dissociation catalyst for blocked isocyanates according to claim 1, wherein X represents a nitrogen atom.

6. A blocked isocyanate composition comprising the blocking agent dissociation catalyst of claim 1 and a blocked isocyanate compound.

7. The blocked isocyanate composition according to claim 6, wherein the blocked isocyanate compound is a blocked isocyanate compound blocked with at least one blocking agent selected from the group consisting of alcohol compounds, phenol compounds, amine compounds, lactam compounds, oxime compounds, keto-enol compounds, active methylene compounds, pyrazole compounds, triazole compounds, imide compounds, mercaptan compounds, imine compounds, urea compounds, and diaryl compounds.

8. The blocked isocyanate composition according to claim 6, wherein the blocked isocyanate compound is a blocked isocyanate compound blocked with a fluorinated alcohol compound.

9. A thermosetting resin composition comprising the blocked isocyanate composition of claim 6 and a compound having an isocyanate-reactive group.

10. The thermosetting resin composition according to claim 9, wherein the compound having an isocyanate-reactive group is a polyol compound.

11. A cured product obtained by curing the thermosetting resin composition of claim 9.

12. A method for producing a cured product, comprising curing the thermosetting resin composition of claim 9 by heating.

13. A zwitterionic compound represented by the following formula (1): 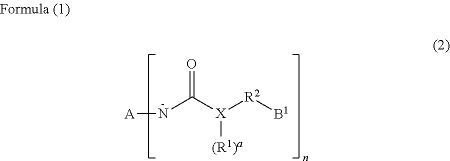
wherein
R¹ represents a hydrogen atom or a substituted or unsubstituted hydrocarbon group,
X represents a nitrogen atom or an oxygen atom,
a represents 0 or 1, wherein a represents 1 when X represents a nitrogen atom, and a represents 0 when X represents an oxygen atom,
R² represents a substituted or unsubstituted divalent hydrocarbon group,
B¹ represents a cationic group represented by formula (2) or formula (3),
n represents an integer of 1 or more, and
A represents an n-valent substituted or unsubstituted hydrocarbon group; 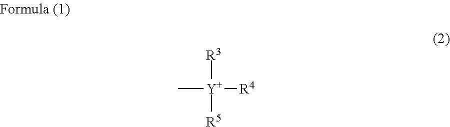
wherein
Y⁺ represents a nitrogen cation or a phosphorus cation, and
R³, R⁴, and R⁵ are the same or different, and each represents a substituted or unsubstituted hydrocarbon group, wherein some or all of R³, R⁴, and R⁵ may be bonded together to form one or more ring structures; 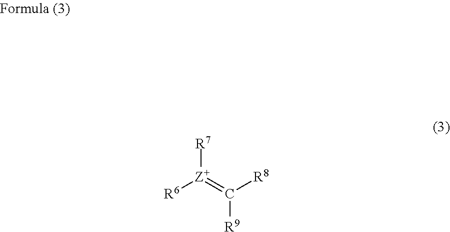
wherein
R⁸ represents a bonding hand with R², a hydrogen atom, a substituted or unsubstituted hydrocarbon group, or a substituted or unsubstituted hydrocarbon group with a bonding hand with R²,
R⁶, R⁷, and R⁹ are the same or different, and each represents a bonding hand with R², a substituted or unsubstituted hydrocarbon group, or a substituted or unsubstituted hydrocarbon group with a bonding hand with R², wherein any one of R⁶, R⁷, R⁸, and R⁹ represents a bonding hand with R² or a substituted or unsubstituted hydrocarbon group with a bonding hand with R², wherein some or all of R⁶, R⁷, R⁸, and R⁹ may be bonded together to form one or more ring structures, and wherein when some or all of R⁶, R⁷, R⁸, and R⁹ are bonded together to form one or more ring structures, and when a group or groups that do not form the ring structures from among R⁶, R⁷, R⁸, and R⁹ do not represent a bonding hand with R² or a substituted or unsubstituted hydrocarbon group with a bonding hand with R², then one of the ring structures has a bonding hand for binding to R², and
Z⁺ represents a nitrogen cation or a phosphorus cation.

14. The zwitterionic compound according to claim 13,
wherein
Y⁺ represents a nitrogen cation, or
the cationic group represented by formula (3) is a group represented by formula (3-1), formula (3-2), formula (3-1a), or formula (3-2a): 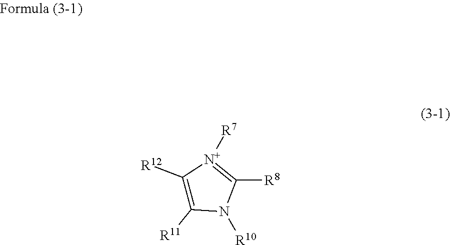
wherein
R⁷ represents a bonding hand with R², a substituted or unsubstituted hydrocarbon group, or a substituted or unsubstituted hydrocarbon group with a bonding hand with R², and
R⁸, R¹⁰, R¹¹, and R¹² are the same or different, and each represents a bonding hand with R², a hydrogen atom, a substituted or unsubstituted hydrocarbon group, or a substituted or unsubstituted hydrocarbon group with a bonding hand with R², wherein any one of R⁷, R⁸, R¹⁰, R¹¹, and R¹² represents a bonding hand for binding to R² or a substituted or unsubstituted hydrocarbon group with a bonding hand with R², and wherein when R⁷, R⁸, R¹⁰, R¹¹, and R¹² each represent a substituted or unsubstituted hydrocarbon group, some or all of R⁷, R⁸, R¹⁰, R¹¹, and R¹² may be bonded together to form one or more ring structures; 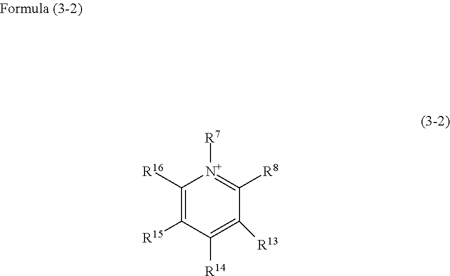
wherein
R⁷ represents a bonding hand with R², a substituted or unsubstituted hydrocarbon group, or a substituted or unsubstituted hydrocarbon group with a bonding hand with R², and
R⁸, R¹³, R¹⁴, R¹⁵, and R¹⁶ are the same or different, and each represents a bonding hand with R², a hydrogen atom, a substituted or unsubstituted hydrocarbon group, or a substituted or unsubstituted hydrocarbon group with a bonding hand with R², wherein any one of R⁷, R⁸, R¹³, R¹⁴, R¹⁵, and R¹⁶ represents a bonding hand for binding to R² or a substituted or unsubstituted hydrocarbon group with a bonding hand with R², and wherein when R⁷, R⁸, R¹³, R¹⁴, R¹⁵, and R¹⁶ each represent a substituted or unsubstituted hydrocarbon group, some or all of R⁷, R⁸, R¹³, R¹⁴, R¹⁵, and R¹⁶ may be bonded together to form one or more ring structures; 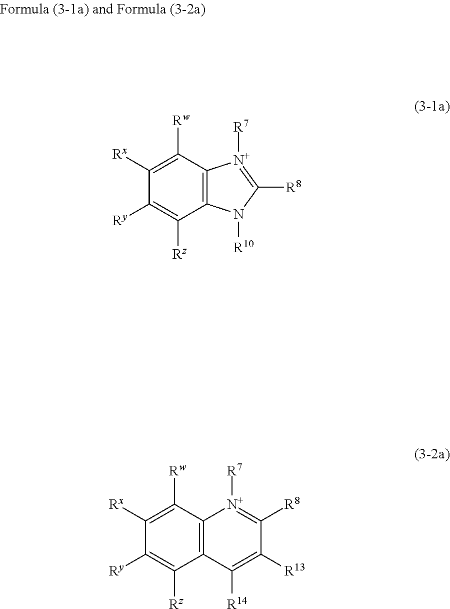
wherein
R⁷, R⁸, R¹⁰, R¹³, and R¹⁴ are as defined above, and
R^{w}, R^{x}, R^{y}, and R^{z} each represent a bonding hand with R², a hydrogen atom, or a C₁-C₂₀ hydrocarbon group.

15. The zwitterionic compound according to claim 13, wherein A represents an n-valent substituted or unsubstituted aliphatic hydrocarbon group, an n-valent substituted or unsubstituted alicyclic hydrocarbon group, an n-valent substituted or unsubstituted aromatic hydrocarbon group, or an n-valent substituted or unsubstituted aromatic aliphatic hydrocarbon group.

16. The zwitterionic compound according to claim 13, where n represents 1 to 20.

17. The zwitterionic compound according to claim 13, wherein X represents a nitrogen atom.

18. The zwitterionic compound according to claim 13, wherein R² represents a substituted or unsubstituted divalent alkylene group.

19. A method for producing a zwitterionic compound represented by formula (1), comprising stirring a compound represented by formula (8) in a solvent: 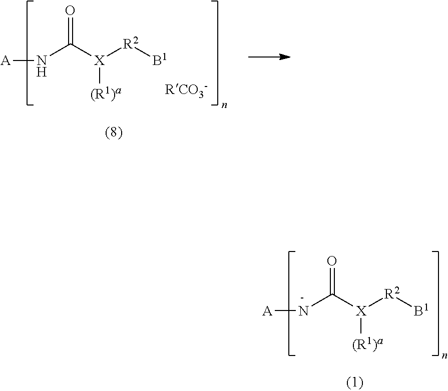
wherein
R′ represents a hydrogen atom or a substituted or unsubstituted hydrocarbon group,
R¹ represents a hydrogen atom or a substituted or unsubstituted hydrocarbon group,
X represents a nitrogen atom or an oxygen atom,
a represents 0 or 1, wherein a represents 1 when X represents a nitrogen atom, and a represents 0 when X represents an oxygen atom,
R² represents a substituted or unsubstituted divalent hydrocarbon group,
B¹ represents a cationic group represented by formula (2) or formula (3),
n represents an integer of 1 or more, and
A represents an n-valent substituted or unsubstituted hydrocarbon group; 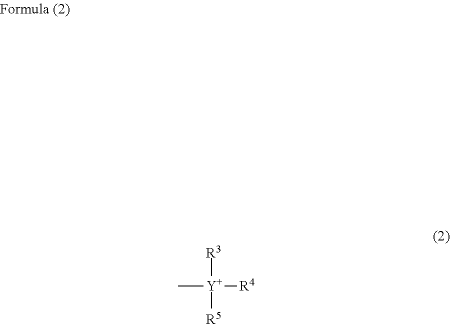
wherein
Y⁺ represents a nitrogen cation or a phosphorus cation, and
R³, R⁴, and R⁵ are the same or different, and each represents a substituted or unsubstituted hydrocarbon group, wherein some or all of R³, R⁴, and R⁵ may be bonded together to form one or more ring structures; 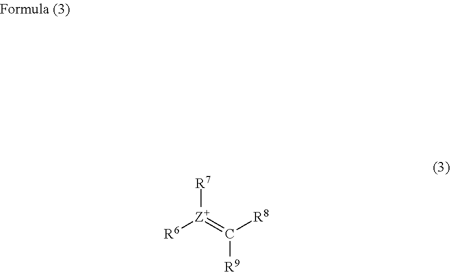
wherein
R⁸ represents a bonding hand with R², a hydrogen atom, a substituted or unsubstituted hydrocarbon group, or a substituted or unsubstituted hydrocarbon group with a bonding hand with R²,
R⁶, R⁷, and R⁹ are the same or different, and each represents a bonding hand with R², a substituted or unsubstituted hydrocarbon group, or a substituted or unsubstituted hydrocarbon group with a bonding hand with R², wherein any one of R⁶, R⁷, R⁸, and R⁹ represents a bonding hand with R² or a substituted or unsubstituted hydrocarbon group with a bonding hand with R², wherein some or all of R⁶, R⁷, R⁸, and R⁹ may be bonded together to form one or more ring structures, and wherein when some or all of R⁶, R⁷, R⁸, and R⁹ are bonded together to form one or more ring structures, and when a group or groups that do not form the ring structures from among R⁶, R⁷, R⁸, and R⁹ do not represent a bonding hand with R² or a substituted or unsubstituted hydrocarbon group with a bonding hand with R², then one of the ring structures has a bonding hand for binding to R², and
Z⁺ represents a nitrogen cation or a phosphorus cation.

20. The method according to claim 19, further comprising reacting a compound represented by formula (6) with an ester carbonate compound represented by formula (7a) or formula (7b) to produce the compound represented by formula (8): 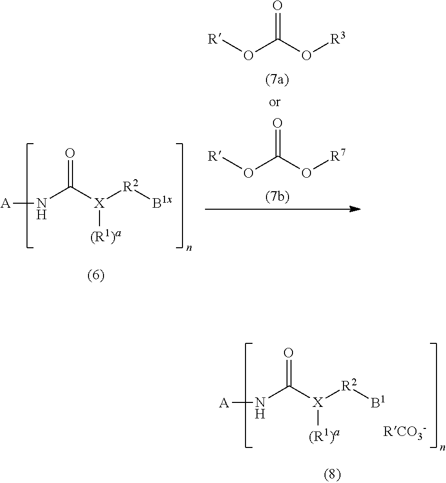
wherein
B^{1x} represents a group represented by the following formula (10) or formula (11), wherein when B^{1x} represents a group represented by formula (10), an ester carbonate compound represented by formula (7a) is used, and when B^{1x} represents a group represented by the following formula (11), an ester carbonate compound represented by formula (7b) is used,
R′ represents a hydrogen atom, a substituted or unsubstituted hydrocarbon group, R³, or R⁷,
R³ represents a substituted or unsubstituted hydrocarbon group,
R⁷ represents a substituted or unsubstituted hydrocarbon group, and
R¹, R², B¹, A, a, X, and n are as defined above; 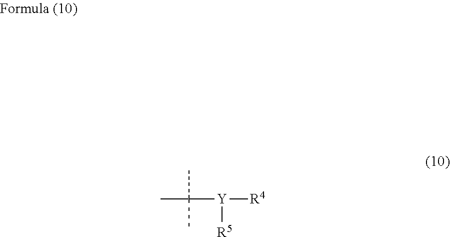
wherein
Y represents a nitrogen atom or a phosphorus atom, and
R⁴ and R⁵ are as defined above; 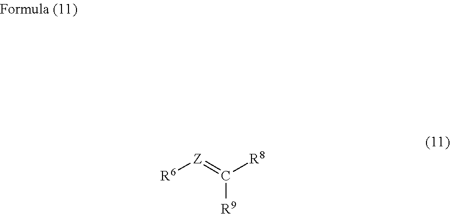
wherein
Z represents a nitrogen atom or a phosphorus atom, and
R⁶, R⁸, and R⁹ are as defined above.

21. A compound represented by the following formula (8): 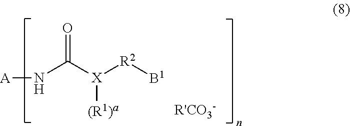
wherein
R′ represents a hydrogen atom or a substituted or unsubstituted hydrocarbon group,
R¹ represents a hydrogen atom or a substituted or unsubstituted hydrocarbon group,
X represents a nitrogen atom or an oxygen atom,
a represents 0 or 1, wherein a represents 1 when X represents a nitrogen atom, and a represents 0 when X represents an oxygen atom,
R² represents a substituted or unsubstituted divalent hydrocarbon group,
B¹ represents a cationic group represented by formula (2) or formula (3),
n represents an integer of 1 or more, and
A represents an n-valent substituted or unsubstituted hydrocarbon group; 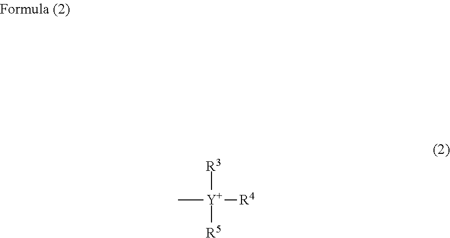
wherein
Y⁺ represents a nitrogen cation or a phosphorus cation, and
R³, R⁴, and R⁵ are the same or different, and each represents a substituted or unsubstituted hydrocarbon group, wherein some or all of R³, R⁴, and R⁵ may be bonded together to form one or more ring structures; 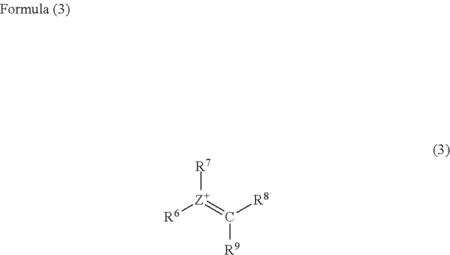
wherein
R⁸ represents a bonding hand with R², a hydrogen atom, a substituted or unsubstituted hydrocarbon group, or a substituted or unsubstituted hydrocarbon group with a bonding hand with R²,
R⁶, R⁷, and R⁹ are the same or different, and each represents a bonding hand with R², a substituted or unsubstituted hydrocarbon group, or a substituted or unsubstituted hydrocarbon group with a bonding hand with R², wherein any one of R⁶, R⁷, R⁸, and R⁹ represents a bonding hand with R² or a substituted or unsubstituted hydrocarbon group with a bonding hand with R², wherein some or all of R⁶, R⁷, R⁸, and R⁹ may be bonded together to form one or more ring structures, and wherein when some or all of R⁶, R⁷, R⁸, and R⁹ are bonded together to form one or more ring structures, and when a group or groups that do not form the ring structures from among R⁶, R⁷, R⁸, and R⁹ do not represent a bonding hand with R² or a substituted or unsubstituted hydrocarbon group with a bonding hand with R², then one of the ring structures has a bonding hand for binding to R², and
Z⁺ represents a nitrogen cation or a phosphorus cation.
